SEQUENCE LISTING (109 sequences)

SEQ ID NO: 1 - protein, 5 aa, synthetic construct
  source 1..5: note = Anti-TfR antibody (1026) heavy chain CDR-H1
  REGION 1..5: note = Anti-TfR antibody (1026) heavy chain CDR-H1
SYAMS

SEQ ID NO: 2 - protein, 16 aa, synthetic construct
  source 1..16: note = Anti-TfR antibody (1026) heavy chain CDR-H2
  REGION 1..16: note = Anti-TfR antibody (1026) heavy chain CDR-H2
YIWSGGSTDYASWAKS

SEQ ID NO: 3 - protein, 17 aa, synthetic construct
  source 1..17: note = Anti-TfR antibody (1026) heavy chain CDR-H3
  REGION 1..17: note = Anti-TfR antibody (1026) heavy chain CDR-H3
RYGTSYPDYGDASGFDP

SEQ ID NO: 4 - protein, 11 aa, synthetic construct
  source 1..11: note = Anti-TfR antibody (1026) light chain CDR-L1
  REGION 1..11: note = Anti-TfR antibody (1026) light chain CDR-L1
RASQSISSYLA

SEQ ID NO: 5 - protein, 7 aa, synthetic construct
  source 1..7: note = Anti-TfR antibody (1026) light chain CDR-L2
  REGION 1..7: note = Anti-TfR antibody (1026) light chain CDR-L2
RASTLAS

SEQ ID NO: 6 - protein, 12 aa, synthetic construct
  source 1..12: note = Anti-TfR antibody (1026) light chain CDR-L3
  REGION 1..12: note = Anti-TfR antibody (1026) light chain CDR-L3
QQNYASSNVDNT

SEQ ID NO: 7 - protein, 122 aa, synthetic construct
  source 1..122: note = Anti-TfR antibody (1026) heavy chain variable domain
  REGION 1..122: note = Anti-TfR antibody (1026) heavy chain variable domain
QSMQESGPGLVKPSQTLSLTCTVSGFSLSSYAMSWIRQHPGKGLEWIGYIWSGGSTDYASWAKSRVTISKTSTTVSLKLSSVTAADTAVYYCARRYGTSYPDYGDASGFDPWGQGTLVTVSS

SEQ ID NO: 8 - protein, 110 aa, synthetic construct
  source 1..110: note = Anti-TfR antibody (1026) light chain variable domain
  REGION 1..110: note = Anti-TfR antibody (1026) light chain variable domain
AIQLTQSPSSLSASVGDRVTITCRASQSISSYLAWYQQKPGKAPKLLIYRASTLASGVPSRFSGSGSGTDFTLTISSLQPEDFATYYCQQNYASSNVDNTFGGGTKVEIK

SEQ ID NO: 9 - protein, 5 aa, synthetic construct
  source 1..5: note = Anti-TfR antibody (51A165) heavy chain CDR-H1
  REGION 1..5: note = Anti-TfR antibody (51A165) heavy chain CDR-H1
SYVMH

SEQ ID NO: 10 - protein, 17 aa, synthetic construct
  source 1..17: note = Anti-TfR antibody (51A165) heavy chain CDR-H2
  REGION 1..17: note = Anti-TfR antibody (51A165) heavy chain CDR-H2
YVNPYNDDTKYNEKFRG

SEQ ID NO: 11 - protein, 9 aa, synthetic construct
  source 1..9: note = Anti-TfR antibody (51A165) heavy chain CDR-H3
  REGION 1..9: note = Anti-TfR antibody (51A165) heavy chain CDR-H3
SYYYYSMDN

SEQ ID NO: 12 - protein, 10 aa, synthetic construct
  source 1..10: note = Anti-TfR antibody (51A165) light chain CDR-L1
  REGION 1..10: note = Anti-TfR antibody (51A165) light chain CDR-L1
SASSSIRYMH

SEQ ID NO: 13 - protein, 7 aa, synthetic construct
  source 1..7: note = Anti-TfR antibody (51A165) light chain CDR-L2
  REGION 1..7: note = Anti-TfR antibody (51A165) light chain CDR-L2
DTSELAS

SEQ ID NO: 14 - protein, 9 aa, synthetic construct
  source 1..9: note = Anti-TfR antibody (51A165) light chain CDR-L3
  REGION 1..9: note = Anti-TfR antibody (51A165) light chain CDR-L3
HQRSSYPFT

SEQ ID NO: 15 - protein, 118 aa, synthetic construct
  source 1..118: note = Anti-TfR-antibody (51A165) heavy chain variable domain
  REGION 1..118: note = Anti-TfR-antibody (51A165) heavy chain variable domain
EVQLQQSGPELVKPGASVKMSCKASGYTFTSYVMHWVKQKPGQGLEWIGYVNPYNDDTKYNEKFRGKATLTSDKSSSTAYMELSSLTSEDSAVYYCARSYYYYSMDNWGQGTSVTVSS

SEQ ID NO: 16 - protein, 106 aa, synthetic construct
  source 1..106: note = Anti-TfR-antibody (51A165) light chain variable domain
  REGION 1..106: note = Anti-TfR-antibody (51A165) light chain variable domain
QIVLTQSPAIMSASPGEKVTVTCSASSSIRYMHWYQQKPGTSPKRWIYDTSELASGVPARFSGSGSGTSYSLTINSMEAEDAATYYCHQRSSYPFTFGGGTKLEIK

SEQ ID NO: 17 - protein, 5 aa, synthetic construct
  source 1..5: note = Anti-PD1-antibody (0103-0312) heavy chain CDR-H1
  REGION 1..5: note = Anti-PD1-antibody (0103-0312) heavy chain CDR-H1
SYTMS

SEQ ID NO: 18 - protein, 17 aa, synthetic construct
  REGION 1..17: note = Anti-PD1-antibody (0103-0312) heavy chain CDR-H2
TISGGGRDIYYPDSVKG

SEQ ID NO: 19 - protein, 11 aa, synthetic construct
  source 1..11: note = Anti-PD1-antibody (0103-0312) heavy chain CDR-H3
  REGION 1..11: note = Anti-PD1-antibody (0103-0312) heavy chain CDR-H3
LTGRVYFALDS

SEQ ID NO: 20 - protein, 15 aa, synthetic construct
  source 1..15: note = Anti-PD1-antibody (0103-0312) light chain CDR-L1
  REGION 1..15: note = Anti-PD1-antibody (0103-0312) light chain CDR-L1
KASESVDTSDNSFIH

SEQ ID NO: 21 - protein, 7 aa, synthetic construct
  source 1..7: note = Anti-PD1-antibody (0103-0312) light chain CDR-L2
  REGION 1..7: note = Anti-PD1-antibody (0103-0312) light chain CDR-L2
RSSTLES

SEQ ID NO: 22 - protein, 9 aa, synthetic construct
  REGION 1..9: note = Anti-PD1-antibody (0103-0312) light chain CDR-L3
QQNYDVPWT

SEQ ID NO: 23 - protein, 120 aa, synthetic construct
  REGION 1..120: note = Anti-PD1-antibody (0103-0312) heavy chain variable domain
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSS

SEQ ID NO: 24 - protein, 111 aa, synthetic construct
  REGION 1..111: note = Anti-PD1-antibody (0103-0312) light chain variable domain
DIVMTQSPDSLAVSLGERATINCKASESVDTSDNSFIHWYQQKPGQSPKLLIYRSSTLESGVPDRFSGSGSGTDFTLTISSLQAEDVAVYYCQQNYDVPWTFGQGTKVEIK

SEQ ID NO: 25 - protein, 5 aa, synthetic construct
  REGION 1..5: note = Pembrolizumab anti-PD1 antibody heavy chain CDR-H1
NYYMY

SEQ ID NO: 26 - protein, 17 aa, synthetic construct
  REGION 1..17: note = Pembrolizumab anti-PD1 antibody heavy chain CDR-H2
GINPSNGGTNFNEKFKN

SEQ ID NO: 27 - protein, 11 aa, synthetic construct
  REGION 1..11: note = Pembrolizumab anti-PD1 antibody heavy chain CDR-H3
RDYRFDMGFDY

SEQ ID NO: 28 - protein, 15 aa, synthetic construct
  REGION 1..15: note = Pembrolizumab anti-PD1 antibody light chain CDR-L1
RASKGVSTSGYSYLH

SEQ ID NO: 29 - protein, 7 aa, synthetic construct
  REGION 1..7: note = Pembrolizumab anti-PD1 antibody light chain CDR-L2
LASYLES

SEQ ID NO: 30 - protein, 9 aa, synthetic construct
  REGION 1..9: note = Pembrolizumab anti-PD1 antibody light chain CDR-L3
QHSRDLPLT

SEQ ID NO: 31 - protein, 120 aa, synthetic construct [sequence omitted]

SEQ ID NO: 32 - protein, 111 aa, synthetic construct [sequence omitted]

SEQ ID NO: 33 - protein, 120 aa, synthetic construct
  REGION 1..120: note = Nada anti-Nada antibody heavy chain variable domain
EVQLVESGGGLVQPGGSLRLSCAASGFSIAGTAIHWVRQAPGKGLEWVASISPGGGSTAYADSVKGRFTISADTSKNTAYLQMNSLRAEDTAVYYCSRSGGSGASAMDYWGQGTLVTVSS

SEQ ID NO: 34 - protein, 107 aa, synthetic construct
  REGION 1..107: note = Nada anti-Nada antibody light chain variable domain
DIQMTQSPSSLSASVGDRVTITCRASQDVGSGVAWYQQKPGKAPKLLIGSASGLYSGVPSRFSGSRSGTDFTLTISSLQPEDFATYYCQQSASGGSTFGQGTKVEIK

SEQ ID NO: 35 - protein, 450 aa, synthetic construct
  REGION 1..450: note = Anti-TfR (1026) Full length heavy chain (Hole, EE charge)
QSMQESGPGLVKPSQTLSLTCTVSGFSLSSYAMSWIRQHPGKGLEWIGYIWSGGSTDYASWAKSRVTISKTSTTVSLKLSSVTAADTAVYYCARRYGTSYPDYGDASGFDPWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 36 - protein, 217 aa, synthetic construct
  REGION 1..217: note = Anti-TfR antibody (1026) full length light chain (RK charge)
AIQLTQSPSSLSASVGDRVTITCRASQSISSYLAWYQQKPGKAPKLLIYRASTLASGVPSRFSGSGSGTDFTLTISSLQPEDFATYYCQQNYASSNVDNTFGGGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - protein, 446 aa, synthetic construct
  REGION 1..446: note = Anti-TfR antibody (51A165) full length heavy chain (Hole, EEcharge)
EVQLQQSGPELVKPGASVKMSCKASGYTFTSYVMHWVKQKPGQGLEWIGYVNPYNDDTKYNEKFRGKATLTSDKSSSTAYMELSSLTSEDSAVYYCARSYYYYSMDNWGQGTSVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 38 - protein, 213 aa, synthetic construct
  REGION 1..213: note = Anti-TfR-antibody (51A165) full length light chain (RK charge)
QIVLTQSPAIMSASPGEKVTVTCSASSSIRYMHWYQQKPGTSPKRWIYDTSELASGVPARFSGSGSGTSYSLTINSMEAEDAATYYCHQRSSYPFTFGGGTKLEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - protein, 441 aa, synthetic construct
  REGION 1..441: note = Anti-PD1-antibody (0103-0312) full length heavy chain (cross,Knob, light chain variable domain)
DIVMTQSPDSLAVSLGERATINCKASESVDTSDNSFIHWYQQKPGQSPKLLIYRSSTLESGVPDRFSGSGSGTDFTLTISSLQAEDVAVYYCQQNYDVPWTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 40 - protein, 227 aa, synthetic construct
  REGION 1..227: note = Anti-PD1-antibody (0103-0312) full length light chain (cross,heavy chain variable domain)
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 441 aa, synthetic construct
  REGION 1..441: note = Anti-PD1-antibody (Pembrolizumab) full length heavy chain (cross,knob, light chain variable region)
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 42 - protein, 227 aa, synthetic construct
  REGION 1..227: note = Anti-PD1-antibody (Pembrolizumab) full length light chain (cross,heavy chain variable domain)
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 437 aa, synthetic construct
  REGION 1..437: note = Anti-Nada-antibody full length heavy chain (cross, Knob, lightchain variable domain)
DIQMTQSPSSLSASVGDRVTITCRASQDVGSGVAWYQQKPGKAPKLLIGSASGLYSGVPSRFSGSRSGTDFTLTISSLQPEDFATYYCQQSASGGSTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 44 - protein, 227 aa, synthetic construct
  REGION 1..227: note = Anti-Nada-antibody full length light chain (cross, heavy chainvariable region)
EVQLVESGGGLVQPGGSLRLSCAASGFSIAGTAIHWVRQAPGKGLEWVASISPGGGSTAYADSVKGRFTISADTSKNTAYLQMNSLRAEDTAVYYCSRSGGSGASAMDYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 448 aa, synthetic construct
  REGION 1..448: note = Anti-Nada full-length heavy chain (hole, EE charge)
EVQLVESGGGLVQPGGSLRLSCAASGFSIAGTAIHWVRQAPGKGLEWVASISPGGGSTAYADSVKGRFTISADTSKNTAYLQMNSLRAEDTAVYYCSRSGGSGASAMDYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 46 - protein, 214 aa, synthetic construct
  REGION 1..214: note = Anti-Nada full length light chain (RK charge)
DIQMTQSPSSLSASVGDRVTITCRASQDVGSGVAWYQQKPGKAPKLLIGSASGLYSGVPSRFSGSRSGTDFTLTISSLQPEDFATYYCQQSASGGSTFGQGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - protein, 706 aa, synthetic construct
  REGION 1..706: note = Anti-TfR (51A165) full length heavy chain (knob, anti-biotin scFvattached to the C-terminus)
EVQLQQSGPELVKPGASVKMSCKASGYTFTSYVMHWVKQKPGQGLEWIGYVNPYNDDTKYNEKFRGKATLTSDKSSSTAYMELSSLTSEDSAVYYCARSYYYYSMDNWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSDIQMTQSPSSLSASVGDRVTITCRASGNIHNYLSWYQQKPGKVPKLLIYSAKTLADGVPSRFSGSGSGTDFTLTISSLQPEDVATYYCQHFWSSIYTFGCGTKLEIKRGGGGSGGGGSGGGGSGGGGSQVQLVQSGAEVKKPGSSVKVSCKSSGFNNKDTFFQWVRQAPGQCLEWMGRIDPANGFTKYAQKFQGRVTITADTSTSTAYMELSSLRSEDTAVYYCARWDTYGAAWFAYWGQGTLVTVSS

SEQ ID NO: 48 - protein, 213 aa, synthetic construct
  REGION 1..213: note = Anti-TfR (51A165) Full length light chain
QIVLTQSPAIMSASPGEKVTVTCSASSSIRYMHWYQQKPGTSPKRWIYDTSELASGVPARFSGSGSGTSYSLTINSMEAEDAATYYCHQRSSYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - protein, 712 aa, synthetic construct
  REGION 1..712: note = Anti-PD1 (Pembrolizumab) Full length heavy chain (cross, hole)with anti-biotin scFv attached to the C-terminus
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSDIQMTQSPSSLSASVGDRVTITCRASGNIHNYLSWYQQKPGKVPKLLIYSAKTLADGVPSRFSGSGSGTDFTLTISSLQPEDVATYYCQHFWSSIYTFGCGTKLEIKRGGGGSGGGGSGGGGSGGGGSQVQLVQSGAEVKKPGSSVKVSCKSSGFNNKDTFFQWVRQAPGQCLEWMGRIDPANGFTKYAQKFQGRVTITADTSTSTAYMELSSLRSEDTAVYYCARWDTYGAAWFAYWGQGTLVTVSS

SEQ ID NO: 50 - protein, 216 aa, synthetic construct
  REGION 1..216: note = Anti-PD1 (Pembrolizumab) full length light chain
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 51 - protein, 708 aa, synthetic construct
  REGION 1..708: note = Anti-Nada full length heavy chain (knob) with anti-biotin scFvattached to the C-terminus
EVQLVESGGGLVQPGGSLRLSCAASGFSIAGTAIHWVRQAPGKGLEWVASISPGGGSTAYADSVKGRFTISADTSKNTAYLQMNSLRAEDTAVYYCSRSGGSGASAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSDIQMTQSPSSLSASVGDRVTITCRASGNIHNYLSWYQQKPGKVPKLLIYSAKTLADGVPSRFSGSGSGTDFTLTISSLQPEDVATYYCQHFWSSIYTFGCGTKLEIKRGGGGSGGGGSGGGGSGGGGSQVQLVQSGAEVKKPGSSVKVSCKSSGFNNKDTFFQWVRQAPGQCLEWMGRIDPANGFTKYAQKFQGRVTITADTSTSTAYMELSSLRSEDTAVYYCARWDTYGAAWFAYWGQGTLVTVSS

SEQ ID NO: 52 - protein, 214 aa, synthetic construct
  REGION 1..214: note = Anti-Nada full length light chain
DIQMTQSPSSLSASVGDRVTITCRASQDVGSGVAWYQQKPGKAPKLLIGSASGLYSGVPSRFSGSRSGTDFTLTISSLQPEDFATYYCQQSASGGSTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 53 - protein, 712 aa, synthetic construct
  REGION 1..712: note = Anti-Nada full length heavy chain (cross, hole) with anti-biotinscFv attached to the C-terminus
EVQLVESGGGLVQPGGSLRLSCAASGFSIAGTAIHWVRQAPGKGLEWVASISPGGGSTAYADSVKGRFTISADTSKNTAYLQMNSLRAEDTAVYYCSRSGGSGASAMDYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSDIQMTQSPSSLSASVGDRVTITCRASGNIHNYLSWYQQKPGKVPKLLIYSAKTLADGVPSRFSGSGSGTDFTLTISSLQPEDVATYYCQHFWSSIYTFGCGTKLEIKRGGGGSGGGGSGGGGSGGGGSQVQLVQSGAEVKKPGSSVKVSCKSSGFNNKDTFFQWVRQAPGQCLEWMGRIDPANGFTKYAQKFQGRVTITADTSTSTAYMELSSLRSEDTAVYYCARWDTYGAAWFAYWGQGTLVTVSS

SEQ ID NO: 54 - protein, 212 aa, synthetic construct
  REGION 1..212: note = Anti-Nada full length light chain (cross)
DIQMTQSPSSLSASVGDRVTITCRASQDVGSGVAWYQQKPGKAPKLLIGSASGLYSGVPSRFSGSRSGTDFTLTISSLQPEDFATYYCQQSASGGSTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 55 - protein, 704 aa, synthetic construct
  REGION 1..704: note = Anti CD33 full length heavy chain with anti-biotin scFv attachedto the C-terminus
EVQLVQSGAEVKKPGSSVKVSCKASGYTITDSNIHWVRQAPGQSLEWIGYIYPYNGGTDYNQKFKNRATLTVDNPTNTAYMELSSLRSEDTAFYYCVNGNPWLAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSDIQMTQSPSSLSASVGDRVTITCRASGNIHNYLSWYQQKPGKVPKLLIYSAKTLADGVPSRFSGSGSGTDFTLTISSLQPEDVATYYCQHFWSSIYTFGCGTKLEIKRGGGGSGGGGSGGGGSGGGGSQVQLVQSGAEVKKPGSSVKVSCKSSGFNNKDTFFQWVRQAPGQCLEWMGRIDPANGFTKYAQKFQGRVTITADTSTSTAYMELSSLRSEDTAVYYCARWDTYGAAWFAYWGQGTLVTVSS

SEQ ID NO: 56 - protein, 218 aa, synthetic construct
  REGION 1..218: note = Anti-CD33 full length light chain
DIQLTQSPSTLSASVGDRVTITCRASESLDNYGIRFLTWFQQKPGKAPKLLMYAASNQGSGVPSRFSGSGSGTEFTLTISSLQPDDFATYYCQQTKEVPWSFGQGTKVEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 57 - protein, 227 aa, synthetic construct; identical to SEQ ID NO: 40
  REGION 1..227: note = Anti-PD1 (0103-0312) full length light chain (cross, heavy chainvariable domain)

SEQ ID NO: 58 - protein, 213 aa, synthetic construct; identical to SEQ ID NO: 38
  REGION 1..213: note = Anti-TfR (51A165) full length light chain (RK charge)

SEQ ID NO: 59 - protein, 675 aa, synthetic construct
  REGION 1..675: note = Anti-PD1 (0103-0312) full length heavy chain (cross, hole, lightchain variable domain) with anti-TfR(51A165) Fab attached to theC-terminus of the CH3 domain (BBB-like)
DIVMTQSPDSLAVSLGERATINCKASESVDTSDNSFIHWYQQKPGQSPKLLIYRSSTLESGVPDRFSGSGSGTDFTLTISSLQAEDVAVYYCQQNYDVPWTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSEVQLQQSGPELVKPGASVKMSCKASGYTFTSYVMHWVKQKPGQGLEWIGYVNPYNDDTKYNEKFRGKATLTSDKSSSTAYMELSSLTSEDSAVYYCARSYYYYSMDNWGQGTSVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCD

SEQ ID NO: 60 - protein, 441 aa, synthetic construct; identical to SEQ ID NO: 39
  REGION 1..441: note = Anti-PD1 (0103-0312) full length heavy chain (cross, knob, lightchain variable domain)

SEQ ID NO: 61 - protein, 673 aa, synthetic construct
  REGION 1..673: note = Anti-PD1 (0103-312) full length heavy chain (cross, hole, lightchain variable domain) with anti-TfR(51A165) Fab insertedinbetween anti-PD1 and hinge region(TCB-like)
DIVMTQSPDSLAVSLGERATINCKASESVDTSDNSFIHWYQQKPGQSPKLLIYRSSTLESGVPDRFSGSGSGTDFTLTISSLQAEDVAVYYCQQNYDVPWTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLQQSGPELVKPGASVKMSCKASGYTFTSYVMHWVKQKPGQGLEWIGYVNPYNDDTKYNEKFRGKATLTSDKSSSTAYMELSSLTSEDSAVYYCARSYYYYSMDNWGQGTSVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 62 - protein, 214 aa, synthetic construct; identical to SEQ ID NO: 46
  REGION 1..214: note = Anti-Nada full length light chain (RK charge)

SEQ ID NO: 63 - protein, 677 aa, synthetic construct
  REGION 1..677: note = Anti-PD1 (0103-0312) full length heavy chain (cross, hole, lightchain variable domain) with anti-Nada Fab attached to theC-terminus of the CH3 domain (BBB-like)
DIVMTQSPDSLAVSLGERATINCKASESVDTSDNSFIHWYQQKPGQSPKLLIYRSSTLESGVPDRFSGSGSGTDFTLTISSLQAEDVAVYYCQQNYDVPWTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSEVQLVESGGGLVQPGGSLRLSCAASGFSIAGTAIHWVRQAPGKGLEWVASISPGGGSTAYADSVKGRFTISADTSKNTAYLQMNSLRAEDTAVYYCSRSGGSGASAMDYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCD

SEQ ID NO: 64 - protein, 675 aa, synthetic construct
  REGION 1..675: note = Anti-PD1 (0103-312) full length heavy chain (cross, hole, lightchain variable domain) with anti-Nada Fab inserted inbetweenanti-PD1 and hinge region(TCB-like)
DIVMTQSPDSLAVSLGERATINCKASESVDTSDNSFIHWYQQKPGQSPKLLIYRSSTLESGVPDRFSGSGSGTDFTLTISSLQAEDVAVYYCQQNYDVPWTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLVESGGGLVQPGGSLRLSCAASGFSIAGTAIHWVRQAPGKGLEWVASISPGGGSTAYADSVKGRFTISADTSKNTAYLQMNSLRAEDTAVYYCSRSGGSGASAMDYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 65 - protein, 268 aa, Homo sapiens
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 66 - protein, 760 aa, Homo sapiens
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 67 - protein, 107 aa, Homo sapiens [sequence omitted]

SEQ ID NO: 68 - protein, 105 aa, Homo sapiens
QPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 69 - protein, 328 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 70 - protein, 327 aa, synthetic construct
  REGION 1..327: note = Exemplary human heavy chain constant region derived from IgG1with mutations L234A, L235A and P329G (Fc gamma receptorsilenced)
ASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKAGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 71 - protein, 6 aa, synthetic construct
  REGION 1..6: note = MYPPPY hexapeptide motif that is critical for binding of CTLA4 toB7-1 and B7-2
MYPPPY

SEQ ID NO: 72 - protein, 5 aa, synthetic construct
  REGION 1..5: note = anti mTfR antibody 001 heavy chain CDR-H1
NYGMH

SEQ ID NO: 73 - protein, 17 aa, synthetic construct
  REGION 1..17: note = anti mTfR antibody 001 heavy chain CDR-H2
MIYYDSSKMNYADTVKG

SEQ ID NO: 74 - protein, 9 aa, synthetic construct
  REGION 1..9: note = anti mTfR antibody 001 heavy chain CDR-H3
PTSHYVVDV

SEQ ID NO: 75 - protein, 11 aa, synthetic construct
  REGION 1..11: note = anti mTfR antibody 001 light chain CDR-L1
QASQDIGNWLA

SEQ ID NO: 76 - protein, 7 aa, synthetic construct
  REGION 1..7: note = anti mTfR antibody 001 light chain CDR-L2
GATSLAD

SEQ ID NO: 77 - protein, 9 aa, synthetic construct
  REGION 1..9: note = anti mTfR antibody 001 light chain CDR-L3
LQAYNTPWT

SEQ ID NO: 78 - protein, 118 aa, synthetic construct
  REGION 1..118: note = anti mTfR antibody 001 heavy chain variable region VH
EVQLVESGGGLVQPGNSLTLSCVASGFTFSNYGMHWIRQAPKKGLEWIAMIYYDSSKMNYADTVKGRFTISRDNSKNTLYLEMNSLRSEDTAMYYCAVPTSHYVVDVWGQGVSVTVSS

SEQ ID NO: 79 - protein, 107 aa, synthetic construct
  REGION 1..107: note = anti mTfR antibody 001 light chain variable region VL
DIQMTQSPASLSASLEEIVTITCQASQDIGNWLAWYQQKPGKSPQLLIYGATSLADGVPSRFSGSRSGTQFSLKISRVQVEDIGIYYCLQAYNTPWTFGGGTKVEIK

SEQ ID NO: 80 - protein, 5 aa, synthetic construct
  REGION 1..5: note = anti PD1 antibody 478 heavy chain CDR-H1
SYTMS

SEQ ID NO: 81 - protein, 17 aa, synthetic construct
  REGION 1..17: note = anti PD1 antibody 478 heavy chain CDR-H2
TISGGGRDIYYPDSVKG

SEQ ID NO: 82 - protein, 11 aa, synthetic construct
  REGION 1..11: note = anti PD1 antibody 478 heavy chain CDR-H3
LTGRVYFALDS

SEQ ID NO: 83 - protein, 15 aa, synthetic construct
  REGION 1..15: note = anti PD1 antibody 478 light chain CDR-L1
RASESVDTSDNSFIH

SEQ ID NO: 84 - protein, 7 aa, synthetic construct
  REGION 1..7: note = anti PD1 antibody 478 light chain CDR-L2
RSSTLES

SEQ ID NO: 85 - protein, 9 aa, synthetic construct
  REGION 1..9: note = anti PD1 antibody 478 light chain CDR-L3
QQNYDVPWT

SEQ ID NO: 86 - protein, 120 aa, synthetic construct
EVILVESGGGLVKPGGSLKLSCAASGFSFSSYTMSWVRQTPEKRLDWVATISGGGRDIYYPDSVKGRFTISRDNAKNTLYLEMSSLMSEDTALYYCVLLTGRVYFALDSWGQGTSVTVSS

SEQ ID NO: 87 - protein, 111 aa, synthetic construct
  REGION 1..111: note = anti PD1 antibody 478 light chain variable region VL
KIVLTQSPASLPVSLGQRATISCRASESVDTSDNSFIHWYQQRPGQSPKLLIYRSSTLESGVPARFSGSGSRTDFTLTIDPVEADDVATYYCQQNYDVPWTFGGGTKLEIK

SEQ ID NO: 88 - protein, 5 aa, synthetic construct
  REGION 1..5: note = anti CD44 antibody 005 heavy chain CDR_H1
GTAIH

SEQ ID NO: 89 - protein, 17 aa, synthetic construct
  REGION 1..17: note = anti CD44 antibody 005 heavy chain CDR_H2
SISPGGGSTAYADSVKG

SEQ ID NO: 90 - protein, 11 aa, synthetic construct
  REGION 1..11: note = anti CD44 antibody 005 heavy chain CDR_H3
SGGSGASAMDY

SEQ ID NO: 91 - protein, 11 aa, synthetic construct
  REGION 1..11: note = anti CD44 antibody 005 light chain CDR_L1
RASQDVGSGVA

SEQ ID NO: 92 - protein, 7 aa, synthetic construct
  REGION 1..7: note = anti CD44 antibody 005 light chain CDR_L2
SASGLYS

SEQ ID NO: 93 - protein, 9 aa, synthetic construct
  REGION 1..9: note = anti CD44 antibody 005 light chain CDR_L3
QQSASGGST

SEQ ID NO: 94 - protein, 120 aa, synthetic construct; identical to SEQ ID NO: 33
  REGION 1..120: note = anti CD44 antibody 005 heavy chain variable region VH

SEQ ID NO: 95 - protein, 107 aa, synthetic construct; identical to SEQ ID NO: 34
  REGION 1..107: note = anti CD44 antibody 005 light chain variable region VL

SEQ ID NO: 96 - protein, 227 aa, synthetic construct
  REGION 1..227: note = anti PD1 crossmab 478 light chain
EVILVESGGGLVKPGGSLKLSCAASGFSFSSYTMSWVRQTPEKRLDWVATISGGGRDIYYPDSVKGRFTISRDNAKNTLYLEMSSLMSEDTALYYCVLLTGRVYFALDSWGQGTSVTVSSASDAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 97 - protein, 437 aa, synthetic construct
  REGION 1..437: note = anti PD1 crossmab 478 heavy chain
KIVLTQSPASLPVSLGQRATISCRASESVDTSDNSFIHWYQQRPGQSPKLLIYRSSTLESGVPARFSGSGSRTDFTLTIDPVEADDVATYYCQQNYDVPWTFGGGTKLEIKSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKEQMAKDKVSLTCMITNFFPEDITVEWQWNGQPAENYDNTQPIMDTDGSYFVYSDLNVQKSNWEAGNTFTCSVLHEGLHNHHTEKSLSHSPGK

SEQ ID NO: 98 - protein, 214 aa, synthetic construct
  REGION 1..214: note = anti mTfR antibody 001 light chain
DIQMTQSPASLSASLEEIVTITCQASQDIGNWLAWYQQKPGKSPQLLIYGATSLADGVPSRFSGSRSGTQFSLKISRVQVEDIGIYYCLQAYNTPWTFGGGTKVEIKRADAAPTVSIFPPSSRKLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 99 - protein, 667 aa, synthetic construct
  REGION 1..667: note = anti mTfR antibody 001 heavy chain with N-terminal anti PD1-478 crossmab in TCB format
KIVLTQSPASLPVSLGQRATISCRASESVDTSDNSFIHWYQQRPGQSPKLLIYRSSTLESGVPARFSGSGSRTDFTLTIDPVEADDVATYYCQQNYDVPWTFGGGTKLEIKSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGGGGSGGGGSEVQLVESGGGLVQPGNSLTLSCVASGFTFSNYGMHWIRQAPKKGLEWIAMIYYDSSKMNYADTVKGRFTISRDNSKNTLYLEMNSLRSEDTAMYYCAVPTSHYVVDVWGQGVSVTVSSAKTTPPSVYPLAPGSAAQTNSMVTLGCLVEGYFPEPVTVTWNSGSLSSGVHTFPAVLQSDLYTLSSSVTVPSSTWPSQTVTCNVAHPASSTKVDEKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKKQMAKDKVSLTCMITNFFPEDITVEWQWNGQPAENYKNTQPIMKTDGSYFVYSKLNVQKSNWEAGNTFTCSVLHEGLHNHHTEKSLSHSPGK

SEQ ID NO: 100 - protein, 667 aa, synthetic construct
  REGION 1..667: note = anti PD1 478 crossmab heavy chain with C-terminal mTfR-001 Fab in BBB format
KIVLTQSPASLPVSLGQRATISCRASESVDTSDNSFIHWYQQRPGQSPKLLIYRSSTLESGVPARFSGSGSRTDFTLTIDPVEADDVATYYCQQNYDVPWTFGGGTKLEIKSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKKQMAKDKVSLTCMITNFFPEDITVEWQWNGQPAENYKNTQPIMKTDGSYFVYSKLNVQKSNWEAGNTFTCSVLHEGLHNHHTEKSLSHSPGKGGGGSGGGGSEVQLVESGGGLVQPGNSLTLSCVASGFTFSNYGMHWIRQAPKKGLEWIAMIYYDSSKMNYADTVKGRFTISRDNSKNTLYLEMNSLRSEDTAMYYCAVPTSHYVVDVWGQGVSVTVSSAKTTPPSVYPLAPGSAAQTNSMVTLGCLVEGYFPEPVTVTWNSGSLSSGVHTFPAVLQSDLYTLSSSVTVPSSTWPSQTVTCNVAHPASSTKVDEKIVPRDC

SEQ ID NO: 101 - protein, 214 aa, synthetic construct
  REGION 1..214: note = anti Nada (CD44) light chain
DIQMTQSPSSLSASVGDRVTITCRASQDVGSGVAWYQQKPGKAPKLLIGSASGLYSGVPSRFSGSRSGTDFTLTISSLQPEDFATYYCQQSASGGSTFGQGTKVEIKRADAAPTVSIFPPSSRKLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 102 - protein, 669 aa, synthetic construct
  REGION 1..669: note = anti Nada (CD44) antibody heavy chain with N-terminal anti PD1 478 crossmab in TCB format
KIVLTQSPASLPVSLGQRATISCRASESVDTSDNSFIHWYQQRPGQSPKLLIYRSSTLESGVPARFSGSGSRTDFTLTIDPVEADDVATYYCQQNYDVPWTFGGGTKLEIKSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGGGGSGGGGSEVQLVESGGGLVQPGGSLRLSCAASGFSIAGTAIHWVRQAPGKGLEWVASISPGGGSTAYADSVKGRFTISADTSKNTAYLQMNSLRAEDTAVYYCSRSGGSGASAMDYWGQGTLVTVSSAKTTPPSVYPLAPGSAAQTNSMVTLGCLVEGYFPEPVTVTWNSGSLSSGVHTFPAVLQSDLYTLSSSVTVPSSTWPSQTVTCNVAHPASSTKVDEKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKKQMAKDKVSLTCMITNFFPEDITVEWQWNGQPAENYKNTQPIMKTDGSYFVYSKLNVQKSNWEAGNTFTCSVLHEGLHNHHTEKSLSHSPGK

SEQ ID NO: 103 - protein, 669 aa, synthetic construct
  REGION 1..669: note = anti PD-1 CrossMab 478 heavy chain with C-terminal anti Nada Fab in BBB format
KIVLTQSPASLPVSLGQRATISCRASESVDTSDNSFIHWYQQRPGQSPKLLIYRSSTLESGVPARFSGSGSRTDFTLTIDPVEADDVATYYCQQNYDVPWTFGGGTKLEIKSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKKQMAKDKVSLTCMITNFFPEDITVEWQWNGQPAENYKNTQPIMKTDGSYFVYSKLNVQKSNWEAGNTFTCSVLHEGLHNHHTEKSLSHSPGKGGGGSGGGGSEVQLVESGGGLVQPGGSLRLSCAASGFSIAGTAIHWVRQAPGKGLEWVASISPGGGSTAYADSVKGRFTISADTSKNTAYLQMNSLRAEDTAVYYCSRSGGSGASAMDYWGQGTLVTVSSAKTTPPSVYPLAPGSAAQTNSMVTLGCLVEGYFPEPVTVTWNSGSLSSGVHTFPAVLQSDLYTLSSSVTVPSSTWPSQTVTCNVAHPASSTKVDEKIVPRDC

SEQ ID NO: 104 - protein, 227 aa, synthetic construct
  REGION 1..227: note = anti Nada (CD44) crossmab light chain
EVQLVESGGGLVQPGGSLRLSCAASGFSIAGTAIHWVRQAPGKGLEWVASISPGGGSTAYADSVKGRFTISADTSKNTAYLQMNSLRAEDTAVYYCSRSGGSGASAMDYWGQGTLVTVSSASDAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 105 - protein, 433 aa, synthetic construct
  REGION 1..433: note = anti Nada (CD44) crossmab heavy chain
DIQMTQSPSSLSASVGDRVTITCRASQDVGSGVAWYQQKPGKAPKLLIGSASGLYSGVPSRFSGSRSGTDFTLTISSLQPEDFATYYCQQSASGGSTFGQGTKVEIKSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKEQMAKDKVSLTCMITNFFPEDITVEWQWNGQPAENYDNTQPIMDTDGSYFVYSDLNVQKSNWEAGNTFTCSVLHEGLHNHHTEKSLSHSPGK

SEQ ID NO: 106 - protein, 663 aa, synthetic construct
  REGION 1..663: note = anti mTfR antibody 001 heavy chain with N-terminal anti Nada crossmab in TCB format
DIQMTQSPSSLSASVGDRVTITCRASQDVGSGVAWYQQKPGKAPKLLIGSASGLYSGVPSRFSGSRSGTDFTLTISSLQPEDFATYYCQQSASGGSTFGQGTKVEIKSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGGGGSGGGGSEVQLVESGGGLVQPGNSLTLSCVASGFTFSNYGMHWIRQAPKKGLEWIAMIYYDSSKMNYADTVKGRFTISRDNSKNTLYLEMNSLRSEDTAMYYCAVPTSHYVVDVWGQGVSVTVSSAKTTPPSVYPLAPGSAAQTNSMVTLGCLVEGYFPEPVTVTWNSGSLSSGVHTFPAVLQSDLYTLSSSVTVPSSTWPSQTVTCNVAHPASSTKVDEKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKKQMAKDKVSLTCMITNFFPEDITVEWQWNGQPAENYKNTQPIMKTDGSYFVYSKLNVQKSNWEAGNTFTCSVLHEGLHNHHTEKSLSHSPGK

SEQ ID NO: 107 - protein, 663 aa, synthetic construct
  REGION 1..663: note = anti Nada (CD44) crossmab heavy chain with C-terminal anti mTfR Fab 001 in BBB format
DIQMTQSPSSLSASVGDRVTITCRASQDVGSGVAWYQQKPGKAPKLLIGSASGLYSGVPSRFSGSRSGTDFTLTISSLQPEDFATYYCQQSASGGSTFGQGTKVEIKSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKKQMAKDKVSLTCMITNFFPEDITVEWQWNGQPAENYKNTQPIMKTDGSYFVYSKLNVQKSNWEAGNTFTCSVLHEGLHNHHTEKSLSHSPGKGGGGSGGGGSEVQLVESGGGLVQPGNSLTLSCVASGFTFSNYGMHWIRQAPKKGLEWIAMIYYDSSKMNYADTVKGRFTISRDNSKNTLYLEMNSLRSEDTAMYYCAVPTSHYVVDVWGQGVSVTVSSAKTTPPSVYPLAPGSAAQTNSMVTLGCLVEGYFPEPVTVTWNSGSLSSGVHTFPAVLQSDLYTLSSSVTVPSSTWPSQTVTCNVAHPASSTKVDEKIVPRDC

SEQ ID NO: 108 - protein, 20 aa, synthetic construct
GGGGSGGGGSGGGGSGGGGS

SEQ ID NO: 109 - protein, 10 aa, synthetic construct
GGGGSGGGGS

(19) United States
(12) Patent Application Publication
(10) Pub. No.: US 2025/0215103 A1
(43) Pub. Date: Jul. 3, 2025

(54) BISPECIFIC ANTIBODIES AND METHODS OF USE

(71) Applicant: Hoffmann-La Roche Inc., Little Falls, NJ (US)

(72) Inventors: Ulrich BRINKMANN, Weilheim (DE); Laura CODARRI DEAK, Au (CH); Christian KLEIN, Zurich (CH); Annette Stephanie INDLEKOFER, Penzberg (DE); Daniela SCHMID, Lenggries (DE); Patrick Alexander Aaron WEBER, Aargau (CH)

(73) Assignee: Hoffmann-La Roche Inc., Little Falls, NJ (US)

(21) Appl. No.: 18/294,139

(22) PCT Filed: Aug. 2, 2022
(86) PCT No.: PCT/EP2022/071661

§ 371 Date: Jan. 31, 2024

(30) Foreign Application Priority Data
Aug. 3, 2021 (EP) 21189372.2

(51) Int. Cl. C07K 16/28; A61K 39/00; A61P 35/00

(52) U.S. Cl. CPC C07K 16/2881; A61P 35/00; C07K 16/2818; A61K 2039/505; C07K 2317/31; C07K 2317/35; C07K 2317/55; C07K 2317/77; C07K 2317/92

(57) ABSTRACT

The invention relates to bispecific antibodies comprising a first antigen-binding domain that specifically binds to TfR and a second, and optionally a third, antigen-binding domain that specifically binds to PD1. The invention further relates to methods of producing these molecules, to methods of using the same, to pharmaceutical compositions thereof, and their use as medicaments for the treatment of cancer, of acute and chronic infections and of Graft-versus-host disease.

### TECHNICAL FIELD

The present invention relates to bispecific antibodies comprising a first antigen-binding domain that specifically binds to TfR and a second, and optionally a third, antigen-binding domain that specifically binds to PD1. The invention further relates to immunoconjugates comprising the bispecific antibody and to methods of using the bispecific antibody or the immunoconjugate.

### BACKGROUND

The protein Programmed Death 1 (PD1 or CD279) is an inhibitory member of the CD28 family of cell surface receptors which also includes CD28, CTLA-4, ICOS and BTLA, and binds to PD-L1 and PD-L2 (Greenwald R. J. et al. Annu Rev Immunol. (2005) 23:515-48; Freeman G. J. et al. J Exp Med. (2000) 192:1027-34; Latchman Y. et al. Nat Immunol. (2001) 2:261-8), forming the so-called PD1/PD-L1 axis. PD1 is expressed on activated B cells, T cells, and myeloid cells (Agata et al, supra; Okazaki et al (2002) Curr. Opin. Immunol. 14:391779-82; Bennett et al. (2003) J Immunol 170:711-8). The PD1 gene is a 55 kDa type I transmembrane protein that is part of the Ig gene superfamily (Agata et al. (1996) Int Immunol 8:765-72). PD1 contains a membrane proximal immunoreceptor tyrosine inhibitory motif (ITIM) and a membrane distal tyrosine-based switch motif (ITSM) (Thomas, M. L. (1995) J Exp Med 181:1: 1953-6; Vivier, E. and Daeron, M. (1997) Immunol Today 18:286-91). Although structurally similar to CTLA-4, PD1 lacks the MYPPPY motif (SEQ ID NO: 71) that is critical for B7-1 and B7-2 binding. Two ligands for PD1 have been identified, PD-L1 (CD274) and PD-L2 (CD273), that have been shown to downregulate T cell activation upon binding to PD1 (Freeman et al. (2000) J Exp Med 192:1027-34; Latchman et al (2001) Nat Immunol 2:261-8; Carter et al. (2002) Eur J Immunol 32:634-43). Both PD-L1 and PD-L2 are B7 homologs that bind to PD1, but do not bind to other CD28 family members. One ligand for PD1, PD-L1 is abundant in a variety of human cancers (Dong et al (2002) Nat. Med 8:787-9). Targeting the PD1/PD-L1 immunological checkpoint with monoclonal antibodies and small molecular drugs has become a major focus in immunooncology.

In addition to its role as inhibitory member of the CD28 family, PD1 has been found to play a role in autoimmune encephalomyelitis, systemic lupus erythematosus, graft-versus-host disease (GVHD), type I diabetes, and rheumatoid arthritis (Salama et al. (2003) J Exp Med 198:71-78; Prokunina and Alarcon-Riquelme (2004) Hum Mol Genet 13: R143; Nielsen et al. (2004) Lupus 13:510). In a murine B cell tumor line, the ITSM of PD1 was shown to be essential in blocking B-cell receptor-mediated Ca²⁺-flux and tyrosine phosphorylation of downstream effector molecules (Okazaki et al. (2001) PNAS 98:13866-71).

Various patent applications disclose production of anti-PD1 antibodies and/or methods of enhancing immune responses with an agent (including an anti-PD1 antibody) that interferes with PD-L1 binding and/or PD1 signaling, including the following: US2003/0039653, US2004/0213795, US2006/0110383, US2007/0065427, US2007/0122378, US2012/237522, WO2004/072286, WO2006/121168, WO2006/133396, WO2007/005874, WO2008/083174, WO2008/156712, WO2009/024531, WO2009/014708, WO2009/114335, WO2010/027828, WO2010/027423, WO2010/036959, WO2010/029435, WO2010/029434, WO2010/063011, WO2010/089411, WO2011/066342, WO2011/110604, WO2011/110621, and WO2012/145493.

Transferrin Receptor (TfR) is a membrane receptor that is involved in iron transport into the cell by binding the iron-transferrin complex and internalizing it by receptor-mediated endocytosis. TfR is an attractive target for therapeutic approaches of intracellular delivery due to its fast internalization and recycling rate. However, delivery in vivo is mostly inefficient and unspecific due to the vast TfR expression throughout the body.

The effect of the PD1 antibodies described in the art relies on blocking the interaction between PD-L1 and PD1 by binding to PD1. Since even the most avid antibody-binding of an anti-PD1-antibody to PD1 is non-covalent and therefore transient, there is a need to develop new compounds targeting PD1 that have improved efficacy and longer-lasting effect than the known anti-PD1 antibodies.

### SUMMARY

In one aspect, the invention provides novel bispecific antibodies comprising a first antigen-binding domain that specifically binds to TfR and a second, and optionally a third, antigen-binding domain that specifically binds to a molecule of the PD1/PD-L1 axis. In one aspect, the PD1/PD-L1 axis molecule is selected from the group consisting of PD1, PD-L1 and PD-L2. In one particular aspect, the PD1/PD-L1 axis molecule is PD1 or PD-L1. In one particular aspect, the PD1/PD-L1 axis molecule is PD1. The anti-TfR anti-PD1 bispecific antibodies of the invention have particularly advantageous properties such as functionally optimized binding affinity, increased biological activity, specific targeting of certain T cells and high targeting efficiency.

In another aspect, the bispecific antibody binds to the TfR and PD1 receptors on the surface of a cell which expresses and displays TfR and PD1 on its surface. In a preferred aspect, the binding of the antibody to TfR and PD1 is simultaneous. Upon the binding of the bispecific antibody to TfR and PD1 on the cell surface, PD1 is depleted from the surface of said cell expressing TfR and PD1, preferably by the internalization of the complex that is formed by the bispecific antibody with TfR and PD1 into said cell. PD1 is consequently depleted from the cell surface, preferably together with TfR and the bound bispecific antibody. The invention is at least in part based on the finding that the anti-PD1 anti-TfR bispecific antibodies of the invention have the advantageous effect of inhibiting the interaction between PD1 and PD-L1 by removing PD1 from the cell surface, which is more effective and/or durable than an inhibition that is achievable by mere binding of an anti-PD1 blocking antibody which does not lead to internalization of PD1 and depletion of PD1 from the cell surface.

In one aspect, the invention provides a bispecific antibody comprising a first antigen-binding domain that specifically binds to TfR, a second antigen-binding domain that specifically binds to PD1 and a third antigen-binding domain that specifically binds to PD1. This bispecific antibody has thus one antigen-binding domain specific for TfR and two antigen-binding domains specific for PD1. Such a molecule having two binding domains for a first target and one binding domain for a second target is also called 2+1 format or 2+1 format antibody. In one aspect these molecules are based on IgG class Fab fragments, and optionally also IgG class Fc regions, that may be covalently bound to each other in different conformations resulting in different 2+1 format antibodies. Examples for different 2+1 formats with different conformations of the antigen-binding domains are shown in FIGS. 1-4. Further conformations are described in the art (Brinkmann and Kontermann (2017) MAbs 9 (2): 182-212; Kontermann and Brinkmann (2015) Drug Discov Today 20 (7): 838-47; Bacac M et al. (2018) Clin Cancer Res. 24 (19): 4785-4797; Rius Ruiz et al. (2018) Sci Transl Med 10 (461): eaat1445; Seckinger et al. (2017) Cancer Cell. 31 (3): 396-410; Bacac et al. (2016) Oncoimmunology. 5 (8): e1203498; Bacac et al (2016) Clin Cancer Res. 22 (13): 3286-97; Weber et al. (2018) Cell Rep. 22 (1): 149-162; Niewoehner et al. (2014) Neuron. 81 (1): 49-60).

Surprisingly, it has been found that such an anti-TfR anti-PD1 2+1 format antibody, i.e. a bispecific antibody with a 2:1 stoichiometry of the binding domains targeting anti-PD1 and anti-TfR, respectively, or in other words, a bispecific antibody comprising a first antigen-binding domain that specifically binds to TfR and a second and a third antigen-binding domain that specifically bind to PD1, shows improved biological activity and leads to better inhibition of the interaction between PD1 and PD-L1 than a monospecific, bivalent PD1 antibody.

In one aspect of the bispecific antibody comprising a first antigen-binding domain that specifically binds to TfR and a second, and optionally a third, antigen-binding domain that specifically binds to PD1, the first, the second and/or, where present, the third antigen-binding domain is a Fab fragment. In a further aspect, the invention relates to a bispecific antibody comprising a first antigen-binding domain that specifically binds to TfR and a second, and optionally a third, antigen-binding domain that specifically binds to PD1, wherein the bispecific antibody comprises an Fc domain that is composed of a first and a second subunit. In a particular aspect, one or more of the Fab fragments comprised by the bispecific antibody are fused to the Fc domain. In another aspect, the Fab fragments are fused to the Fc domain via a peptidic linker. In an additional aspect, the Fc domain is an IgG Fc domain, particularly an IgG1 Fc domain or an IgG4 Fc domain. In a particular aspect, the heavy chain of the bispecific antibody is of the γ type (IgG), particularly of the γ1 type. In another particular aspect, the light chain of the bispecific antibody is of the kappa (κ) and/or lambda (λ) subtype, based on the amino acid sequence of its constant domain.

In one aspect, the invention provides a bispecific antibody comprising a first antigen-binding domain that specifically binds to TfR and a second, and optionally a third, antigen-binding domain that specifically binds to PD1 wherein the bispecific antibody comprises an Fc domain, a first Fab fragment comprising the antigen-binding domain that specifically binds to TfR and a second, and optionally a third, Fab fragment comprising the antigen-binding domain that specifically binds to PD1. In a particular aspect, the bispecific antibody comprises an Fc domain, a first Fab fragment comprising an antigen-binding domain that specifically binds to TfR and a second and optionally a third Fab fragment comprising an antigen-binding domain that specifically binds to PD1, wherein the Fab fragments are fused to the Fc domain. In one aspect, the bispecific antibody comprises exactly one (monovalent) antigen-binding domain that specifically binds to TfR and exactly two (monovalent) antigen-binding domains that specifically bind to PD1. In particular, the Fc domain is an IgG Fc domain, more particularly an IgG1 Fc domain or an IgG4 Fc domain. In one particular aspect, the heavy chain of the bispecific antibody is of the γ type (IgG), particularly of the γ1 (IgG1) subtype. In another particular aspect, the light chain of the bispecific antibody is of the kappa (κ) and/or lambda (λ) subtype, based on the amino acid sequence of its constant domain. In one aspect, the bispecific antibody does not comprise a J-chain. In another aspect, the bispecific antibody does not comprise hybrid IgA/IgG antibody sequences and/or hybrid IgM/IgG antibody sequences. In a further aspect the bispecific antibody is essentially in monomeric form, i.e. it does not form dimeric or multimeric (e.g. pentameric) structures comprising more than one bispecific antibody of the invention. In a particular aspect, at least 90%, more particularly at least 95%, preferably at least 98%, more preferably at least 99% of the antibody are in monomeric form.

In one aspect, the invention relates to a bispecific antibody comprising a first antigen-binding domain that specifically binds to TfR and a second, and optionally a third, antigen-binding domain that specifically binds to PD1 wherein the Fc domain comprises one or more amino acid substitutions that reduce binding to an Fc receptor, in particular towards Fcγ receptor. In particular, the Fc domain is of human IgG1 subclass with the amino acid mutations L234A, L235A and P329G (numbering according to Kabat EU index).

In another aspect, the invention relates to a bispecific antibody comprising a first antigen-binding domain that specifically binds to TfR and a second, and optionally a third, antigen-binding domain that specifically binds to PD1, wherein the Fc domain comprises a modification promoting the association of the first and second subunit of the Fc domain. In one aspect, the invention relates to a bispecific antibody comprising a first antigen-binding domain that specifically binds to TfR and a second, and optionally a third, antigen-binding domain that specifically binds to PD1, wherein the first subunit of the Fc domain comprises knobs and the second subunit of the Fc domain comprises holes according to the knobs into holes method. In a particular aspect, the bispecific antibody is one wherein the first subunit of the Fc domain comprises the amino acid substitutions S354C and T366W (numbering according to Kabat EU index) and the second subunit of the Fc domain comprises the amino acid substitutions Y349C, T366S and Y407V (numbering according to Kabat EU index).

In a further aspect, the invention provides a bispecific antibody comprising a first antigen-binding domain that specifically binds to TfR and a second, and optionally a third, antigen-binding domain that specifically binds to PD1, wherein the first, the second and, where present, the third antigen-binding domain are each a Fab fragment and wherein in one or two of the Fab fragments
a) the variable domains VL and VH are replaced by each other so that the VH domain is part of the light chain and the VL domain is part of the heavy chain, or
b) the constant domains CL and CH1 are replaced by each other so that the CH1 domain is part of the light chain and the CL domain is part of the heavy chain.

In a preferred aspect, the variable domains VL and VH are replaced by each other so that the VH domain is part of the light chain and the VL domain is part of the heavy chain. In a particular aspect, the bispecific antibody is one wherein in the Fab fragment(s) comprising the antigen-binding domain that specifically binds to PD1 either the variable domains VL and VH or the constant domains CL and CH1 are replaced by each other. In a particularly preferred aspect, the variable domains VL and VH are replaced by each other in the antigen-binding domain that specifically binds to PD1. In one aspect the bispecific antibody comprises exactly one (monovalent) antigen-binding domain that specifically binds to TfR and exactly two (monovalent) antigen-binding domains that specifically bind to PD1.

In an additional aspect, the invention relates to a bispecific antibody comprising a first antigen-binding domain that specifically binds to TfR and a second, and optionally a third, antigen-binding domain that specifically binds to PD1, wherein the first, the second and, where present, the third antigen-binding domain are each a Fab fragment and wherein in one or two of the Fab fragments in the constant domain CL the amino acid at position 124 is substituted independently by lysine (K), arginine (R) or histidine (H) (numbering according to Kabat EU Index), and in the constant domain CH1 the amino acids at positions 147 and 213 are substituted independently by glutamic acid (E) or aspartic acid (D) (numbering according to Kabat EU index). In a particular aspect the invention relates to a bispecific antibody comprising a first antigen-binding domain that specifically binds to TfR and a second, and optionally a third, antigen-binding domain that specifically binds to PD1, wherein in the Fab fragment comprising the antigen-binding domain that specifically binds to TfR, in the constant domain CL the amino acid at position 124 is substituted independently by lysine (K), arginine (R) or histidine (H) (numbering according to Kabat EU Index), and in the constant domain CH1 the amino acids at positions 147 and 213 are substituted independently by glutamic acid (E) or aspartic acid (D) (numbering according to Kabat EU index).

In a particular aspect the invention provides a bispecific antibody comprising a first antigen-binding domain that specifically binds to TfR and a second, and optionally a third, antigen-binding domain that specifically binds to PD1, wherein in the second and, where present, the third Fab fragment comprising the antigen-binding domain that specifically binds to PD1, the amino acid at position 124 in the constant domain CL is substituted independently by lysine (K), arginine (R) or histidine (H) (numbering according to Kabat EU Index), and the amino acids at positions 147 and 213 in the constant domain CH1 are substituted independently by glutamic acid (E) or aspartic acid (D) (numbering according to Kabat EU index).

In one aspect, the bispecific antibody comprising a first antigen-binding domain that specifically binds to TfR and a second antigen-binding domain that specifically binds to PD1 is a bivalent antibody comprising
a) a first light chain and a first heavy chain of an antibody specifically binding to a first antigen, and
b) a second light chain and a second heavy chain of an antibody specifically binding to a second antigen, wherein the variable domains VL and VH of the second light chain and the second heavy chain are replaced by each other, and wherein the constant domains CL and CH1 of the second light chain and the second heavy chain are replaced by each other.

The two subunits of the antibody under a) do not contain a modification as reported under b) and the heavy chain and the light chain under a) are isolated chains. In two subunits of the antibody under b) within the light chain the variable light chain domain VL is replaced by the variable heavy chain domain VH of said antibody, and the constant light chain domain CL is replaced by the constant heavy chain domain CH1 of said antibody; and within the heavy chain the variable heavy chain domain VH is replaced by the variable light chain domain VL of said antibody, and the constant heavy chain domain CH1 is replaced by the constant light chain domain CL of said antibody.

In one aspect, the bispecific antibody comprising a first antigen-binding domain that specifically binds to TfR and a second antigen-binding domain that specifically binds to PD1 is a bivalent antibody comprising
a) a first light chain and a first heavy chain of an antibody specifically binding to a first antigen, and
b) a second light chain and a second heavy chain of an antibody specifically binding to a second antigen, wherein the variable domains VL and VH of the second light chain and the second heavy chain are replaced by each other.

The two subunits of the antibody under a) do not contain a modification as reported under b) and the heavy chain and the light chain under a) are isolated chains. In two subunits of the antibody under b) within the light chain the variable light chain domain VL is replaced by the variable heavy chain domain VH of said antibody; and within the heavy chain the variable heavy chain domain VH is replaced by the variable light chain domain VL of said antibody.

In one aspect, the bispecific antibody comprising a first antigen-binding domain that specifically binds to TfR and a second antigen-binding domain that specifically binds to PD1 is a bivalent antibody comprising
a) a first light chain and a first heavy chain of an antibody specifically binding to a first antigen, and
b) a second light chain and a second heavy chain of an antibody specifically binding to a second antigen, wherein the constant domains CL and CH1 of the second light chain and the second heavy chain are replaced by each other.

The two subunits of the antibody under a) do not contain a modification as reported under b) and the heavy chain and the light chain under a) are isolated chains. In the two subunits of the antibody under b) the constant light chain domain CL is replaced within the light chain by the constant heavy chain domain CH1 of said antibody; and the constant heavy chain domain CH1 is replaced within the heavy chain by the constant light chain domain CL of said antibody.

In one further aspect, the invention provides a bispecific antibody comprising a first antigen-binding domain that specifically binds to TfR and a second, and optionally a third, antigen-binding domain that specifically binds to PD1, wherein the first and the second and, where present, the third antigen-binding domain are each a Fab fragment and either (i) the second antigen-binding domain is fused at the C-terminus of its Fab heavy chain to the N-terminus of the Fab heavy chain of the first antigen-binding domain, or (ii) the first antigen-binding domain is fused at the C-terminus of its Fab heavy chain to the N-terminus of the Fab heavy chain of the second antigen-binding domain. In other words, the bispecific antibody is comprised of Fab fragments that are fused to each other. The third antigen-binding domain, where present, is fused to the bispecific antibody either at the C-terminus of its Fab heavy chain to the free N-terminus of one of the two other Fab heavy chains or at the N-terminus of its Fab heavy chain to the free C-terminus of one of the two other Fab heavy chains (see also FIG. 4 for exemplary conformations). In one aspect, the bispecific antibody comprises exactly one (monovalent) antigen-binding domain that specifically binds to TfR and exactly two (monovalent) antigen-binding domains that specifically bind to PD1.

In one aspect, the bispecific antibody comprises a first antigen-binding domain that specifically binds to TfR and a second and a third antigen-binding domain that specifically bind to PD1, wherein said bispecific antibody is a trivalent antibody comprising
a) a full length antibody consisting of two antibody heavy chains and two antibody light chains and comprising two antigen-binding domains specifically binding to PD1,
b) a first polypeptide consisting of
   i) an antibody heavy chain variable domain (VH), or
   ii) an antibody heavy chain variable domain (VH) and an antibody constant domain 1 (CH1),
wherein said first polypeptide is fused with the N-terminus of its VH domain via a peptidic linker to the C-terminus of one of the two heavy chains of said full length antibody, and
c) a second polypeptide consisting of
   i) an antibody light chain variable domain (VL), or
   ii) an antibody light chain variable domain (VL) and an antibody light chain constant domain (CL),
wherein said second polypeptide is optionally fused with the N-terminus of the VL domain or of the CL domain via a peptidic linker to the C-terminus of the other of the two heavy chains of said full length antibody, and
wherein the antibody heavy chain variable domain (VH) of the first polypeptide and the antibody light chain variable domain (VL) of the second polypeptide together form an antigen-binding domain specifically binding to TfR.

In another aspect, the bispecific antibody comprises a first antigen-binding domain that specifically binds to TfR and a second and a third antigen-binding domain that specifically bind to PD1, wherein said bispecific antibody is a trivalent antibody comprising
a) a full length antibody consisting of two antibody heavy chains and two antibody light chains and comprising a first antigen-binding domain specifically binding to PD1 and a second antigen-binding domain specifically binding to TfR,
b) a first polypeptide consisting of
   i) an antibody heavy chain variable domain (VH), or
   ii) an antibody heavy chain variable domain (VH) and an antibody constant domain 1 (CH1),
wherein said first polypeptide is fused with the C-terminus of its VH domain or, where present, of its constant domain 1 (CH1) via a peptidic linker to the N-terminus of one of the two heavy chains of said full length antibody, and
c) a second polypeptide consisting of
   i) an antibody light chain variable domain (VL), or
   ii) an antibody light chain variable domain (VL) and an antibody light chain constant domain (CL),
wherein said second polypeptide is optionally fused with the C-terminus of the VL domain or, where present, of the CL domain via a peptidic linker to the N-terminus of the other of the two heavy chains of said full length antibody, and
wherein the antibody heavy chain variable domain (VH) of the first polypeptide and the antibody light chain variable domain (VL) of the second polypeptide together form an antigen-binding domain specifically binding to PD1. In a particular aspect, the first and optionally the second polypeptide are fused to the N-terminus of the heavy chain that comprises the VH domain of the antigen-binding domains specifically binding to TfR.

In one aspect, the invention relates to a bispecific antibody comprising a first antigen-binding domain that specifically binds to TfR and a second, and optionally a third, antigen-binding domain that specifically binds to PD1, wherein the first, the second and, where present, the third antigen-binding domain are each a Fab fragment and the antibody comprises an Fc domain composed of a first and a second subunit; and wherein either
(i) the second antigen-binding domain is fused at the C-terminus of its Fab heavy chain to the N-terminus of the Fab heavy chain of the first antigen-binding domain and the first antigen-binding domain is fused at the C-terminus of its Fab heavy chain to the N-terminus of the first subunit of the Fc domain, or
(ii) the first antigen-binding domain is fused at the C-terminus of its Fab heavy chain to the N-terminus of the Fab heavy chain of the second antigen-binding domain and the second antigen-binding domain is fused at the C-terminus of the Fab heavy chain to the N-terminus of the first subunit of the Fc domain; and
(iii) the third antigen-binding domain, where present, is fused at the C-terminus of its Fab heavy chain to the N-terminus of the second subunit of the Fc domain. In one aspect, the bispecific antibody is a trivalent antibody. In another aspect, the bispecific antibody comprises exactly one (monovalent) antigen-binding domain that specifically binds to TfR and exactly two (monovalent) antigen-binding domains that specifically bind to PD1. In certain aspects, the bispecific antibody is of the IgG class. In another aspect, the Fab fragments and/or the Fc region of the bispecific antibody are of the IgG class. In certain aspects, the bispecific antibody is of the IgG₁ isotype. In another aspect, the Fab fragments and/or the Fc region of the bispecific antibody are of the IgG₁ isotype.

In a further aspect, the invention provides a bispecific antibody comprising a first antigen-binding domain that specifically binds to TfR and a second, and optionally a third, antigen-binding domain that specifically binds to PD1, wherein the first, the second and, where present, the third antigen-binding domain are each a Fab fragment and the antibody comprises an Fc domain composed of a first and a second subunit; and wherein
i) the first antigen-binding domain is fused at the N-terminus of its Fab heavy chain to the C-terminus of the first or second subunit of the Fc domain, the second antigen-binding domain is fused at the C-terminus of its Fab heavy chain to the N-terminus of the first subunit of the Fc domain and, where present, the third antigen-binding domain is fused at the C-terminus of its Fab heavy chain to the N-terminus of the second subunit of the Fc domain or
ii) the first antigen-binding domain is fused at the C-terminus of its Fab heavy chain to the N-terminus of the first subunit of the Fc domain, the second antigen-binding domain is fused at the C-terminus of its Fab heavy chain to the N-terminus of the second subunit of the Fc domain and, where present, the third antigen-binding domain is fused at the N-terminus of its Fab heavy chain to the C-terminus of the first or second subunit of the Fc domain. In one aspect, the bispecific antibody is a trivalent antibody. In another aspect, the bispecific antibody comprises exactly one (monovalent) antigen-binding domain that specifically binds to TfR and exactly two (monovalent) antigen-binding domains that specifically bind to PD1. In certain aspects, the bispecific antibody is of the IgG class. In an additional aspect, the Fab fragments and/or the Fc region of the bispecific antibody are of the IgG class. In certain aspects, the bispecific antibody is of the IgG₁ isotype. In yet another aspect, the Fab fragments and/or the Fc region of the bispecific antibody are of the IgG₁ isotype.

In one aspect, the bispecific antibody is a trivalent antibody comprising
a) one Fab fragment comprising a first antigen-binding domain that specifically binds to TfR,
b) two CrossFab fragments comprising an antigen-binding domain that specifically binds to PD1 in which the CH1 and the CL domain are exchanged for each other,
c) one Fc-region comprising a first Fc-region heavy chain and a second Fc region heavy chain,
wherein the C-terminus of the CH1 domain of the Fab fragment is connected to the N-terminus of one of the heavy chain Fc-region polypeptides and the C-terminus of the CH1-domain of one CrossFab fragment is connected to the N-terminus of the other heavy chain Fc-region polypeptide, and wherein the C-terminus of the CH1 domain of the other CrossFab fragment is connected to the N-terminus of the VH domain of the Fab fragment or to the N-terminus of the VH domain of the CrossFab fragment. In a particular aspect, the connection is via a peptidic linker. In another aspect, the bispecific antibody comprises exactly one (monovalent) antigen-binding domain that specifically binds to TfR and exactly two (monovalent) antigen-binding domains that specifically bind to PD1. In certain aspects, the bispecific antibody is of the IgG class. In an additional aspect, the Fab fragments and/or the Fc region of the bispecific antibody are of the IgG class. In certain aspects, the bispecific antibody is of the IgG₁ isotype. In yet another aspect, the Fab fragments and/or the Fc region of the bispecific antibody are of the IgG₁ isotype.

In one aspect, the bispecific antibody comprising a first antigen-binding domain that specifically binds to TfR and a second, and optionally a third, antigen-binding domain that specifically binds to PD1 comprises
a) a full length antibody specifically binding to PD1 and consisting of two antibody heavy chains and two antibody light chains, wherein within the light chain the variable light chain domain VL is replaced by the variable heavy chain domain VH of said antibody, and within the heavy chain fragment the variable heavy chain domain VH is replaced by the variable light chain domain VL of said antibody, and
b) a Fab fragment specifically binding to TfR,
wherein the N-terminus of the Fab fragment heavy chain is connected to the C-terminus of one of the two heavy chains of the full length antibody. In another aspect, the bispecific antibody comprises exactly one (monovalent) antigen-binding domain that specifically binds to TfR and exactly two (monovalent) antigen-binding domains that specifically bind to PD1. In certain aspects, the bispecific antibody is of the IgG class. In an additional aspect, the Fab fragments and/or the full-length antibody are of the IgG class. In certain aspects, the bispecific antibody is of the IgG₁ isotype. In yet another aspect, the Fab fragments and/or the full-length antibody are of the IgG₁ isotype.

In a further aspect the bispecific antibody comprises a first antigen-binding domain that specifically binds to TfR and a second, and optionally a third, antigen-binding domain that specifically binds to PD1, wherein
the first antigen-binding domain specifically binding to TfR comprises
   a heavy chain variable domain (VH) comprising (a) CDR-H1 comprising the amino acid sequence of SEQ ID NO: 1, (b) CDR-H2 comprising the amino acid sequence of SEQ ID NO: 2, and (c) CDR-H3 comprising the amino acid sequence of SEQ ID NO: 3, and
   a light chain variable domain (VL) comprising (d) CDR-L1 comprising the amino acid sequence of SEQ ID NO: 4, (e) CDR-L2 comprising the amino acid sequence of SEQ ID NO: 5, and (f) CDR-L3 comprising the amino acid sequence of SEQ ID NO: 6,
   or
   a heavy chain variable domain (VH) comprising (a) CDR-H1 comprising the amino acid sequence of SEQ ID NO: 9, (b) CDR-H2 comprising the amino acid sequence of SEQ ID NO: 10, and (c) CDR-H3 comprising the amino acid sequence of SEQ ID NO: 11, and a light chain variable domain (VL) comprising (d) CDR-L1 comprising the amino acid sequence of SEQ ID NO: 12, (e) CDR-L2 comprising the amino acid sequence of SEQ ID NO: 13, and (f) CDR-L3 comprising the amino acid sequence of SEQ ID NO: 14.

In one embodiment, the second and/or, where present, the third antigen-binding domain specifically binding to PD1 of the bispecific antibody comprising a first antigen-binding domain that specifically binds to TfR and a second, and optionally a third, antigen-binding domain that specifically binds to PD1, comprises
a heavy chain variable domain (VH) comprising (a) CDR-H1 comprising the amino acid sequence of SEQ ID NO: 17, (b) CDR-H2 comprising the amino acid sequence of SEQ ID NO: 18, and (c) CDR-H3 comprising the amino acid sequence of SEQ ID NO: 19, and a light chain variable domain (VL) comprising (d) CDR-L1 comprising the amino acid sequence of SEQ ID NO: 20, (e) CDR-L2 comprising the amino acid sequence of SEQ ID NO: 21, and (f) CDR-L3 comprising the amino acid sequence of SEQ ID NO: 22
or
a heavy chain variable domain (VH) comprising (a) CDR-H1 comprising the amino acid sequence of SEQ ID NO: 25, (b) CDR-H2 comprising the amino acid sequence of SEQ ID NO: 26, and (c) CDR-H3 comprising the amino acid sequence of SEQ ID NO: 27, and
a light chain variable domain (VL) comprising (d) CDR-L1 comprising the amino acid sequence of SEQ ID NO: 28, (e) CDR-L2 comprising the amino acid sequence of SEQ ID NO: 29, and (f) CDR-L3 comprising the amino acid sequence of SEQ ID NO: 30.

In a particular aspect, the invention provides a bispecific antibody comprising a first antigen-binding domain that specifically binds to TfR and a second, and optionally a third, antigen-binding domain that specifically binds to PD1 wherein the bispecific antibody simultaneously binds to TfR and PD1 and upon simultaneous binding of the bispecific antibody the complex formed by the bispecific antibody, TfR and PD1 is internalized into the cell and PD1 is depleted from the cell surface, and wherein the bispecific antibody comprises
a first antigen-binding domain specifically binding to TfR comprising
   a heavy chain variable domain (VH) comprising (a) CDR-H1 comprising the amino acid sequence of SEQ ID NO: 1, (b) CDR-H2 comprising the amino acid sequence of SEQ ID NO: 2, and (c) CDR-H3 comprising the amino acid sequence of SEQ ID NO: 3, and
   a light chain variable domain (VL) comprising (d) CDR-L1 comprising the amino acid sequence of SEQ ID NO: 4, (e) CDR-L2 comprising the amino acid sequence of SEQ ID NO: 5, and (f) CDR-L3 comprising the amino acid sequence of SEQ ID NO: 6;
   or
   a heavy chain variable domain (VH) comprising (a) CDR-H1 comprising the amino acid sequence of SEQ ID NO: 9, (b) CDR-H2 comprising the amino acid sequence of SEQ ID NO: 10, and (c) CDR-H3 comprising the amino acid sequence of SEQ ID NO: 11, and
   a light chain variable domain (VL) comprising (d) CDR-L1 comprising the amino acid sequence of SEQ ID NO: 12, (e) CDR-L2 comprising the amino acid sequence of SEQ ID NO: 13, and (f) CDR-L3 comprising the amino acid sequence of SEQ ID NO: 14; and
a second, and/or where present, a third antigen-binding domain specifically binding to PD1 comprising
   a heavy chain variable domain (VH) comprising (a) CDR-H1 comprising the amino acid sequence of SEQ ID NO: 17, (b) CDR-H2 comprising the amino acid sequence of SEQ ID NO: 18, and (c) CDR-H3 comprising the amino acid sequence of SEQ ID NO: 19, and
   a light chain variable domain (VL) comprising (d) CDR-L1 comprising the amino acid sequence of SEQ ID NO: 20, (e) CDR-L2 comprising the amino acid sequence of SEQ ID NO: 21, and (f) CDR-L3 comprising the amino acid sequence of SEQ ID NO: 22
   or
   a heavy chain variable domain (VH) comprising (a) CDR-H1 comprising the amino acid sequence of SEQ ID NO: 25, (b) CDR-H2 comprising the amino acid sequence of SEQ ID NO: 26, and (c) CDR-H3 comprising the amino acid sequence of SEQ ID NO: 27, and
   a light chain variable domain (VL) comprising (d) CDR-L1 comprising the amino acid sequence of SEQ ID NO: 28, (e) CDR-L2 comprising the amino acid sequence of SEQ ID NO: 29, and (f) CDR-L3 comprising the amino acid sequence of SEQ ID NO: 30.

In a further aspect the bispecific antibody comprises a first antigen-binding domain that specifically binds to TfR and a second, and optionally a third, antigen-binding domain that specifically binds to PD1 wherein
the first antigen-binding domain specifically binding to TfR comprises
a VH domain comprising the amino acid sequence of SEQ ID NO: 7 and a VL domain comprising the amino acid sequence of SEQ ID NO: 8, or
a VH domain comprising the amino acid sequence of SEQ ID NO: 15 and a VL domain comprising the amino acid sequence of SEQ ID NO: 16,
and the second and/or, where present, the third antigen-binding domain specifically binding to PD1 comprises
a VH domain comprising the amino acid sequence of SEQ ID NO: 23 and a VL domain comprising the amino acid sequence of SEQ ID NO: 24, or
a VH domain comprising the amino acid sequence of SEQ ID NO: 31 and a VL domain comprising the amino acid sequence of SEQ ID NO: 32.

In a further aspect the bispecific antibody comprising a first antigen-binding domain that specifically binds to TfR and a second, and optionally a third, antigen-binding domain that specifically binds to PD1 is a monoclonal antibody.

In a particular aspect, the bispecific antibody comprising a first antigen-binding domain that specifically binds to TfR and a second, and optionally a third, antigen-binding domain that specifically binds to PD1 is a humanized or chimeric antibody.

In another aspect, the invention relates to a bispecific antibody comprising a first antigen-binding domain that specifically binds to TfR and a second, and optionally a third, antigen-binding domain that specifically binds to PD1, which comprises
a first heavy chain comprising an amino acid sequence with at least 95% sequence identity to the sequence of SEQ ID NO: 35, a first light chain comprising an amino acid sequence with at least 95% sequence identity to the sequence of SEQ ID NO: 36, and
a second heavy chain comprising an amino acid sequence with at least 95% sequence identity to the sequence of SEQ ID NO: 39, and a second light chain comprising an amino acid sequence with at least 95% sequence identity to the sequence of SEQ ID NO: 40,
or
a first heavy chain comprising an amino acid sequence with at least 95% sequence identity to the sequence of SEQ ID NO: 37, a first light chain comprising an amino acid sequence with at least 95% sequence identity to the sequence of SEQ ID NO: 38, and
a second heavy chain comprising an amino acid sequence with at least 95% sequence identity to the sequence of SEQ ID NO: 39, and a second light chain comprising an amino acid sequence with at least 95% sequence identity to the sequence of SEQ ID NO: 40,
or
a first heavy chain comprising an amino acid sequence with at least 95% sequence identity to the sequence of SEQ ID NO: 35, a first light chain comprising an amino acid sequence with at least 95% sequence identity to the sequence of SEQ ID NO: 36, and
a second heavy chain comprising an amino acid sequence with at least 95% sequence identity to the sequence of SEQ ID NO: 41, and a second light chain comprising an amino acid sequence with at least 95% sequence identity to the sequence of SEQ ID NO: 42,
or
a first heavy chain comprising an amino acid sequence with at least 95% sequence identity to the sequence of SEQ ID NO: 37, a first light chain comprising an amino acid sequence with at least 95% sequence identity to the sequence of SEQ ID NO: 38, and
a second heavy chain comprising an amino acid sequence with at least 95% sequence identity to the sequence of SEQ ID NO: 41, and a second light chain comprising an amino acid sequence with at least 95% sequence identity to the sequence of SEQ ID NO: 42.

In another aspect, the invention relates to a bispecific antibody comprising a first antigen-binding domain that specifically binds to TfR and a second and a third antigen-binding domain that specifically binds to PD1, which comprises
a first heavy chain comprising an amino acid sequence with at least 95% sequence identity to the sequence of SEQ ID NO: 59, a second heavy chain comprising an amino acid sequence with at least 95% sequence identity to the sequence of SEQ ID NO: 60, a first light chain comprising an amino acid sequence with at least 95% sequence identity to the sequence of SEQ ID NO: 57, and a second light chain comprising an amino acid sequence with at least 95% sequence identity to the sequence of SEQ ID NO: 58,
or
a first heavy chain comprising an amino acid sequence with at least 95% sequence identity to the sequence of SEQ ID NO: 61, a second heavy chain comprising an amino acid sequence with at least 95% sequence identity to the sequence of SEQ ID NO: 60, a first light chain comprising an amino acid sequence with at least 95% sequence identity to the sequence of SEQ ID NO: 57, and a second light chain comprising an amino acid sequence with at least 95% sequence identity to the sequence of SEQ ID NO: 58.

According to another aspect of the invention, the bispecific antibody comprising a first antigen-binding domain that specifically binds to TfR and a second, and optionally a third, antigen-binding domain that specifically binds to PD1, is one wherein the antibody binds to both TfR and to PD1 with affinities in the nM to sub-nM ranges as determined by state-of-the art methods as described herein and known to the skilled person.

According to another aspect of the invention, the bispecific antibody comprising a first antigen-binding domain that specifically binds to TfR and a second, and optionally a third, antigen-binding domain that specifically binds to PD1 is characterized independently by one or more of the following properties: the anti-PD1 anti-TfR bispecific antibody
i) reduces PD1/PD-L1-mediated inhibition of TCR signaling by more than 2-fold at a concentration of 1 nM or by more than 4-fold at a concentration of 100 nM (as detected using a NFAT response element operably linked to a luciferase reporter system in a co-culture assay according to Example 4); and/or
ii) internalizes into activated T cells upon contacting with activated T cells by more than 25%, preferably by more than 40%, more preferable by more than 50% (in a internalization assay according to Example 6)
iii) enhances the Granzyme B secretion by allogenic stimulated T cells (in a minimal mixed lymphocyte reaction according to Example 14).

In one aspect, the bispecific antibody comprising a first antigen-binding domain that specifically binds to TfR and a second, and optionally a third, antigen-binding domain that specifically binds to PD1 is a multispecific antibody.

In one particular aspect, the invention provides a bispecific antibody comprising a first antigen-binding domain that specifically binds to TfR and a second, and optionally a third, antigen-binding domain that specifically binds to PD1, which comprises a first heavy chain of SEQ ID NO: 35, a first light chain of SEQ ID NO: 36, a second heavy chain of SEQ ID NO: 39 and a second light chain of SEQ ID NO: 40.

In one particular aspect, the invention provides a bispecific antibody comprising a first antigen-binding domain that specifically binds to TfR and a second, and optionally a third, antigen-binding domain that specifically binds to PD1, which comprises a first heavy chain of SEQ ID NO: 37, a first light chain of SEQ ID NO: 38, a second heavy chain of SEQ ID NO: 39 and a second light chain of SEQ ID NO: 40.

In one particular aspect, the invention provides a bispecific antibody comprising a first antigen-binding domain that specifically binds to TfR and a second, and optionally a third, antigen-binding domain that specifically binds to PD1, which comprises a first heavy chain of SEQ ID NO: 35, a first light chain of SEQ ID NO: 36, a second heavy chain of SEQ ID NO: 41 and a second light chain of SEQ ID NO: 42.

In one particular aspect, the invention provides a bispecific antibody comprising a first antigen-binding domain that specifically binds to TfR and a second, and optionally a third, antigen-binding domain that specifically binds to PD1, which comprises a first heavy chain of SEQ ID NO: 37, a first light chain of SEQ ID NO: 38, a second heavy chain of SEQ ID NO: 41 and a second light chain of SEQ ID NO: 42.

In one particular aspect, the invention provides a bispecific antibody comprising a first antigen-binding domain that specifically binds to TfR and a second, and optionally a third, antigen-binding domain that specifically binds to PD1, which comprises a first heavy chain of SEQ ID NO: 59 a second heavy chain of SEQ ID NO: 60, a first light chain of SEQ ID NO: 57, and a second light chain of SEQ ID NO: 58.

In one particular aspect, the invention provides a bispecific antibody comprising a first antigen-binding domain that specifically binds to TfR and a second, and optionally a third, antigen-binding domain that specifically binds to PD1, which comprises a first heavy chain of SEQ ID NO: 61 a second heavy chain of SEQ ID NO: 60, a first light chain of SEQ ID NO: 57 and a second light chain of SEQ ID NO: 58.

In another aspect, the invention provides an immunoconjugate comprising the bispecific antibody comprising a first antigen-binding domain that specifically binds to TfR and a second, and optionally a third, antigen-binding domain that specifically binds to PD1, and a cytotoxic agent. In a particular aspect, the cytotoxic agent is Pseudomonas Exotoxin A or an Amatoxin.

In one further aspect, the bispecific antibody is a multispecific antibody comprising
a) a full length bispecific antibody comprising a first antigen-binding domain that specifically binds to TfR and a second, and optionally a third, antigen-binding domain that specifically binds to PD1 and consisting of two antibody heavy chains and two antibody light chains, and
b) one, two, three or four single chain Fab fragments specifically binding to one to four further antigens (i.e. a third and/or fourth and/or fifth and/or a sixth antigen, preferably specifically binding to one further antigen, i.e. a third antigen),
wherein said single chain Fab fragments under b) are fused to said full length antibody under a) via a peptidic linker at the C- or N-terminus of the heavy or light chain of said full length antibody. In an additional aspect the multispecific antibody comprises exactly one (monovalent) antigen-binding domain that specifically binds to TfR and exactly two (monovalent) antigen-binding domains that specifically bind to PD1.

In one further aspect, the bispecific antibody is a multispecific antibody comprising
a) a full length bispecific antibody comprising a first antigen-binding domain that specifically binds to TfR and a second, and optionally a third, antigen-binding domain that specifically binds to PD1 and consisting of two antibody heavy chains and two antibody light chains, and
b) one, two, three or four single chain Fab fragments specifically binding to biotin,
wherein said single chain Fab fragments under b) are fused to said full length antibody under a) via a peptidic linker at the C- or N-terminus of the heavy or light chain of said full length antibody.

In one aspect, one or two identical single chain Fab fragments binding to a third antigen are fused to the full length antibody via a peptidic linker at the C terminus of the heavy or light chains of said full length antibody. In a preferred aspect, the third antigen is Biotin.

In one aspect, one or two identical single chain Fab fragments binding to a third antigen are fused to the full length antibody via a peptidic linker at the C terminus of the heavy chains of said full length antibody. In a preferred aspect, the third antigen is Biotin.

In one aspect, one or two identical single chain Fab fragments binding to a third antigen are fused to the full length antibody via a peptidic linker at the C terminus of the light chains of said full length antibody. In a preferred aspect, the third antigen is Biotin.

In one aspect, two identical single chain Fab fragments binding to a third antigen are fused to the full length antibody via a peptidic linker at the C-terminus of each heavy or light chain of said full length antibody. In a preferred aspect, the third antigen is Biotin.

In one aspect, two identical single chain Fab fragments binding to a third antigen are fused to the full length antibody via a peptidic linker at the C-terminus of each heavy chain of said full length antibody.

In one aspect, two identical single chain Fab fragments binding to a third antigen are fused to the full length antibody via a peptidic linker at the C-terminus of each light chain of said full length antibody.

In one particular aspect, the invention provides a trispecific antibody comprising a first antigen-binding domain that specifically binds to TfR, a second antigen-binding domain that specifically binds to PD1 and a third antigen-binding domain that specifically binds Biotin. In one particular aspect, the invention provides a trispecific antibody comprising a first antigen-binding domain that specifically binds to TfR, a second antigen-binding domain that specifically binds to PD1 and a third antigen-binding domain that specifically binds Biotin, which comprises a first heavy chain of SEQ ID NO: 47 and a first light chain of SEQ ID NO: 48, a second heavy chain of SEQ ID NO: 49 and a second light chain of SEQ ID NO: 50. In another aspect, the third antigen-binding domain of the trispecific antibody that specifically binds to biotin is used to bind a payload conjugated to Biotin to the trispecific antibody. In a particular aspect, the payload is a cytotoxic agent, preferably Pseudomonas Exotoxin A or an Amatoxin.

In one aspect, the invention provides an isolated nucleic acid encoding the bispecific antibody comprising a first antigen-binding domain that specifically binds to TfR and a second, and optionally a third, antigen-binding domain that specifically binds to PD1. The invention also provides an isolated nucleic acid encoding an immunoconjugate comprising a first antigen-binding domain that specifically binds to TfR and a second, and optionally a third, antigen-binding domain that specifically binds to PD1. In a further aspect, the invention provides a host cell comprising said nucleic acid.

In another aspect, the invention concerns a method of producing the bispecific antibody comprising a first antigen-binding domain that specifically binds to TfR and a second, and optionally a third, antigen-binding domain that specifically binds to PD1 or the immunoconjugate comprising said bispecific antibody, the method comprising the step of culturing a host cell comprising a nucleic acid encoding said bispecific antibody or said immunoconjugate under conditions suitable for the expression of the antibody. In a particular aspect, the method further comprises recovering the antibody from the host cell. In a further aspect, the invention also concerns a bispecific antibody produced by such a method.

In one aspect, the invention provides a pharmaceutical composition comprising a bispecific antibody comprising a first antigen-binding domain that specifically binds to TfR and a second, and optionally a third, antigen-binding domain that specifically binds to PD1 or the immunoconjugate comprising said bispecific antibody and a pharmaceutically acceptable carrier. In an additional aspect, the invention relates to a pharmaceutical composition comprising a first antigen-binding domain that specifically binds to TfR and a second, and optionally a third, antigen-binding domain that specifically binds to PD1 or the immunoconjugate comprising said bispecific antibody, a pharmaceutically acceptable carrier and an additional therapeutic agent.

In another aspect, the invention provides a bispecific antibody comprising a first antigen-binding domain that specifically binds to TfR and a second, and optionally a third, antigen-binding domain that specifically binds to PD1, the immunoconjugate comprising the bispecific antibody, or the pharmaceutical composition comprising the bispecific antibody for use as a medicament.

Also encompassed by the invention is a bispecific antibody comprising a first antigen-binding domain that specifically binds to TfR and a second, and optionally a third, antigen-binding domain that specifically binds to PD1, the immunoconjugate comprising the bispecific antibody, or the pharmaceutical composition comprising the bispecific antibody for use
i) in the modulation of immune responses, such as restoring T cell activity,
ii) in stimulating an immune response or function,
iii) in the prevention or treatment of cancer,
iv) in delaying progression of cancer,
v) in prolonging the survival of a patient suffering from cancer,
vi) acute infections,
vii) chronic and acute viral infections, and/or
viii) other conditions which depend on PD1 expression and PD1-mediated immune-modulation.

Immunoconjugates or trispecific antibodies carrying a cytotoxic payload are also useful
i) in treating graft-versus-host disease, and/or
ii) in the prevention or treatment of autoimmune diseases.

In another aspect, the invention provides a bispecific antibody comprising a first antigen-binding domain that specifically binds to TfR and a second, and optionally a third, antigen-binding domain that specifically binds to PD1, the immunoconjugate comprising the bispecific antibody, or the pharmaceutical composition comprising the bispecific antibody for use in the prevention or treatment of cancer wherein the bispecific antibody is administered in combination with a chemotherapeutic agent, radiation and/or other agents for use in cancer immunotherapy.

Furthermore, provided is a method of inhibiting the growth of tumor cells in a an individual comprising administering to the individual an effective amount of the bispecific antibody comprising a first antigen-binding domain that specifically binds to TfR and a second, and optionally a third, antigen-binding domain that specifically binds to PD1, the immunoconjugate comprising the bispecific antibody, or the pharmaceutical composition comprising the bispecific antibody to inhibit the growth of the tumor cells.

In a particular aspect, the invention concerns the use of the bispecific antibody comprising a first antigen-binding domain that specifically binds to TfR and a second, and optionally a third, antigen-binding domain that specifically binds to PD1, the immunoconjugate comprising the bispecific antibody, or the pharmaceutical composition comprising the bispecific antibody in the manufacture of a medicament for treatment of
i. cancer,
ii. infections or
iii. graft-versus-host disease.

Furthermore, the invention discloses the use of the bispecific antibody comprising a first antigen-binding domain that specifically binds to TfR and a second, and optionally a third, antigen-binding domain that specifically binds to PD1, the immunoconjugate comprising the bispecific antibody, or the pharmaceutical composition comprising the bispecific antibody in the manufacture of a medicament for
i) modulating an immune response, such as restoring T cell activity
ii) stimulating an immune response or function
iii) delaying progression of cancer, and/or
iv) prolonging the survival or a patient suffering from cancer.

In one aspect, the invention provides a method of treating an individual having graft-versus-host disease comprising administering to the individual an effective amount of the bispecific antibody comprising a first antigen-binding domain that specifically binds to TfR and a second, and optionally a third, antigen-binding domain that specifically binds to PD1, the immunoconjugate comprising the bispecific antibody, or the pharmaceutical composition comprising the bispecific antibody. In an additional aspect, provided is a method of treating an individual having graft-versus-host disease comprising administering to the individual an effective amount of the bispecific antibody comprising a first antigen-binding domain that specifically binds to TfR and a second, and optionally a third, antigen-binding domain that specifically binds to PD1, the immunoconjugate comprising the bispecific antibody, or the pharmaceutical composition comprising the bispecific antibody, further comprising administering an additional therapeutic agent to the individual. The additional therapeutic agent is preferably selected from the group consisting of chemotherapeutic agents, checkpoint inhibitors, radiation and/or other agents for use in cancer immunotherapy, such as immunocytokines, IL-2 and variants thereof, IL-7, IL-12, PD1-IL2v, costimulatory molecules, e.g. FAP-4-1BBL/OX40/CD40, TLR agonists, antibody drug conjugates (ADCs) and cytotoxic fusion proteins that can be used as potential ‘primers’ for immunotherapy and for “cold-to-hot” conversion of tumors.

Furthermore, provided is a method of inhibiting PD1 function in an individual comprising administering to the individual an effective amount of a bispecific antibody comprising a first antigen-binding domain that specifically binds to TfR and a second, and optionally a third, antigen-binding domain that specifically binds to PD1, the immunoconjugate comprising the bispecific antibody, or the pharmaceutical composition comprising the bispecific antibody to inhibit PD1 function. The individual is preferably a mammal, particularly a human.

### BRIEF DESCRIPTION OF THE FIGURES

FIG. 1: Schematic representation of exemplary configurations of the bispecific antibodies of the invention with a 1+1 stoichiometry of the binding domains specific for anti-PD1 and anti-TfR. The two different binding domains are distinguished by their patterns. For each configuration, two possible orientations of the charge variants (indicated by ++ or −−) that foster heterodimerization are shown, one where the charges are in the Fab (upper row) and one in the CrossFab (lower row). (A, F) Illustration of the “1+1 CrossMab VH-VL” molecule. (B, G) Illustration of the “one-armed 1+1 IgG CrossMab VH-VL” molecule with alternative order of CrossFab and Fab components. (C, H) Illustration of the “one-armed 1+1 IgG CrossMab VH-VL” molecule. (D, I) Illustration of the “Fab-CrossFab VH-VL” fusion molecule. (E, J) Illustration of the “CrossFab-Fab VH-VL” fusion molecule. Black dot: optional modification in the Fc domain promoting heterodimerization. ++, −−: amino acids of opposite charges optionally introduced in the CH1 and CL domains. CrossFab molecules are depicted as comprising an exchange of VH and VL regions, but may—in aspects wherein no charge modifications are introduced in CH1 and CL domains—alternatively comprise an exchange of the CH1 and CL domains.

FIG. 2: Schematic representation of exemplary configurations of the bispecific antibodies of the invention with a 2+1 stoichiometry of the binding domains specific for anti-PD1 and anti-TfR wherein one binding domain is attached to the N-terminus of the heavy chain of one Fab (“TCB-format”). The different binding domains are distinguished by their patterns. The binding domain which is present twice is the anti-PD1 binding domain. The binding domain which is present once is the TfR binding domain. For each configuration of the CrossMab, two possible orientations of the charge variants (indicated by ++ or −−) that foster heterodimerization are shown, one where the charges are in the Fab (upper row) and one in the CrossFab (lower row). (A, E) Illustration of the “2+1 IgG CrossMab VH-VL” molecule. (B, F) Illustration of the “2+1 IgG CrossMab VH-VL” molecule with two CrossFabs and one Fab which is fused via the C-terminus of its CH1 domain to the N-terminus of the VL domain of one of the CrossFabs. (C, G) Illustration of the “2+1 IgG CrossMab VH-VL” molecule with two CrossFabs and alternative order of CrossFab and Fab components (“inverted”). (D, H) Illustration of the “2+1 IgG CrossMab” molecule (“inverted”). Black dot: optional modification in the Fc domain promoting heterodimerization. ++, −−: amino acids of opposite charges optionally introduced in the CH1 and CL domains. CrossFab molecules are depicted as comprising an exchange of VH and VL regions, but may—in aspects wherein no charge modifications are introduced in CH1 and CL domains-alternatively comprise an exchange of the CH1 and CL domains.

FIG. 3: Schematic representation of exemplary configurations of the bispecific antibodies of the invention with a 2+1 stoichiometry of the binding domains specific for anti-PD1 and anti-TfR wherein one binding domain is attached to the C-terminus of one Fc heavy chain (“BBB-format”). The different binding domains are distinguished in this schematic by their patterns. The binding domain which is present twice is the anti-PD1 binding domain. The binding domain which is present once is the TfR binding domain. For each configuration, two possible orientations of the charge variants (indicated by ++ or −−) that foster heterodimerization are shown, one where the charges are in the Fab (upper row) and one in the CrossFab (lower row). (A, E) Illustration of the “(CrossFab)₂-Fc-Fab” molecule. (B, F) Illustration of the “(Fab)₂-Fc-CrossFab” molecule. (C, G) Illustration of the “(Fab+CrossFab)-Fc-Fab” molecule. (D, H) Illustration of the “(Fab+CrossFab)-Fc-CrossFab” molecule. Black dot: optional modification in the Fc domain promoting heterodimerization. ++, −−: amino acids of opposite charges optionally introduced in the CH1 and CL domains. CrossFab molecules are depicted as comprising an exchange of VH and VL regions, but may—in aspects wherein no charge modifications are introduced in CH1 and CL domains—alternatively comprise an exchange of the CH1 and CL domains.

FIG. 4: Schematic representation of exemplary configurations of the bispecific antibodies of the invention with a 2+1 stoichiometry of the binding domains specific for anti-PD1 and anti-TfR wherein the three Fab molecules are bound covalently to each other via peptide linkers as shown. The different binding domains are distinguished by their patterns. The binding domain which is present twice is the anti-PD1 binding domain. The binding domain which is present once is the TfR binding domain. For each configuration of the fusion molecule, two possible orientations of the charge variants (indicated by ++ or −−) that foster heterodimerization are shown, one where the charges are in the Fab (upper row) and one in the CrossFab (lower row). (A, E) Illustration of the “(Fab)₂-Crossfab” molecule. (B, F) Illustration of the “CrossFab-(Fab)₂” molecule. (C, G) Illustration of the “(CrossFab)₂-Fab”. (D, H) Illustration of the “Fab-(CrossFab)₂” molecule. ++, −−: amino acids of opposite charges optionally introduced in the CH1 and CL domains. CrossFab molecules are depicted as comprising an exchange of VH and VL regions, but may—in aspects wherein no charge modifications are introduced in CH1 and CL domains—alternatively comprise an exchange of the CH1 and CL domains.

FIG. 5A: Schematic representation of a bispecific 1+1 CrossMab with binding domains for TfR and PD1.

FIG. 5B: Schematic representation of a bispecific CrossMab with a third binding domain binding specifically to biotin for payload delivery to active immune cells.

FIG. 6: Schematic illustration of the 2+1 antibodies (Blood brain barrier shuttle (BBB)-format) used in the Examples. The tested 2+1 format antibody molecules 8156 and 8158 were produced as “2+1 IgG CrossMab VH-VL, inverted” with charge modifications (i.e. VH/VL exchange in PD1 binders, charge modifications in TfR binder: EE=147E, 213E; RK=123R, 124K). (B-E) Components for the assembly of the antibody: light chain of anti-PD1 crossover Fab domain (A), light chain of anti-TfR Fab domain with charge modifications in CL (B), heavy chain of anti-PD1 crossover with hole and PG LALA mutations in Fc region and with the N-terminus of the heavy chain of the anti-TfR Fab molecule C-terminally attached to the Fc region (H), heavy chain with anti-PD1 crossover Fab and with knob and PG LALA mutations in Fc region (K). For control molecule 8158, the variable antibody regions of the light and heavy chain of the TfR-binding arm were replaced by a non-binding sequence (“Nada”).

FIG. 7: Schematic illustration of the 2+1 antibodies (T cell bispecific antibody (TCB)-format) used in the Examples. The tested antibody molecules 8157 and 8159 were produced as “2+1 (CrossFab)₂-Fc-Fab VH-VL” with charge modifications (i.e. VH/VL exchange in PD1 binders, charge modifications in TfR binder: EE=147E, 213E; RK=123R, 124K). (B-E). Components for the assembly of the antibody: light chain of anti-PD1 crossover Fab molecule (A), light chain of anti-TfR Fab molecule with charge modifications in CL (B), heavy chain of anti-TfR molecule with hole and PG LALA mutations in Fc region and with the C-terminus of the heavy chain of the anti-PD1 crossover Fab molecule attached to the N-terminus of the anti-PD1 Fab (H), heavy chain of anti-PD1 crossover with knob and PG LALA mutations in Fc region (K). For control molecule 8159, the variable antibody regions of the light and heavy chain of the TfR-binding arm were replaced by a non-binding sequence (“Nada”).

FIG. 8: Blocking of PD1/PD-L1 signaling in a co-culture assay.

FIG. 8A: PD1-expressing Jurkat-PD1-NFAT cells were pre-incubated with antibodies for 30 min at 37° C., washed with media once and then added to the activator cells (PD-L1-expressing CHO-K1 cells adhered overnight) for 5 h. Inhibition of the TCR activation by PD1 signaling was measured by the luminescent signal after addition of Bio-Glo™ Luciferase Assay Substrate (representative for 3 independent experiments).

FIG. 8B: Cell viability during the assay following addition of antibodies. The cell viability in the co-culture assay was not affected by the addition of any antibody at the concentrations applied.

FIG. 9: SPR curves of the trispecific anti-PD1 anti-TfR anti-biotin CrossMab molecule (1129) and the anti-PD1 anti-Nada anti-biotin control molecule (9904).

FIG. 10: Avidity-enhanced binding of a trispecific anti-PD1 anti-TfR anti-biotin CrossMab depends on PD1 expression.

FIG. 10A: Expression levels of TfR and PD1 on PD1-transduced NFAT-bla Jurkat cells analyzed by flow cytometry.

FIG. 10B: Quantification of PE-labelled antibodies bound to the surface of PD1-transduced NFAT-bla Jurkat cells (n=3±standard error of the mean (SEM)).

FIG. 10C: Binding of CrossMab to PD1-transduced NFAT-bla Jurkat cells detected by bio-Cy5 (representative for three independent experiments). Tri-specific anti-PD1 anti-TfR anti-biotin CrossMab 1129 and controls were incubated with Jurkat cells expressing different levels of PD1 on its surface (wildtype WT, PD1 low, PD1 high). Antibodies were detected using biotinylated Cy5 and detected via flow cytometry measuring Median APC. Binding was stronger on cells that expressed a higher level of PD1 on their cell surface.

FIG. 11: Internalization of anti-PD1 anti-TfR CrossMab by activated T cells.

FIG. 11A and B: Anti-PD1 anti-TfR bispecific antibodies 8012, 8013, 8017 and 8018 show internalization, similar to the TfR Nada control antibodies (8015, 8016). Antibodies carrying only PD1 binding domains, but no TfR binding domains, do not show internalization (PD1-0103-0312, 8014, 8019).

FIG. 12: Internalization and co-localization of mEGFP-PD1 and Bio-Cy5 payload in mEGFP-PD1 transduced Jurkat cells. mEGFP-PD1 Jurkats were incubated with 10 nM anti-TfR/anti-PD1/anti-Biotin trispecific CrossMab or control antibodies complexed with Bio-Cy5 for 3 h. The localization of GFP-PD1 and Bio-Cy5 was assessed by confocal microscopy.

FIG. 13: Internalization of mEGFP-PD1 into transduced Jurkat cells. mEGFP-PD1-transduced Jurkat cells were incubated with 10 nM Pembrolizumab (bivalent anti PD1-antibody), anti-TfR/anti-PD1 bispecific antibody or anti-CD33 non-binding control antibody for 60 min. The localization of mEGFP-PD1 was assessed by confocal microscopy.

FIGS. 14A and B: Antibody-mediated decrease and recovery of transduced mEGFP-PD1 in Jurkat cells.

FIG. 14A: mEGFP-PD1 transduced Jurkat cells were treated with 10 nM trispecific antibody or control molecules, and assessed for their GFP median fluorescence after 1, 3, 24 and 48 h.

FIG. 14B: Cells were treated with 10 nM trispecific antibodies for 24 h to achieve maximal GFP-PD1 downregulation and GFP signal was monitored over 24 h.

FIGS. 15A and B: Avidity-enhanced delivery of biotinylated pseudomonas exotoxin PE25.

FIG. 15A: Viability of PD1-transduced NFAT-bla Jurkat cells treated with trispecific antibody complexed with bio-PE25 or toxin-only control for 48 h measured by CellTiter-Glo® assay.

FIG. 15B: Viability of PD1-transduced NFAT-bla Jurkat cells after treatment with control antibodies carrying no bio-PE25 for 48 h.

FIGS. 16A and B: Avidity-enhanced binding and internalization in activated human T cells.

FIG. 16A: Binding of trispecific antibody to activated T cells (anti-CD3/CD28) detected by biotinylated Cy5. PD1 was quantified to ˜8000 molecules per cell, TfR to ˜200 000 molecules per cell. The trispecific antibody was detected by biotinylated Cy5 at concentrations at which the control antibodies showed only very little binding to activated T cells.

FIG. 16B: Relative fluorescence (t=1 h versus t=0 h) of anti-IgG on the cell surface to biotinylated Cy5 data from two donors±standard deviation. Through post-staining of IgG at time point 0 and 1 h, internalization was observed for the anti-TfR containing antibodies, but not for the Nada/anti-PD1 antibody.

FIG. 17: Co-expression of PD1 and TfR on T cells in a model of Graft-versus-Host disease (GvHD) and avidity enhanced killing of host-infiltrated T cells

FIG. 17A: Mice engrafted with human PBMCs usually develop and die of GvHD in due course.

FIG. 17B: Cells from mouse spleens were analyzed by flow cytometry and gated on single human CD3 cells. Infiltrated human CD4 and CD8 positive cells were analyzed for TfR and PD1 expression. More than 70% of the human T cells (including CD4 and CD8 T cells) detected amongst the murine spleenocytes were double-positive for TfR and PD1.

FIG. 17C: Treatment of the spleenocytes with the anti-PD1/TfR antibody complexed with PE25 showed a 10-1000 fold reduction in dose to reduce the number of human T cells in this pool of cells.

FIGS. 18A and 18B: Comparative representation of different 2+1 formats of a bispecific anti-TfR/anti-PD1 antibody with bivalent binding to PD1, and of various control constructs, used in the examples. From left to right they correspond to (A) molecules 8157, 8156, (B) PD1-0103-0312, 8159 and 8158. “N” denotes an anti-Nada binding domain.

FIG. 18A: The first 2+1 format (anti-TfR binding domain between anti-PD1 binding domain and hinge region; “TCB-format”; on the left) contains one PD1-binding entity as a regular Fab arm in IgG configuration and a second PD1-binding Fab arm on ‘top’ (i.e. N-terminally) of a TfR-binding CrossFab which precedes the hinge of the other side of an knob-into-hole heterodimer. The second 2+1 format (anti-TfR binding domain c-terminally fused to CH3 of the Fc domain; “BBB-format”; on the right) contains as PD1-binding arms regular Fab arms of an IgG, with a TfR-binding Fab attached as CrossFab format to the C-terminus of the asymmetrical (knob-into-hole) CH3 domain.

FIG. 18B: On the left a “classical” bivalent, blocking anti-PD1 antibody (anti-PD1-IgG) is shown. In order to compare the impact of TfR binding and internalization to anti-PD1 antibodies, two controls were constructed wherein TfR was replaced with a non-antigen-binding Fab fragments (anti Nada binding domain between anti-PD1 binding domain and hinge region and anti-Nada binding domain c-terminally fused to CH3 of the Fc domain).

FIG. 19: Internalization of two bispecific antibodies binding to PD1 in a bivalent and to TfR in a monovalent manner, 8156 and 8157 (different 2+1 formats) in comparison to an anti-PD1 antibody and the two PD1 Nada control antibodies. The control molecules that bind PD1 in a bivalent manner yet do not contain a TfR binder (anti-PD1, 8158, 8159) showed rather poor internalization, while the two 2+1 bispecific antibody formats that bind PD1 in a bivalent and TfR in a monovalent manner (8157, 8156) showed strikingly increased internalization rates

FIGS. 20A and 20B: Effect of tested antibodies on cytotoxic Granzyme B release by human CD4 T cells co-cultured with allogeneic mature dendritic cells (Mixed lymphocyte reaction). The EC₅₀ values reached by anti-PD1 anti-Tfr bispecific antibodies 8012 and 8013 are comparable to those achieved with the bivalent PD1-0103-0312 binder.

Monovalent anti-PD1 construct PD1-0103-0312/Nada (8014) and Pembrolizumab/Nada (8019) led only to modest Granzyme B secretion, and monovalent TfR binding by the binders Nada/51A165 (8015) and Nada/1026 (8016) did not induce any Granzyme B secretion at all.

FIG. 21: Effect of tested antibodies on cytotoxic Granzyme B release by human CD4 T cells co-cultured with allogeneic mature dendritic cells (Mixed lymphocyte reaction). The TCB and BBB formats (8156 & 8157) showed lower EC₅₀ values than the bivalent parental anti-PD1 antibody and also compared to the respective controls (8158 & 8159), resulting in an increased Granzyme B secretion and therefore increased T cell effector functions induced by of these formats.

FIGS. 22A, 22B and 22C: Effect of tested antibodies on cytotoxic Granzyme B release by human CD4 T cells co-cultured with allogeneic mature dendritic cells (Mixed lymphocyte reaction).

FIG. 22A: The EC₅₀ values reached by monovalent anti-PD1 anti-Tfr bispecific antibodies 8012 and 8013 were comparable to those achieved with the bivalent PD1-0103-0312 binder. Monovalent anti-PD1 construct PD1-0103-0312/Nada (8014) on the other hand led only to modest Granzyme B secretion.

FIG. 22B: The EC₅₀ values reached by monovalent anti-PD1 anti-Tfr bispecific antibodies 8017 and 8018 were comparable to those achieved with Pembrolizumab. Monovalent anti-PD1 construct Pembrolizumab/Nada (8019) on the other hand led only to modest Granzyme B secretion.

FIG. 22C The EC₅₀ values reached by bivalent anti-PD1 anti-Tfr bispecific antibodies 8157 (TCB-format) and 8156 (BBB-format) were lower than those achieved with Pembrolizumab or the bivalent PD1-0103-0312 binder. Bivalent anti-PD 1 constructs PD1-0103-0312/Nada TCB-format (8159) and BBB-format (8158) on the other hand led only to modest Granzyme B secretion.

FIG. 23 The internalization of anti-PD1 anti-TfR murinized molecules was tested using the BA/F3 cell line (RNCB Accession ID: CL003201) which expresses murine TfR on its cell surface. Both molecules containing a TfR binding domain, 6768 (mTfR-001/huPD1-478 TCB format) and 6794 (mTfR-001/Nada TCB format), showed good internalization rates of approximately 70% after three hours, while huPD1/Nada (P1AG6769) shows no internalization.

FIG. 24 Blocking of PD1/PD-L1 mediated signaling of anti-PD1 anti-TfR murinized molecules was tested in a co-culture assay. Molecules 6768 (mTfR-001/huPD1-478, TCB format) and 6769 (Nada/huPD1-478, TCB format) like anti PD1-antibody PD1-0103-0312 contain bivalent anti-PD1 binding domains. All these molecules demonstrated a comparable functionality in terms of blocking the PD1-PDL1 signaling pathway. Control molecule 6794 (mTfR-001/Nada, TCB format), which does not contain an anti-PD1 antigen-binding domain, did not show any blocking functionality.

### DETAILED DESCRIPTION

In one aspect, the invention is based, in part, on the finding that the selected anti-TfR anti-PD1 bispecific antibodies of the invention bind to TfR and to PD1, and have the ability to internalize into cells that express and display TfR and PD1 on their surface.

In a further aspect, the invention is based, in part, on the finding that an anti-TfR anti-PD1 2+1 format antibody, i.e. a bispecific antibody comprising a first antigen-binding domain that specifically binds to TfR and a second and a third antigen-binding domain that specifically bind to PD1, shows improved biological activity and leads to better inhibition of the interaction between PD1 and PD-L1 than a monospecific, bivalent PD1 antibody. In one aspect these molecules comprise IgG class Fab fragments, and optionally also IgG class Fc regions, that are covalently bound to each other, resulting in 2+1 format antibodies of different conformations described herein.

It has been found that contacting cells expressing PD1 and TfR with the anti-TfR anti-PD1 bispecific antibodies of the invention leads to a depletion of PD1 from the surface of these cells, particularly of T cells, and thus prevents PD-L1 from binding to the PD1 receptor on the surface of those cells. The anti-TfR anti-PD1 bispecific antibodies inhibit PD1/PD-L1 mediated T cell receptor signaling, e.g. they increase immune-modulating cytokines (e.g. interferon gamma and Granzyme B release/secretion). The inhibition that is achieved through the depletion of PD-1 from the cell surface is more effective and/or more permanent than what can be achieved by the transient binding of an anti-PD1 blocking antibody. Other immune-modulating cytokines which may be increased by using the antibodies of the invention are e.g. tumor necrosis factor alpha (TNF alpha) secretion and IL-12. As used herein, the terms interferon-gamma (IFN-gamma), tumor necrosis factor alpha (TNF alpha), IL-12 etc. refer to the human cytokines.

### I. Definitions

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some aspects, the number of amino acid changes are 10 or less, 9 or less, 8 or less, 7 or less, 6 or less, 5 or less, 4 or less, 3 or less, or 2 or less. In some aspects, the VL acceptor human framework is identical in sequence to the VL human immunoglobulin framework sequence or human consensus framework sequence.

“Affinity” refers to the strength of the sum total of noncovalent interactions between a single binding domain of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured by common methods known in the art, including those described herein. Specific illustrative and exemplary methods for measuring binding affinity are described in the following.

An “affinity matured” antibody refers to an antibody with one or more alterations in one or more complementary determining regions (CDRs), compared to a parent antibody which does not possess such alterations, such alterations resulting in an improvement in the affinity of the antibody for antigen.

The terms “anti-TfR antibody” and “an antibody or antigen-binding domain that specifically binds to TfR” refer to an antibody or antigen-binding domain that is capable of binding TfR with sufficient affinity such that the antibody or the antigen-binding domain is useful as a diagnostic and/or therapeutic agent in targeting TfR. In one aspect, the extent of binding of an anti-TfR antibody or an antibody or antigen-binding domain that specifically binds to TfR to an unrelated, non-TfR protein is less than about 10% of the binding of anti-TfR antibody or the antigen-binding domain to TfR as measured, e.g., by surface plasmon resonance (SPR). In certain aspects, an antibody comprising an antigen-binding domain that binds to TfR has a dissociation constant (KD) of ≤1 μM, ≤100 nM, ≤10 nM, ≤1 nM, ≤0.1 nM, ≤0.01 nM, or ≤0.001 nM (e.g., 10−8 M or less, e.g., from 10⁻⁸ M to 10⁻¹³ M, e.g., from 10⁻⁹ M to 10⁻¹³ M). An antibody or an antigen-binding domain is said to “specifically bind” to TfR when the antibody has a K_{D} of 1 μM or less. In certain aspects, an anti-TfR antibody binds to an epitope of TfR that is conserved among TfR from different species.

The term “anti-PD1 antibody” and “an antibody or antigen-binding domain that specifically binds to PD1” refer to an antibody or antigen-binding domain that is capable of binding PD1 with sufficient affinity such that the antibody or the antigen-binding domain is useful as a diagnostic and/or therapeutic agent in targeting PD1. In one aspect, the extent of binding of an antibody or an antigen-binding domain that specifically binds to PD1 to an unrelated, non-PD1 protein is less than about 10% of the binding of the antibody or antigen-binding domain to PD1 as measured, e.g., by surface plasmon resonance (SPR). In certain aspects, an antibody or an antigen-binding domain that binds to PD1 has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M). An antibody or an antigen-binding domain is said to “specifically bind” to PD1 when the antibody has a K_{D} of 1 μM or less. In certain aspects, an anti-PD1 antibody or an antibody or antigen-binding domain that specifically binds to PD1 binds to an epitope of PD1 that is conserved among PD1 from different species. The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity. Various patent applications disclose production of anti-PD1 antibodies and/or methods of enhancing immune responses with an agent (including an anti-PD1 antibody) that interferes with PD-L1 binding and/or PD1 signaling, including the following: US2003/0039653, US2004/0213795, US2006/0110383, US2007/0065427, US2007/0122378, US2012/237522, WO2004/072286, WO2006/121168, WO2006/133396, WO2007/005874, WO2008/083174, WO2008/156712, WO2009/024531, WO2009/014708, WO2009/114335, WO2010/027828, WO2010/027423, WO2010/036959, WO2010/029435, WO2010/029434, WO2010/063011, WO2010/089411, WO2011/066342, WO2011/110604, WO2011/110621, and WO2012/145493.

A “blocking” antibody or an “antagonist” antibody is one that inhibits or reduces a biological activity of the antigen it binds. In some embodiments, blocking antibodies or antagonist antibodies substantially or completely inhibit the biological activity of the antigen. For example, the bispecific antibodies of the invention block the signaling through PD1 and PD-L1 to restore a functional response by T cells (e.g., proliferation, cytokine production, target cell killing) from a dysfunctional state to antigen stimulation.

The term “monospecific” antibody as used herein denotes an antibody that has one or more binding domains, each of which bind to the same epitope of the same antigen. The term “bispecific” means that the antibody is able to specifically bind to at least two distinct antigenic determinants, for example via two binding domains each formed by a pair of an antibody heavy chain variable domain (VH) and an antibody light chain variable domain (VL) binding to different antigens or to different epitopes on the same antigen. Herein, such a bispecific antibody is also called a 1+1 format or 1+1 format antibody. Other bispecific antibody formats are called 2+1 formats or 2+1 format antibodies (comprising two binding domains for a first antigen or epitope and one binding domain for a second antigen or epitope) or 2+2 formats or 2+2 format antibodies (comprising two binding domains for a first antigen or epitope and two binding domains for a second antigen or epitope) herein.

The term “a bispecific antibody comprising a first antigen-binding domain that specifically binds to TfR and a second, and optionally a third, antigen-binding domain that specifically binds to PD1”, “a bispecific antibody that specifically binds TfR and PD1”, “bispecific antigen-binding molecule specific for TfR and PD1” and “anti-TfR anti-PD1 bispecific antibody” are used interchangeably herein and refer to a bispecific antibody that is capable of binding TfR and PD1 with sufficient affinity such that the antibody is useful as a diagnostic and/or therapeutic agent in targeting TfR and PD1.

The term “valent” as used within the current application denotes the presence of a specified number of binding domains in an antigen-binding molecule. As such, the terms “bivalent”, “tetravalent”, and “hexavalent” denote the presence of two binding domains, four binding domains, and six binding domains, respectively, in an antigen-binding molecule. The bispecific antibodies according to the invention are at least “bivalent” and may be “trivalent” or “multivalent” (e.g. “tetravalent” or “hexavalent”). In a particular aspect, the antibodies of the present invention have two or more binding domains and are bispecific. That is, the antibodies may be bispecific even in cases where there are more than two binding domains (i.e. that the antibody is trivalent or multivalent). In particular, the invention relates to bispecific bivalent and trivalent antibodies, having one or two binding domains for each antigen they specifically bind.

The terms “full length antibody”, “intact antibody”, and “whole antibody” are used herein interchangeably to refer to an antibody having a structure substantially similar to a native antibody structure. “Native antibodies” refer to naturally occurring immunoglobulin molecules with varying structures. For example, native IgG-class antibodies are heterotetrameric glycoproteins of about 150,000 Da (Daltons), composed of two light chains and two heavy chains that are disulfide-bonded. The individual polypeptide chains that the antibodies, antibody fragments and antibody-like molecules disclosed herein are formed of are sometimes also referred to herein as “subunits”, e.g. the subunits of a Fab fragment or the subunits of an Fc domain. From N- to C-terminus, each heavy chain has a variable region (VH), also called a variable heavy domain or a heavy chain variable domain, followed by three constant domains (CH1, CH2, and CH3), also called a heavy chain constant region. Similarly, from N- to C-terminus, each light chain has a variable region (VL), also called a variable light domain or a light chain variable domain, followed by a light chain constant domain (CL), also called a light chain constant region. The heavy chain of an antibody may be assigned to one of five types, called α (IgA), δ (IgD), ε (IgE), γ (IgG), or μ (IgM), some of which may be further divided into subtypes, e.g. γ1 (IgG1), γ2 (IgG2), γ3 (IgG3), γ4 (IgG4), α1 (IgA1) and α2 (IgA2). The light chain of an antibody may be assigned to one of two types, called kappa (κ) and lambda (λ), based on the amino acid sequence of its constant domain.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2; diabodies; linear antibodies; single-chain antibody molecules (e.g., scFv, and scFab); single domain antibodies (dAbs); and multispecific antibodies formed from antibody fragments. For a review of certain antibody fragments, see Holliger and Hudson, Nature Biotechnology 23:1126-1136 (2005). For a review of scFv fragments, see e.g. Plückthun, in The Pharmacology of Monoclonal Antibodies, vol. 113, Rosenburg and Moore eds., Springer-Verlag, New York, pp. 269-315 (1994); see also WO 93/16185; and U.S. Pat. Nos. 5,571,894 and 5,587,458. For discussion of Fab and F(ab′)2 fragments comprising salvage receptor binding epitope residues and having increased in vivo half-life, see U.S. Pat. No. 5,869,046. Diabodies are antibody fragments with two antigen-binding domains that may be bivalent or bispecific, see, for example, EP 404,097; WO 1993/01161; Hudson et al., Nat Med 9, 129-134 (2003); and Hollinger et al., Proc Natl Acad Sci USA 90, 6444-6448 (1993). Triabodies and tetrabodies are also described in Hudson et al., Nat Med 9, 129-134 (2003). Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, MA; see e.g. U.S. Pat. No. 6,248,516 B1). In addition, antibody fragments comprise single chain polypeptides having the characteristics of a VH domain, namely being able to assemble together with a VL domain, or of a VL domain, namely being able to assemble together with a VH domain to a functional antigen-binding domain and thereby providing the antigen-binding property of full-length antibodies. Antibody fragments can be made by various techniques, including but not limited to proteolytic digestion of an intact antibody as well as production by recombinant host cells (e.g. E. coli or phage), as described herein.

Papain digestion of intact antibodies produces two identical antigen-binding fragments, called “Fab” fragments containing each the heavy- and light-chain variable domains and also the constant domain of the light chain and the first constant domain (CH1) of the heavy chain. As used herein, thus, the term “Fab fragment” refers to an antibody fragment comprising a light chain fragment comprising a VL domain and a constant domain of a light chain (CL) and a VH domain and a first constant domain (CH1) of a heavy chain, and includes the three CDRs in the VH and the three CDRs in the VL. The terms “Fab”, “Fab fragment”, “Fab molecule”, and “Fab domain” are used herein interchangeably to refer to an antibody fragment containing a heavy- and a light-chain variable domain and a constant domain of a light chain and the first constant domain (CH1) of a heavy chain.

Fab′ fragments differ from Fab fragments by the addition of a few residues at the carboxy terminus of the heavy chain CH1 domain including one or more cysteines from the antibody hinge region. Fab′-SH are Fab′ fragments wherein the cysteine residue(s) of the constant domains bear a free thiol group. Pepsin treatment yields an F(ab′)2 fragment that has two antigen-combining sites (two Fab fragments) and a part of the Fc region.

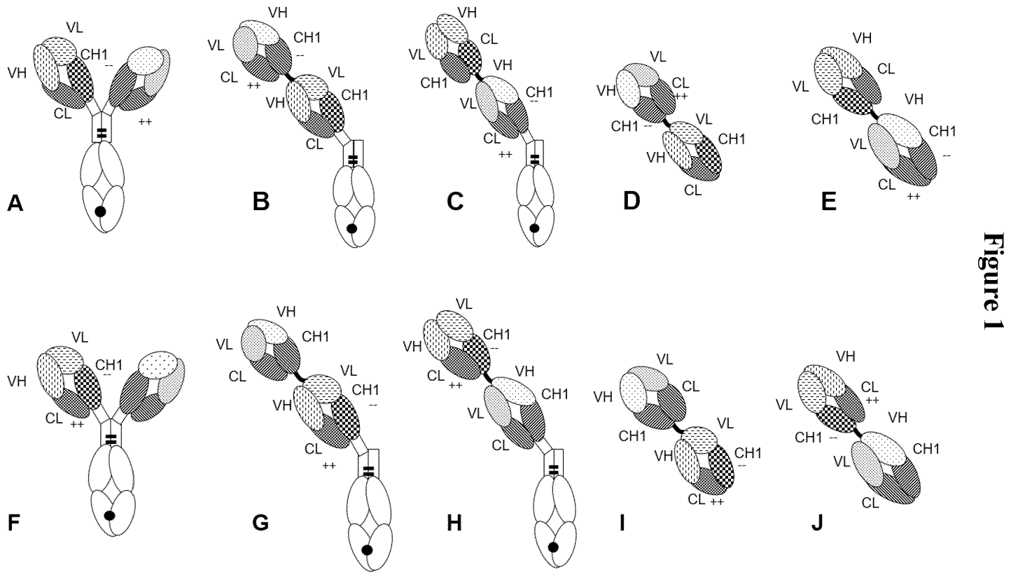

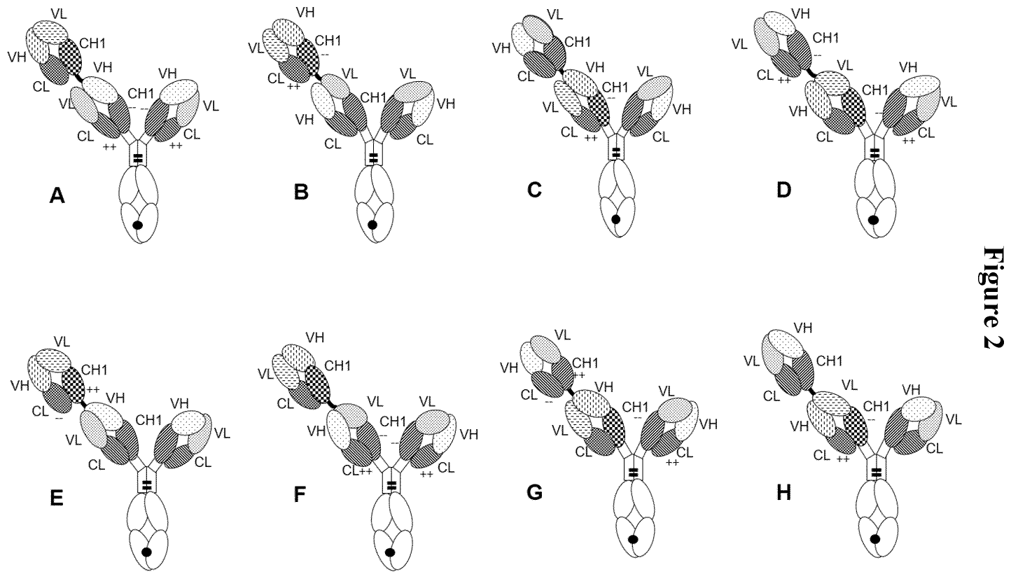

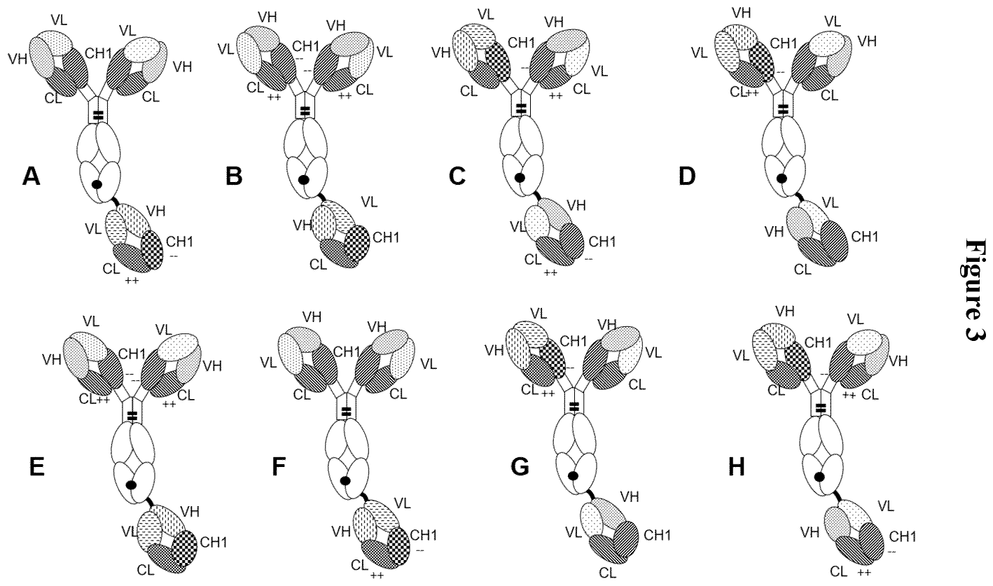

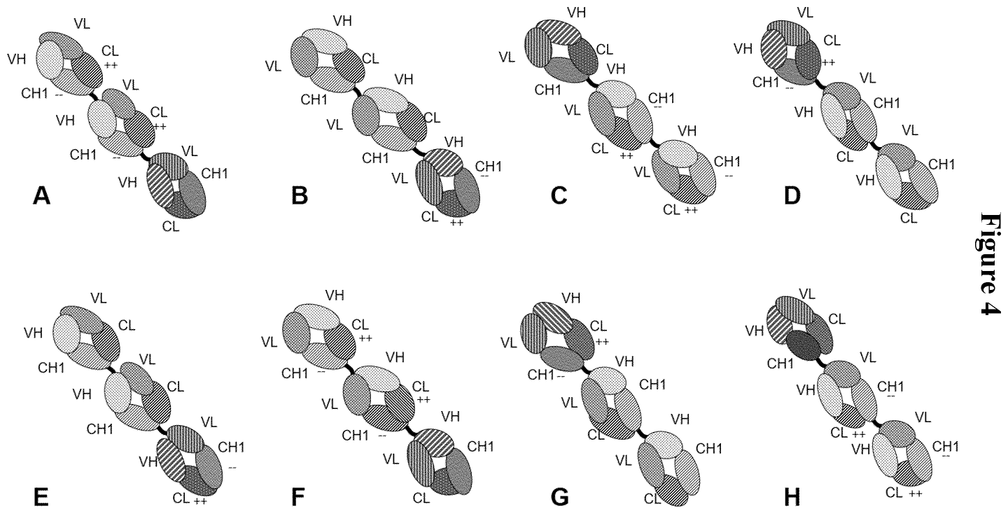

The term “CrossFab” or “Cross-Fab fragment” or “xFab fragment” or “crossover Fab fragment” refers to a Fab fragment, wherein either the variable regions or the constant regions of the heavy and light chain are exchanged. With regard to these Fab fragment variants and to the Fab fragments discussed in the following paragraphs, the terms “fragment”, “molecule” and “domain” are also used interchangeably herein. Two different chain compositions of a crossover Fab fragment are possible and comprised in the bispecific antibodies of the invention: On the one hand, the variable regions of the Fab heavy and light chain are exchanged, i.e. the crossover Fab fragment comprises a peptide chain composed of the light chain variable region (VL) and the heavy chain constant region (CH1), and a peptide chain composed of the heavy chain variable region (VH) and the light chain constant region (CL). This crossover Fab fragment is also referred to as CrossFab (VLVH). On the other hand, when the constant regions of the Fab heavy and light chain are exchanged, the crossover Fab fragment comprises a peptide chain composed of the heavy chain variable region (VH) and the light chain constant region (CL), and a peptide chain composed of the light chain variable region (VL) and the heavy chain constant region (CH1). This crossover Fab fragment is also referred to as CrossFab (CLCH1). To further reduce mispairing of heavy and light chains from different Fab molecules and thus increase the purity and yield of the desired (bispecific) antibody, charged amino acids with opposite charges may be introduced at specific amino acid positions in the CH1 and CL domains of either the Fab molecule binding to the first antigen (TfR), or the Fab molecule(s) binding to the second antigen (PD1), as further described herein. Charge modifications are made either in the conventional Fab molecule(s) comprised in the (bispecific) antibody (such as shown e.g. in FIG. 1 A-E, FIG. 2A-D, FIG. 3A-D, FIG. 4A-D), or in the VH/VL crossover Fab molecule(s) comprised in the (bispecific) antibody (such as shown e.g. in FIG. 1 F-J FIG. 2 E-H, FIG. 3 E-H, FIG. 4 E-H) (but not in both).

A “single chain Fab fragment” or “scFab” is a polypeptide consisting of an antibody heavy chain variable domain (VH), an antibody constant domain 1 (CH1), an antibody light chain variable domain (VL), an antibody light chain constant domain (CL) and a linker, wherein said antibody domains and said linker have one of the following orders in N-terminal to C-terminal direction: a) VH-CH1-linker-VL-CL, b) VL-CL-linker-VH-CH1, c) VH-CL-linker-VL-CH1 or d) VL-CH1-linker-VH-CL; and wherein said linker is a polypeptide of at least 30 amino acids, preferably between 32 and 50 amino acids. Said single chain Fab fragments are stabilized via the natural disulfide bond between the CL domain and the CH1 domain. In addition, these single chain Fab molecules might be further stabilized by generation of interchain disulfide bonds via insertion of cysteine residues (e.g. position 44 in the variable heavy chain and position 100 in the variable light chain according to Kabat numbering).

A “crossover single chain Fab fragment” or “x-scFab” is a is a polypeptide consisting of an antibody heavy chain variable domain (VH), an antibody constant domain 1 (CH1), an antibody light chain variable domain (VL), an antibody light chain constant domain (CL) and a linker, wherein said antibody domains and said linker have one of the following orders in N-terminal to C-terminal direction: a) VH-CL-linker-VL-CH1 and b) VL-CH1-linker-VH-CL; wherein VH and VL form together an antigen-binding domain which binds specifically to an antigen and wherein said linker is a polypeptide of at least 30 amino acids. In addition, these x-scFab molecules might be further stabilized by generation of interchain disulfide bonds via insertion of cysteine residues (e.g. position 44 in the variable heavy chain and position 100 in the variable light chain according to Kabat numbering).

A “single-chain variable fragment (scFv)” is a fusion protein of the variable regions of the heavy (VH) and light chains (VL) of an antibody, connected with a short linker peptide of ten to about 25 amino acids. The linker is usually rich in glycine for flexibility, as well as serine or threonine for solubility, and can either connect the N-terminus of the VH with the C-terminus of the VL, or vice versa. This protein retains the specificity of the original antibody, despite removal of the constant regions and the introduction of the linker. scFv antibodies are e.g. described in Houston, J. S., Methods in Enzymol. 203 (1991) 46-96). In addition, antibody fragments comprise single chain polypeptides having the characteristics of a VH domain, namely being able to assemble together with a VL domain, or of a VL domain, namely being able to assemble together with a VH domain to a functional antigen-binding domain and thereby providing the antigen-binding property of full-length antibodies.

“Scaffold antigen-binding proteins” are known in the art, for example, fibronectin and designed ankyrin repeat proteins (DARPins) have been used as alternative scaffolds for antigen-binding domains, see, e.g., Gebauer and Skerra, Engineered protein scaffolds as next-generation antibody therapeutics. Curr Opin Chem Biol 13:245-255 (2009) and Stumpp et al., Darpins: A new generation of protein therapeutics. Drug Discovery Today 13:695-701 (2008). In one aspect of the invention, a scaffold antigen-binding protein is selected from the group consisting of CTLA-4 (Evibody), Lipocalins (Anticalin), a Protein A-derived molecule such as Z-domain of Protein A (Affibody), an A-domain (Avimer/Maxibody), a serum transferrin (trans-body); a designed ankyrin repeat protein (DARPin), a variable domain of antibody light chain or heavy chain (single-domain antibody, sdAb), a variable domain of antibody heavy chain (nanobody, aVH), VNAR fragments, a fibronectin (AdNectin), a C-type lectin domain (Tetranectin); a variable domain of a new antigen receptor beta-lactamase (VNAR fragments), a human gamma-crystallin or ubiquitin (Affilin molecules); a kunitz type domain of human protease inhibitors, microbodies such as the proteins from the knottin family, peptide aptamers and fibronectin (adnectin).

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. In certain aspects, the antibody is of the IgG class. IgG class antibodies and also IgG like antibody molecules are generally easy to manufacture and purify in large quantities, and they frequently have pharmacological properties similar to those of a conventional IgG1. In certain aspects, the antibody is of the IgG₁ isotype. In certain aspects, the antibody is of the IgG₁ isotype with the P329G, L234A and L235A mutation to reduce Fc-region effector function. In other aspects, the antibody is of the IgG2 isotype. In certain aspects, the antibody is of the IgG4 isotype with the S228P mutation in the hinge region to improve stability of IgG4 antibody. The heavy chain constant domains that correspond to the different classes of immunoglobulins are called α, δ, ε, γ, and μ, respectively. The light chain of an antibody may be assigned to one of two types, called kappa (κ) and lambda (λ), based on the amino acid sequence of its constant domain.

The terms “constant region derived from human origin” or “human constant region” as used in the current application denotes a constant heavy chain region of a human antibody of the subclass IgG1, IgG2, IgG3, or IgG4 and/or a constant light chain kappa or lambda region. Such constant regions are well known in the state of the art and e.g. described by Kabat, E. A., et al., Sequences of Proteins of Immunological Interest, 5th ed., Public Health Service, National Institutes of Health, Bethesda, MD (1991) (see also e.g. Johnson, G., and Wu, T. T., Nucleic Acids Res. 28 (2000) 214-218; Kabat, E. A., et al., Proc. Natl. Acad. Sci. USA 72 (1975) 2785-2788). Unless otherwise specified herein, numbering of amino acid residues in the constant region is according to the EU numbering system, also called the EU index of Kabat, as described in Kabat, E. A. et al., Sequences of Proteins of Immunological Interest, 5th ed., Public Health Service, National Institutes of Health, Bethesda, MD (1991), NIH Publication 91-3242.

The term “depletion” as used herein refers to a significant reduction in the number of receptor molecules displayed on the surface of a cell which expresses said receptor molecule when said cell is contacted with an antibody of the invention. The depletion is expressed as a ratio of receptor molecules on the cell surface versus to the number of receptor molecules that are present on the surface of a control cell that was not contacted with an antibody of the invention. A cell in which a receptor molecule was depleted from the cell surface has preferably more than 10%, 20%, 30%, 40%, 50%, 60%, 70%, 80% or 90% reduction, more preferably more than 95%, 98% or 99% percent reduction, in the number of receptor molecules displayed on the surface of the cell compared to an untreated control cell. Those ranges can be determined by state-of-the art methods as described herein and known to the skilled person.

“Effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g., B cell receptor); and B cell activation.

An “effective amount” of an agent, e.g., a pharmaceutical composition, refers to an amount effective, at dosages and for periods of time necessary, to achieve the desired therapeutic or prophylactic result.

The term “Fc region” herein is used to define a C-terminal region of an IgG class immunoglobulin heavy chain that contains at least a portion of the constant region. The term includes native sequence Fc regions and variant Fc regions. In one aspect, a human IgG heavy chain Fc region extends from Cys226, or from Pro230, to the carboxyl-terminus of the heavy chain. However, antibodies produced by host cells may undergo post-translational cleavage of one or more, particularly one or two, amino acids from the C-terminus of the heavy chain. Therefore, an antibody produced by a host cell by expression of a specific nucleic acid molecule encoding a full-length heavy chain may include the full-length heavy chain, or it may include a cleaved variant of the full-length heavy chain. This may be the case where the final two C-terminal amino acids of the heavy chain are glycine (G446) and lysine (K447, EU numbering system). Therefore, the C-terminal lysine (Lys447), or the C-terminal glycine (Gly446) and lysine (Lys447), of the Fc region may or may not be present. Amino acid sequences of heavy chains including an Fc region are denoted herein without C-terminal glycine-lysine dipeptide if not indicated otherwise. In one aspect, a heavy chain including an Fc region as specified herein, comprised in an antibody according to the invention, comprises an additional C-terminal glycine-lysine dipeptide (G446 and K447, EU numbering system). In one aspect, a heavy chain including an Fc region as specified herein, comprised in an antibody according to the invention, comprises an additional C-terminal glycine residue (G446, numbering according to EU index). Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991.

“Framework” or “FR” refers to variable domain residues other than complementary determining regions (CDRs). The FR of a variable domain generally consists of four FR domains: FR1, FR2, FR3, and FR4. Accordingly, the CDR and FR sequences generally appear in the following sequence in VH (or VL): FR1-CDR-H1 (CDR-L1)-FR2-CDR-H2 (CDR-L2)-FR3-CDR-H3 (CDR-L3)-FR4.

According to the Kabat numbering system, as is used herein, framework and CDR regions are located at the following regions of the variable domains:

| | FR1 | CDR-1 | FR2 | CDR2 | FR3 | CDR3 | FR4 |
|---|---|---|---|---|---|---|---|
| V_{H} | 1-30 | 31-35b* | 36-49 | 50-65 | 66-94 | 95-102 | 103-113 |
| V_{L} | 1-23 | 24-34 | 35-49 | 50-56 | 57-88 | 89-97 | 98-107 |

*in CDR-H1 additional amino acids between position 35b and 36 may be present, herein referred to as positions “35c”, “35d” and “35e”.

The terms “full length antibody”, “intact antibody”, and “whole antibody” are used herein interchangeably to refer to an antibody having a structure substantially similar to a native antibody structure or having heavy chains that contain an Fc region as defined herein.

The terms “host cell”, “host cell line”, and “host cell culture” are used interchangeably and refer to cells into which exogenous nucleic acid has been introduced, including the progeny of such cells. Host cells include “transformants” and “transformed cells”, which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda MD (1991), vols. 1-3. In one aspect, for the VL, the subgroup is subgroup kappa I as in Kabat et al., supra. In one aspect, for the VH, the subgroup is subgroup III as in Kabat et al., supra.

A “humanized” antibody refers to a chimeric antibody comprising amino acid residues from non-human CDRs and amino acid residues from human FRs. In certain aspects, a humanized antibody will comprise substantially all of at least one, and typically two, variable domains, in which all or substantially all of the CDRs correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR” as used herein refers to each of the regions of an antibody variable domain which are hypervariable in sequence and which determine antigen-binding specificity, for example “complementarity determining regions” (“CDRs”).

Generally, antibodies comprise six CDRs: three in the VH (CDR-H1, CDR-H2, CDR-H3), and three in the VL (CDR-L1, CDR-L2, CDR-L3). Exemplary CDRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991)); and
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262:732-745 (1996)).

TABLE 1
CDR Definitions¹

| CDR | Kabat | Chothia | AbM² |
|---|---|---|---|
| V_{H} CDR1 | 31-35 | 26-32 | 26-35 |
| V_{H} CDR2 | 50-65 | 52-58 | 50-58 |
| V_{H} CDR3 | 95-102 | 95-102 | 95-102 |
| V_{L} CDR1 | 24-34 | 26-32 | 24-34 |
| V_{L} CDR2 | 50-56 | 50-52 | 50-56 |
| V_{L} CDR3 | 89-97 | 91-96 | 89-97 |

¹Numbering of all CDR definitions in Table 1 is according to the numbering conventions set forth by Kabat et al. (see above).
²“AbM” with a lowercase “b” as used in Table 1 refers to the CDRs as defined by Oxford Molecular's “AbM” antibody modeling software.

Unless otherwise indicated, the CDRs are determined according to Kabat et al., supra. One of skill in the art will understand that the CDR designations can also be determined according to Chothia, supra, McCallum, supra, or any other scientifically accepted nomenclature system.

The terms “cell surface receptor”, “membrane receptor”, and “transmembrane receptor” are used interchangeably herein. Cell surface receptors are specialized integral membrane proteins that allow communication between the cell and the extracellular space. They are embedded in the plasma membrane of cells and act in cell signaling and signal transduction by binding to extracellular molecules, such as cytokines, growth factors, cell adhesion molecules, hormones, neurotransmitters, nutrients, and by triggering a response in the cell through a sequence of molecular switches to internal signaling pathways. PD1 and TfR are examples for such cell surface receptors.

An “immunoconjugate” is an antibody conjugated to one or more heterologous molecule(s), including but not limited to a cytotoxic agent.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain aspects, the individual or subject is a human.

The term “internalization” or “receptor internalization”, as used herein, refers to a biological process also termed endocytosis, i.e. a process by which cells absorb molecules (such as proteins) by engulfing them, resulting in the transport of the molecule from the outside to the inside of a cell. The internalized molecule can be located in an intracellular compartment, e.g. a vacuole, an endosome, a lysosome, the endoplasmic reticulum, the Golgi apparatus, or in the cytosol. An antibody that is “internalized” or “internalizing” refers to an antibody that is capable of being transported from the outside to the inside of a target cell, e.g. by binding to an internalizing cell surface receptor such as the Transferrin receptor.

An “isolated” antibody is one which has been separated from a component of its natural environment. In some aspects, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC) methods. For a review of methods for assessment of antibody purity, see, e.g., Flatman et al., J. Chromatogr. B 848:79-87 (2007).

The terms “linker peptide”, “peptide linker” or “peptidic linker” are used interchangeably and refer to short to medium-length polypeptides of preferably ten to about 25 amino acids. A linker peptide is usually rich in glycine for flexibility, as well as serine or threonine for solubility. “Linker peptide”, “peptide linker” or “peptidic linker” refers to synthetic amino acid sequences that connect or link two polypeptide sequences, e.g., that link two polypeptide domains. As used herein, the term “synthetic” refers to amino acid sequences that are not naturally occurring. Linker peptides of the invention connect two amino acid sequences via peptide bonds. Typically, a linker peptide connects a biologically active moiety to a second moiety in a linear sequence. In the context of polypeptides, a “linear sequence” or a “sequence” is the order of amino acids in a polypeptide in an amino to carboxyl terminal direction in which residues that neighbor each other in the sequence are contiguous in the primary structure of the polypeptide. As used herein, the terms “linked,” “connected”, “covalently bound”, “fused”, or “fusion”, are used interchangeably. In one aspect, the linker consists primarily or fully of Gly and Ser. In a further aspect, the linker has the sequence of SEQ ID NO: 108 or SEQ ID NO: 109.

The term “nucleic acid molecule” or “polynucleotide” includes any compound and/or substance that comprises a polymer of nucleotides. Each nucleotide is composed of a base, specifically a purine- or pyrimidine base (i.e. cytosine (C), guanine (G), adenine (A), thymine (T) or uracil (U)), a sugar (i.e. deoxyribose or ribose), and a phosphate group. Often, the nucleic acid molecule is described by the sequence of bases, whereby said bases represent the primary structure (linear structure) of a nucleic acid molecule. The sequence of bases is typically represented from 5′ to 3′. Herein, the term nucleic acid molecule encompasses deoxyribonucleic acid (DNA) including e.g., complementary DNA (cDNA) and genomic DNA, ribonucleic acid (RNA), in particular messenger RNA (mRNA), synthetic forms of DNA or RNA, and mixed polymers comprising two or more of these molecules. The nucleic acid molecule may be linear or circular. In addition, the term nucleic acid molecule includes both, sense and antisense strands, as well as single stranded and double stranded forms. Moreover, the herein described nucleic acid molecule can contain naturally occurring or non-naturally occurring nucleotides. Examples of non-naturally occurring nucleotides include modified nucleotide bases with derivatized sugars or phosphate backbone linkages or chemically modified residues. Nucleic acid molecules also encompass DNA and RNA molecules which are suitable as a vector for direct expression of an antibody of the invention in vitro and/or in vivo, e.g., in a host or patient. Such DNA (e.g., cDNA) or RNA (e.g., mRNA) vectors can be unmodified or modified. For example, mRNA can be chemically modified to enhance the stability of the RNA vector and/or expression of the encoded molecule so that mRNA can be injected into a subject to generate the antibody in vivo (see e.g., Stadler et al, Nature Medicine 2017, published online 12 Jun. 2017, doi:10.1038/nm.4356 or EP 2 101 823 B1).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an anti-TfR or anti-PD1 antibody” refers to one or more nucleic acid molecules encoding anti-TfR or anti-PD1 antibody heavy and light chains (or fragments thereof), including such nucleic acid molecule(s) in a single vector or separate vectors, and such nucleic acid molecule(s) present at one or more locations in a host cell.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

A “naked antibody” refers to an antibody that is not conjugated to a heterologous moiety (e.g., a cytotoxic moiety) or radiolabel. The naked antibody may be present in a pharmaceutical composition.

“Native antibodies” refer to naturally occurring immunoglobulin molecules with varying structures. For example, native IgG antibodies are heterotetrameric glycoproteins of about 150,000 Daltons, composed of two identical light chains and two identical heavy chains that are disulfide-bonded. From N- to C-terminus, each heavy chain has a variable domain (VH), also called a variable heavy domain or a heavy chain variable region, followed by three constant heavy domains (CH1, CH2, and CH3). Similarly, from N- to C-terminus, each light chain has a variable domain (VL), also called a variable light domain or a light chain variable region, followed by a constant light (CL) domain.

The term “package insert” is used to refer to instructions customarily included in commercial packages of therapeutic products, that contain information about the indications, usage, dosage, administration, combination therapy, contraindications and/or warnings concerning the use of such therapeutic products.

As used herein, the term “payload” refers to a therapeutic agent that acts on a target (e.g., a target cell) and can be any naturally occurring or artificially synthesized pharmaceutically active molecule that can be introduced into an exosome and/or a producer cell. It includes therapeutic agents such as, nucleotides, nucleic acids, amino acids, polypeptides, lipids, carbohydrates, viruses and viral particles and small molecules.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity for the purposes of the alignment. Alignment for purposes of determining percent amino acid sequence identity can be achieved in various ways that are within the skill in the art, for instance, using publicly available computer software such as BLAST, BLAST-2, Clustal W, Megalign (DNASTAR) software or the FASTA program package. Those skilled in the art can determine appropriate parameters for aligning sequences, including any algorithms needed to achieve maximal alignment over the full length of the sequences being compared. Alternatively, the percent identity values can be generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087 and is described in WO 2001/007611.

Unless otherwise indicated, for purposes herein, percent amino acid sequence identity values are generated using the ggsearch program of the FASTA package version 36.3.8c or later with a BLOSUM50 comparison matrix. The FASTA program package was authored by W. R. Pearson and D. J. Lipman (1988), “Improved Tools for Biological Sequence Analysis”, PNAS 85:2444-2448; W. R. Pearson (1996) “Effective protein sequence comparison” Meth. Enzymol. 266:227-258; and Pearson et. al. (1997) Genomics 46:24-36 and is publicly available from www.fasta.bioch.virginia.edu/fasta_www2/fasta_down.shtml or www.ebi.ac.uk/Tools/sss/fasta. Alternatively, a public server accessible at fasta.bioch.virginia.edu/fasta_www2/index.cgi can be used to compare the sequences, using the ggsearch (global protein: protein) program and default options (BLOSUM50; open: −10; ext: −2; Ktup=2) to ensure a global, rather than local, alignment is performed. Percent amino acid identity is given in the output alignment header.

The term “pharmaceutical composition” or “pharmaceutical formulation” refers to a preparation which is in such form as to permit the biological activity of an active ingredient contained therein to be effective, and which contains no additional components which are unacceptably toxic to a subject to which the pharmaceutical composition would be administered.

A “pharmaceutically acceptable carrier” refers to an ingredient in a pharmaceutical composition or formulation, other than an active ingredient, which is nontoxic to a subject. A pharmaceutically acceptable carrier includes, but is not limited to, a buffer, excipient, stabilizer, or preservative.

The term “TfR” or “Transferrin receptor”, as used herein, refers to any native TfR or Transferrin receptor from any vertebrate source, including mammals such as primates (e.g., humans) and rodents (e.g., mice and rats), unless otherwise indicated. The term encompasses “full-length”, unprocessed TfR as well as any form of TfR that results from processing in the cell. The term also encompasses naturally occurring variants of TfR, e.g., splice variants or allelic variants. The amino acid sequence of an exemplary human TfR is shown in SEQ ID NO: 66 in Table 10.

The term “PD1” or “Programmed cell death protein 1”, as used herein, refers to any native PD1 or Programmed cell death protein 1 from any vertebrate source, including mammals such as primates (e.g., humans) and rodents (e.g., mice and rats), unless otherwise indicated. The term encompasses “full-length”, unprocessed PD1 as well as any form of PD1 that results from processing in the cell. The term also encompasses naturally occurring variants of PD1, e.g., splice variants or allelic variants. The amino acid sequence of an exemplary human PD1 is shown in SEQ ID NO: 65 in Table 10.

As used herein, “treatment” (and grammatical variations thereof such as “treat” or “treating”) refers to clinical intervention in an attempt to alter the natural course of a disease in the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some aspects, antibodies of the invention are used to delay development of a disease or to slow the progression of a disease.

The terms “cancer” and “cancerous” refer to or describe the physiological condition in mammals that is typically characterized by unregulated cell growth. Aspects of cancer include solid tumor cancers and non-solid tumor cancers. Examples of cancer include but are not limited to, carcinoma, lymphoma, blastoma, sarcoma, and leukemia or lymphoid malignancies. More particular examples of such cancers include, but are not limited to, bladder cancer (e.g., urothelial carcinoma (UC), including metastatic UC (mUC); muscle-invasive bladder cancer (MIBC), and non-muscle-invasive bladder cancer (NMIBC)); kidney or renal cancer (e.g., renal cell carcinoma (RCC)); lung cancer, including small-cell lung cancer, non-small cell lung cancer, adenocarcinoma of the lung, and squamous carcinoma of the lung; cancer of the urinary tract; breast cancer (e.g., HER2+ breast cancer and triple-negative breast cancer (TNBC), which are estrogen receptors (ER−), progesterone receptors (PR−), and HER2 (HER2−) negative); prostate cancer, such as castration-resistant prostate cancer (CRPC); cancer of the peritoneum; hepatocellular cancer; gastric or stomach cancer, including gastrointestinal cancer and gastrointestinal stromal cancer; pancreatic cancer (e.g., pancreatic ductal adenocarcinoma (PDAC)); glioblastoma; cervical cancer; ovarian cancer; liver cancer (e.g., hepatocellular carcinoma (HCC)); hepatoma; colon cancer; rectal cancer; colorectal cancer; endometrial or uterine carcinoma; salivary gland carcinoma; prostate cancer; vulval cancer; thyroid cancer; hepatic carcinoma; anal carcinoma; penile carcinoma; melanoma, including superficial spreading melanoma, lentigo malignant melanoma, acral lentiginous melanomas, and nodular melanomas; multiple myeloma and B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); acute myologenous leukemia (AML); hairy cell leukemia; chronic myeloblastic leukemia (CML); post-transplant lymphoproliferative disorder (PTLD); and myelodysplastic syndromes (MDS), as well as abnormal vascular proliferation associated with phakomatoses, edema (such as that associated with brain tumors), Meigs' syndrome, brain cancer, head and neck cancer, and associated metastases.

“Tumor,” as used herein, refers to all neoplastic cell growth and proliferation, whether malignant or benign, and all pre-cancerous and cancerous cells and tissues. The terms “cancer”, “cancerous”, “cell proliferative disorder”, “proliferative disorder,” and “tumor” are not mutually exclusive as referred to herein.

The terms “cell proliferative disorder” and “proliferative disorder” refer to disorders that are associated with some degree of abnormal cell proliferation. In one embodiment, the cell proliferative disorder is cancer. In another embodiment, the cell proliferative disorder is a tumor.

The terms “B cell proliferative disorder” or “B cell malignancy” refer to disorders that are associated with some degree of abnormal B cell proliferation and include, for example, lymphomas, leukemias, myelomas, and myelodysplastic syndromes. In one embodiment, the B cell proliferative disorder is a lymphoma, such as non-Hodgkin's lymphoma (NHL), including, for example, DLBCL (e.g., relapsed or refractory DLBCL), FL (e.g., relapsed or refractory FL or transformed FL), or MCL. In another embodiment, the B cell proliferative disorder is a leukemia, such as chronic lymphocytic leukemia (CLL). In yet another embodiment, the B cell proliferative disorder is a central nervous system lymphoma (CNSL).

### Bladder Cancer

The term “bladder cancer” includes, but is not limited to, urothelial carcinoma (UC), and which may be, for example, locally advanced or metastatic. The methods described herein are suitable for treatment of various stages of cancer, including cancers that are locally advanced and/or metastatic. In cancer staging, locally advanced is generally defined as cancer that has spread from a localized area to nearby tissues and/or lymph nodes. In the Roman numeral staging system, locally advanced usually is classified in Stage II or III. Cancer which is metastatic is a stage where the cancer spreads throughout the body to distant tissues and organs (stage IV).

The term “upper tract UC” refers to UC of the renal pelvis or ureter. The upper tract UC may be upper tract metastatic UC. A minority of cases (e.g., about 5-10%) of UC are upper tract UC.

The term “lower tract UC” refers to UC of the bladder or urethra. The lower tract UC may be lower tract metastatic UC. The majority of cases (e.g., about 90-95%) of UC are lower tract UC.

As used herein, the terms “inoperable” and “unresectable” are used interchangeably to refer to a cancer (e.g., bladder cancer (e.g., UC, including locally advanced or metastatic UC)) for which surgical resection is not possible or cannot be safely performed. In some embodiments, a bladder cancer (e.g., UC, including locally advanced or metastatic UC) is inoperable because of involvement of pelvic sidewall or adjacent viscera (clinical Stage T4b) or bulky nodal metastasis (N2-N3).

The term “eligible for treatment with a platinum-based chemotherapy” means that the subject is eligible for treatment with a platinum-based chemotherapy, either in the attending clinician's judgment or according to standardized criteria for eligibility for platinum-based chemotherapy that are known in the art. For example, the criteria set forth in Galsky et al. Lancet Oncol. 12 (3): 211-4, 2011 may be used to determine whether a subject is eligible for cisplatin-based chemotherapy. Galsky et al. describe a consensus definition of patients with metastatic UC (mUC) in which patients meeting at least one of the following are considered unfit for cisplatin-based chemotherapy: (i) a World Health Association (WHO) or Eastern Cooperative Oncology Group (ECOG) performance status of 2, or Karnofsky performance status of 60-70%; (ii) creatinine clearance (calculated or measured) less than 1 mL/s; (iii) National Cancer Institute (NCI) Common Terminology Criteria for Adverse Events (CTCAE) v4.0 Grade≥2 audiometric hearing loss; (iv) CTCAE v.4.0 Grade≥2 peripheral neuropathy; and/or New York Heart Association (NYHA) class III heart failure. In one example, a patient is considered unfit for cisplatin-based chemotherapy if they have one or more of the following: impaired renal function (e.g., glomerular filtration rate (GFR)>30 but <60 mL/min); GFR may be assessed by direct measurement (i.e., creatinine clearance or ethyldediaminetetra-acetate) or, if not available, by calculation from serum/plasma creatinine (Cockcroft-Gault formula)); hearing loss (e.g., National Cancer Institute (NCI) Common Terminology Criteria for Adverse Events (CTCAE) v4.0 Grade≥2 audiometric hearing loss of 25 decibels at two contiguous frequencies); peripheral neuropathy (e.g., NCI CTCAE v4.0 Grade≥2 peripheral neuropathy (i.e., sensory alteration or paresthesia, including tingling)); and/or ECOG performance status assessment (see Oken et al. Am. J. Clin. Oncol. 5:649-655, 1982) (e.g., an ECOG performance status of 2). In some embodiments, a subject having one of the following may be eligible for carboplatin-based chemotherapy: impaired renal function (e.g., glomerular filtration rate (GFR)>30 but <60 mL/min); GFR may be assessed by direct measurement (i.e., creatinine clearance or ethyldediaminetetra-acetate) or, if not available, by calculation from serum/plasma creatinine (Cockcroft-Gault formula)); hearing loss (e.g., CTCAE v4.0 Grade≥2 audiometric hearing loss of 25 decibels at two contiguous frequencies); peripheral neuropathy (e.g., NCI CTCAE v4.0 Grade≥2 peripheral neuropathy (i.e., sensory alteration or paresthesia, including tingling)); and/or ECOG performance status assessment (e.g., an ECOG performance status of 2).

Chemotherapeutic agents also include “platinum-based” chemotherapeutic agents, which comprise an organic compound which contains platinum as an integral part of the molecule. Typically, platinum-based chemotherapeutic agents are coordination complexes of platinum. Platinum-based chemotherapeutic agents are sometimes called “platins” in the art. Examples of platinum-based chemotherapeutic agents include, but are not limited to, cisplatin, carboplatin, oxaliplatin, nedaplatin, triplatin tetranitrate, phenanthriplatin, picoplatin, lipoplatin, and satraplatin. Platinum-based chemotherapeutic agents (e.g., cisplatin or carboplatin) may be administered in combination with one or more additional chemotherapeutic agents, e.g., a nucleoside analog (e.g., gemcitabine).

A “platinum-based chemotherapy,” as used herein, refers to a chemotherapy regimen that includes a platinum-based chemotherapeutic agent. For example, a platinum-based chemotherapy may include a platinum-based chemotherapeutic agent (e.g., cisplatin or carboplatin) in combination with one or more additional chemotherapeutic agents, e.g., a nucleoside analog (e.g., gemcitabine).

A “nucleoside analog,” as used herein, refers to a nucleoside that includes a nucleic acid analog and a sugar. Nucleoside analogs may function as antimetabolites. Exemplary nucleoside analogues include but are not limited to gemcitabine, cytarabine, fludarabine, and cladribine.

### Breast Cancer

The term “breast cancer” includes, but is not limited to, HER2+ breast cancer and triple-negative breast cancer (TNBC), which is a form of breast cancer in which the cancer cells are negative for estrogen receptors (ER−), progesterone receptors (PR−), and HER2 (HER2−), and which may be locally advanced, unresectable, and/or metastatic (e.g., metastatic triple-negative breast cancer (mTNBC)).

As used herein, the terms “early TNBC” and “eTNBC” refer to early-stage TNBC, including Stage I-Stage III TNBC. Early TNBC accounts for 10% to 20% of all new early breast cancer diagnoses, with a 3-year event-free survival rate of 74% to 76% after treatment with neoadjuvant anthracycline and taxane therapy.

As used herein, “pathologic complete response” or “pCR” refers to the absence of invasive tumor from both breast and lymph nodes. The term pCR includes absence of invasive cancer in the breast and axillary nodes, irrespective of ductal carcinoma in situ (i.e., ypT0/is ypN0); absence of invasive cancer and in situ cancer in the breast and axillary nodes (i.e., ypT0 ypN0); and absence of invasive cancer in the breast irrespective of ductal carcinoma in situ or nodal involvement (i.e., ypT0/is). In particular aspects, pCR refers to absence of invasive cancer in the breast and axillary nodes, irrespective of ductal carcinoma in situ (i.e., ypT0/is ypN0).

A “taxane” as used herein is an agent (e.g., a diterpene) which may bind to tubulin, promoting microtubule assembly and stabilization and/or prevent microtubule depolymerization. Exemplary taxanes include, but are not limited to, paclitaxel (i.e., TAXOL®, CAS #33069-62-4), docetaxel (i.e., TAXOTERE®, CAS #114977-28-5), larotaxel, cabazitaxel, milataxel, tesetaxel, and/or orataxel. Taxanes included herein also include taxoid 10-deacetylbaccatin III and/or derivatives thereof. In some embodiments, the taxane is an albumin-coated nanoparticle (e.g., nano-albumin bound (nab)-paclitaxel, i.e., ABRAXANE® and/or nab-docetaxel, ABI-008). In some embodiments, the taxane is nab-paclitaxel (ABRAXANE®). In some embodiments, the taxane is formulated in CREMAPHOR® (e.g., TAXOL®) and/or in TWEEN® such as polysorbate 80 (e.g., TAXOTERE®). In some embodiments, the taxane is liposome-encapsulated taxane. In some embodiments, the taxane is a prodrug form and/or conjugated form of taxane (e.g., DHA covalently conjugated to paclitaxel, paclitaxel poliglumex, and/or linoleyl carbonate-paclitaxel). In some embodiments, the paclitaxel is formulated with substantially no surfactant (e.g., in the absence of CREMAPHOR® and/or TWEEN®, such as TOCOSOL® paclitaxel).

An “anthracycline” as used herein refers to a class of antibiotic compounds that exhibit cytotoxic activity. Anthracyclines may cause cytotoxicity via DNA intercalation, topoisomerase-II-mediated toxicity, generation of reactive oxygen species, and/or DNA adduct formation. Exemplary anthracyclines include, but are not limited, to doxorubicin, epirubicin, idarubicin, daunorubicin, mitoxantrone, and valrubicin. In some aspects, the anthracycline is doxorubicin or epirubicin. In some specific aspects, the anthracycline is doxorubicin. In other specific aspects, the anthracycline is epirubicin.

An “alkylating agent” as used herein refers to a class of chemotherapy agents that attaches an alklyl group to a nucleotide, e.g., DNA. Typically, the alkyl group is attached to the guanine base of DNA. Exemplary alkylating agents include, but are not limited to, a nitrogen mustard derivative (e.g., cyclophosphamide, chlorambucil, uramustine, melphalan, or bendamustine), a nitrosourea (e.g., carmustine, lomustine, or streptozocin), an alkyl sufolnate (e.g., busulfan), a triazine (e.g., dacarbazine or temozolomide, and an ethylenimine (e.g., altretamine or thiotepa).

### Kidney Cancer

In some embodiments, the cancer is kidney cancer. In particular embodiments, the kidney cancer is renal cell carcinoma (RCC) (e.g., advanced RCC or metastatic RCC (mRCC), including previously untreated RCC). In some embodiments, the kidney cancer is sarcomatoid kidney cancer (e.g., sarcomatoid RCC (e.g., sarcomatoid advanced or mRCC)).

The term “sarcomatoid” refers to a cancer (e.g., a kidney cancer (e.g., an RCC)) that is characterized by sarcomatoid morphology, for example, as assessed by histology. Sarcomatoid kidney cancer (e.g., sarcomatoid RCC) is associated with aggressive behavior and poor prognosis. In some embodiments, a sarcomatoid kidney cancer includes or consists of atypical spindle-shaped cells and/or resembles any form of sarcoma. See, e.g., El Mouallem et al. Urol. Oncol. 36:265-271, 2018, which is incorporated herein by reference in its entirety. Sarcomatoid RCC can occur in any subtype of RCC, including clear cell RCC, chromophobe RCC, collecting duct carcinoma, renal medullary carcinoma, fumarate hydratase (FH)-deficient RCC, and succinate dehydrogenase (SDH)-deficient RCC. The incidence of sarcomatoid RCC varies among subtypes, but is typically higher in clear cell RCC (approximately 5-8%) and chromophobe RCC (approximately 8-10%). The histology of the sarcomatoid component can be variable, and may include a fibrosarcoma-like pattern, a pleomorphic undifferentiated sarcoma-like pattern, or other heterologous sarcomatoid patterns (e.g., osteosarcoma-, chondrosarcoma-, or rhabdomyosarcoma-like patterns). Necrosis is typically present in a large majority (about 90%) of cases. In some embodiments, there is no minimum amount or percentage of sarcomatoid differentiation for an individual's kidney cancer to be classified as sarcomatoid. Sarcomatoid RCC may be assessed as described in Example 1. In other embodiments, sarcomatoid RCC may be characterized as described by the 2012 International Society of Urological Pathology (ISUP) Vancouver consensus (see Srigley et al. Am. J. Surg. Pathol. 37:1469-89, 2013, which is incorporated herein by reference in its entirety).

The term “Memorial Sloan Kettering Cancer Center (MSKCC) risk score” refers to a scoring system based on set of prognostic factors associated with survival in kidney cancer (e.g., RCC, e.g., mRCC) patients. See, e.g., Motzer et al. J. Clin. Oncol. 17 (8): 2530-2540, 1999 and Motzer et al. J. Clin. Oncol. 20 (1): 289-296, 2002, which are incorporated herein by reference in their entirety. In some embodiments, a MSKCC risk score can be calculated based on the following factors, as described in Example 1: (i) a time from nephrectomy to treatment (e.g., systemic treatment) of less than one year, a lack of a nephrectomy, or an initial diagnosis with metastatic disease; (ii) a hemoglobin level less than the lower limit of normal (LLN), optionally wherein the normal range for hemoglobin is between 13.5 and 17.5 g/dL for men and between 12 and 15.5 g/dL for women; (iii) a serum corrected calcium level greater than 10 mg/dL, optionally wherein the serum corrected calcium level is the serum calcium level (mg/dL)+0.8 (4−serum albumin (g/dL)); (iv) a serum lactate dehydrogenase (LDH) level greater than 1.5 times the upper limit of normal (ULN), optionally wherein the ULN is 140 U/L; and/or (v) a Karnofsky Performance Status (KPS) score of <80. In some embodiments, an individual has a favorable MSKCC risk score if the individual has zero of the preceding characteristics. In some embodiments, an individual has an intermediate MSKCC risk score if the individual has one or two of the preceding characteristics. In some embodiments, an individual has a poor MSKCC risk score if the individual has three or more of the preceding characteristics. In some embodiments, an individual's MSKCC risk score may be used to identify whether the individual may benefit from an anti-cancer therapy, e.g., an anti-cancer therapy that includes a VEGF antagonist (e.g., an anti-VEGF antibody such as bevacizumab) and a PD-L1 axis binding antagonist (e.g., an anti-PD-L1 antibody such as atezolizumab).

As used herein, “deterioration-free rate” or “DFR” refers to the probability that a patient will experience a clinically meaningful deterioration in a length of time, e.g., the time from onset of a therapy to a patient's first ≥2-point increase above baseline on the MD Anderson Symptom Inventory (MDASI) interference scale.

The “MD Anderson Symptom Inventory (MDASI) interference scale” refers to a patient-reported outcome measurement scoring system that assesses the severity and impact of multiple symptoms related to cancer and its treatment (see, e.g., Mendoza et al. Clin. Breast Cancer 13:325-334, 2013; Jones et al. Clin. Genitourin. Cancer 12:41-49, 2014; and Shi et al. Pain 158:1108-1112, 2017). In the MDASI interference scale, a patient rates the degree to which symptoms interfered with various aspects of life during the past 24 hours. Each interference item (work, general activity, walking, relations with others, enjoyment of life, and mood) is rated on a 0-10 scale, with 0 representing “did not interfere” and 10 representing “interfered completely.”

### Liver Cancer

As used herein, the term “unresectable” refers to a cancer (e.g., liver cancer (e.g., HCC, including locally advanced or metastatic and/or unresectable HCC)) for which surgical resection is not possible or cannot be safely performed. For example, with respect to liver cancer, the term “unresectable” indicates that the cancer cannot be safely removed by partial hepatectomy, for example, because the tumor is too large to be safely removed, the tumor is positioned in the part of the liver that makes it difficult to remove (e.g., such as close to a large blood vessel), there are several tumors or the cancer has spread throughout the liver and/or outside the liver, and/or the subject has underlying health issues (e.g., cirrhosis) that preclude resection.

As used herein, “time to radiographic progression” (TTRP) refers to a length of time between a first event (e.g., randomization into a clinical trial or administration of a first dose of a treatment regimen) and objective progression as assessed by radiology. In some embodiments, radiographic progression is defined according to the Response Evaluation Criteria in Solid Tumors (RECIST) criteria, e.g., RECIST v1.1 or mRECIST (e.g., HCC mRECIST).

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three complementary determining regions (CDRs). (See, e.g., Kindt et al. Kuby Immunology, 6^{th} ed., W.H. Freeman and Co., binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “vector”, as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors”.

### II. Compositions and Methods

In one aspect, the invention is based, in part, on the finding that combining a first antigen-binding domain that binds specifically to TfR on the one hand and a second, and optionally a third, antigen-binding domain that binds specifically to PD1 on the other hand in a single bispecific antibody results in internalization of the bispecific antibody, when contacted with a cell that expresses and/or displays TfR and PD1 on its surface. Such cells may for example be activated T cells. In a further aspect, the invention is based, in part, on the finding that anti-TfR anti-PD1 2+1 format antibodies, i.e. bispecific antibodies comprising a first antigen-binding domain that specifically binds to TfR and a second and a third antigen-binding domain that specifically bind to PD1, show improved biological activity and lead to better inhibition of the interaction between PD1 and PD-L1 than a monospecific, bivalent PD1 antibody. In one aspect these molecules comprise IgG class Fab fragments, and optionally also IgG class Fc regions, that are covalently bound to each other resulting in the 2+1 format antibodies disclosed herein. Without wishing to be bound by theory, the inventors believe that these antibodies deplete PD1 from the surface of T cells and thus interrupt binding of PD1 to PD1 ligand (PD-L1), which is located on the surface of tumor cells, more permanently than could be achieved by mere binding of an anti-PD1 blocking antibody to PD1. The antibodies of the invention thus lead to a stronger inhibitory effect on PD1/PD-L1 mediated signaling than anti-PD1 antibodies known in the art. The usefulness of the antibodies described herein is believed to be related to the ability of those antibodies to form a complex with the two receptors TfR and PD1 on the cell surface, upon which said complex is internalized into the cell in its entirety.

The antibodies according to the invention can also be used to transport a payload specifically to T cells, either by conjugating the payload directly to the antibody or by attaching an additional antigen-binding domain binding to the antibody, which can then specifically bind to the payload. In certain aspects, bispecific antibodies that bind to PD1 and TfR are provided. Antibodies of the invention are for example useful for the delivery of agents such as small molecules or RNA to T cells for cancer therapy.

In another aspect, the invention is based, in part, on the finding that a bispecific anti-TfR anti-PD1 antibody with a 2+1 stoichiometry of the binding domains targeting anti-PD1 and anti-TfR, i.e. a bispecific antibody comprising a first antigen-binding domain that specifically binds to TfR and a second and a third antigen-binding domain that specifically bind to PD1, leads to increased internalization of PD1 as compared to a monospecific anti-PD1-antibody or a bispecific anti-TfR anti-PD1 antibody with a 1+1 stoichiometry of the binding domains targeting anti-PD1 and anti-TfR, i.e. a bispecific antibody comprising one antigen-binding domain that specifically binds to TfR and only one antigen-binding domain that specifically binds to PD1. Antibodies of the invention are useful, e.g., for inhibiting the growth of tumor cells in an individual or for the diagnosis or treatment of tumors. The antibodies of the invention can be used for every kind of medical treatment for which anti-PD1 antibodies known in the art are being used for.

A. Exemplary Bispecific Antibodies that Bind to TfR and PD1

In one aspect, the invention provides a bispecific antibody comprising a first antigen-binding domain that specifically binds to TfR and a second, and optionally a third, antigen-binding domain that specifically binds to PD1. In one aspect, provided are isolated bispecific antibodies comprising a first antigen-binding domain that specifically binds to TfR and a second, and optionally a third, antigen-binding domain that specifically binds to PD1. In one aspect, the invention provides antibodies that specifically bind to TfR and to PD1. In certain aspects, a bispecific antibody comprising a first antigen-binding domain that specifically binds to TfR and a second, and optionally a third, antigen-binding domain that specifically binds to PD1 causes the depletion of PD1 from the cell surface and causes the internalization of PD1 into the cell. The cell is preferably a T cell, more preferably an activated T cell. The internalization of PD1 into the cell inhibits the binding of PD1 to its ligands, preferably the binding to PD-L1. Preferably, this inhibition of binding between PD1 and its ligands due to the internalization of PD1 into the cell has a longer-lasting effect than the blocking achieved by mere binding of an antigen-binding domain to PD1. The bispecific antibody comprising a first antigen-binding domain that specifically binds to TfR and a second, and optionally a third, antigen-binding domain that specifically binds to PD1 preferably
i. binds simultaneously to TfR and PD1,
ii. by simultaneously binding to TfR and PD1 leads to the internalization of the complex formed by the bispecific antibody, TfR and PD1 into the cell and the depletion of PD1 from the cell surface preventing PD-L1 from accessing PD1, and/or
iii. is able to block PD1-PD-L1 interaction and/or to prevent PD1 signaling by binding to PD1 and achieving PD1 depletion from the cell surface, preferably leading to a more effective and/or permanent inhibition of PD1/PD-L1 mediated signaling than conventional anti-PD1 antibodies.

In one aspect, the Fab fragments, and optionally also the IgG class Fc regions, that are comprised by the bispecific antibody are covalently bound to each other, resulting in 2+1 format antibodies of different conformations. In a further aspect the bispecific antibody is essentially in monomeric form, i.e. it does not form dimeric or multimeric (e.g. pentameric) structures comprising more than one bispecific antibody of the invention. In a particular aspect, at least 90%, more particularly at least 95%, preferably at least 98%, more preferably at least 99% of the antibody are in monomeric form.

In one aspect, the invention provides a bispecific antibody comprising
A) a first antigen-binding domain that specifically binds to TfR comprising
   a. at least one, at least two, at least three, at least four, at least five, or all six CDRs selected from (a) CDR-H1 comprising the amino acid sequence of SEQ ID NO: 1; (b) CDR-H2 comprising the amino acid sequence of SEQ ID NO: 2; (c) CDR-H3 comprising the amino acid sequence of SEQ ID NO: 3; (d) CDR-L1 comprising the amino acid sequence of SEQ ID NO: 4; (e) CDR-L2 comprising the amino acid sequence of SEQ ID NO: 5; and (f) CDR-L3 comprising the amino acid sequence of SEQ ID NO: 6, or
   b. at least one, at least two, at least three, at least four, at least five, or all six CDRs selected from (a) CDR-H1 comprising the amino acid sequence of SEQ ID NO: 9; (b) CDR-H2 comprising the amino acid sequence of SEQ ID NO: 10; (c) CDR-H3 comprising the amino acid sequence of SEQ ID NO: 11; (d) CDR-L1 comprising the amino acid sequence of SEQ ID NO: 12; (e) CDR-L2 comprising the amino acid sequence of SEQ ID NO: 13; and (f) CDR-L3 comprising the amino acid sequence of SEQ ID NO: 14, and
B) a second, and optionally a third, antigen-binding domain that specifically binds to PD1 comprising
   a. at least one, at least two, at least three, at least four, at least five, or all six CDRs selected from (a) CDR-H1 comprising the amino acid sequence of SEQ ID NO: 17; (b) CDR-H2 comprising the amino acid sequence of SEQ ID NO: 18; (c) CDR-H3 comprising the amino acid sequence of SEQ ID NO: 19; (d) CDR-L1 comprising the amino acid sequence of SEQ ID NO: 20; (e) CDR-L2 comprising the amino acid sequence of SEQ ID NO: 21; and (f) CDR-L3 comprising the amino acid sequence of SEQ ID NO: 22, or
   b. at least one, at least two, at least three, at least four, at least five, or all six CDRs selected from (a) CDR-H1 comprising the amino acid sequence of SEQ ID NO: 25; (b) CDR-H2 comprising the amino acid sequence of SEQ ID NO: 26; (c) CDR-H3 comprising the amino acid sequence of SEQ ID NO: 27; (d) CDR-L1 comprising the amino acid sequence of SEQ ID NO: 28; (e) CDR-L2 comprising the amino acid sequence of SEQ ID NO: 29; and (f) CDR-L3 comprising the amino acid sequence of SEQ ID NO: 30.

In one aspect, the invention provides a bispecific antibody comprising
A) a first antigen-binding domain that specifically binds to TfR comprising
   a. at least one, at least two, or all three VH CDR sequences selected from (a) CDR-H1 comprising the amino acid sequence of SEQ ID NO: 1; (b) CDR-H2 comprising the amino acid sequence of SEQ ID NO: 2; and (c) CDR-H3 comprising the amino acid sequence of SEQ ID NO: 3, or
   b. at least one, at least two, or all three VH CDR sequences selected from (a) CDR-H1 comprising the amino acid sequence of SEQ ID NO: 9; (b) CDR-H2 comprising the amino acid sequence of SEQ ID NO: 10; and (c) CDR-H3 comprising the amino acid sequence of SEQ ID NO: 11, and
B) a second, and optionally a third, antigen-binding domain that specifically binds to PD1 comprising
   a. at least one, at least two, or all three VH CDR sequences selected from (a) CDR-H1 comprising the amino acid sequence of SEQ ID NO: 17; (b) CDR-H2 comprising the amino acid sequence of SEQ ID NO: 18; and (c) CDR-H3 comprising the amino acid sequence of SEQ ID NO: 19, or
   b. at least one, at least two, or all three VH CDR sequences selected from (a) CDR-H1 comprising the amino acid sequence of SEQ ID NO: 25; (b) CDR-H2 comprising the amino acid sequence of SEQ ID NO: 26; and (c) CDR-H3 comprising the amino acid sequence of SEQ ID NO: 27.

In one aspect, the bispecific antibody comprises a CDR-H3 comprising the amino acid sequence of SEQ ID NO: 3 and a CDR-H3 comprising the amino acid sequence of SEQ ID NO: 19. In one aspect, the bispecific antibody comprises a CDR-H3 comprising the amino acid sequence of SEQ ID NO: 3 and a CDR-H3 comprising the amino acid sequence of SEQ ID NO: 27.

In one aspect, the bispecific antibody comprises a CDR-H3 comprising the amino acid sequence of SEQ ID NO: 11 and a CDR-H3 comprising the amino acid sequence of SEQ ID NO: 19. In one aspect, the bispecific antibody comprises a CDR-H3 comprising the amino acid sequence of SEQ ID NO: 11 and a CDR-H3 comprising the amino acid sequence of SEQ ID NO: 27.

In another aspect, the bispecific antibody comprises
A) a first antigen-binding domain that specifically binds to TfR comprising
   a. CDR-H3 comprising the amino acid sequence of SEQ ID NO: 3 and CDR-L3 comprising the amino acid sequence of SEQ ID NO: 6, or
   b. CDR-H3 comprising the amino acid sequence of SEQ ID NO: 11 and CDR-L3 comprising the amino acid sequence of SEQ ID NO: 14, and
B) a second, and optionally a third, antigen-binding domain that specifically binds to PD1 comprising
   a. CDR-H3 comprising the amino acid sequence of SEQ ID NO: 19 and CDR-L3 comprising the amino acid sequence of SEQ ID NO: 22, or
   b. CDR-H3 comprising the amino acid sequence of SEQ ID NO: 27 and CDR-L3 comprising the amino acid sequence of SEQ ID NO: 30.

In a further aspect, the antibody comprises
A) a first antigen-binding domain that specifically binds to TfR comprising
   a. CDR-H3 comprising the amino acid sequence of SEQ ID NO: 3, CDR-L3 comprising the amino acid sequence of SEQ ID NO: 6, and CDR-H2 comprising the amino acid sequence of SEQ ID NO: 2, or
   b. CDR-H3 comprising the amino acid sequence of SEQ ID NO: 11, CDR-L3 comprising the amino acid sequence of SEQ ID NO: 14, and CDR-H2 comprising the amino acid sequence of SEQ ID NO: 10, and
B) a second, and optionally a third, antigen-binding domain that specifically binds to PD1 comprising
   a. CDR-H3 comprising the amino acid sequence of SEQ ID NO: 19, CDR-L3 comprising the amino acid sequence of SEQ ID NO: 22, and CDR-H2 comprising the amino acid sequence of SEQ ID NO: 18, or
   b. CDR-H3 comprising the amino acid sequence of SEQ ID NO: 27, CDR-L3 comprising the amino acid sequence of SEQ ID NO: 30, and CDR-H2 comprising the amino acid sequence of SEQ ID NO: 26.

In a further aspect, the antibody comprises
A) a first antigen-binding domain that specifically binds to TfR comprising
   a. CDR-H1 comprising the amino acid sequence of SEQ ID NO: 1; CDR-H2 comprising the amino acid sequence of SEQ ID NO: 2; and CDR-H3 comprising the amino acid sequence of SEQ ID NO: 3, or
   b. CDR-H1 comprising the amino acid sequence of SEQ ID NO: 9; CDR-H2 comprising the amino acid sequence of SEQ ID NO: 10; and CDR-H3 comprising the amino acid sequence of SEQ ID NO: 11, and
B) a second, and optionally a third, antigen-binding domain that specifically binds to PD1 comprising
   a. CDR-H1 comprising the amino acid sequence of SEQ ID NO: 17; CDR-H2 comprising the amino acid sequence of SEQ ID NO: 18; and CDR-H3 comprising the amino acid sequence of SEQ ID NO: 19, or
   b. CDR-H1 comprising the amino acid sequence of SEQ ID NO: 25; CDR-H2 comprising the amino acid sequence of SEQ ID NO: 26; and CDR-H3 comprising the amino acid sequence of SEQ ID NO: 27.

In another aspect, the invention provides a bispecific antibody comprising
A) a first antigen-binding domain that specifically binds to TfR comprising
   a. at least one, at least two, or all three VL CDR sequences selected from
      (a) CDR-L1 comprising the amino acid sequence of SEQ ID NO: 4;
      (b) CDR-L2 comprising the amino acid sequence of SEQ ID NO: 5;
      and (c) CDR-L3 comprising the amino acid sequence of SEQ ID NO: 6, or
   b. at least one, at least two, or all three VL CDR sequences selected from
      (a) CDR-L1 comprising the amino acid sequence of SEQ ID NO: 12;
      (b) CDR-L2 comprising the amino acid sequence of SEQ ID NO: 13;
      and (c) CDR-L3 comprising the amino acid sequence of SEQ ID NO: 14, and
B) a second, and optionally a third, antigen-binding domain that specifically binds to PD1 comprising
   a. at least one, at least two, or all three VL CDR sequences selected from
      (a) CDR-L1 comprising the amino acid sequence of SEQ ID NO: 20;
      (b) CDR-L2 comprising the amino acid sequence of SEQ ID NO: 21;
      and (c) CDR-L3 comprising the amino acid sequence of SEQ ID NO: 22, or
   b. at least one, at least two, or all three VL CDR sequences selected from
      (a) CDR-L1 comprising the amino acid sequence of SEQ ID NO: 28;
      (b) CDR-L2 comprising the amino acid sequence of SEQ ID NO: 29;
      and (c) CDR-L3 comprising the amino acid sequence of SEQ ID NO: 30.

In one aspect, the bispecific antibody comprises
A) a first antigen-binding domain that specifically binds to TfR comprising
   a. (a) CDR-L1 comprising the amino acid sequence of SEQ ID NO: 4;
      (b) CDR-L2 comprising the amino acid sequence of SEQ ID NO: 5;
      and (c) CDR-L3 comprising the amino acid sequence of SEQ ID NO: 6, or
   b. (a) CDR-L1 comprising the amino acid sequence of SEQ ID NO: 12;
      (b) CDR-L2 comprising the amino acid sequence of SEQ ID NO: 13;
      and (c) CDR-L3 comprising the amino acid sequence of SEQ ID NO: 14, and
B) a second, and optionally a third, antigen-binding domain that specifically binds to PD1 comprising
   a. (a) CDR-L1 comprising the amino acid sequence of SEQ ID NO: 20;
      (b) CDR-L2 comprising the amino acid sequence of SEQ ID NO: 21;
      and (c) CDR-L3 comprising the amino acid sequence of SEQ ID NO: 22, or
   b. (a) CDR-L1 comprising the amino acid sequence of SEQ ID NO: 28;
      (b) CDR-L2 comprising the amino acid sequence of SEQ ID NO: 29;
      and (c) CDR-L3 comprising the amino acid sequence of SEQ ID NO: 30.

In another aspect, a bispecific antibody of the invention comprises
A) a first antigen-binding domain that specifically binds to TfR comprising
   a. a VH domain comprising at least one, at least two, or all three VH CDR sequences selected from (i) CDR-H1 comprising the amino acid sequence of SEQ ID NO: 1, (ii) CDR-H2 comprising the amino acid sequence of SEQ ID NO: 2, and (iii) CDR-H3 comprising the amino acid sequence of SEQ ID NO: 3; and a VL domain comprising at least one, at least two, or all three VL CDR sequences selected from (i) CDR-L1 comprising the amino acid sequence of SEQ ID NO: 4, (ii) CDR-L2 comprising the amino acid sequence of SEQ ID NO: 5, and (c) CDR-L3 comprising the amino acid sequence of SEQ ID NO: 6,
      or
   b. a VH domain comprising at least one, at least two, or all three VH CDR sequences selected from (i) CDR-H1 comprising the amino acid sequence of SEQ ID NO: 9, (ii) CDR-H2 comprising the amino acid sequence of SEQ ID NO: 10, and (iii) CDR-H3 comprising the amino acid sequence of SEQ ID NO: 11; and a VL domain comprising at least one, at least two, or all three VL CDR sequences selected from (i) CDR-L1 comprising the amino acid sequence of SEQ ID NO: 12, (ii) CDR-L2 comprising the amino acid sequence of SEQ ID NO: 13, and (c) CDR-L3 comprising the amino acid sequence of SEQ ID NO: 14, and
B) a second, and optionally a third, antigen-binding domain that specifically binds to PD1 comprising
   a. a VH domain comprising at least one, at least two, or all three VH CDR sequences selected from (i) CDR-H1 comprising the amino acid sequence of SEQ ID NO: 17, (ii) CDR-H2 comprising the amino acid sequence of SEQ ID NO: 18, and (iii) CDR-H3 comprising the amino acid sequence of SEQ ID NO: 19; and a VL domain comprising at least one, at least two, or all three VL CDR sequences selected from (i) CDR-L1 comprising the amino acid sequence of SEQ ID NO: 20, (ii) CDR-L2 comprising the amino acid sequence of SEQ ID NO: 21, and (c) CDR-L3 comprising the amino acid sequence of SEQ ID NO: 22,
      or
   b. a VH domain comprising at least one, at least two, or all three VH CDR sequences selected from (i) CDR-H1 comprising the amino acid sequence of SEQ ID NO: 25, (ii) CDR-H2 comprising the amino acid sequence of SEQ ID NO: 26, and (iii) CDR-H3 comprising the amino acid sequence of SEQ ID NO: 27; and a VL domain comprising at least one, at least two, or all three VL CDR sequences selected from (i) CDR-L1 comprising the amino acid sequence of SEQ ID NO: 28, (ii) CDR-L2 comprising the amino acid sequence of SEQ ID NO: 29, and (c) CDR-L3 comprising the amino acid sequence of SEQ ID NO: 30.

In another aspect, the invention provides a bispecific antibody comprising
A) a first antigen-binding domain comprising (a) CDR-H1 comprising the amino acid sequence of SEQ ID NO: 1; (b) CDR-H2 comprising the amino acid sequence of SEQ ID NO: 2; (c) CDR-H3 comprising the amino acid sequence of SEQ ID NO: 3; (d) CDR-L1 comprising the amino acid sequence of SEQ ID NO: 4; (e) CDR-L2 comprising the amino acid sequence of SEQ ID NO: 5; and (f) CDR-L3 comprising the amino acid sequence of SEQ ID NO: 6 and
B) a second, and optionally a third, antigen-binding domain comprising (a) CDR-H1 comprising the amino acid sequence of SEQ ID NO: 17; (b) CDR-H2 comprising the amino acid sequence of SEQ ID NO: 18; (c) CDR-H3 comprising the amino acid sequence of SEQ ID NO: 19; (d) CDR-L1 comprising the amino acid sequence of SEQ ID NO: 20; (e) CDR-L2 comprising the amino acid sequence of SEQ ID NO: 21; and (f) CDR-L3 comprising the amino acid sequence of SEQ ID NO: 22.

In another aspect, the invention provides a bispecific antibody comprising
C) a first antigen-binding domain comprising (a) CDR-H1 comprising the amino acid sequence of SEQ ID NO: 1; (b) CDR-H2 comprising the amino acid sequence of SEQ ID NO: 2; (c) CDR-H3 comprising the amino acid sequence of SEQ ID NO: 3; (d) CDR-L1 comprising the amino acid sequence of SEQ ID NO: 4; (e) CDR-L2 comprising the amino acid sequence of SEQ ID NO: 5; and (f) CDR-L3 comprising the amino acid sequence of SEQ ID NO: 6 and
D) a second, and optionally a third, antigen-binding domain comprising (a) CDR-H1 comprising the amino acid sequence of SEQ ID NO: 25; (b) CDR-H2 comprising the amino acid sequence of SEQ ID NO: 26; (c) CDR-H3 comprising the amino acid sequence of SEQ ID NO: 27; (d) CDR-L1 comprising the amino acid sequence of SEQ ID NO: 28; (e) CDR-L2 comprising the amino acid sequence of SEQ ID NO: 29; and (f) CDR-L3 comprising the amino acid sequence of SEQ ID NO: 30.

In another aspect, the invention provides a bispecific antibody comprising
E) a first antigen-binding domain comprising (a) CDR-H1 comprising the amino acid sequence of SEQ ID NO: 9; (b) CDR-H2 comprising the amino acid sequence of SEQ ID NO: 10; (c) CDR-H3 comprising the amino acid sequence of SEQ ID NO: 11; (d) CDR-L1 comprising the amino acid sequence of SEQ ID NO: 12; (e) CDR-L2 comprising the amino acid sequence of SEQ ID NO: 13; and (f) CDR-L3 comprising the amino acid sequence of SEQ ID NO: 14 and
F) a second, and optionally a third, antigen-binding domain comprising (a) CDR-H1 comprising the amino acid sequence of SEQ ID NO: 17; (b) CDR-H2 comprising the amino acid sequence of SEQ ID NO: 18; (c) CDR-H3 comprising the amino acid sequence of SEQ ID NO: 19; (d) CDR-L1 comprising the amino acid sequence of SEQ ID NO: 20; (e) CDR-L2 comprising the amino acid sequence of SEQ ID NO: 21; and (f) CDR-L3 comprising the amino acid sequence of SEQ ID NO: 22.

In another aspect, the invention provides a bispecific antibody comprising
G) a first antigen-binding domain comprising (a) CDR-H1 comprising the amino acid sequence of SEQ ID NO: 9; (b) CDR-H2 comprising the amino acid sequence of SEQ ID NO: 10; (c) CDR-H3 comprising the amino acid sequence of SEQ ID NO: 11; (d) CDR-L1 comprising the amino acid sequence of SEQ ID NO: 12; (e) CDR-L2 comprising the amino acid sequence of SEQ ID NO: 13; and (f) CDR-L3 comprising the amino acid sequence of SEQ ID NO: 14 and
H) a second, and optionally a third, antigen-binding domain comprising (a) CDR-H1 comprising the amino acid sequence of SEQ ID NO: 25; (b) CDR-H2 comprising the amino acid sequence of SEQ ID NO: 26; (c) CDR-H3 comprising the amino acid sequence of SEQ ID NO: 27; (d) CDR-L1 comprising the amino acid sequence of SEQ ID NO: 28; (e) CDR-L2 comprising the amino acid sequence of SEQ ID NO: 29; and (f) CDR-L3 comprising the amino acid sequence of SEQ ID NO: 30.

In any of the aspects provided herein, a bispecific antibody comprising a first antigen-binding domain that specifically binds to TfR and a second, and optionally a third, antigen-binding domain that specifically binds to PD1 is humanized. In one aspect, an anti-TfR anti-PD1 bispecific antibody further comprises an acceptor human framework, e.g. a human immunoglobulin framework or a human consensus framework. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence.

In another aspect, a bispecific antibody comprising a first antigen-binding domain that specifically binds to TfR and a second, and optionally a third, antigen-binding domain that specifically binds to PD1 comprises one or more of the CDR sequences of the VH of SEQ ID NO: 7 and one or more of the CDR sequences of the VH of SEQ ID NO: 23. In another embodiment, a bispecific antibody comprising a first antigen-binding domain that specifically binds to TfR and a second, and optionally a third, antigen-binding domain that specifically binds to PD1 comprises one or more of the CDR sequences of the VL of SEQ ID NO: 8 and one or more of the CDR sequences of the VL of SEQ ID NO: 24. In another embodiment, a bispecific antibody comprising a first antigen-binding domain that specifically binds to TfR and a second, and optionally a third, antigen-binding domain that specifically binds to PD1 comprises the CDR sequences of the VH of SEQ ID NO: 7 and the CDR sequences of the VL of SEQ ID NO: 8 and the CDR sequences of the VH of SEQ ID NO: 23 and the CDR sequences of the VL of SEQ ID NO: 24.

In another aspect, a bispecific antibody comprising a first antigen-binding domain that specifically binds to TfR and a second, and optionally a third, antigen-binding domain that specifically binds to PD1 comprises one or more of the CDR sequences of the VH of SEQ ID NO: 7 and one or more of the CDR sequences of the VH of SEQ ID NO: 31. In another embodiment, a bispecific antibody comprising a first antigen-binding domain that specifically binds to TfR and a second, and optionally a third, antigen-binding domain that specifically binds to PD1 comprises one or more of the CDR sequences of the VL of SEQ ID NO: 8 and one or more of the CDR sequences of the VL of SEQ ID NO: 32. In another embodiment, a bispecific antibody comprising a first antigen-binding domain that specifically binds to TfR and a second, and optionally a third, antigen-binding domain that specifically binds to PD1 comprises the CDR sequences of the VH of SEQ ID NO: 7 and the CDR sequences of the VL of SEQ ID NO: 8 and the CDR sequences of the VH of SEQ ID NO: 31 and the CDR sequences of the VL of SEQ ID NO: 32.

In another aspect, a bispecific antibody comprising a first antigen-binding domain that specifically binds to TfR and a second, and optionally a third, antigen-binding domain that specifically binds to PD1 comprises one or more of the CDR sequences of the VH of SEQ ID NO: 15 and one or more of the CDR sequences of the VH of SEQ ID NO: 23. In another embodiment, a bispecific antibody comprising a first antigen-binding domain that specifically binds to TfR and a second, and optionally a third, antigen-binding domain that specifically binds to PD1 comprises one or more of the CDR sequences of the VL of SEQ ID NO: 16 and one or more of the CDR sequences of the VL of SEQ ID NO: 24. In another embodiment, a bispecific antibody comprising a first antigen-binding domain that specifically binds to TfR and a second, and optionally a third, antigen-binding domain that specifically binds to PD1 comprises the CDR sequences of the VH of SEQ ID NO: 15 and the CDR sequences of the VL of SEQ ID NO: 16 and the CDR sequences of the VH of SEQ ID NO: 23 and the CDR sequences of the VL of SEQ ID NO: 24.

In another aspect, a bispecific antibody comprising a first antigen-binding domain that specifically binds to TfR and a second, and optionally a third, antigen-binding domain that specifically binds to PD1 comprises one or more of the CDR sequences of the VH of SEQ ID NO: 15 and one or more of the CDR sequences of the VH of SEQ ID NO: 31. In another embodiment, a bispecific antibody comprising a first antigen-binding domain that specifically binds to TfR and a second, and optionally a third, antigen-binding domain that specifically binds to PD1 comprises one or more of the CDR sequences of the VL of SEQ ID NO: 16 and one or more of the CDR sequences of the VL of SEQ ID NO: 32. In another embodiment, a bispecific antibody comprising a first antigen-binding domain that specifically binds to TfR and a second, and optionally a third, antigen-binding domain that specifically binds to PD1 comprises the CDR sequences of the VH of SEQ ID NO: 15 and the CDR sequences of the VL of SEQ ID NO: 16 and the CDR sequences of the VH of SEQ ID NO: 31 and the CDR sequences of the VL of SEQ ID NO: 32.

In a further aspect, a bispecific antibody comprising a first antigen-binding domain that specifically binds to TfR and a second, and optionally a third, antigen-binding domain that specifically binds to PD1 comprises the CDR-H1, CDR-H2 and CDR-H3 amino acid sequences of the VH domain of SEQ ID NO: 7 and the CDR-L1, CDR-L2 and CDR-L3 amino acid sequences of the VL domain of SEQ ID NO: 8 and the CDR-H1, CDR-H2 and CDR-H3 amino acid sequences of the VH domain of SEQ ID NO: 23 and the CDR-L1, CDR-L2 and CDR-L3 amino acid sequences of the VL domain of SEQ ID NO: 24.

In a further aspect, a bispecific antibody comprising a first antigen-binding domain that specifically binds to TfR and a second, and optionally a third, antigen-binding domain that specifically binds to PD1 comprises the CDR-H1, CDR-H2 and CDR-H3 amino acid sequences of the VH domain of SEQ ID NO: 7 and the CDR-L1, CDR-L2 and CDR-L3 amino acid sequences of the VL domain of SEQ ID NO: 8 and the CDR-H1, CDR-H2 and CDR-H3 amino acid sequences of the VH domain of SEQ ID NO: 31 and the CDR-L1, CDR-L2 and CDR-L3 amino acid sequences of the VL domain of SEQ ID NO: 32.

In a further aspect, a bispecific antibody comprising a first antigen-binding domain that specifically binds to TfR and a second, and optionally a third, antigen-binding domain that specifically binds to PD1 comprises the CDR-H1, CDR-H2 and CDR-H3 amino acid sequences of the VH domain of SEQ ID NO: 15 and the CDR-L1, CDR-L2 and CDR-L3 amino acid sequences of the VL domain of SEQ ID NO: 16 and the CDR-H1, CDR-H2 and CDR-H3 amino acid sequences of the VH domain of SEQ ID NO: 23 and the CDR-L1, CDR-L2 and CDR-L3 amino acid sequences of the VL domain of SEQ ID NO: 24.

In a further aspect, a bispecific antibody comprising a first antigen-binding domain that specifically binds to TfR and a second, and optionally a third, antigen-binding domain that specifically binds to PD1 comprises the CDR-H1, CDR-H2 and CDR-H3 amino acid sequences of the VH domain of SEQ ID NO: 15 and the CDR-L1, CDR-L2 and CDR-L3 amino acid sequences of the VL domain of SEQ ID NO: 16 and the CDR-H1, CDR-H2 and CDR-H3 amino acid sequences of the VH domain of SEQ ID NO: 31 and the CDR-L1, CDR-L2 and CDR-L3 amino acid sequences of the VL domain of SEQ ID NO: 32.

In one aspect, a bispecific antibody comprising a first antigen-binding domain that specifically binds to TfR and a second, and optionally a third, antigen-binding domain that specifically binds to PD1 comprises a) one or more of the heavy chain CDR amino acid sequences of the VH domain of SEQ ID NO: 7 and a framework of at least 85%, 86%, 87%, 88%, 89%, 90%, 91%, 92%, 93%, 94%, 95%, 96%, 97%, 98%, 99% sequence identity to the framework amino acid sequence of the VH domain of SEQ ID NO: 7, and b) one or more of the heavy chain CDR amino acid sequences of the VH domain of SEQ ID NO: 23 and a framework of at least 85%, 86%, 87%, 88%, 89%, 90%, 91%, 92%, 93%, 94%, 95%, 96%, 97%, 98%, 99% sequence identity to the framework amino acid sequence of the VH domain of SEQ ID NO: 23.

In one aspect, the bispecific antibody comprising a first antigen-binding domain that specifically binds to TfR and a second, and optionally a third, antigen-binding domain that specifically binds to PD1 antibody comprises a) the three heavy chain CDR amino acid sequences of the VH domain of SEQ ID NO: 7 and a framework of at least 85%, 86%, 87%, 88%, 89%, 90%, 91%, 92%, 93%, 94%, 95%, 96%, 97%, 98%, 99% sequence identity to the framework amino acid sequence of the VH domain of SEQ ID NO: 7 and b) the three heavy chain CDR amino acid sequences of the VH domain of SEQ ID NO: 23 and a framework of at least 85%, 86%, 87%, 88%, 89%, 90%, 91%, 92%, 93%, 94%, 95%, 96%, 97%, 98%, 99% sequence identity to the framework amino acid sequence of the VH domain of SEQ ID NO: 23.

In one aspect, the bispecific antibody comprising a first antigen-binding domain that specifically binds to TfR and a second, and optionally a third, antigen-binding domain that specifically binds to PD1 comprises a) the three heavy chain CDR amino acid sequences of the VH domain of SEQ ID NO: 7 and a framework of at least 95% sequence identity to the framework amino acid sequence of the VH domain of SEQ ID NO: 7 and b) the three heavy chain CDR amino acid sequences of the VH domain of SEQ ID NO: 23 and a framework of at least 95% sequence identity to the framework amino acid sequence of the VH domain of SEQ ID NO: 23. In another aspect, the bispecific antibody comprising a first antigen-binding domain that specifically binds to TfR and a second, and optionally a third, antigen-binding domain that specifically binds to PD1 comprises a) the three heavy chain CDR amino acid sequences of the VH domain of SEQ ID NO: 7 and a framework of at least 98% sequence identity to the framework amino acid sequence of the VH domain of SEQ ID NO: 23 and b) the three heavy chain CDR amino acid sequences of the VH domain of SEQ ID NO: 7 and a framework of at least 98% sequence identity to the framework amino acid sequence of the VH domain of SEQ ID NO: 23.

In one aspect, a bispecific antibody comprising a first antigen-binding domain that specifically binds to TfR and a second, and optionally a third, antigen-binding domain that specifically binds to PD1 comprises a) one or more of the heavy chain CDR amino acid sequences of the VH domain of SEQ ID NO: 7 and a framework of at least 85%, 86%, 87%, 88%, 89%, 90%, 91%, 92%, 93%, 94%, 95%, 96%, 97%, 98%, 99% sequence identity to the framework amino acid sequence of the VH domain of SEQ ID NO: 7, and b) one or more of the heavy chain CDR amino acid sequences of the VH domain of SEQ ID NO: 31 and a framework of at least 85%, 86%, 87%, 88%, 89%, 90%, 91%, 92%, 93%, 94%, 95%, 96%, 97%, 98%, 99% sequence identity to the framework amino acid sequence of the VH domain of SEQ ID NO: 31.

In one aspect, the bispecific antibody comprising a first antigen-binding domain that specifically binds to TfR and a second, and optionally a third, antigen-binding domain that specifically binds to PD1 antibody comprises a) the three heavy chain CDR amino acid sequences of the VH domain of SEQ ID NO: 7 and a framework of at least 85%, 86%, 87%, 88%, 89%, 90%, 91%, 92%, 93%, 94%, 95%, 96%, 97%, 98%, 99% sequence identity to the framework amino acid sequence of the VH domain of SEQ ID NO: 7 and b) the three heavy chain CDR amino acid sequences of the VH domain of SEQ ID NO: 31 and a framework of at least 85%, 86%, 87%, 88%, 89%, 90%, 91%, 92%, 93%, 94%, 95%, 96%, 97%, 98%, 99% sequence identity to the framework amino acid sequence of the VH domain of SEQ ID NO: 31. In one aspect, the bispecific antibody comprising a first antigen-binding domain that specifically binds to TfR and a second, and optionally a third, antigen-binding domain that specifically binds to PD1 comprises a) the three heavy chain CDR amino acid sequences of the VH domain of SEQ ID NO: 7 and a framework of at least 95% sequence identity to the framework amino acid sequence of the VH domain of SEQ ID NO: 7 and b) the three heavy chain CDR amino acid sequences of the VH domain of SEQ ID NO: 31 and a framework of at least 95% sequence identity to the framework amino acid sequence of the VH domain of SEQ ID NO: 31. In another aspect, the bispecific antibody comprising a first antigen-binding domain that specifically binds to TfR and a second, and optionally a third, antigen-binding domain that specifically binds to PD1 comprises a) the three heavy chain CDR amino acid sequences of the VH domain of SEQ ID NO: 7 and a framework of at least 98% sequence identity to the framework amino acid sequence of the VH domain of SEQ ID NO:7 and b) the three heavy chain CDR amino acid sequences of the VH domain of SEQ ID NO: 31 and a framework of at least 98% sequence identity to the framework amino acid sequence of the VH domain of SEQ ID NO: 31.

In one aspect, a bispecific antibody comprising a first antigen-binding domain that specifically binds to TfR and a second, and optionally a third, antigen-binding domain that specifically binds to PD1 comprises a) one or more of the heavy chain CDR amino acid sequences of the VH domain of SEQ ID NO: 15 and a framework of at least 85%, 86%, 87%, 88%, 89%, 90%, 91%, 92%, 93%, 94%, 95%, 96%, 97%, 98%, 99% sequence identity to the framework amino acid sequence of the VH domain of SEQ ID NO: 15, and b) one or more of the heavy chain CDR amino acid sequences of the VH domain of SEQ ID NO: 23 and a framework of at least 85%, 86%, 87%, 88%, 89%, 90%, 91%, 92%, 93%, 94%, 95%, 96%, 97%, 98%, 99% sequence identity to the framework amino acid sequence of the VH domain of SEQ ID NO: 23.

In one aspect, the bispecific antibody comprising a first antigen-binding domain that specifically binds to TfR and a second, and optionally a third, antigen-binding domain that specifically binds to PD1 antibody comprises a) the three heavy chain CDR amino acid sequences of the VH domain of SEQ ID NO: 15 and a framework of at least 85%, 86%, 87%, 88%, 89%, 90%, 91%, 92%, 93%, 94%, 95%, 96%, 97%, 98%, 99% sequence identity to the framework amino acid sequence of the VH domain of SEQ ID NO: 15 and b) the three heavy chain CDR amino acid sequences of the VH domain of SEQ ID NO: 23 and a framework of at least 85%, 86%, 87%, 88%, 89%, 90%, 91%, 92%, 93%, 94%, 95%, 96%, 97%, 98%, 99% sequence identity to the framework amino acid sequence of the VH domain of SEQ ID NO: 23. In one aspect, the bispecific antibody comprising a first antigen-binding domain that specifically binds to TfR and a second, and optionally a third, antigen-binding domain that specifically binds to PD1 comprises a) the three heavy chain CDR amino acid sequences of the VH domain of SEQ ID NO: 15 and a framework of at least 95% sequence identity to the framework amino acid sequence of the VH domain of SEQ ID NO: 15 and b) the three heavy chain CDR amino acid sequences of the VH domain of SEQ ID NO: 23 and a framework of at least 95% sequence identity to the framework amino acid sequence of the VH domain of SEQ ID NO: 23. In another aspect, the bispecific antibody comprising a first antigen-binding domain that specifically binds to TfR and a second, and optionally a third, antigen-binding domain that specifically binds to PD1 comprises a) the three heavy chain CDR amino acid sequences of the VH domain of SEQ ID NO: 15 and a framework of at least 98% sequence identity to the framework amino acid sequence of the VH domain of SEQ ID NO: 15 and b) the three heavy chain CDR amino acid sequences of the VH domain of SEQ ID NO: 23 and a framework of at least 98% sequence identity to the framework amino acid sequence of the VH domain of SEQ ID NO: 23.

In one aspect, a bispecific antibody comprising a first antigen-binding domain that specifically binds to TfR and a second, and optionally a third, antigen-binding domain that specifically binds to PD1 comprises a) one or more of the heavy chain CDR amino acid sequences of the VH domain of SEQ ID NO: 15 and a framework of at least 85%, 86%, 87%, 88%, 89%, 90%, 91%, 92%, 93%, 94%, 95%, 96%, 97%, 98%, 99% sequence identity to the framework amino acid sequence of the VH domain of SEQ ID NO: 15, and b) one or more of the heavy chain CDR amino acid sequences of the VH domain of SEQ ID NO: 31 and a framework of at least 85%, 86%, 87%, 88%, 89%, 90%, 91%, 92%, 93%, 94%, 95%, 96%, 97%, 98%, 99% sequence identity to the framework amino acid sequence of the VH domain of SEQ ID NO: 31.

In one aspect, the bispecific antibody comprising a first antigen-binding domain that specifically binds to TfR and a second, and optionally a third, antigen-binding domain that specifically binds to PD1 comprises a) the three heavy chain CDR amino acid sequences of the VH domain of SEQ ID NO: 15 and a framework of at least 85%, 86%, 87%, 88%, 89%, 90%, 91%, 92%, 93%, 94%, 95%, 96%, 97%, 98%, 99% sequence identity to the framework amino acid sequence of the VH domain of SEQ ID NO: 15 and b) the three heavy chain CDR amino acid sequences of the VH domain of SEQ ID NO: 31 and a framework of at least 85%, 86%, 87%, 88%, 89%, 90%, 91%, 92%, 93%, 94%, 95%, 96%, 97%, 98%, 99% sequence identity to the framework amino acid sequence of the VH domain of SEQ ID NO: 31. In one aspect, the bispecific antibody comprising a first antigen-binding domain that specifically binds to TfR and a second, and optionally a third, antigen-binding domain that specifically binds to PD1 comprises a) the three heavy chain CDR amino acid sequences of the VH domain of SEQ ID NO: 15 and a framework of at least 95% sequence identity to the framework amino acid sequence of the VH domain of SEQ ID NO: 15 and b) the three heavy chain CDR amino acid sequences of the VH domain of SEQ ID NO: 31 and a framework of at least 95% sequence identity to the framework amino acid sequence of the VH domain of SEQ ID NO: 31. In another aspect, the bispecific antibody comprising a first antigen-binding domain that specifically binds to TfR and a second, and optionally a third, antigen-binding domain that specifically binds to PD1 comprises a) the three heavy chain CDR amino acid sequences of the VH domain of SEQ ID NO: 15 and a framework of at least 98% sequence identity to the framework amino acid sequence of the VH domain of SEQ ID NO: 15 and b) the three heavy chain CDR amino acid sequences of the VH domain of SEQ ID NO: 31 and a framework of at least 98% sequence identity to the framework amino acid sequence of the VH domain of SEQ ID NO: 31.

In one aspect, the bispecific antibody comprising a first antigen-binding domain that specifically binds to TfR and a second, and optionally a third, antigen-binding domain that specifically binds to PD1 comprises
A) a first antigen-binding domain specifically binding to TfR comprising (a) CDR-H1 comprising the amino acid sequence of SEQ ID NO: 1; (b) CDR-H2 comprising the amino acid sequence of SEQ ID NO: 2; (c) CDR-H3 comprising the amino acid sequence of SEQ ID NO: 3; (d) CDR-L1 comprising the amino acid sequence of SEQ ID NO: 4; (e) CDR-L2 comprising the amino acid sequence of SEQ ID NO: 5; and (f) CDR-L3 comprising the amino acid sequence of SEQ ID NO: 6, and a VH domain having at least 90%, 91%, 92%, 93%, 94%, 95%, 96%, 97%, 98%, 99%, or 100% sequence identity to the amino acid sequence of SEQ ID NO:7, and a VL domain having at least 90%, 91%, 92%, 93%, 94%, 95%, 96%, 97%, 98%, 99%, or 100% sequence identity to the amino acid sequence of SEQ ID NO: 8, and
B) a second and/or, where present, a third antigen-binding domain specifically binding to PD1 comprising (a) CDR-H1 comprising the amino acid sequence of SEQ ID NO: 17; (b) CDR-H2 comprising the amino acid sequence of SEQ ID NO: 18; (c) CDR-H3 comprising the amino acid sequence of SEQ ID NO: 19; (d) CDR-L1 comprising the amino acid sequence of SEQ ID NO: 20; (e) CDR-L2 comprising the amino acid sequence of SEQ ID NO: 21; and (f) CDR-L3 comprising the amino acid sequence of SEQ ID NO: 22, and a VH domain having at least 90%, 91%, 92%, 93%, 94%, 95%, 96%, 97%, 98%, 99%, or 100% sequence identity to the amino acid sequence of SEQ ID NO: 23, and a VL domain having at least 90%, 91%, 92%, 93%, 94%, 95%, 96%, 97%, 98%, 99%, or 100% sequence identity to the amino acid sequence of SEQ ID NO: 24.

In one aspect, the first VH domain has at least 95% sequence identity to the amino acid sequence of SEQ ID NO: 7 and the second VH domain has at least 95% sequence identity to the amino acid sequence of SEQ ID NO: 23. In one aspect, the first VL domain has at least 95% sequence identity to the amino acid sequence of SEQ ID NO: 8 and the second VL domain has at least 95% sequence identity to the amino acid sequence of SEQ ID NO: 24.

In one aspect, the bispecific antibody comprising a first antigen-binding domain that specifically binds to TfR and a second antigen-binding domain that specifically binds to PD1 comprises
A) a first antigen-binding domain specifically binding to TfR comprising (a) CDR-H1 comprising the amino acid sequence of SEQ ID NO: 1; (b) CDR-H2 comprising the amino acid sequence of SEQ ID NO: 2; (c) CDR-H3 comprising the amino acid sequence of SEQ ID NO: 3; (d) CDR-L1 comprising the amino acid sequence of SEQ ID NO: 4; (e) CDR-L2 comprising the amino acid sequence of SEQ ID NO: 5; and (f) CDR-L3 comprising the amino acid sequence of SEQ ID NO: 6, and a VH domain having at least 90%, 91%, 92%, 93%, 94%, 95%, 96%, 97%, 98%, 99%, or 100% sequence identity to the amino acid sequence of SEQ ID NO: 7, and a VL domain having at least 90%, 91%, 92%, 93%, 94%, 95%, 96%, 97%, 98%, 99%, or 100% sequence identity to the amino acid sequence of SEQ ID NO: 8, and
B) a second and/or, where present, a third antigen-binding domain specifically binding to PD1 comprising (a) CDR-H1 comprising the amino acid sequence of SEQ ID NO: 25; (b) CDR-H2 comprising the amino acid sequence of SEQ ID NO: 26; (c) CDR-H3 comprising the amino acid sequence of SEQ ID NO: 27; (d) CDR-L1 comprising the amino acid sequence of SEQ ID NO: 28; (e) CDR-L2 comprising the amino acid sequence of SEQ ID NO: 29; and (f) CDR-L3 comprising the amino acid sequence of SEQ ID NO: 30, and a VH domain having at least 90%, 91%, 92%, 93%, 94%, 95%, 96%, 97%, 98%, 99%, or 100% sequence identity to the amino acid sequence of SEQ ID NO: 31, and a VL domain having at least 90%, 91%, 92%, 93%, 94%, 95%, 96%, 97%, 98%, 99%, or 100% sequence identity to the amino acid sequence of SEQ ID NO: 32.

In one aspect, the first VH domain has at least 95% sequence identity to the amino acid sequence of SEQ ID NO: 7 and the second VH domain has at least 95% sequence identity to the amino acid sequence of SEQ ID NO: 31. In one aspect, the first VL domain has at least 95% sequence identity to the amino acid sequence of SEQ ID NO: 8 and the second VL domain has at least 95% sequence identity to the amino acid sequence of SEQ ID NO: 32.

In one aspect, the bispecific antibody comprising a first antigen-binding domain that specifically binds to TfR and a second, and optionally a third, antigen-binding domain that specifically binds to PD1 comprises
A) a first antigen-binding domain specifically binding to TfR comprising (a) CDR-H1 comprising the amino acid sequence of SEQ ID NO: 9; (b) CDR-H2 comprising the amino acid sequence of SEQ ID NO: 10; (c) CDR-H3 comprising the amino acid sequence of SEQ ID NO: 11; (d) CDR-L1 comprising the amino acid sequence of SEQ ID NO: 12; (e) CDR-L2 comprising the amino acid sequence of SEQ ID NO: 13; and (f) CDR-L3 comprising the amino acid sequence of SEQ ID NO: 14, and a VH domain having at least 90%, 91%, 92%, 93%, 94%, 95%, 96%, 97%, 98%, 99%, or 100% sequence identity to the amino acid sequence of SEQ ID NO: 15, and a VL domain having at least 90%, 91%, 92%, 93%, 94%, 95%, 96%, 97%, 98%, 99%, or 100% sequence identity to the amino acid sequence of SEQ ID NO: 16, and
B) a second and/or, where present, a third antigen-binding domain specifically binding to PD1 comprising (a) CDR-H1 comprising the amino acid sequence of SEQ ID NO: 17; (b) CDR-H2 comprising the amino acid sequence of SEQ ID NO: 18; (c) CDR-H3 comprising the amino acid sequence of SEQ ID NO: 19; (d) CDR-L1 comprising the amino acid sequence of SEQ ID NO: 20; (e) CDR-L2 comprising the amino acid sequence of SEQ ID NO: 21; and (f) CDR-L3 comprising the amino acid sequence of SEQ ID NO: 22, and a VH domain having at least 90%, 91%, 92%, 93%, 94%, 95%, 96%, 97%, 98%, 99%, or 100% sequence identity to the amino acid sequence of SEQ ID NO: 23, and a VL domain having at least 90%, 91%, 92%, 93%, 94%, 95%, 96%, 97%, 98%, 99%, or 100% sequence identity to the amino acid sequence of SEQ ID NO: 24.

In one aspect, the first VH domain has at least 95% sequence identity to the amino acid sequence of SEQ ID NO: 15 and the second VH domain has at least 95% sequence identity to the amino acid sequence of SEQ ID NO: 23. In one aspect, the first VL domain has at least 95% sequence identity to the amino acid sequence of SEQ ID NO: 16 and the second VL domain has at least 95% sequence identity to the amino acid sequence of SEQ ID NO: 24.

In one aspect, the bispecific antibody comprising a first antigen-binding domain that specifically binds to TfR and a second, and optionally a third, antigen-binding domain that specifically binds to PD1 comprises
A) a first antigen-binding domain specifically binding to TfR comprising (a) CDR-H1 comprising the amino acid sequence of SEQ ID NO: 9; (b) CDR-H2 comprising the amino acid sequence of SEQ ID NO: 10; (c) CDR-H3 comprising the amino acid sequence of SEQ ID NO: 11; (d) CDR-L1 comprising the amino acid sequence of SEQ ID NO: 12; (e) CDR-L2 comprising the amino acid sequence of SEQ ID NO: 13; and (f) CDR-L3 comprising the amino acid sequence of SEQ ID NO: 14, and a VH domain having at least 90%, 91%, 92%, 93%, 94%, 95%, 96%, 97%, 98%, 99%, or 100% sequence identity to the amino acid sequence of SEQ ID NO: 15, and a VL domain having at least 90%, 91%, 92%, 93%, 94%, 95%, 96%, 97%, 98%, 99%, or 100% sequence identity to the amino acid sequence of SEQ ID NO: 16, and
B) a second and/or, where present, a third antigen-binding domain specifically binding to PD1 comprising (a) CDR-H1 comprising the amino acid sequence of SEQ ID NO: 25; (b) CDR-H2 comprising the amino acid sequence of SEQ ID NO: 26; (c) CDR-H3 comprising the amino acid sequence of SEQ ID NO: 27; (d) CDR-L1 comprising the amino acid sequence of SEQ ID NO: 28; (e) CDR-L2 comprising the amino acid sequence of SEQ ID NO: 29; and (f) CDR-L3 comprising the amino acid sequence of SEQ ID NO: 30, and a VH domain having at least 90%, 91%, 92%, 93%, 94%, 95%, 96%, 97%, 98%, 99%, or 100% sequence identity to the amino acid sequence of SEQ ID NO: 31, and a VL domain having at least 90%, 91%, 92%, 93%, 94%, 95%, 96%, 97%, 98%, 99%, or 100% sequence identity to the amino acid sequence of SEQ ID NO: 32.

In one aspect, the first VH domain has at least 95% sequence identity to the amino acid sequence of SEQ ID NO: 15 and the second VH domain has at least 95% sequence identity to the amino acid sequence of SEQ ID NO: 31. In one aspect, the first VL domain has at least 95% sequence identity to the amino acid sequence of SEQ ID NO: 16 and the second VL domain has at least 95% sequence identity to the amino acid sequence of SEQ ID NO: 32.

In another aspect, a bispecific antibody comprising a first antigen-binding domain that specifically binds to TfR and a second, and optionally a third, antigen-binding domain that specifically binds to PD1 comprises
A) a first heavy chain variable domain (VH) sequence having at least 90%, 91%, 92%, 93%, 94%, 95%, 96%, 97%, 98%, 99%, or 100% sequence identity to an amino acid sequence selected from the group consisting of SEQ ID NO: 7 and SEQ ID NO: 15, and
B) a second heavy chain variable domain (VH) sequence having at least 90%, 91%, 92%, 93%, 94%, 95%, 96%, 97%, 98%, 99%, or 100% sequence identity to an amino acid sequence selected from the group consisting of SEQ ID NO: 23 and SEQ ID NO: 31.

In one aspect, a bispecific antibody comprising a first antigen-binding domain that specifically binds to TfR and a second, and optionally a third, antigen-binding domain that specifically binds to PD1 comprises
A) a heavy chain variable domain (VH) sequence having at least 95%, sequence identity to an amino acid sequence selected from the group consisting of SEQ ID NO: 7 and SEQ ID NO: 15, and
B) a heavy chain variable domain (VH) sequence having at least 95%, sequence identity to an amino acid sequence selected from the group consisting of SEQ ID NO: 23 and SEQ ID NO: 31.

In certain aspects, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but a bispecific antibody comprising a first antigen-binding domain that specifically binds to TfR and a second, and optionally a third, antigen-binding domain that specifically binds to PD1 comprising that sequence retains the ability to bind to TfR and/or PD1. In certain aspects, a total of 1 to 10 amino acids have been substituted, inserted and/or deleted in SEQ ID NO: 7, SEQ ID NO: 15, SEQ ID NO: 23, and/or SEQ ID NO: 31.

In certain aspects, substitutions, insertions, or deletions occur in regions outside the CDRs (i.e., in the FRs). Optionally, the bispecific antibody comprising a first antigen-binding domain that specifically binds to TfR and a second, and optionally a third, antigen-binding domain that specifically binds to PD1 comprises a first heavy chain variable domain (VH) sequence selected from the group consisting of SEQ ID NO: 7 and SEQ ID NO: 15 and a second heavy chain variable domain (VH) sequence selected from the group consisting of SEQ ID NO: 23 and SEQ ID NO: 31, including post-translational modifications of those sequences.

In another aspect, a bispecific antibody comprising a first antigen-binding domain that specifically binds to TfR and a second, and optionally a third, antigen-binding domain that specifically binds to PD1 comprises
A) a first light chain variable domain (VL) sequence having at least 90%, 91%, 92%, 93%, 94%, 95%, 96%, 97%, 98%, 99%, or 100% sequence identity to an amino acid sequence selected from the group consisting of SEQ ID NO: 8 and SEQ ID NO: 16, and
B) a second light chain variable domain (VL) sequence having at least 90%, 91%, 92%, 93%, 94%, 95%, 96%, 97%, 98%, 99%, or 100% sequence identity to an amino acid sequence selected from the group consisting of SEQ ID NO: 24 and SEQ ID NO: 32.

In one aspect, a bispecific antibody comprising a first antigen-binding domain that specifically binds to TfR and a second, and optionally a third, antigen-binding domain that specifically binds to PD1 comprises
A) a light chain variable domain (VL) sequence having at least 95%, sequence identity to an amino acid sequence selected from the group consisting of SEQ ID NO: 8 and SEQ ID NO: 16, and
B) a light chain variable domain (VL) sequence having at least 95%, sequence identity to an amino acid sequence selected from the group consisting of SEQ ID NO: 24 and SEQ ID NO: 32.

In certain aspects, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but a bispecific antibody comprising a first antigen-binding domain that specifically binds to TfR and a second, and optionally a third, antigen-binding domain that specifically binds to PD1 comprising that sequence retains the ability to bind to TfR and/or PD1. In certain aspects, a total of 1 to 10 amino acids have been substituted, inserted and/or deleted in SEQ ID NO: 8, SEQ ID NO: 16, SEQ ID NO: 24 and/or SEQ ID NO: 32. In certain aspects, the substitutions, insertions, or deletions occur in regions outside the CDRs (i.e., in the FRs). Optionally, the bispecific antibody comprising a first antigen-binding domain that specifically binds to TfR and a second, and optionally a third, antigen-binding domain that specifically binds to PD1 a first light chain variable domain (VL) sequence selected from the group consisting of SEQ ID NO: 8 and SEQ ID NO: 16 and a second light chain variable domain (VL) sequence selected from the group consisting of SEQ ID NO: 24 and SEQ ID NO: 32, including post-translational modifications of that sequence.

In another aspect, a bispecific antibody comprising a first antigen-binding domain that specifically binds to TfR and a second, and optionally a third, antigen-binding domain that specifically binds to PD1 is provided, wherein the antibody comprises a VH sequence as in any of the aspects provided above, and a VL sequence as in any of the aspects provided above. In one aspect, the bispecific antibody comprises
A) a first antigen-binding domain specifically binding to TfR comprising the VH and VL sequences in
   a. SEQ ID NO: 7 and SEQ ID NO: 8, or
   b. SEQ ID NO: 13 and SEQ ID NO: 14, and
B) a second and/or, where present, a third antigen-binding domain specifically binding to PD1 comprising the VH and VL sequences
   a. SEQ ID NO: 23 and SEQ ID NO: 24, or
   b. SEQ ID NO: 31 and SEQ ID NO: 32,
      respectively, including post-translational modifications of those sequences.

In a further aspect of the invention, a bispecific antibody comprising a first antigen-binding domain that specifically binds to TfR and a second, and optionally a third, antigen-binding domain that specifically binds to PD1 according to any of the above aspects is a monoclonal antibody, including a chimeric, humanized or human antibody. In one aspect, a bispecific antibody comprising a first antigen-binding domain that specifically binds to TfR and a second, and optionally a third, antigen-binding domain that specifically binds to PD1 comprises at least one antibody fragment, e.g., a Fv, Fab, Fab′, scFv, diabody, or F(ab′)₂ fragment.

In another aspect, the antibody is a full-length antibody, e.g., an intact IgG1 antibody or other antibody class or isotype as defined herein. In certain aspects, the antibody is of the IgG class. In another aspect, the Fab fragments and/or Fc regions of the antibody are of the IgG class. In certain aspects, the antibody is of the IgG₁ isotype. In another aspect, the Fab fragments and/or Fc regions of the antibody are of the IgG₁ isotype.

In a further aspect, the antibody as described herein is of IgG1 isotype/subclass and comprises a constant heavy chain domain of SEQ ID NO: 69 or SEQ ID NO: 70 or the constant parts of the heavy chain amino acid sequence of SEQ ID NO: 35, SEQ ID NO: 37, SEQ ID NO: 39 or SEQ ID NO: 41. In one aspect, additionally the C-terminal glycine (Gly446) is present. In one aspect, additionally the C-terminal glycine (Gly446) and the C-terminal lysine (Lys447) is present.

In another aspect, the invention relates to a bispecific antibody comprising a first antigen-binding domain that specifically binds to TfR and a second antigen-binding domain that specifically binds to PD1, which comprises a first heavy chain comprising an amino acid sequence with at least 95% sequence identity to the sequence of SEQ ID NO: 35, a first light chain comprising an amino acid sequence with at least 95% sequence identity to the sequence of SEQ ID NO: 36, and a second heavy chain comprising an amino acid sequence with at least 95% sequence identity to the sequence of SEQ ID NO: 39, and a second light chain comprising an amino acid sequence with at least 95% sequence identity to the sequence of SEQ ID NO: 40.

In another aspect, the invention relates to a bispecific antibody comprising a first antigen-binding domain that specifically binds to TfR and a second antigen-binding domain that specifically binds to PD1, which comprises a first heavy chain comprising an amino acid sequence with at least 95% sequence identity to the sequence of SEQ ID NO: 37, a first light chain comprising an amino acid sequence with at least 95% sequence identity to the sequence of SEQ ID NO: 38, and a second heavy chain comprising an amino acid sequence with at least 95% sequence identity to the sequence of SEQ ID NO: 39, and a second light chain comprising an amino acid sequence with at least 95% sequence identity to the sequence of SEQ ID NO: 40.

In another aspect, the invention relates to a bispecific antibody comprising a first antigen-binding domain that specifically binds to TfR and a second antigen-binding domain that specifically binds to PD1, which comprises a first heavy chain comprising an amino acid sequence with at least 95% sequence identity to the sequence of SEQ ID NO: 35, a first light chain comprising an amino acid sequence with at least 95% sequence identity to the sequence of SEQ ID NO: 36, and a second heavy chain comprising an amino acid sequence with at least 95% sequence identity to the sequence of SEQ ID NO: 41, and a second light chain comprising an amino acid sequence with at least 95% sequence identity to the sequence of SEQ ID NO: 42.

In another aspect, the invention relates to a bispecific antibody comprising a first antigen-binding domain that specifically binds to TfR and a second antigen-binding domain that specifically binds to PD1, which comprises a first heavy chain comprising an amino acid sequence with at least 95% sequence identity to the sequence of SEQ ID NO: 37, a first light chain comprising an amino acid sequence with at least 95% sequence identity to the sequence of SEQ ID NO: 38, and a second heavy chain comprising an amino acid sequence with at least 95% sequence identity to the sequence of SEQ ID NO: 41, and a second light chain comprising an amino acid sequence with at least 95% sequence identity to the sequence of SEQ ID NO: 42.

In another aspect, the invention relates to a bispecific antibody comprising a first antigen-binding domain that specifically binds to TfR and a second and a third antigen-binding domain that specifically binds to PD1, which comprises
a first heavy chain comprising an amino acid sequence with at least 95% sequence identity to the sequence of SEQ ID NO: 59 (Chain H), a second heavy chain comprising an amino acid sequence with at least 95% sequence identity to the sequence of SEQ ID NO: 60 (Chain K), a first light chain comprising an amino acid sequence with at least 95% sequence identity to the sequence of SEQ ID NO: 57 (Chain A), and a second light chain comprising an amino acid sequence with at least 95% sequence identity to the sequence of SEQ ID NO: 58 (Chain B).

In a further aspect, the invention relates to a bispecific antibody comprising a first antigen-binding domain that specifically binds to TfR and a second and a third antigen-binding domain that specifically binds to PD1, which comprises
a first heavy chain comprising an amino acid sequence with at least 95% sequence identity to the sequence of SEQ ID NO: 61 (Chain H), a second heavy chain comprising an amino acid sequence with at least 95% sequence identity to the sequence of SEQ ID NO: 60 (Chain K), a first light chain comprising an amino acid sequence with at least 95% sequence identity to the sequence of SEQ ID NO: 57 (Chain A), and a second light chain comprising an amino acid sequence with at least 95% sequence identity to the sequence of SEQ ID NO: 58 (Chain B).

In a further aspect, a bispecific antibody comprising a first antigen-binding domain that specifically binds to TfR and a second, and optionally a third, antigen-binding domain that specifically binds to PD1 according to any of the above aspects may incorporate any of the features, singly or in combination, as described in Sections 1-8 below:

### 1. Antibody Affinity

In certain aspects, an antibody provided herein has a dissociation constant (KD) of ≤1 μM, ≤100 nM, ≤10 nM, ≤1 nM, ≤0.1 nM, ≤0.01 nM, or ≤0.001 nM (e.g., 10−8 M or less, e.g., from 10⁻⁸ M to 10⁻¹³ M, e.g., from 10⁻⁹ M to 10⁻¹³ M).

In one aspect, K_{D} is measured using a BIACORE® surface plasmon resonance assay. For example, an assay using a BIACORE®-2000 or a BIACORE®-3000 (BIAcore, Inc., Piscataway, NJ) is performed at 25° C. with immobilized antigen CM5 chips at ˜10 response units (RU). In one aspect, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 μl/min. Association rates (kₒₙ) and dissociation rates (k_{off}) are calculated using a simple one-to-one Langmuir binding model (BIACORE & Evaluation Software version 3.2) by simultaneously fitting the association and dissociation sensorgrams. The equilibrium dissociation constant (K_{D}) is calculated as the ratio k_{off}/kₒₙ. See, e.g., Chen et al., J. Mol. Biol. 293:865-881 (1999). If the on-rate exceeds 10⁶ M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

In an alternative method, K_{D} is measured by a radiolabeled antigen-binding assay (RIA). In one aspect, an RIA is performed with the Fab version of an antibody of interest and its antigen. For example, solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881 (1999)). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 PM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 μl/well of scintillant (MICROSCINT-20 ™; Packard) is added, and the plates are counted on a TOPCOUNT™ gamma counter (Packard) for ten minutes. Concentrations of each Fab that give less than or equal to 20% of maximal binding are chosen for use in competitive binding assays.

### 2. Antibody Fragments

In certain aspects, an antibody provided herein is an antibody fragment.

In one aspect, the antibody fragment is a Fab, Fab′, Fab′-SH, or F(ab′)₂ fragment, in particular a Fab fragment. Papain digestion of intact antibodies produces two identical antigen-binding fragments, called “Fab” fragments containing each the heavy- and light-chain variable domains (VH and VL, respectively), including the three CDRs in the VH (CDR-H1, CDR-H2, CDR-H3), and the three CDRs in the VL (CDR-L1, CDR-L2, CDR-L3), and also the constant domain of the light chain (CL) and the first constant domain of the heavy chain (CH1). The term “Fab fragment” thus refers to an antibody fragment comprising a light chain comprising a VL domain and a CL domain, and a heavy chain fragment comprising a VH domain and a CH1 domain. “Fab′ fragments” differ from Fab fragments by the addition of residues at the carboxy terminus of the CH1 domain including one or more cysteines from the antibody hinge region. Fab′-SH are Fab′ fragments in which the cysteine residue(s) of the constant domains bear a free thiol group. Pepsin treatment yields an F(ab′)₂ fragment that has two antigen-binding domains (two Fab fragments) and a part of the Fc region. For discussion of Fab and F(ab′)₂ fragments comprising salvage receptor binding epitope residues and having increased in vivo half-life, see U.S. Pat. No. 5,869,046. In one aspect, the Fab fragments are of the IgG class.

In one aspect, the antibody fragment is a diabody, a triabody or a tetrabody. “Diabodies” are antibody fragments with two antigen-binding domains that may be bivalent or bispecific. See, for example, EP 404,097; WO 1993/01161; Hudson et al., Nat. Med. 9:129-134 (2003); and Hollinger et al., Proc. Natl. Acad. Sci. USA 90:6444-6448 (1993). Triabodies and tetrabodies are also described in Hudson et al., Nat. Med. 9:129-134 (2003).

In a further aspect, the antibody fragment is a single chain Fab fragment. A “single chain Fab fragment” or “scFab” is a polypeptide consisting of an antibody heavy chain variable domain (VH), an antibody heavy chain constant domain 1 (CH1), an antibody light chain variable domain (VL), an antibody light chain constant domain (CL) and a linker, wherein said antibody domains and said linker have one of the following orders in N-terminal to C-terminal direction: a) VH-CH1-linker-VL-CL, b) VL-CL-linker-VH-CH1, c) VH-CL-linker-VL-CH1 or d) VL-CH1-linker-VH-CL. In particular, said linker is a polypeptide of at least 30 amino acids, preferably between 32 and 50 amino acids. Said single chain Fab fragments are stabilized via the natural disulfide bond between the CL domain and the CH1 domain. In addition, these single chain Fab fragments might be further stabilized by generation of interchain disulfide bonds via insertion of cysteine residues (e.g., position 44 in the variable heavy chain and position 100 in the variable light chain according to Kabat numbering).

In another aspect, the antibody fragment is single-chain variable fragment (scFv). A “single-chain variable fragment” or “scFv” is a fusion protein of the variable domains of the heavy (VH) and light chains (VL) of an antibody, connected by a peptidic linker. In particular, the linker is a short polypeptide of 10 to 25 amino acids and is usually rich in glycine for flexibility, as well as serine or threonine for solubility, and can either connect the N-terminus of the VH with the C-terminus of the VL, or vice versa. This protein retains the specificity of the original antibody, despite removal of the constant regions and the introduction of the linker. For a review of scFv fragments, see, e.g., Plückthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458.

In another aspect, the antibody fragment is a single-domain antibody. “Single-domain antibodies” are antibody fragments comprising all or a portion of the heavy chain variable domain or all or a portion of the light chain variable domain of an antibody. In certain aspects, a single-domain antibody is a human single-domain antibody (Domantis, Inc., Waltham, MA; see, e.g., U.S. Pat. No. 6,248,516 B1).

Antibody fragments can be made by various techniques, including but not limited to proteolytic digestion of an intact antibody as well as recombinant production by recombinant host cells (e.g., E. coli), as described herein.

### 3. Chimeric and Humanized Antibodies

In certain aspects, an antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain aspects, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which the CDRs (or portions thereof) are derived from a non-human antibody, and FRs (or portions thereof) are derived from human antibody sequences. A humanized antibody optionally will also comprise at least a portion of a human constant region. In some aspects, some FR residues in a humanized antibody are substituted with corresponding residues from a non-human antibody (e.g., the antibody from which the CDR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

### 4. Human Antibodies

In certain aspects, an antibody provided herein is a human antibody. Human antibodies can be produced using various techniques known in the art. Human antibodies are described generally in van Dijk and van de Winkel, Curr. Opin. Pharmacol. 5:368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133:3001 (1984); Brodeur et al., Monoclonal Antibody Production Techniques and Applications, pp. 51-63 (Marcel Dekker, Inc., New York, 1987); and Boerner et al., J. Immunol., 147:86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26 (4): 265-268 (2006) (describing human-human hybridomas). Human hybridoma technology (Trioma technology) is also described in Vollmers and Brandlein, Histology and Histopathology, 20 (3): 927-937 (2005) and Vollmers and Brandlein, Methods and Findings in Experimental and Clinical Pharmacology, 27 (3): 185-91 (2005).

Human antibodies may also be generated by isolating variable domain sequences selected from human-derived phage display libraries. Such variable domain sequences may then be combined with a desired human constant domain. Techniques for selecting human antibodies from antibody libraries are described below.

### 5. Library-Derived Antibodies

In certain aspects, an antibody provided herein is derived from a library. Antibodies of the invention may be isolated by screening combinatorial libraries for antibodies with the desired activity or activities. Methods for screening combinatorial libraries are reviewed, e.g., in Lerner et al. in Nature Reviews 16:498-508 (2016). For example, a variety of methods are known in the art for generating phage display libraries and screening such libraries for antibodies possessing the desired binding characteristics. Such methods are reviewed, e.g., in Frenzel et al. in mAbs 8:1177-1194 (2016); Bazan et al. in Human Vaccines and Immunotherapeutics 8:1817-1828 (2012) and Zhao et al. in Critical Reviews in Biotechnology 36:276-289 (2016) as well as in Hoogenboom et al. in Methods in Molecular Biology 178:1-37 (O'Brien et al., ed., Human Press, Totowa, NJ, 2001) and in Marks and Bradbury in Methods in Molecular Biology 248:161-175 (Lo, ed., Human Press, Totowa, NJ, 2003).

In certain phage display methods, repertoires of VH and VL genes are separately cloned by polymerase chain reaction (PCR) and recombined randomly in phage libraries, which can then be screened for antigen-binding phage as described in Winter et al. in Annual Review of Immunology 12:433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al. in EMBO Journal 12:725-734 (1993). Furthermore, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter in Journal of Molecular Biology 227:381-388 (1992). Patent publications describing human antibody phage libraries include, for example: U.S. Pat. Nos. 5,750,373; 7,985,840; 7,785,903 and 8,679,490 as well as US Patent Publication Nos. 2005/0079574, 2007/0117126, 2007/0237764 and 2007/0292936.

Further examples of methods known in the art for screening combinatorial libraries for antibodies with a desired activity or activities include ribosome and mRNA display, as well as methods for antibody display and selection on bacteria, mammalian cells, insect cells or yeast cells. Methods for yeast surface display are reviewed, e.g., in Scholler et al. in Methods in Molecular Biology 503:135-56 (2012) and in Cherf et al. in Methods in Molecular biology 1319:155-175 (2015) as well as in Zhao et al. in Methods in Molecular Biology 889:73-84 (2012). Methods for ribosome display are described, e.g., in He et al. in Nucleic Acids Research 25:5132-5134 (1997) and in Hanes et al. in PNAS 94:4937-4942 (1997).

Antibodies or antibody fragments isolated from human antibody libraries are considered human antibodies or human antibody fragments herein.

### 6. Multispecific Antibodies

In some aspects, the bispecific antibody provided herein is a multispecific antibody, e.g. a trispecific or tetraspecific antibody. “Multispecific antibodies” are monoclonal antibodies that have binding specificities for at least two different sites, i.e., different epitopes on different antigens or different epitopes on the same antigen. In certain aspects, the multispecific antibody has three or more binding specificities. In certain aspects, one of the binding specificities is for TfR, one of the binding specificities is for PD1 and the third specificity is for any other antigen. In certain aspects, bispecific antibodies may bind to two (or more) different epitopes of TfR and/or PD1. Multispecific (e.g., bispecific) antibodies may also be used to localize cytotoxic agents or cells to cells which express PD1 and/or TfR. Multispecific antibodies may be prepared as full length antibodies or antibody fragments.

Techniques for making multispecific antibodies include, but are not limited to, recombinant co-expression of two immunoglobulin heavy chain-light chain pairs having different specificities (see Milstein and Cuello, Nature 305:537 (1983)) and “knob-in-hole” engineering (see, e.g., U.S. Pat. No. 5,731,168, and Atwell et al., J. Mol. Biol. 270:26 (1997)). Multi-specific antibodies may also be made by engineering electrostatic steering effects for making antibody Fc-heterodimeric molecules (see, e.g., WO 2009/089004); cross-linking two or more antibodies or fragments (see, e.g., U.S. Pat. No. 4,676,980, and Brennan et al., Science, 229:81 (1985)); using leucine zippers to produce bi-specific antibodies (see, e.g., Kostelny et al., J. Immunol., 148 (5): 1547-1553 (1992) and WO 2011/034605); using the common light chain technology for circumventing the light chain mis-pairing problem (see, e.g., WO 98/50431); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g., Gruber et al., J. Immunol., 152:5368 (1994)); and preparing trispecific antibodies as described, e.g., in Tutt et al. J. Immunol. 147:60 (1991).

Engineered antibodies with three or more antigen-binding domains, including for example, “Octopus antibodies”, or DVD-Ig are also included herein (see, e.g., WO 2001/77342 and WO 2008/024715). Other examples of multispecific antibodies with three or more antigen-binding domains can be found in WO 2010/115589, WO 2010/112193, WO 2010/136172, WO 2010/145792, and WO 2013/026831. The bispecific antibody or antigen-binding fragment thereof also includes a “Dual Acting FAb” or “DAF” comprising a first antigen-binding domain that binds to TfR and a second antigen-binding domain that binds to PD1 as well as another different antigen, or two different epitopes of TfR and/or PD1 (see, e.g., US 2008/0069820 and WO 2015/095539).

Multi-specific antibodies may also be provided in an asymmetric form with a domain crossover in one or more binding arms of the same antigen specificity, i.e. by exchanging the VH/VL domains (see e.g., WO 2009/080252 and WO 2015/150447), the CH1/CL domains (see e.g., WO 2009/080253) or the complete Fab arms (see e.g., WO 2009/080251, WO 2016/016299, also see Schaefer et al, PNAS, 108 (2011) 1187-1191, and Klein at al., MAbs 8 (2016) 1010-20). In one aspect, the multispecific antibody comprises a cross-Fab fragment. The term “cross-Fab fragment” or “xFab fragment” or “crossover Fab fragment” refers to a Fab fragment, wherein either the variable regions or the constant regions of the heavy and light chain are exchanged. A cross-Fab fragment comprises a polypeptide chain composed of the light chain variable region (VL) and the heavy chain constant region 1 (CH1), and a polypeptide chain composed of the heavy chain variable region (VH) and the light chain constant region (CL). Asymmetrical Fab arms can also be engineered by introducing charged or non-charged amino acid mutations into domain interfaces to direct correct Fab pairing. See e.g., WO 2016/172485.

Various further molecular formats for multispecific antibodies are known in the art and are included herein (see e.g., Spiess et al., Mol Immunol 67 (2015) 95-106).

Examples of bispecific antibody formats that may be useful for this purpose include, but are not limited to, the so-called “BiTE” (bispecific T cell engager) molecules wherein two scFv molecules are fused by a flexible linker (see, e.g., WO 2004/106381, WO 2005/061547, WO 2007/042261, and WO 2008/119567, Nagorsen and Bäuerle, Exp Cell Res 317, 1255-1260 (2011)); diabodies (Holliger et al., Prot Eng 9, 299-305 (1996)) and derivatives thereof, such as tandem diabodies (“TandAb”; Kipriyanov et al., J Mol Biol 293, 41-56 (1999)); “DART” (dual affinity retargeting) molecules which are based on the diabody format but feature a C-terminal disulfide bridge for additional stabilization (Johnson et al., J Mol Biol 399, 436-449 (2010)), and so-called triomabs, which are whole hybrid mouse/rat IgG molecules (reviewed in Seimetz et al., Cancer Treat Rev 36, 458-467 (2010)). Particular T cell bispecific antibody formats included herein are described in WO 2013/026833, WO 2013/026839, WO 2016/020309; Bacac et al., Oncoimmunology 5 (8) (2016) e1203498.

In one aspect, the bispecific antibody comprising a first antigen-binding domain that specifically binds to TfR and a second, and optionally a third, antigen-binding domain that specifically binds to PD1 is a trispecific or tetraspecific antibody, comprising
a) a first light chain and a first heavy chain of a full length antibody which specifically binds to TfR, and
b) a second (modified) light chain and a second (modified) heavy chain of a full length antibody which specifically binds to PD1, wherein the variable domains VL and VH are replaced by each other, and/or wherein the constant domains CL and CH1 are replaced by each other, and
c) wherein one to four antigen-binding domains which specifically bind to one or two further antigens (i.e. to a third and/or fourth antigen) are fused via a peptidic linker to the C- or N-terminus of the light chains or heavy chains of a) and/or b).

In one aspect, the bispecific antibody comprising a first antigen-binding domain that specifically binds to TfR and a second, and optionally a third, antigen-binding domain that specifically binds to PD1 is a trispecific or tetraspecific antibody, comprising
a) a first light chain and a first heavy chain of a full length antibody which specifically binds to PD1, and
b) a second (modified) light chain and a second (modified) heavy chain of a full length antibody which specifically binds to TfR, wherein the variable domains VL and VH are replaced by each other, and/or wherein the constant domains CL and CH1 are replaced by each other, and
c) wherein one to four antigen-binding domains which specifically bind to one or two further antigens (i.e. to a third and/or fourth antigen) are fused via a peptidic linker to the C- or N-terminus of the light chains or heavy chains of a) and/or b).

The antibody under a) does not contain a modification as reported under b) and the heavy chain and the light chain under a) are isolated chains.

In one aspect, the trispecific or tetraspecific antibody comprises under c) one or two antigen-binding domains which specifically bind to one or two further antigens.

In one aspect, the antigen-binding domains are selected from the group of a Fab fragment, a scFv fragment and a scFab fragment. In one aspect, the antigen-binding domains are Fab fragments. In one aspect, the antigen-binding domains are scFv fragments. In one aspect, the antigen-binding domains are scFab fragments.

In one aspect, the antigen-binding domains are fused to the C-terminus of the heavy chains of a) and/or b).

In one aspect, the trispecific or tetraspecific antibody comprises under c) one or two antigen-binding domains which specifically bind to one further antigen.

In one aspect, the trispecific or tetraspecific antibody comprises under c) two identical antigen-binding domains which specifically bind to a third antigen. In one preferred embodiment such two identical antigen-binding domains are fused both via the same peptidic linker to the C-terminus of the heavy chains of a) and b). In one preferred embodiment the two identical antigen-binding domains are either a Fab fragment, a scFv fragment or a scFab fragment.

In one aspect, the trispecific or tetraspecific antibody comprises under c) two antigen-binding domains which specifically bind to a third and a fourth antigen. In one embodiment said two antigen-binding domains are fused both via the same peptide connector to the C-terminus of the heavy chains of a) and b). In one preferred embodiment said two antigen-binding domains are either a Fab fragment, a scFv fragment or a scFab fragment.

In one aspect, the bispecific antibody is a bispecific, tetravalent antibody comprising
a) two light chains and two heavy chains of an antibody, which specifically bind to a first antigen (and comprise two Fab fragments),
b) two additional Fab fragments of an antibody, which specifically bind to a second antigen, wherein said additional Fab fragments are fused both via a peptidic linker either to the C- or N-termini of the heavy chains of a), and
wherein in the Fab fragments the following modifications were performed
(i) in both Fab fragments of a), or in both Fab fragments of b), the variable domains VL and VH are replaced by each other, and/or the constant domains CL and CH1 are replaced by each other, or
(ii) in both Fab fragments of a) the variable domains VL and VH are replaced by each other, and the constant domains CL and CH1 are replaced by each other, and in both Fab fragments of b) the variable domains VL and VH are replaced by each other, or the constant domains CL and CH1 are replaced by each other, or
(iii) in both Fab fragments of a) the variable domains VL and VH are replaced by each other, or the constant domains CL and CH1 are replaced by each other, and in both Fab fragments of b) the variable domains VL and VH are replaced by each other, and the constant domains CL and CH1 are replaced by each other, or
(iv) in both Fab fragments of a) the variable domains VL and VH are replaced by each other, and in both Fab fragments of b) the constant domains CL and CH1 are replaced by each other, or
(v) in both Fab fragments of a) the constant domains CL and CH1 are replaced by each other, and in both Fab fragments of b) the variable domains VL and VH are replaced by each other.

In one aspect, said additional Fab fragments are fused both via a peptidic linker either to the C-termini of the heavy chains of a), or to the N-termini of the heavy chains of a).

In one aspect, said additional Fab fragments are fused both via a peptidic linker either to the C-termini of the heavy chains of a).

In one aspect, said additional Fab fragments are fused both via a peptide connector to the N-termini of the heavy chains of a).

In one aspect, in the Fab fragments the following modifications are performed: in both Fab fragments of a), or in both Fab fragments of b), the variable domains VL and VH are replaced by each other, and/or the constant domains CL and CH1 are replaced by each other.

In one aspect, the bispecific antibody is a tetravalent antibody comprising:
a) a (modified) heavy chain of a first antibody, which specifically binds to a first antigen and comprises a first VH-CH1 domain pair, wherein to the C terminus of said heavy chain the N-terminus of a second VH-CH1 domain pair of said first antibody is fused via a peptidic linker,
b) two light chains of said first antibody of a),
c) a (modified) heavy chain of a second antibody, which specifically binds to a second antigen and comprises a first VH-CL domain pair, wherein to the C-terminus of said heavy chain the N-terminus of a second VH-CL domain pair of said second antibody is fused via a peptidic linker, and
d) two (modified) light chains of said second antibody of c), each comprising a CL-CH1 domain pair.

In one aspect, the bispecific antibody comprises
a) the heavy chain and the light chain of a first full length antibody that specifically binds to a first antigen, and
b) the heavy chain and the light chain of a second full length antibody that specifically binds to a second antigen, wherein the N-terminus of the heavy chain is connected to the C-terminus of the light chain via a peptidic linker.

The antibody under a) does not contain a modification as reported under b) and the heavy chain and the light chain are isolated chains.

In one aspect, the bispecific antibody comprises
a) a full length antibody specifically binding to a first antigen and consisting of two antibody heavy chains and two antibody light chains, and
b) an Fv fragment specifically binding to a second antigen comprising a VH2 domain and a VL2 domain, wherein both domains are connected to each other via a disulfide bridge,
wherein only either the VH2 domain or the VL2 domain is fused via a peptidic linker to the heavy or light chain of the full length antibody specifically binding to a first antigen.

In the bispecific antibody the heavy chains and the light chains under a) are isolated chains.

In one aspect, the other of the VH2 domain or the VL2 domain is not fused via a peptide linker to the heavy or light chain of the full length antibody specifically binding to a first antigen.

In some aspects, the first light chain comprises a VL domain and a CL domain and the first heavy chain comprises a VH domain, a CH1 domain, a hinge region, a CH2 domain and a CH3 domain.

In one aspect, the bispecific antibody is a trivalent antibody comprising
a) two Fab fragments that specifically binds to a first antigen,
b) one CrossFab fragment that specifically binds to a second antigen in which the CH1 and the CL domain are exchanged for each other,
c) one Fc-region comprising a first Fc-region heavy chain and a second Fc region heavy chain,
wherein the C-termini of the CH1 domains of the two Fab fragments are connected to the N-termini of the heavy chain Fc-region polypeptides, and wherein the N-terminus of the VH domain of the CrossFab fragment is connected to the C-terminus of the VH domain of one of the Fab fragments.

In one aspect, the bispecific antibody is a trivalent antibody comprising
a) one Fab fragment that specifically binds to a first antigen,
b) two CrossFab fragments that specifically bind to a second antigen in which the CH1 and the CL domain are exchanged for each other,
c) one Fc-region comprising a first Fc-region heavy chain and a second Fc region heavy chain,
wherein the C-terminus of CH1 domain of the Fab fragment is connected to the N-terminus of one of the heavy chain Fc-region polypeptides and the C-terminus of the CL-domain of one of the two CrossFab fragments is connected to the N-terminus of the other heavy chain Fc-region polypeptide, and wherein the C-terminus of the CH1 domain of the other of the two CrossFab fragments is connected to the N-terminus of the VH domain of the Fab fragment or to the N-terminus of the VH domain of the CrossFab fragment.

In one aspect, the bispecific antibody comprises
a) a bivalent full length antibody specifically binding to a first antigen and consisting of two antibody heavy chains and two antibody light chains, and
b) a Fab fragment specifically binding to a second antigen comprising a VH2 domain and a VL2 domain comprising a heavy chain fragment and a light chain fragment, wherein within the light chain fragment the variable light chain domain VL2 is replaced by the variable heavy chain domain VH2 of said antibody, and within the heavy chain fragment the variable heavy chain domain VH2 is replaced by the variable light chain domain VL2 of said antibody
wherein the heavy chain Fab fragment is inserted between the CH1 domain of one of the heavy chains of the full length antibody and the respective Fc-region of the full length antibody, and the N-terminus of the light chain Fab fragment is conjugated to the C-terminus of the light chain of the full length antibody that is paired with the heavy chain of the full length antibody into which the heavy chain Fab fragment has been inserted.

In one aspect, the bispecific antibody comprises
a) a bivalent full length antibody specifically binding to a first antigen and consisting of two antibody heavy chains and two antibody light chains, and
b) a Fab fragment specifically binding to a second antigen comprising a VH2 domain and a VL2 domain comprising a heavy chain fragment and a light chain fragment, wherein within the light chain fragment the variable light chain domain VL2 is replaced by the variable heavy chain domain VH2 of said antibody, and within the heavy chain fragment the variable heavy chain domain VH2 is replaced by the variable light chain domain VL2 of said antibody and wherein the C-terminus of the heavy chain fragment of the Fab fragment is conjugated to the N-terminus of one of the heavy chains of the full length antibody and the C-terminus of the light chain fragment of the Fab fragment is conjugated to the N-terminus of the light chain of the full length antibody that pairs with the heavy chain of the full length antibody to which the heavy chain fragment of the Fab fragment is conjugated.

In one particular aspect, a bispecific antibody is provided that is a trivalent antibody comprising
a) one Fab fragment that specifically binds to TfR,
b) two CrossFab fragments that specifically bind to PD1 in which the CH1 and the CL domain are exchanged for each other,
c) one Fc-region comprising a first Fc-region heavy chain and a second Fc region heavy chain,
wherein the C-terminus of CH1 domain of the Fab fragment is connected to the N-terminus of one of the heavy chain Fc-region polypeptides and the C-terminus of the CH1-domain of the first CrossFab fragment is connected to the N-terminus of the other heavy chain Fc-region polypeptide, and wherein the C-terminus of the CH1 domain of the second CrossFab fragment is connected to the N-terminus of the VH domain of the Fab fragment or to the N-terminus of the VL domain of the CrossFab fragment. In an additional aspect, the Fc domain is an IgG Fc domain, particularly an IgG1 Fc domain or an IgG4 Fc domain. In a particular aspect, the heavy chain of the bispecific antibody is of the γ type (IgG), particularly of the γ1 type. In another particular aspect, the light chain of the bispecific antibody is of the kappa (κ) and/or lambda (λ) subtype, based on the amino acid sequence of its constant domain.

In one particular aspect a bispecific antibody is provided that is a trivalent antibody comprising
a) two CrossFab fragments that specifically bind to PD1 in which the VL and the VH domain are exchanged for each other,
b) one Fab fragment that specifically binds to TfR,
c) one Fc-region comprising a first Fc-region heavy chain and a second Fc region heavy chain,
wherein the C-terminus of CH1 domain of the first CrossFab fragment is connected to the N-terminus of one of the heavy chain Fc-region polypeptides and the C-terminus of the CH1-domain of the Fab fragment is connected to the N-terminus of the other heavy chain Fc-region polypeptide, and wherein the C-terminus of the CH1 domain of the second CrossFab fragment is connected to the N-terminus of the VL domain of the CrossFab fragment or to the N-terminus of the VH domain of the Fab fragment. In an additional aspect, the Fc domain is an IgG Fc domain, particularly an IgG1 Fc domain or an IgG4 Fc domain. In a particular aspect, the heavy chain of the bispecific antibody is of the γ type (IgG), particularly of the γ1 type. In another particular aspect, the light chain of the bispecific antibody is of the kappa (κ) and/or lambda (λ) subtype, based on the amino acid sequence of its constant domain.

In one aspect, a bispecific antibody is provided that is a trivalent antibody comprising
a) a full length antibody specifically binding to PD1 and consisting of two antibody heavy chains and two antibody light chains, wherein within the light chain the variable light chain domain VL is replaced by the variable heavy chain domain VH of said antibody, and within the heavy chain fragment the variable heavy chain domain VH is replaced by the variable light chain domain VL of said antibody, and
b) a Fab fragment specifically binding to TfR,
wherein the N-terminus of the heavy chain Fab fragment is conjugated to the C-terminus of one of the two heavy chains of the full length antibody. In an additional aspect, the antibody and/or the Fab fragment are of the IgG class, particularly of the IgG1 or IgG4 isotype. In a particular aspect, the heavy chain of the bispecific antibody is of the γ type (IgG), particularly of the γ1 type. In another particular aspect, the light chain of the bispecific antibody is of the kappa (κ) and/or lambda (λ) subtype, based on the amino acid sequence of its constant domain.

In one aspect, a bispecific antibody is provided that is a trivalent antibody comprising
a) a full length antibody specifically binding to PD1 and consisting of two antibody heavy chains and two antibody light chains, and
b) a CrossFab fragment specifically binding to TfR in which the CH1 and the CL domain are exchanged for each other,
wherein the N-terminus of the heavy chain CrossFab fragment is conjugated to the C-terminus of one of the two heavy chains of the full length antibody.

### 7. Antibody Variants

In certain aspects, amino acid sequence variants of the antibodies provided herein are contemplated. For example, it may be desirable to alter the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

### a) Substitution, Insertion, and Deletion Variants

In certain aspects, antibody variants having one or more amino acid substitutions are provided. Sites of interest for substitutional mutagenesis include the CDRs and FRs. Conservative substitutions are shown in Table 1 under the heading of “preferred substitutions”. More substantial changes are provided in Table 2 under the heading of “exemplary substitutions”, and as further described below in reference to amino acid side chain classes. Amino acid substitutions may be introduced into an antibody of interest and the products screened for a desired activity, e.g., retained/improved antigen-binding, decreased immunogenicity, or improved ADCC or CDC.

TABLE 2

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for a member of another class.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more. CDR residues are mutated and the variant antibodies displayed on phage and screened for a particular biological activity (e.g., binding affinity).

Alterations (e.g., substitutions) may be made in CDRs, e.g., to improve antibody affinity. Such alterations may be made in CDR “hotspots”, i.e., residues encoded by codons that undergo mutation at high frequency during the somatic maturation process (see, e.g., Chowdhury, Methods Mol. Biol. 207:179-196 (2008)), and/or residues that contact antigen, with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, NJ, (2001).) In some aspects of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves CDR-directed approaches, in which several CDR residues (e.g., 4-6 residues at a time) are randomized. CDR residues involved in antigen-binding may be specifically identified, e.g., using alanine scanning mutagenesis or modeling. CDR-H3 and CDR-L3 in particular are often targeted.

In certain aspects, substitutions, insertions, or deletions may occur within one or more CDRs so long as such alterations do not substantially reduce the ability of the antibody to bind antigen. For example, conservative alterations (e.g., conservative substitutions as provided herein) that do not substantially reduce binding affinity may be made in the CDRs. Such alterations may, for example, be outside of antigen contacting residues in the CDRs. In certain variant VH and VL sequences provided above, each CDR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex may be used to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT (antibody directed enzyme prodrug therapy)) or a polypeptide which increases the serum half-life of the antibody.

### b) Glycosylation Variants

In certain aspects, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the oligosaccharide attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some aspects, modifications of the oligosaccharide in an antibody of the invention may be made in order to create antibody variants with certain improved properties.

In one aspect, antibody variants are provided having a non-fucosylated oligosaccharide, i.e. an oligosaccharide structure that lacks fucose attached (directly or indirectly) to an Fc region. Such non-fucosylated oligosaccharide (also referred to as “afucosylated” oligosaccharide) particularly is an N-linked oligosaccharide which lacks a fucose residue attached to the first GlcNAc in the stem of the biantennary oligosaccharide structure. In one aspect, antibody variants are provided having an increased proportion of non-fucosylated oligosaccharides in the Fc region as compared to a native or parent antibody. For example, the proportion of non-fucosylated oligosaccharides may be at least about 20%, at least about 40%, at least about 60%, at least about 80%, or even about 100% (i.e. no fucosylated oligosaccharides are present). The percentage of non-fucosylated oligosaccharides is the (average) amount of oligosaccharides lacking fucose residues, relative to the sum of all oligosaccharides attached to Asn 297 (e.g. complex, hybrid and high mannose structures) as measured by MALDI-TOF mass spectrometry, as described in WO 2006/082515, for example. Asn297 refers to the asparagine residue located at about position 297 in the Fc region (EU numbering of Fc region residues); however, Asn297 may also be located about ±3 amino acids upstream or downstream of position 297, i.e., between positions 294 and 300, due to minor sequence variations in antibodies. Such antibodies having an increased proportion of non-fucosylated oligosaccharides in the Fc region may have improved FcγRIIIa receptor binding and/or improved effector function, in particular improved ADCC function. See, e.g., US 2003/0157108; US 2004/0093621.

Examples of cell lines capable of producing antibodies with reduced fucosylation include Lec13 CHO cells deficient in protein fucosylation (Ripka et al. Arch. Biochem. Biophys. 249:533-545 (1986); US 2003/0157108; and WO 2004/056312, especially at Example 11), and knockout cell lines, such as alpha-1,6-fucosyltransferase gene, FUT8, knockout CHO cells (see, e.g., Yamane-Ohnuki et al. Biotech. Bioeng. 87:614-622 (2004); Kanda, Y. et al., Biotechnol. Bioeng., 94 (4): 680-688 (2006); and WO 2003/085107), or cells with reduced or abolished activity of a GDP-fucose synthesis or transporter protein (see, e.g., US2004259150, US2005031613, US2004132140, US2004110282).

In a further aspect, antibody variants are provided with bisected oligosaccharides, e.g., in which a biantennary oligosaccharide attached to the Fc region of the antibody is bisected by GlcNAc. Such antibody variants may have reduced fucosylation and/or improved ADCC function as described above.

Examples of such antibody variants are described, e.g., in Umana et al., Nat Biotechnol 17, 176-180 (1999); Ferrara et al., Biotechn Bioeng 93, 851-861 (2006); WO 99/54342; WO 2004/065540, WO 2003/011878.

Antibody variants with at least one galactose residue in the oligosaccharide attached to the Fc region are also provided. Such antibody variants may have improved CDC function. Such antibody variants are described, e.g., in WO 1997/30087; WO 1998/58964; and WO 1999/22764.

### c) Fc Region Variants

In certain aspects, one or more amino acid modifications may be introduced into the Fc region of an antibody provided herein, thereby generating an Fc region variant. The Fc region variant may comprise a human Fc region sequence (e.g., a human IgG₁, IgG₂, IgG₃ or IgG₄ Fc region) comprising an amino acid modification (e.g., a substitution) at one or more amino acid positions.

In certain aspects, the invention contemplates an antibody variant that possesses some but not all effector functions, which make it a desirable candidate for applications in which the half life of the antibody in vivo is important yet certain effector functions (such as complement-dependent cytotoxicity (CDC) and antibody-dependent cell-mediated cytotoxicity (ADCC)) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g., Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, CA; and CytoTox 96® non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. Proc. Nat'l Acad. Sci. USA 95:652-656 (1998). Clq binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half life determinations can also be performed using methods known in the art (see, e.g., Petkova, S. B. et al., Int'l. Immunol. 18 (12): 1759-1769 (2006); WO 2013/120929 A1).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem. 9 (2): 6591-6604 (2001).)

In certain aspects, an antibody variant comprises an Fc region with one or more amino acid substitutions which improve ADCC, e.g., substitutions at positions 298, 333, and/or 334 of the Fc region (EU numbering of residues).

In certain aspects, an antibody variant comprises an Fc region with one or more amino acid substitutions which diminish FcγR binding, e.g., substitutions at positions 234 and 235 of the Fc region (EU numbering of residues). In one aspect, the substitutions are L234A and L235A (LALA). In certain aspects, the antibody variant further comprises D265A and/or P329G in an Fc region derived from a human IgG1 Fc region. In one aspect, the substitutions are L234A, L235A and P329G (LALA-PG) in an Fc region derived from a human IgG₁ Fc region. (See, e.g., WO 2012/130831). In another aspect, the substitutions are L234A, L235A and D265A (LALA-DA) in an Fc region derived from a human IgG₁ Fc region.

In some aspects, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164:4178-4184 (2000).

Antibodies with increased half-lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934 (Hinton et al.). Those antibodies comprise an Fc region with one or more substitutions therein which improve binding of the Fc region to FcRn. Such Fc variants include those with substitutions at one or more of Fc region residues: 238, 252, 254, 256, 265, 272, 286, 303, 305, 307, 311, 312, 317, 340, 356, 360, 362, 376, 378, 380, 382, 413, 424 or 434, e.g., substitution of Fc region residue 434 (See, e.g., U.S. Pat. No. 7,371,826; Dall'Acqua, W. F., et al. J. Biol. Chem. 281 (2006) 23514-23524).

Fc region residues critical to the mouse Fc-mouse FcRn interaction have been identified by site-directed mutagenesis (see e.g. Dall'Acqua, W. F., et al. J. Immunol 169 (2002) 5171-5180). Residues I253, H310, H433, N434, and H435 (EU numbering of residues) are involved in the interaction (Medesan, C., et al., Eur. J. Immunol. 26 (1996) 2533; Firan, M., et al., Int. Immunol. 13 (2001) 993; Kim, J. K., et al., Eur. J. Immunol. 24 (1994) 542). Residues I253, H310, and H435 were found to be critical for the interaction of human Fc with murine FcRn (Kim, J. K., et al., Eur. J. Immunol. 29 (1999) 2819). Studies of the human Fc-human FcRn complex have shown that residues I253, S254, H435, and Y436 are crucial for the interaction (Firan, M., et al., Int. Immunol. 13 (2001) 993; Shields, R. L., et al., J. Biol. Chem. 276 (2001) 6591-6604). In Yeung, Y. A., et al. (J. Immunol. 182 (2009) 7667-7671) various mutants of residues 248 to 259 and 301 to 317 and 376 to 382 and 424 to 437 have been reported and examined.

In certain aspects, an antibody variant comprises an Fc region with one or more amino acid substitutions, which reduce FcRn binding, e.g., substitutions at positions 253, and/or 310, and/or 435 of the Fc-region (EU numbering of residues). In certain aspects, the antibody variant comprises an Fc region with the amino acid substitutions at positions 253, 310 and 435. In one aspect, the substitutions are I253A, H310A and H435A in an Fc region derived from a human IgG1 Fc-region. See, e.g., Grevys, A., et al., J. Immunol. 194 (2015) 5497-5508.

In certain aspects, an antibody variant comprises an Fc region with one or more amino acid substitutions, which reduce FcRn binding, e.g., substitutions at positions 310, and/or 433, and/or 436 of the Fc region (EU numbering of residues). In certain aspects, the antibody variant comprises an Fc region with the amino acid substitutions at positions 310, 433 and 436. In one aspect, the substitutions are H310A, H433A and Y436A in an Fc region derived from a human IgG1 Fc-region. (See, e.g., WO 2014/177460 A1).

In certain aspects, an antibody variant comprises an Fc region with one or more amino acid substitutions which increase FcRn binding, e.g., substitutions at positions 252, and/or 254, and/or 256 of the Fc region (EU numbering of residues). In certain aspects, the antibody variant comprises an Fc region with amino acid substitutions at positions 252, 254, and 256. In one aspect, the substitutions are M252Y, S254T and T256E in an Fc region derived from a human IgG₁ Fc-region. See also Duncan & Winter, Nature 322:738-40 (1988); U.S. Pat. Nos. 5,648,260; 5,624,821; and WO 94/29351 concerning other examples of Fc region variants.

The C-terminus of the heavy chain of the antibody as reported herein can be a complete C-terminus ending with the amino acid residues PGK. The C-terminus of the heavy chain can be a shortened C-terminus in which one or two of the C terminal amino acid residues have been removed. In one preferred aspect, the C-terminus of the heavy chain is a shortened C-terminus ending PG. In one aspect of all aspects as reported herein, an antibody comprising a heavy chain including a C-terminal CH3 domain as specified herein, comprises the C-terminal glycine-lysine dipeptide (G446 and K447, EU index numbering of amino acid positions). In one aspect of all aspects as reported herein, an antibody comprising a heavy chain including a C-terminal CH3 domain, as specified herein, comprises a C-terminal glycine residue (G446, EU index numbering of amino acid positions).

### d) Cysteine Engineered Antibody Variants

In certain aspects, it may be desirable to create cysteine engineered antibodies, e.g., THIOMAB™ antibodies, in which one or more residues of an antibody are substituted with cysteine residues. In particular aspects, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. Cysteine engineered antibodies may be generated as described, e.g., in U.S. Pat. Nos. 7,521,541, 8,30,930, 7,855,275, 9,000,130, or WO 2016040856.

### e) Antibody Derivatives

In certain aspects, an antibody provided herein may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone) polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

### 8. Immunoconjugates

The invention also provides immunoconjugates comprising a bispecific antibody comprising a first antigen-binding domain that specifically binds to TfR and a second, and optionally a third, antigen-binding domain that specifically binds to PD1 herein conjugated (chemically bonded) to one or more therapeutic agents such as cytotoxic agents, chemotherapeutic agents, drugs, growth inhibitory agents, toxins (e.g., protein toxins, enzymatically active toxins of bacterial, fungal, plant, or animal origin, or fragments thereof), or radioactive isotopes.

In one aspect, an immunoconjugate is an antibody-drug conjugate (ADC) in which an antibody is conjugated to one or more of the therapeutic agents mentioned above. The antibody is typically conjugated to one or more of the therapeutic agents using linkers. An overview of ADC technology including examples of therapeutic agents and drugs and linkers is set forth in Pharmacol Review 68:3-19 (2016).

In another aspect, an immunoconjugate comprises an antibody as described herein conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

In another aspect, an immunoconjugate comprises an antibody as described herein conjugated to a radioactive atom to form a radioconjugate. A variety of radioactive isotopes are available for the production of radioconjugates. Examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. When the radioconjugate is used for detection, it may comprise a radioactive atom for scintigraphic studies, for example tc99m or I123, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

Conjugates of an antibody and cytotoxic agent may be made using a variety of bifunctional protein coupling agents such as N-succinimidyl-3-(2-pyridyldithio) (SPDP), succinimidyl-4-(N-maleimidomethyl) cyclohexane-1-propionate carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO 94/11026. The linker may be a “cleavable linker” facilitating release of a cytotoxic drug in the cell. For example, an acid-labile linker, peptidase-sensitive linker, photolabile linker, dimethyl linker or disulfide-containing linker (Chari et al., Cancer Res. 52:127-131 (1992); U.S. Pat. No. 5,208,020) may be used.

The immunoconjugates or ADCs herein expressly contemplate, but are not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, IL., U.S.A).

### B. Recombinant Methods and Compositions

Antibodies may be produced using recombinant methods and compositions, e.g., as described in U.S. Pat. No. 4,816,567. For these methods one or more isolated nucleic acid(s) encoding an antibody are provided.

In case of a native antibody or native antibody fragment two nucleic acids are required, one for the light chain or a fragment thereof and one for the heavy chain or a fragment thereof. Such nucleic acid(s) encode an amino acid sequence comprising the VL and/or an amino acid sequence comprising the VH of the antibody (e.g., the light and/or heavy chain(s) of the antibody). These nucleic acids can be on the same expression vector or on different expression vectors.

In case of a bispecific antibody with heterodimeric heavy chains four nucleic acids are required, one for the first light chain, one for the first heavy chain comprising the first heteromonomeric Fc-region polypeptide, one for the second light chain, and one for the second heavy chain comprising the second heteromonomeric Fc-region polypeptide. The four nucleic acids can be comprised in one or more nucleic acid molecules or expression vectors. Such nucleic acid(s) encode an amino acid sequence comprising the first VL and/or an amino acid sequence comprising the first VH including the first heteromonomeric Fc-region and/or an amino acid sequence comprising the second VL and/or an amino acid sequence comprising the second VH including the second heteromonomeric Fc-region of the antibody (e.g., the first and/or second light and/or the first and/or second heavy chains of the antibody). These nucleic acids can be on the same expression vector or on different expression vectors, normally these nucleic acids are located on two or three expression vectors, i.e. one vector can comprise more than one of these nucleic acids. Examples of these bispecific antibodies are CrossMabs (see, e.g., Schaefer, W. et al, PNAS, 108 (2011) 11187-1191). For example, one of the heteromonomeric heavy chain comprises the so-called “knob mutations” (T366W and optionally one of S354C or Y349C) and the other comprises the so-called “hole mutations” (T366S, L368A and Y407V and optionally Y349C or S354C) (see, e.g., Carter, P. et al., Immunotechnol. 2 (1996) 73) according to EU index numbering.

In one aspect, isolated nucleic acids encoding an antibody as used in the methods as reported herein are provided.

In one aspect, a method of making a bispecific antibody comprising a first antigen-binding domain that specifically binds to TfR and a second, and optionally a third, antigen-binding domain that specifically binds to PD1 is provided, wherein the method comprises culturing a host cell comprising nucleic acid(s) encoding the antibody, as provided above, under conditions suitable for expression of the antibody, and optionally recovering the antibody from the host cell (or host cell culture medium).

For recombinant production of a bispecific antibody comprising a first antigen-binding domain that specifically binds to TfR and a second, and optionally a third, antigen-binding domain that specifically binds to PD1, nucleic acids encoding the antibody, e.g., as described above, are isolated and inserted into one or more vectors for further cloning and/or expression in a host cell. Such nucleic acids may be readily isolated and sequenced using conventional procedures (e.g., by using oligonucleotide probes that are capable of binding specifically to genes encoding the heavy and light chains of the antibody) or produced by recombinant methods or obtained by chemical synthesis.

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, K. A., In: Methods in Molecular Biology, Vol. 248, Lo, B. K. C. (ed.), Humana Press, Totowa, NJ (2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized”, resulting in the production of an antibody with a partially or fully human glycosylation pattern. See Gerngross, T. U., Nat. Biotech. 22 (2004) 1409-1414; and Li, H. et al., Nat. Biotech. 24 (2006) 210-215.

Suitable host cells for the expression of (glycosylated) antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293T cells as described, e.g., in Graham, F. L. et al., J. Gen Virol. 36 (1977) 59-74); baby hamster kidney cells (BHK); mouse sertoli cells (TM4 cells as described, e.g., in Mather, J. P., Biol. Reprod. 23 (1980) 243-252); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells (as described, e.g., in Mather, J. P. et al., Annals N.Y. Acad. Sci. 383 (1982) 44-68); MRC 5 cells; and FS4 cells. Other useful mammalian host cell lines include Chinese hamster ovary (CHO) cells, including DHFR-CHO cells (Urlaub, G. et al., Proc. Natl. Acad. Sci. USA 77 (1980) 4216-4220); and myeloma cell lines such as Y0, NS0 and Sp2/0. For a review of certain mammalian host cell lines suitable for antibody production, see, e.g., Yazaki, P. and Wu, A. M., Methods in Molecular Biology, Vol. 248, Lo, B. K. C. (ed.), Humana Press, Totowa, NJ (2004), pp. 255-268.

In one aspect, the host cell is eukaryotic, e.g., a Chinese Hamster Ovary (CHO) cell or lymphoid cell (e.g., Y0, NS0, Sp20 cell).

### C. Assays

Bispecific antibodies comprising a first antigen-binding domain that specifically binds to TfR and a second, and optionally a third, antigen-binding domain that specifically binds to PD1 provided herein may be identified, screened for, or characterized for their physical/chemical properties and/or biological activities by various assays known in the art.

### 1. Binding Assays and Other Assays

In one aspect, an antibody of the invention is tested for its antigen-binding activity, e.g., by known methods such as ELISA, Western blot, etc.

In another aspect, competition assays may be used to identify an antibody that competes with mouse anti-human transferrin-receptor antibody 128.1 (for variable region sequences see WO93/01819 and SEQ ID NO: 64 and 65) for binding to TfR. In certain aspects, such a competing antibody binds to the same epitope (e.g., a linear or a conformational epitope) that is bound by mouse anti-human transferrin-receptor antibody 128.1. Detailed exemplary methods for mapping an epitope to which an antibody binds are provided in Morris (1996) “Epitope Mapping Protocols”, in Methods in Molecular Biology vol. 66 (Humana Press, Totowa, NJ).

In an exemplary competition assay, immobilized TfR is incubated in a solution comprising a first labeled antibody that binds to TfR (e.g., mouse anti-human transferrin-receptor antibody 128.1) and a second unlabeled antibody that is being tested for its ability to compete with the first antibody for binding to TfR. The second antibody may be present in a hybridoma supernatant. As a control, immobilized TfR is incubated in a solution comprising the first labeled antibody but not the second, unlabeled antibody. After incubation under conditions permissive for binding of the first antibody to TfR, excess unbound antibody is removed, and the amount of label associated with immobilized TfR is measured. If the amount of label associated with immobilized TfR is substantially reduced in the test sample relative to the control sample, then that indicates that the second antibody is competing with the first antibody for binding to TfR. See Harlow and Lane (1988) Antibodies: A Laboratory Manual ch. 14 (Cold Spring Harbor Laboratory, Cold Spring Harbor, NY).

In another aspect, competition assays may be used to identify an antibody that competes with e.g. nivolumab or pembrolizumab for binding to PD1. In certain aspects, such a competing antibody binds to the same epitope (e.g., a linear or a conformational epitope) that is bound by e.g. nivolumab or pembrolizumab. Detailed exemplary methods for mapping an epitope to which an antibody binds are provided in Morris (1996) “Epitope Mapping Protocols”, in Methods in Molecular Biology vol. 66 (Humana Press, Totowa, NJ).

In an exemplary competition assay, immobilized PD1 is incubated in a solution comprising a first labeled antibody that binds to PD1 (e.g., nivolumab or pembrolizumab) and a second unlabeled antibody that is being tested for its ability to compete with the first antibody for binding to PD1. The second antibody may be present in a hybridoma supernatant. As a control, immobilized PD1 is incubated in a solution comprising the first labeled antibody but not the second, unlabeled antibody. After incubation under conditions permissive for binding of the first antibody to PD1, excess unbound antibody is removed, and the amount of label associated with immobilized PD1 is measured. If the amount of label associated with immobilized PD1 is substantially reduced in the test sample relative to the control sample, then that indicates that the second antibody is competing with the first antibody for binding to PD1. See Harlow and Lane (1988) Antibodies: A Laboratory Manual ch.14 (Cold Spring Harbor Laboratory, Cold Spring Harbor, NY).

In another aspect, a Jurkat cell assay is provided which allows assessment of avidity-enhanced binding of bispecific anti-TfR anti-PD1 antibodies. For that, NFAT-bla Jurkat cells expressing PD1 at different levels are generated by transducing them lentivirally with a PD1 expression construct. The Jurkat cells are contacted with the bispecific antibody and labelled. Flow cytometry is used to assess whether the binding is dependent on PD1 expression levels. The assay is described in more detail in Example 5.

### 2. Activity Assays

In one aspect, assays are provided for identifying bispecific antibodies comprising a first antigen-binding domain that specifically binds to TfR and a second, and optionally a third, antigen-binding domain that specifically binds to PD1 having biological activity. Biological activity may include, e.g., the ability to enhance the activation and/or proliferation of different immune cells, especially T cells, secretion of immune-modulating cytokines such IFNγ or TNF-alpha, blocking the PD1 pathway or killing of tumor cells. Antibodies having such biological activity in vivo and/or in vitro are also provided.

In certain aspects, an antibody of the invention is tested for such biological activity. In one aspect, an immune cell assay is provided which measures the activation of lymphocytes from one individual (donor X) to lymphocytes from another individual (donor Y). The mixed lymphocyte reaction (MLR) can demonstrate the effect of blocking the PD1 pathway to lymphocyte effector cells. T cells in the assay were tested for activation as measured by cytotoxic Granzyme B release in the presence or absence of bispecific antibodies of the invention. The assay is described in more detail in Example 13.

In another aspect, a PD1/PD-L1 blockade co-culture assay is provided which measures blockade of PD1/PD-L1-mediated inhibition of TCR signaling between PD-L1-expressing CHO-K1 and PD1-expressing Jurkat-PD1-NFAT cells. Inhibition of the TCR activation by PD1 signaling is measured by detection of expression of a reporter gene. The assay is described in more detail in Example 4.

In another aspect, an activated T cell-based internalization assay is provided that allows determining the internalization of a bispecific anti-TfR anti-PD1 antibody into the cells. For that, CD3- and CD28-activated CD4 T cells are first exposed to the antibody at 4° C., followed by an incubation at 37° C. to allow for internalization and subsequently the cells are stained and fixated. As a control, half of each sample is immediately washed, stained and fixed after the 4° C.-exposure to the antibody (internalization at 4° C. is negligible). Cells of both conditions (4° C. and 37° C.) are then stained using a fluorescence-labelled antibody that binds specifically to the bispecific anti-TfR anti-PD1 antibody. Fluorescence is detected using flow cytometry. The Geometric Mean Fluorescence Intensity (GMFI) and the frequency of the fluorescence-labelled CD4⁺ T cells is then compared between the cells and the control cells. The percentage of internalization is calculated with the following formula:

$${\%}_{Internalization} = 100 - ( ( {GMFI}^{florescent CD 4 + T {cells}_{37 ° C .}} \div {GMFI}^{fluorescent CD 4 + T {cells}_{4 ° C .}} ) * 100 )$$

The assay is described in more detail in Example 6.

### D. Methods and Compositions for Diagnostics and Detection

In certain aspects, any of the bispecific antibodies comprising a first antigen-binding domain that specifically binds to TfR and a second, and optionally a third, antigen-binding domain that specifically binds to PD1 provided herein is useful for detecting the presence of TfR or PD1 in a biological sample. The term “detecting” as used herein encompasses quantitative or qualitative detection. In certain aspects, a biological sample comprises a cell or tissue, such as immune cell or T cell infiltrates, or a tumor tissue.

In one aspect, a bispecific antibody comprising a first antigen-binding domain that specifically binds to TfR and a second, and optionally a third, antigen-binding domain that specifically binds to PD1 for use in a method of diagnosis or detection is provided. In a further aspect, a method of detecting the presence of a bispecific antibody comprising a first antigen-binding domain that specifically binds to TfR and a second, and optionally a third, antigen-binding domain that specifically binds to PD1 in a biological sample is provided. In certain aspects, the method comprises contacting the biological sample with a bispecific antibody comprising a first antigen-binding domain that specifically binds to TfR and a second, and optionally a third, antigen-binding domain that specifically binds to PD1 as described herein under conditions permissive for binding of the a bispecific antibody comprising a first antigen-binding domain that specifically binds to TfR and a second, and optionally a third, antigen-binding domain that specifically binds to PD1 antibody to TfR and/or PD1, and detecting whether a complex is formed between the a bispecific antibody comprising a first antigen-binding domain that specifically binds to TfR and a second, and optionally a third, antigen-binding domain that specifically binds to TfR and/or PD1. Such method may be an in vitro or in vivo method.

In certain aspects, labeled bispecific antibodies comprising a first antigen-binding domain that specifically binds to TfR and a second, and optionally a third, antigen-binding domain that specifically binds to PD1 are provided. Labels include, but are not limited to, labels or moieties that are detected directly (such as fluorescent, chromophoric, electron-dense, chemiluminescent, and radioactive labels), as well as moieties, such as enzymes or ligands, that are detected indirectly, e.g., through an enzymatic reaction or molecular interaction. Exemplary labels include, but are not limited to, the radioisotopes ³²P, ¹⁴C, ¹²⁵I, ³H, and ¹³¹I, fluorophores such as rare earth chelates or fluorescein and its derivatives, rhodamine and its derivatives, dansyl, umbelliferone, luceriferases, e.g., firefly luciferase and bacterial luciferase (U.S. Pat. No. 4,737,456), luciferin, 2,3-dihydrophthalazinediones, horseradish peroxidase (HRP), alkaline phosphatase, β-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase, or microperoxidase, biotin/avidin, spin labels, bacteriophage labels, stable free radicals, and the like.

### E. Pharmaceutical Compositions

In a further aspect, provided are pharmaceutical compositions comprising any of the antibodies provided herein, e.g., for use in any of the below therapeutic methods. In one aspect, a pharmaceutical composition comprises any of the antibodies provided herein and a pharmaceutically acceptable carrier. In another aspect, a pharmaceutical composition comprises any of the antibodies provided herein and at least one additional therapeutic agent, e.g., as described below.

Pharmaceutical compositions of a bispecific antibody comprising a first antigen-binding domain that specifically binds to TfR and a second, and optionally a third, antigen-binding domain that specifically binds to PD1 as described herein are prepared by mixing such antibody having the desired degree of purity with one or more optional pharmaceutically acceptable carriers (Remington's Pharmaceutical Sciences 16th edition, Osol, A. Ed. (1980)), in the form of lyophilized compositions or aqueous solutions. Pharmaceutically acceptable carriers are generally nontoxic to recipients at the dosages and concentrations employed, and include, but are not limited to: buffers such as histidine, phosphate, citrate, acetate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Halozyme, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody compositions are described in U.S. Pat. No. 6,267,958. Aqueous antibody compositions include those described in U.S. Pat. No. 6,171,586 and WO 2006/044908, the latter compositions including a histidine-acetate buffer.

The pharmaceutical composition herein may also contain more than one active ingredients as necessary for the particular indication being treated, preferably those with complementary activities that do not adversely affect each other. In certain aspects, an additional therapeutic agent is an immunomodulatory agent, a cytostatic agent, an inhibitor of cell adhesion, a cytotoxic agent, an activator of cell apoptosis, or an agent that increases the sensitivity of cells to apoptotic inducers. In a preferred aspect, the additional therapeutic agent is an anti-cancer agent, for example a microtubule disruptor, an antimetabolite, a topoisomerase inhibitor, a DNA intercalator, an alkylating agent, a hormonal therapy, a kinase inhibitor, a receptor antagonist, an activator of tumor cell apoptosis, or an antiangiogenic agent. Such active ingredients are suitably present in combination in amounts that are effective for the purpose intended. Active ingredients may be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nanoparticles and nanocapsules) or in macroemulsions. Such techniques are disclosed in Remington's Pharmaceutical Sciences 16th edition, Osol, A. Ed. (1980).

Pharmaceutical compositions for sustained-release may be prepared. Suitable examples of sustained-release preparations include semipermeable matrices of solid hydrophobic polymers containing the antibody, which matrices are in the form of shaped articles, e.g., films, or microcapsules.

The pharmaceutical compositions to be used for in vivo administration are generally sterile. Sterility may be readily accomplished, e.g., by filtration through sterile filtration membranes.

### F. Therapeutic Methods and Routes of Administration

Any of the anti-TfR anti-PD1 bispecific antibodies provided herein may be used in therapeutic methods.

In one aspect, an anti-TfR anti-PD1 bispecific antibody for use as a medicament is provided. In further aspects, an anti-TfR anti-PD1 bispecific antibody for use in treating cancer is provided. In certain aspects, an anti-TfR anti-PD1 bispecific antibody for use in a method of treatment is provided. In certain aspects, the invention provides an anti-TfR anti-PD1 bispecific antibody for use in a method of treating an individual having cancer comprising administering to the individual an effective amount of the anti-TfR anti-PD1 bispecific antibody. In other aspects, the invention provides an anti-TfR anti-PD1 bispecific antibody for use in a method of treating an individual having an infectious disease, preferably a chronic or an acute infection, e.g. a chronic or an acute viral infection, comprising administering to the individual an effective amount of the anti-TfR anti-PD1 bispecific antibody. In yet another aspect, the invention provides an anti-TfR anti-PD1 bispecific antibody for use in a method of treating an individual having a neurodegenerative disease such as Alzheimer's disease, comprising administering to the individual an effective amount of the anti-TfR anti-PD1 bispecific antibody. In one such aspect, the method further comprises administering to the individual an effective amount of at least one additional therapeutic agent (e.g., one, two, three, four, five, or six additional therapeutic agents), e.g., as described below. In further aspects, the invention provides an anti-TfR anti-PD1 bispecific antibody for use as immunostimulatory agent or stimulating interferon-gamma (IFN-gamma) or tumor necrosis factor alpha (TNF alpha) secretion. In certain embodiments, the invention provides an anti-TfR anti-PD1 bispecific antibody for use in a method of immunostimulation or stimulating interferon-gamma (IFN-gamma) or tumor necrosis factor alpha (TNF alpha) secretion in an individual comprising administering to the individual an effective amount of the anti-TfR anti-PD1 b bispecific antibody for immunostimulation or stimulating interferon-gamma (IFN-gamma)) or tumor necrosis factor alpha (TNF alpha) secretion. An “individual” according to any of the above aspects is preferably a human.

In a further aspect, the invention provides for the use of an anti-TfR anti-PD1 bispecific antibody in the manufacture or preparation of a medicament. In one aspect, the medicament is for treatment of cancer. In a further aspect, the medicament is for use in a method of treating cancer comprising administering to an individual having cancer an effective amount of the medicament. In one such aspect, the method further comprises administering to the individual an effective amount of at least one additional therapeutic agent, e.g., as described below. In a further aspect, the medicament is for inducing cell-mediated lysis of cancer cells. In a further aspect, the medicament is for use in a method of inducing cell mediated lysis of cancer cells in an individual comprising administering to the individual an effective amount of the medicament to induce apoptosis in a cancer cell/or to inhibit cancer cell proliferation. An “individual” according to any of the above aspects may be a human.

In a further aspect, the invention provides a method for treating cancer. In one aspect, the method comprises administering to an individual having such cancer an effective amount of an anti-TfR anti-PD1 bispecific antibody. In one such aspect, the method further comprises administering to the individual an effective amount of at least one additional therapeutic agent, as described below.

An “individual” according to any of the above aspects may be a human.

In a further aspect, the invention provides a method for immunostimulation or stimulating interferon-gamma (IFN-gamma) or tumor necrosis factor alpha (TNF alpha) secretion in an individual. In one aspect, the method comprises administering to the individual an effective amount of an anti-TfR anti-PD1 bispecific antibody for immunostimulation or stimulating interferon-gamma (IFN-gamma) or tumor necrosis factor alpha (TNF alpha) secretion. In one aspect, an “individual” is a human.

In a further aspect, the invention provides pharmaceutical compositions comprising any of the anti-TfR anti-PD1 bispecific antibodies provided herein, e.g., for use in any of the above therapeutic methods. In one aspect, a pharmaceutical composition comprises any of the anti-TfR anti-PD1 bispecific antibodies provided herein and a pharmaceutically acceptable carrier. In another aspect, a pharmaceutical composition comprises any of the anti-TfR anti-PD1 bispecific antibodies provided herein and at least one additional therapeutic agent, e.g., as described below.

Antibodies of the invention can be administered alone or used in a combination therapy. For instance, the combination therapy includes administering an antibody of the invention and administering at least one additional therapeutic agent (e.g. one, two, three, four, five, or six additional therapeutic agents). In certain aspects, the combination therapy comprises administering an antibody of the invention and administering at least one additional therapeutic agent, such as an immunomodulatory agent, a cytostatic agent, an inhibitor of cell adhesion, a cytotoxic agent, an activator of cell apoptosis, or an agent that increases the sensitivity of cells to apoptotic inducers. In a preferred aspect, the additional therapeutic agent is an anti-cancer agent, for example a microtubule disruptor, an antimetabolite, a topoisomerase inhibitor, a DNA intercalator, an alkylating agent, a hormonal therapy, a kinase inhibitor, a receptor antagonist, an activator of tumor cell apoptosis, or an antiangiogenic agent.

Such combination therapies noted above encompass combined administration (where two or more therapeutic agents are included in the same or separate pharmaceutical compositions), and separate administration, in which case, administration of the antibody of the invention can occur prior to, simultaneously, and/or following, administration of the additional therapeutic agent or agents. In one aspect, administration of the anti-TfR anti-PD1 bispecific antibody and administration of an additional therapeutic agent occur within about one month, or within about one, two or three weeks, or within about one, two, three, four, five, or six days, of each other. In one aspect, the antibody and additional therapeutic agent are administered to the patient on Day 1 of the treatment. Antibodies of the invention can also be used in combination with radiation therapy.

An antibody of the invention (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

Antibodies of the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody need not be, but is optionally formulated with one or more agents currently used to prevent or treat the disorder in question. The effective amount of such other agents depends on the amount of antibody present in the pharmaceutical composition, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of an antibody of the invention (when used alone or in combination with one or more other additional therapeutic agents) will depend on the type of disease to be treated, the type of antibody, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments. Depending on the type and severity of the disease, about 1 μg/kg to 15 mg/kg (e.g., 0.1 mg/kg-10 mg/kg) of antibody can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of the antibody would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. Such doses may be administered intermittently, e.g., every week or every three weeks (e.g., such that the patient receives from about two to about twenty, or, e.g., about six doses of the antibody). An initial higher loading dose, followed by one or more lower doses may be administered. An exemplary dosing regimen comprises administering an initial loading dose of about 4 mg/kg, followed by a weekly maintenance dose of about 2 mg/kg of the antibody. However, other dosage regimens may be useful. The progress of this therapy is easily monitored by conventional techniques and assays.

### G. Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is an antibody of the invention. The label or package insert indicates that the composition is used for treating the condition of choice. Moreover, the article of manufacture may comprise (a) a first container with a composition contained therein, wherein the composition comprises an antibody of the invention; and (b) a second container with a composition contained therein, wherein the composition comprises a further cytotoxic or otherwise therapeutic agent. The article of manufacture in this aspect of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

In the following specific embodiments of the invention are listed:
1. A bispecific antibody comprising a first antigen-binding domain that specifically binds to TfR and a second antigen-binding domain that specifically binds to PD1.
2. The bispecific antibody of embodiment 1 wherein the bispecific antibody binds to TfR and to PD1 displayed on the surface of a cell and wherein the bispecific antibody is internalized into the cell.
3. The bispecific antibody of embodiment 2 wherein the cell expresses TfR and PD1 and displays them on its surface.
4. The bispecific antibody of embodiments 2 or 3 wherein PD1 is depleted from the surface of the cell upon binding of the bispecific antibody to TfR and to PD1 displayed on the surface of the cell.
5. The bispecific antibody of one of the preceding embodiments comprising a third antigen-binding domain, wherein the third antigen-binding domain specifically binds to PD1.
6 The bispecific antibody of one of the preceding embodiments, wherein the first, the second and/or, where present, the third antigen-binding domain is a Fab fragment, preferably an IgG derived Fab fragment.
7. The bispecific antibody of one of the preceding embodiments, wherein the first and the second antigen-binding domain are each a Fab fragment and either (i) the second antigen-binding domain is fused at the C-terminus of the Fab heavy chain to the N-terminus of the Fab heavy chain of the first antigen-binding domain, or (ii) the first antigen-binding domain is fused at the C-terminus of the Fab heavy chain to the N-terminus of the Fab heavy chain of the second antigen-binding domain.
8. The bispecific antibody of one of the preceding embodiments, comprising an Fc domain, preferably an IgG derived Fc domain, composed of a first and a second subunit.
9. The bispecific antibody of embodiment 8, wherein one or more of the Fab fragments are fused to the Fc domain.
10. The bispecific antibody of any one of embodiments 7 to 9, wherein one or more of the Fab fragments are fused to the Fc domain via a peptidic linker.
11. A bispecific antibody comprising a first antigen-binding domain that specifically binds to TfR and a second, and optionally a third, antigen-binding domain that specifically binds to PD1, wherein the first, the second and, where present, the third antigen-binding domain are each a Fab fragment and the antibody comprises an Fc domain composed of a first and a second subunit; and wherein either (i) the second antigen-binding domain is fused at the C-terminus of its Fab heavy chain to the N-terminus of the Fab heavy chain of the first antigen-binding domain and the first antigen-binding domain is fused at the C-terminus of its Fab heavy chain to the N-terminus of the first subunit of the Fc domain, or (ii) the first antigen-binding domain is fused at the C-terminus of its Fab heavy chain to the N-terminus of the Fab heavy chain of the second antigen-binding domain and the second antigen-binding domain is fused at the C-terminus of the Fab heavy chain to the N-terminus of the first subunit of the Fc domain; and wherein the third antigen-binding domain, where present, is fused at the C-terminus of its Fab heavy chain to the N-terminus of the second subunit of the Fc domain.
12. A bispecific antibody comprising a first antigen-binding domain that specifically binds to TfR and a second and a third antigen-binding domain that specifically binds to PD1, wherein the first, the second and the third antigen-binding domain are each a Fab fragment and the antibody comprises an Fc domain composed of a first and a second subunit; and wherein
   the first antigen-binding domain is fused at the C-terminus of its Fab heavy chain to the N-terminus of the first subunit of the Fc domain,
   the second antigen-binding domain is fused at the C-terminus of its Fab heavy chain to the N-terminus of the second subunit of the Fc domain and
   the third antigen-binding domain is fused at the N-terminus of its Fab heavy chain to the C-terminus of the first or second subunit of the Fc domain.
13. The bispecific antibody of any one of embodiments 8 to 12, wherein the Fc domain is an IgG Fc domain, particularly an IgG1 Fc domain or an IgG4 Fc domain.
14. The bispecific antibody of any one of embodiments 1 to 13, wherein the bispecific antibody comprises at least two heavy chains and wherein the heavy chains of the bispecific antibody are of the γ type (IgG), in particular of the γ1 type
15. The bispecific antibody of any one of embodiments 1 to 14, wherein the bispecific antibody comprises at least two light chains and wherein the light chains of the bispecific antibody are selected from the kappa (κ) and/or lambda (λ) subtype.
16. The bispecific antibody of any one of embodiments 8 to 15, wherein the Fc domain comprises one or more amino acid substitutions that reduce binding to an Fc receptor, in particular towards Fcγ receptor.
17. The bispecific antibody of any one of embodiments 8 to 16, wherein the Fc domain is of the human IgG1 subclass with the amino acid mutations L234A, L235A and P329G (numbering according to Kabat EU index).
18. The bispecific antibody of any one of embodiments 8 to 17, wherein the Fc domain comprises a modification promoting the association of the first and second subunit of the Fc domain.
19. The bispecific antibody of any one of embodiments 8 to 18, wherein the first subunit of the Fc domain comprises knobs and the second subunit of the Fc domain comprises holes according to the knobs into holes method.
20. The bispecific antibody of any one of embodiments 8 to 19, wherein the first subunit of the Fc domain comprises the amino acid substitutions S354C and T366W (numbering according to Kabat EU index) and the second subunit of the Fc domain comprises the amino acid substitutions Y349C, T366S and Y407V (numbering according to Kabat EU index).
21. The bispecific antibody of any one of embodiments 6 to 20, wherein in one of the Fab fragments the variable domains VL and VH are replaced by each other so that the VH domain is part of the light chain and the VL domain is part of the heavy chain.
22. The bispecific antibody of any one of embodiments 6 to 21, wherein in the second Fab fragment and/or, where present, the third Fab fragment comprising the antigen-binding domain that specifically binds to PD1 the variable domains VL and VH are replaced by each other so that the VH domain is part of the light chain and the VL domain is part of the heavy chain.
23. The bispecific antibody of any one of embodiments 6 to 22, wherein in one of the Fab fragments in the constant domain CL the amino acid at position 124 is substituted independently by lysine (K), arginine (R) or histidine (H) (numbering according to Kabat EU Index), and in the constant domain CH1 the amino acids at positions 147 and 213 are substituted independently by glutamic acid (E) or aspartic acid (D) (numbering according to Kabat EU index).
24. The bispecific antibody of any one of embodiments 6 to 23, wherein in the first Fab fragment comprising the antigen-binding domain that specifically binds to TfR the constant domain CL the amino acid at position 124 is substituted independently by lysine (K), arginine (R) or histidine (H) (numbering according to Kabat EU Index), and in the constant domain CH1 the amino acids at positions 147 and 213 are substituted independently by glutamic acid (E) or aspartic acid (D) (numbering according to Kabat EU index).
25. The bispecific antibody of any one of embodiments 1 to 24, wherein the bispecific antibody does not comprise a J-chain.
26. The bispecific antibody of any one of embodiments 1 to 25, wherein the bispecific antibody does not comprise any hybrid IgA/IgG antibody sequences and/or any hybrid IgM/IgG antibody sequences.
27. The bispecific antibody of any one of embodiments 1 to 26, wherein the bispecific antibody is monomeric.
28. A bispecific antibody comprising a first antigen-binding domain that specifically binds to TfR and a second, and optionally a third, antigen-binding domain that specifically binds to PD1 wherein the bispecific antibody comprises an Fc domain, a first Fab fragment comprising the antigen-binding domain that specifically binds to TfR and a second and, where present, a third Fab fragment comprising the antigen-binding domain that specifically binds to PD1, and wherein the Fab fragments are fused to the Fc domain.
29. A bispecific antibody comprising exactly one (monovalent) antigen-binding domain that specifically binds to TfR and exactly two (monovalent) antigen-binding domains that specifically bind to PD1, wherein the bispecific antibody comprises an Fc domain, a Fab fragment comprising the antigen-binding domain that specifically binds to TfR and two Fab fragments wherein each of the two Fab fragments comprises one antigen-binding domain that specifically binds to PD1, and wherein at least two of the three Fab fragments are fused to the Fc domain, and one of the three Fab fragments is optionally fused to one of the other Fab fragments.
30. A bispecific antibody comprising a first antigen-binding domain that specifically binds to TfR and a second, and optionally a third, antigen-binding domain that specifically binds to PD1, wherein the first, the second and, where present, the third antigen-binding domain are each a Fab fragment and the antibody comprises an Fc domain composed of a first and a second subunit; and wherein either
   (i) the second antigen-binding domain is fused at the C-terminus of its Fab heavy chain to the N-terminus of the Fab heavy chain of the first antigen-binding domain and the first antigen-binding domain is fused at the C-terminus of its Fab heavy chain to the N-terminus of the first subunit of the Fc domain, or
   (ii) the first antigen-binding domain is fused at the C-terminus of its Fab heavy chain to the N-terminus of the Fab heavy chain of the second antigen-binding domain and the second antigen-binding domain is fused at the C-terminus of the Fab heavy chain to the N-terminus of the first subunit of the Fc domain; and wherein, where present, the third antigen-binding domain is fused at the C-terminus of its Fab heavy chain to the N-terminus of the second subunit of the Fc domain.
31. A bispecific antibody comprising a first antigen-binding domain that specifically binds to TfR and a second, and optionally a third, antigen-binding domain that specifically binds to PD1, wherein the first, the second and the third antigen-binding domain are each a Fab fragment and the antibody comprises an Fc domain composed of a first and a second subunit; and wherein the first antigen-binding domain is fused at the C-terminus of its Fab heavy chain to the N-terminus of the first subunit of the Fc domain, the second antigen-binding domain is fused at the C-terminus of its Fab heavy chain to the N-terminus of the second subunit of the Fc domain and the third antigen-binding domain is fused at the N-terminus of its Fab heavy chain to the C-terminus of the first or second subunit of the Fc domain.
32. The bispecific antibody of one of the preceding embodiments, wherein the first antigen-binding domain specifically binding TfR comprises
   i. a heavy chain variable domain (VH) comprising
      a) CDR-H1 comprising the amino acid sequence of SEQ ID NO: 1,
      b) CDR-H2 comprising the amino acid sequence of SEQ ID NO: 2, and
      c) CDR-H3 comprising the amino acid sequence of SEQ ID NO: 3, and
         a light chain variable domain (VL) comprising
      d) CDR-L1 comprising the amino acid sequence of SEQ ID NO: 4,
      e) CDR-L2 comprising the amino acid sequence of SEQ ID NO: 5, and
      f) CDR-L3 comprising the amino acid sequence of SEQ ID NO: 6, or
   ii. a heavy chain variable domain (VH) comprising
      a) CDR-H1 comprising the amino acid sequence of SEQ ID NO: 9,
      b) CDR-H2 comprising the amino acid sequence of SEQ ID NO: 10, and
      c) CDR-H3 comprising the amino acid sequence of SEQ ID NO: 11, and
         a light chain variable domain (VL) comprising
      d) CDR-L1 comprising the amino acid sequence of SEQ ID NO: 12,
      e) CDR-L2 comprising the amino acid sequence of SEQ ID NO: 13, and
      f) CDR-L3 comprising the amino acid sequence of SEQ ID NO: 14.
33. The bispecific antibody of one of the preceding embodiments, wherein the second antigen-binding domain and/or, where present, the third antigen-binding domain specifically binding PD1 comprise(s)
   i. a heavy chain variable domain (VH) comprising
   a) CDR-H1 comprising the amino acid sequence of SEQ ID NO: 17,
   b) CDR-H2 comprising the amino acid sequence of SEQ ID NO: 18, and
   c) CDR-H3 comprising the amino acid sequence of SEQ ID NO: 19, and
      a light chain variable domain (VL) comprising
   d) CDR-L1 comprising the amino acid sequence of SEQ ID NO: 20,
   e) CDR-L2 comprising the amino acid sequence of SEQ ID NO: 21, and
   f) CDR-L3 comprising the amino acid sequence of SEQ ID NO: 22, or
   ii. a heavy chain variable domain (VH) comprising
   a) CDR-H1 comprising the amino acid sequence of SEQ ID NO: 25,
   b) CDR-H2 comprising the amino acid sequence of SEQ ID NO: 26, and
   c) CDR-H3 comprising the amino acid sequence of SEQ ID NO: 27, and
      a light chain variable domain (VL) comprising
   d) CDR-L1 comprising the amino acid sequence of SEQ ID NO: 28,
   e) CDR-L2 comprising the amino acid sequence of SEQ ID NO: 29, and
   f) CDR-L3 comprising the amino acid sequence of SEQ ID NO: 30.
34. The bispecific antibody of one of the preceding embodiments, wherein
   i. said first antigen-binding domain specifically binding to TfR comprises
      a) a VH domain having at least 95% sequence identity to the amino acid sequence of SEQ ID NO: 7 and a VL domain having at least 95% sequence identity to the amino acid sequence of SEQ ID NO: 8, or
      b) a VH domain having at least 95% sequence identity to the amino acid sequence of SEQ ID NO: 15 and a VL domain having at least 95% sequence identity to the amino acid sequence of SEQ ID NO: 16, and
   ii. said second antigen-binding domain and/or, where present, said third antigen-binding domain specifically binding to PD1 comprises
      a) a VH domain having at least 95% sequence identity to the amino acid sequence of SEQ ID NO: 23 and a VL domain having at least 95% sequence identity to the amino acid sequence of SEQ ID NO: 24, or
      b) a VH domain having at least 95% sequence identity to the amino acid sequence of SEQ ID NO: 31 and a VL domain having at least 95% sequence identity to the amino acid sequence of SEQ ID NO: 32.
35. The bispecific antibody of one of the preceding embodiments, wherein
   i. said first antigen-binding domain specifically binding to TfR comprises
      a) a VH domain comprising the amino acid sequence of SEQ ID NO: 7 and a VL domain comprising the amino acid sequence of SEQ ID NO: 8, or
      b) a VH domain comprising the amino acid sequence of SEQ ID NO: 15 and a VL domain comprising the amino acid sequence of SEQ ID NO: 16,
   and
   ii. said second antigen-binding domain and/or, where present, said third antigen-binding domain specifically binding to PD1 comprises
      a) a VH domain comprising the amino acid sequence of SEQ ID NO: 23 and a VL domain comprising the amino acid sequence of SEQ ID NO: 24 or
      b) a VH domain comprising the amino acid sequence of SEQ ID NO: 31 and a VL domain comprising the amino acid sequence of SEQ ID NO: 32.
36. The bispecific antibody of one of the preceding embodiments, which is a monoclonal antibody.
37. The bispecific antibody of one of the preceding embodiments, which is a humanized, or chimeric antibody.
38. The bispecific antibody of one of the preceding embodiments, comprising a first heavy chain comprising an amino acid sequence with at least 95% sequence identity to the sequence of SEQ ID NO: 35, a first light chain comprising an amino acid sequence with at least 95% sequence identity to the sequence of SEQ ID NO: 36, a second heavy chain comprising an amino acid sequence with at least 95% sequence identity to the sequence of SEQ ID NO: 39, and a second light chain comprising an amino acid sequence with at least 95% sequence identity to the sequence 40.
39. The bispecific antibody of one of the preceding embodiments, comprising a first heavy chain comprising an amino acid sequence with at least 95% sequence identity to the sequence of SEQ ID NO: 37, a first light chain comprising an amino acid sequence with at least 95% sequence identity to the sequence of SEQ ID NO: 38, a second heavy chain comprising an amino acid sequence with at least 95% sequence identity to the sequence of SEQ ID NO: 39, and a second light chain comprising an amino acid sequence with at least 95% sequence identity to the sequence 40.
40. The bispecific antibody of one of the preceding embodiments, comprising a first heavy chain comprising an amino acid sequence with at least 95% sequence identity to the sequence of SEQ ID NO: 35, a first light chain comprising an amino acid sequence with at least 95% sequence identity to the sequence of SEQ ID NO: 36, a second heavy chain comprising an amino acid sequence with at least 95% sequence identity to the sequence of SEQ ID NO: 41, and a second light chain comprising an amino acid sequence with at least 95% sequence identity to the sequence 42.
41. The bispecific antibody of one of the preceding embodiments, comprising a first heavy chain comprising an amino acid sequence with at least 95% sequence identity to the sequence of SEQ ID NO: 37, a first light chain comprising an amino acid sequence with at least 95% sequence identity to the sequence of SEQ ID NO: 38, a second heavy chain comprising an amino acid sequence with at least 95% sequence identity to the sequence of SEQ ID NO: 41, and a second light chain comprising an amino acid sequence with at least 95% sequence identity to the sequence 42.
42. The bispecific antibody of one of the preceding embodiments, comprising a first heavy chain comprising an amino acid sequence with at least 95% sequence identity to the sequence of SEQ ID NO: 59, a second heavy chain comprising an amino acid sequence with at least 95% sequence identity to the sequence of SEQ ID NO: 60, a first light chain comprising an amino acid sequence with at least 95% sequence identity to the sequence of SEQ ID NO: 57, and a second light chain comprising an amino acid sequence with at least 95% sequence identity to the sequence 58.
43. The bispecific antibody of one of the preceding embodiments, comprising a first heavy chain comprising an amino acid sequence with at least 95% sequence identity to the sequence of SEQ ID NO: 61, a second heavy chain comprising an amino acid sequence with at least 95% sequence identity to the sequence of SEQ ID NO: 60, a first light chain comprising an amino acid sequence with at least 95% sequence identity to the sequence of SEQ ID NO: 57, and a second light chain comprising an amino acid sequence with at least 95% sequence identity to the sequence 58.
44. The bispecific antibody of one of the preceding embodiments, wherein the antibody binds to both TfR and to PD1 with affinities in the nM to sub-nM ranges.
45. The bispecific antibody of one of the preceding embodiments, wherein the antibody is a multispecific antibody.
46. The bispecific antibody of one of the preceding embodiments comprising a first heavy chain of SEQ ID NO: 37 and a first light chain of SEQ ID NO: 38, a second heavy chain of SEQ ID NO: 39 and a second light chain of SEQ ID NO: 40.
47. The bispecific antibody of one of the preceding embodiments comprising a first heavy chain of SEQ ID NO: 35 and a first light chain of SEQ ID NO: 36, a second heavy chain of SEQ ID NO: 39 and a second light chain of SEQ ID NO: 40.
48. The bispecific antibody of one of the preceding embodiments comprising a first heavy chain of SEQ ID NO: 35 and a first light chain of SEQ ID NO: 36, a second heavy chain of SEQ ID NO: 41 and a second light chain of SEQ ID NO: 42.
49. The bispecific antibody of one of the preceding embodiments comprising a first heavy chain of SEQ ID NO: 37 and a first light chain of SEQ ID NO: 38, a second heavy chain of SEQ ID NO: 41 and a second light chain of SEQ ID NO: 42.
50. The bispecific antibody of one of the preceding embodiments comprising a first heavy chain of SEQ ID NO: 59 a second heavy chain of SEQ ID NO: 60, a first light chain of SEQ ID NO: 57, and a second light chain of SEQ ID NO: 58.
51. The bispecific antibody of one of the preceding embodiments comprising a first heavy chain of SEQ ID NO: 61 a second heavy chain of SEQ ID NO: 60, a first light chain of SEQ ID NO: 57 and a second light chain of SEQ ID NO: 58.
52. An immunoconjugate comprising the bispecific antibody of one of the preceding embodiments and a cytotoxic agent.
53. The immunoconjugate according to embodiment 52 wherein the cytotoxic agent is Pseudomonas Exotoxin A or an Amatoxin.
54. A trispecific antibody comprising the bispecific antibody of any one of embodiments 1 to 51 wherein a further binding domain specifically binding to a third target is fused to the C-terminus of the bispecific antibody.
55. An isolated nucleic acid encoding the bispecific antibody of any of embodiments 1 to 51, or the immunoconjugate of embodiment 52 or 53 or the trispecific antibody of embodiment 48.
56. A host cell comprising the nucleic acid of embodiment 55.
57. A method of producing the bispecific antibody of any of embodiments 1 to 51, or the immunoconjugate of embodiment 52 or 53 or the trispecific antibody of embodiment 54 comprising culturing the host cell of embodiment 56 under conditions suitable for the expression of the antibody.
58. The method of embodiment 57, further comprising recovering the antibody from the host cell.
59. A bispecific antibody produced by the method of embodiment 57 or 58.
60. A pharmaceutical composition comprising the bispecific antibody of any of embodiments 1 to 51, or the immunoconjugate of embodiment 52 or 53 or the trispecific antibody of embodiment 54 and a pharmaceutically acceptable carrier.
61. The pharmaceutical composition of embodiment 60, further comprising an additional therapeutic agent.
62. The bispecific antibody of any one of embodiments 1 to 51 or 59, or the immunoconjugate of embodiment 52 or 53 or the trispecific antibody of embodiment 54 or the pharmaceutical composition of any one of embodiments 60 or 61 for use as a medicament.
63. The bispecific antibody of any one of embodiments 1 to 51 or 59, or the immunoconjugate of embodiment 52 or 53 or the trispecific antibody of embodiment 54 or the pharmaceutical composition of any one of embodiments 60 or 61 for use in treating graft-versus-host disease.
64. The bispecific antibody of any one of embodiments 1 to 51 or 59, or the immunoconjugate of embodiment 52 or 53 or the trispecific antibody of embodiment 54 or the pharmaceutical composition of any one of embodiments 60 or 61 for use
   i. in the modulation of immune responses, such as restoring T cell activity,
   ii. in stimulating an immune response or function,
   iii. in the treatment of infections,
   iv. in the treatment of cancer,
   v. in delaying progression of cancer,
   vi. in prolonging the survival of a patient suffering from cancer.
65. The bispecific antibody of any one of embodiments 1 to 51 or 59, or the immunoconjugate of embodiment 52 or 53 or the trispecific antibody of embodiment 54 or the pharmaceutical composition of any one of embodiments 60 or 61 for use in the prevention or treatment of cancer.
66. The bispecific antibody of any one of embodiments 1 to 51 or 59, or the immunoconjugate of embodiment 52 or 53 or the trispecific antibody of embodiment 54 or the pharmaceutical composition of any one of embodiments 60 or 61 for use in the prevention or treatment of cancer, wherein the bispecific antibody is administered in combination with a chemotherapeutic agent, radiation and/or other agents for use in cancer immunotherapy.
67. A method of inhibiting the growth of tumor cells in a an individual comprising administering to the individual an effective amount of the bispecific antibody of any one of embodiments 1 to 51 or 59, or the immunoconjugate of embodiment 52 or 53 or the trispecific antibody of embodiment 54 or the pharmaceutical composition of any one of embodiments 60 or 61 to inhibit the growth of the tumor cells.
68. Use of the bispecific antibody of any one of embodiments 1 to 51 or 59, or the immunoconjugate of embodiment 52 or 53 or the trispecific antibody of embodiment 54 or the pharmaceutical composition of any one of embodiments 60 or 61 in the manufacture of a medicament for treatment of
   i. graft-versus-host disease,
   ii. infections or
   iii. cancer.
69. Use of the bispecific antibody of any one of embodiments 1 to 51 or 59, or the immunoconjugate of embodiment 52 or 53 or the trispecific antibody of embodiment 54 or the pharmaceutical composition of any one of embodiments 60 or 61 in the manufacture of a medicament for
   i. modulating an immune response, such as restoring T cell activity
   ii. stimulating an immune response or function
   iii. delaying progression of cancer, and/or
   iv. prolonging the survival or a patient suffering from cancer.
70. A method of treating an individual having graft-versus-host disease comprising administering to the individual an effective amount of the bispecific antibody of any one of embodiments 1 to 51 or 59, or the immunoconjugate of embodiment 52 or 53 or the trispecific antibody of embodiment 54 or the pharmaceutical composition of any one of embodiments 60 or 61.
71. The method of embodiments 67 or 70 further comprising administering an additional therapeutic agent to the individual.
72. The method of embodiment 71 wherein the additional therapeutic agent is selected from the group consisting of chemotherapeutic agents, radiation and/or other agents for use in cancer immunotherapy.
73. A method of inhibiting PD1 function in an individual comprising administering to the individual an effective amount of the bispecific antibody of any one of embodiments 1 to 51 or 59, or the immunoconjugate of embodiment 52 or 53 or the trispecific antibody of embodiment 54 or the pharmaceutical composition of any one of embodiments 60 or 61 to inhibit PD1 function.

### Description of the Amino Acid Sequences

TABLE 3
Anti-TfR antigen-binding moieties (variable regions and
hypervariable regions (CDRs))

| | | | | |
|---|---|---|---|---|
| SEQ ID | 1 | CDR-H1 | 1026 | SYAMS |
| NO: | | | | |
| SEQ ID | 2 | CDR-H2 | 1026 | YIWSGGSTDYASWAKS |
| NO: | | | | |
| SEQ ID | 3 | CDR-H3 | 1026 | RYGTSYPDYGDASGFDP |
| NO: | | | | |
| SEQ ID | 4 | CDR-L1 | 1026 | RASQSISSYLA |
| NO: | | | | |
| SEQ ID | 5 | CDR-L2 | 1026 | RASTLAS |
| NO: | | | | |
| SEQ ID | 6 | CDR-L3 | 1026 | QQNYASSNVDNT |
| NO | | | | |
| SEQ ID | 7 | Heavy chain | 1026 | QSMQESGPGLVKPSQTLSLTCTVSGFSLSSYAMSW |
| NO: | | variable domain | | IRQHPGKGLEWIGYIWSGGSTDYASWAKSRVTISK |
| | | VH | | TSTTVSLKLSSVTAADTAVYYCARRYGTSYPDYGD |
| | | | | ASGFDPWGQGTLVTVSS |
| SEQ ID | 8 | Light chain | 1026 | AIQLTQSPSSLSASVGDRVTITCRASQSIS |
| NO: | | variable domain | | SYLAWYQQKPGKAPKLLIYRASTLASGVPS |
| | | VL | | RFSGSGSGTDFTLTISSLQPEDFATYYCQQ |
| | | | | NYASSNVDNTFGGGTKVEIK |
| SEQ ID | 9 | CDR-H1 | 51A165 | SYVMH |
| NO: | | | | |
| SEQ ID | 10 | CDR-H2 | 51A165 | YVNPYNDDTKYNEKFRG |
| NO: | | | | |
| SEQ ID | 11 | CDR-H3 | 51A165 | SYYYYSMDN |
| NO: | | | | |
| SEQ ID | 12 | CDR-L1 | 51A165 | SASSSIRYMH |
| NO: | | | | |
| SEQ ID | 13 | CDR-L2 | 51A165 | DTSELAS |
| NO: | | | | |
| SEQ ID | 14 | CDR-L3 | 51A165 | HQRSSYPFT |
| NO: | | | | |
| SEQ ID | 15 | Heavy chain | 51A165 | EVQLQQSGPELVKPGASVKMSCKASGYTFT |
| NO: | | variable domain | | SYVMHWVKQKPGQGLEWIGYVNPYNDDTKY |
| | | VH | | NEKFRGKATLTSDKSSSTAYMELSSLTSED |
| | | | | SAVYYCARSYYYYSMDNWGQGTSVTVSS |
| SEQ ID | 16 | Light chain | 51A165 | QIVLTQSPAIMSASPGEKVTVTCSASSSIR |
| NO: | | variable domain | | YMHWYQQKPGTSPKRWIYDTSELASGVPAR |
| | | VL | | FSGSGSGTSYSLTINSMEAEDAATYYCHQR |
| | | | | SSYPFTFGGGTKLEIK |

TABLE 4
Anti-PD1 antigen-binding moieties (variable regions and
hypervariable regions (CDRs))

| | | | | |
|---|---|---|---|---|
| SEQ ID | 17 | CDR-H1 | PD1-0103- | SYTMS |
| NO: | | | 0312 | |
| SEQ ID | 18 | CDR-H2 | PD1-0103- | TISGGGRDIYYPDSVKG |
| NO: | | | 0312 | |
| SEQ ID | 19 | CDR-H3 | PD1-0103- | LTGRVYFALDS |
| NO: | | | 0312 | |
| SEQ ID | 20 | CDR-L1 | PD1-0103- | KASESVDTSDNSFIH |
| NO: | | | 0312 | |
| SEQ ID | 21 | CDR-L2 | PD1-0103- | RSSTLES |
| NO: | | | 0312 | |
| SEQ ID | 22 | CDR-L3 | PD1-0103- | QQNYDVPWT |
| NO: | | | 0312 | |
| SEQ ID | 23 | Heavy chain | PD1-0103- | EVQLLESGGGLVQPGGSLRLSCAASGFSFS |
| NO: | | variable domain | 0312 | SYTMSWVRQAPGKGLEWVATISGGGRDIYY |
| | | VH | | PDSVKGRFTISRDNSKNTLYLQMNSLRAED |
| | | | | TAVYYCVLLTGRVYFALDSWGQGTLVTVSS |
| SEQ ID | 24 | Light chain | PD1-0103- | DIVMTQSPDSLAVSLGERATINCKASESVD |
| NO: | | variable domain | 0312 | TSDNSFIHWYQQKPGQSPKLLIYRSSTLES |
| | | VL | | GVPDRFSGSGSGTDFTLTISSLQAEDVAVY |
| | | | | YCQQNYDVPWTFGQGTKVEIK |
| SEQ ID | 25 | CDR-H1 | Pembrolizu- | NYYMY |
| NO: | | | mab | |
| SEQ ID | 26 | CDR-H2 | Pembrolizu- | GINPSNGGTNFNEKFKN |
| NO: | | | mab | |
| SEQ ID | 27 | CDR-H3 | Pembrolizu- | RDYRFDMGFDY |
| NO: | | | mab | |
| SEQ ID | 28 | CDR-L1 | Pembrolizu- | RASKGVSTSGYSYLH |
| NO: | | | mab | |
| SEQ ID | 29 | CDR-L2 | Pembrolizu- | LASYLES |
| NO: | | | mab | |
| SEQ ID | 30 | CDR-L3 | Pembrolizu- | QHSRDLPLT |
| NO: | | | mab | |
| SEQ ID | 31 | Heavy chain | Pembrolizu- | QVQLVQSGVEVKKPGASVKVSCKASGYTFT |
| NO: | | variable domain | mab | NYYMYWVRQAPGQGLEWMGGINPSNGGTNF |
| | | VH | | NEKFKNRVTLTTDSSTTTAYMELKSLQFDD |
| | | | | TAVYYCARRDYRFDMGFDYWGQGTTVTVSS |
| SEQ ID | 32 | Light chain | Pembrolizu- | EIVLTQSPATLSLSPGERATLSCRASKGVS |
| NO: | | variable domain | mab | TSGYSYLHWYQQKPGQAPRLLIYLASYLES |
| | | VL | | GVPARFSGSGSGTDFTLTISSLEPEDFAVY |
| | | | | YCQHSRDLPLTFGGGTKVEIK |

TABLE 5
Non-binding controls (“Nada”)

| | | | | |
|---|---|---|---|---|
| SEQ ID | 33 | Heavy chain | Nada | EVQLVESGGGLVQPGGSLRLSCAASGFSIAG |
| NO: | | variable domain | | TAIHWVRQAPGKGLEWVASISPGGGSTAYAD |
| | | VH | | SVKGRFTISADTSKNTAYLQMNSLRAEDTAV |
| | | | | YYCSRSGGSGASAMDYWGQGTLVTVSS |
| SEQ ID | 34 | Light chain | Nada | DIQMTQSPSSLSASVGDRVTITCRASQDVG |
| NO: | | variable domain | | SGVAWYQQKPGKAPKLLIGSASGLYSGVPS |
| | | VL | | RFSGSRSGTDFTLTISSLQPEDFATYYCQQ |
| | | | | SASGGSTFGQGTKVEIK |

TABLE 6
Designation monospecific unmodified anti-TfR antibodies/antigen-
binding moieties which were used for the anti-TfR bispecific
antibodies described herein

| Antibody | Target | VH (SEQ ID NO:) | VL (SEQ ID NO:) |
|---|---|---|---|
| 1026 | TfR | 7 | 8 |
| 51A165 | TfR | 15 | 16 |
| PD1-0103-0312 | PD1 | 23 | 24 |
| Pembrolizumab | PD1 | 31 | 32 |

TABLE 7A
Designation exemplary bispecific antibodies with
modified VH/VL as CrossMabs (see WO 2016/016299)

| Bispecific | | VH | VL | VH | VL |
|---|---|---|---|---|---|
| anti-TfR/ | | (SEQ | (SEQ | (SEQ | (SEQ |
| anti-PD1 | | ID | ID | ID | ID |
| antibody | Name of variable regions | NO:) | NO:) | NO:) | NO:) |
| 8012 | PD1-0103-0312/51A165 | 23 | 24 | 15 | 16 |
| 8013 | PD1-0103-0312/1026 | 23 | 24 | 7 | 8 |
| 8014 | PD1-0103-0312/Nada | 23 | 24 | 33 | 34 |
| 8015 | Nada/51A165 | 33 | 34 | 15 | 16 |
| 8016 | Nada/1026 | 33 | 34 | 7 | 8 |
| 8017 | Pembrolizumab/1026 | 31 | 32 | 7 | 8 |
| 8018 | Pembrolizumab/51A165 | 31 | 32 | 15 | 16 |
| 8019 | Pembrolizumab/Nada | 31 | 32 | 33 | 34 |

TABLE 7B
Designation VH/VL regions used for exemplary 2 + 1
format antibodies (humanized and mouse surrogate molecules)

| Bispecific | | | | | | |
|---|---|---|---|---|---|---|
| anti-TfR/ | | | VH | VL | VH | VL |
| anti-PD1 | | | (SEQ ID | (SEQ ID | (SEQ ID | (SEQ ID |
| antibody | Format | Name of variable regions | NO:) | NO:) | NO:) | NO:) |
| 8156 | BBB | PD1-0103-0312/TfR(51A165) | 23 | 24 | 15 | 16 |
| 8157 | TCB | PD1-0103-0312/TfR(51A165) | 23 | 24 | 15 | 16 |
| 8158 | BBB | PD1-0103-0312/Nada | 33 | 34 | 15 | 16 |
| 8159 | TCB | PD1-0103-0312/Nada | 33 | 34 | 15 | 16 |
| 6768 | TCB | huPD1-478/mTfR-001 | 86 | 87 | 78 | 79 |
| 6769 | TCB | huPD1-478/Nada | 86 | 87 | 94 | 95 |
| 6794 | TCB | Nada/mTfR-001 | 94 | 95 | 78 | 79 |
| 6757 | BBB | huPD1-478/mTfR-001 | 86 | 87 | 78 | 79 |
| 6761 | BBB | huPD1-478/Nada | 86 | 87 | 94 | 95 |
| 6793 | BBB | Nada/mTfR-001 | 94 | 95 | 78 | 79 |

TABLE 8
Full-length heavy and light chains used for exemplary bispecific
antibodies (1+1 format; hypervariable regions underlined and bold)

| | | | |
|---|---|---|---|
| SEQ | 35 | HC_Hole_EE_ | QSMQESGPGLVKPSQTLSLTCTVSGFSLSSYAMSWIRQHPGKGLEWIGY |
| ID | | charge_TfR_ | IWSGGSTDYASWAKSRVTISKTSTTVSLKLSSVTAADTAVYYCARRYGT |
| NO: | | (1026) | SYPDYGDASGFDPWGQGTLVTVSSASTKGPSVFPLAPSSKSTSGGTAAL |
| | | | GCLVEDYFPEPVTVSWNSGALTSGVHTFPAVLQSSGLYSLSSVVTVPSS |
| | | | SLGTQTYICNVNHKPSNTKVDEKVEPKSCDKTHTCPPCPAPEAAGGPSV |
| | | | FLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALGAPIEKTISK |
| | | | AKGQPREPQVCTLPPSRDELTKNQVSLSCAVKGFYPSDIAVEWESNGQP |
| | | | ENNYKTTPPVLDSDGSFFLVSKLTVDKSRWQQGNVFSCSVMHEALHNHY |
| | | | TQKSLSLSP |
| SEQ | 36 | LC_RK_charge_ | AIQLTQSPSSLSASVGDRVTITCRASQSISSYLAWYQQKPGKAPKLLIY |
| ID | | TfR_(1026) | RASTLASGVPSRFSGSGSGTDFTLTISSLQPEDFATYYCQQNYASSNVD |
| NO: | | | NTFGGGTKVEIKRTVAAPSVFIFPPSDRKLKSGTASVVCLLNNFYPREA |
| | | | KVQWKVDNALQSGNSQESVTEQDSKDSTYSLSSTLTLSKADYEKHKVYA |
| | | | CEVTHQGLSSPVTKSFNRGEC |
| SEQ | 37 | HC_Hole_EE_ | EVQLQQSGPELVKPGASVKMSCKASGYTFTSYVMHWVKQKPGQGLEWIG |
| ID | | charge_TfR_ | YVNPYNDDTKYNEKFRGKATLTSDKSSSTAYMELSSLTSEDSAVYYCAR |
| NO: | | (51A165) | SYYYYSMDNWGQGTSVTVSSASTKGPSVFPLAPSSKSTSGGTAALGCLV |
| | | | EDYFPEPVTVSWNSGALTSGVHTFPAVLQSSGLYSLSSVVTVPSSSLGT |
| | | | QTYICNVNHKPSNTKVDEKVEPKSCDKTHTCPPCPAPEAAGGPSVFLFP |
| | | | PKPKDTLMISRTPEVTCVVVDVSHEDPEVKFNWYVDGVEVHNAKTKPRE |
| | | | EQYNSTYRVVSVLTVLHQDWLNGKEYKCKVSNKALGAPIEKTISKAKGQ |
| | | | PREPQVCTLPPSRDELTKNQVSLSCAVKGFYPSDIAVEWESNGQPENNY |
| | | | KTTPPVLDSDGSFFLVSKLTVDKSRWQQGNVFSCSVMHEALHNHYTQKS |
| | | | LSLSP |
| SEQ | 38 | LC_RK_charge_ | QIVLTQSPAIMSASPGEKVTVTCSASSSIRYMHWYQQKPGTSPKRWIYD |
| ID | | TfR_(51A165) | TSELASGVPARFSGSGSGTSYSLTINSMEAEDAATYYCHQRSSYPFTFG |
| NO: | | | GGTKLEIKRTVAAPSVFIFPPSDRKLKSGTASVVCLLNNFYPREAKVQW |
| | | | KVDNALQSGNSQESVTEQDSKDSTYSLSSTLTLSKADYEKHKVYACEVT |
| | | | HQGLSSPVTKSFNRGEC |
| SEQ | 39 | HC_cross_Knob_ | DIVMTQSPDSLAVSLGERATINCKASESVDTSDNSFIHWYQQKPGQSPK |
| ID | | PD1-0103- | LLIYRSSTLESGVPDRFSGSGSGTDFTLTISSLQAEDVAVYYCQQNYDV |
| NO: | | 0312_VL | PWTFGQGTKVEIKSSASTKGPSVFPLAPSSKSTSGGTAALGCLVKDYFP |
| | | | EPVTVSWNSGALTSGVHTFPAVLQSSGLYSLSSVVTVPSSSLGTQTYIC |
| | | | NVNHKPSNTKVDKKVEPKSCDKTHTCPPCPAPEAAGGPSVFLFPPKPKD |
| | | | TLMISRTPEVTCVVVDVSHEDPEVKFNWYVDGVEVHNAKTKPREEQYNS |
| | | | TYRVVSVLTVLHQDWLNGKEYKCKVSNKALGAPIEKTISKAKGQPREPQ |
| | | | VYTLPPCRDELTKNQVSLWCLVKGFYPSDIAVEWESNGQPENNYKTTPP |
| | | | VLDSDGSFFLYSKLTVDKSRWQQGNVFSCSVMHEALHNHYTQKSLSLSP |
| SEQ | 40 | LC_cross_PD1- | EVQLLESGGGLVQPGGSLRLSCAASGFSFSSYTMSWVRQAPGKGLEWVA |
| ID | | 0103-0312_VH | TISGGGRDIYYPDSVKGRFTISRDNSKNTLYLQMNSLRAEDTAVYYCVL |
| NO: | | | LTGRVYFALDSWGQGTLVTVSSASVAAPSVFIFPPSDEQLKSGTASVVC |
| | | | LLNNFYPREAKVQWKVDNALQSGNSQESVTEQDSKDSTYSLSSTLTLSK |
| | | | ADYEKHKVYACEVTHQGLSSPVTKSFNRGEC |
| SEQ | 41 | HC_cross_Knob_ | EIVLTQSPATLSLSPGERATLSCRASKGVSTSGYSYLHWYQQKPGQAPR |
| ID | | PD1_Pembro_ | LLIYLASYLESGVPARFSGSGSGTDFTLTISSLEPEDFAVYYCQHSRDL |
| NO: | | VL | PLTFGGGTKVEIKSSASTKGPSVFPLAPSSKSTSGGTAALGCLVKDYFP |
| | | | EPVTVSWNSGALTSGVHTFPAVLQSSGLYSLSSVVTVPSSSLGTQTYIC |
| | | | NVNHKPSNTKVDKKVEPKSCDKTHTCPPCPAPEAAGGPSVFLFPPKPKD |
| | | | TLMISRTPEVTCVVVDVSHEDPEVKFNWYVDGVEVHNAKTKPREEQYNS |
| | | | TYRVVSVLTVLHQDWLNGKEYKCKVSNKALGAPIEKTISKAKGQPREPQ |
| | | | VYTLPPCRDELTKNQVSLWCLVKGFYPSDIAVEWESNGQPENNYKTTPP |
| | | | VLDSDGSFFLYSKLTVDKSRWQQGNVFSCSVMHEALHNHYTQKSLSLSP |
| SEQ | 42 | LC_cross_PD1_ | QVQLVQSGVEVKKPGASVKVSCKASGYTFTNYYMYWVRQAPGQGLEWMG |
| ID | | Pembro_VH | GINPSNGGTNFNEKFKNRVTLTTDSSTTTAYMELKSLQFDDTAVYYCAR |
| NO: | | | RDYRFDMGFDYWGQGTTVTVSSASVAAPSVFIFPPSDEQLKSGTASVVC |
| | | | LLNNFYPREAKVQWKVDNALQSGNSQESVTEQDSKDSTYSLSSTLTLSK |
| | | | ADYEKHKVYACEVTHQGLSSPVTKSFNRGEC |
| SEQ | 43 | HC_cross_ | DIQMTQSPSSLSASVGDRVTITCRASQDVGSGVAWYQQKPGKAPKLLIG |
| ID | | Knob_Nada_VL | SASGLYSGVPSRFSGSRSGTDFTLTISSLQPEDFATYYCQQSASGGSTF |
| NO: | | | GQGTKVEIKSSASTKGPSVFPLAPSSKSTSGGTAALGCLVKDYFPEPVT |
| | | | VSWNSGALTSGVHTFPAVLQSSGLYSLSSVVTVPSSSLGTQTYICNVNH |
| | | | KPSNTKVDKKVEPKSCDKTHTCPPCPAPEAAGGPSVFLFPPKPKDTLMI |
| | | | SRTPEVTCVVVDVSHEDPEVKFNWYVDGVEVHNAKTKPREEQYNSTYRV |
| | | | VSVLTVLHQDWLNGKEYKCKVSNKALGAPIEKTISKAKGQPREPQVYTL |
| | | | PPCRDELTKNQVSLWCLVKGFYPSDIAVEWESNGQPENNYKTTPPVLDS |
| | | | DGSFFLYSKLTVDKSRWQQGNVFSCSVMHEALHNHYTQKSLSLSP |
| SEQ | 44 | LC_cross_Nada_ | EVQLVESGGGLVQPGGSLRLSCAASGFSIAGTAIHWVRQAPGKGLEWVA |
| ID | | VH | SISPGGGSTAYADSVKGRFTISADTSKNTAYLQMNSLRAEDTAVYYCSR |
| NO: | | | SGGSGASAMDYWGQGTLVTVSSASVAAPSVFIFPPSDEQLKSGTASVVC |
| | | | LLNNFYPREAKVQWKVDNALQSGNSQESVTEQDSKDSTYSLSSTLTLSK |
| | | | ADYEKHKVYACEVTHQGLSSPVTKSFNRGEC |
| SEQ | 45 | HC_Hole_EE_ | EVQLVESGGGLVQPGGSLRLSCAASGESIAGTAIHWVRQAPGKGLEWVA |
| ID | | charge_Nada | SISPGGGSTAYADSVKGRFTISADTSKNTAYLQMNSLRAEDTAVYYCSR |
| NO: | | | SGGSGASAMDYWGQGTLVTVSSASTKGPSVFPLAPSSKSTSGGTAALGC |
| | | | LVEDYFPEPVTVSWNSGALTSGVHTFPAVLQSSGLYSLSSVVTVPSSSL |
| | | | GTQTYICNVNHKPSNTKVDEKVEPKSCDKTHTCPPCPAPEAAGGPSVEL |
| | | | FPPKPKDTLMISRTPEVTCVVVDVSHEDPEVKFNWYVDGVEVHNAKTKP |
| | | | REEQYNSTYRVVSVLTVLHQDWLNGKEYKCKVSNKALGAPIEKTISKAK |
| | | | GQPREPQVCTLPPSRDELTKNQVSLSCAVKGFYPSDIAVEWESNGQPEN |
| | | | NYKTTPPVLDSDGSFFLVSKLTVDKSRWQQGNVFSCSVMHEALHNHYTQ |
| | | | KSLSLSP |
| SEQ | 46 | LC_RK_charge_ | DIQMTQSPSSLSASVGDRVTITCRASQDVGSGVAWYQQKPGKAPKLLIG |
| ID | | Nada | SASGLYSGVPSRFSGSRSGTDFTLTISSLQPEDFATYYCQQSASGGSTF |
| NO: | | | GQGTKVEIKRTVAAPSVFIFPPSDRKLKSGTASVVCLLNNFYPREAKVQ |
| | | | WKVDNALQSGNSQESVTEQDSKDSTYSLSSTLTLSKADYEKHKVYACEV |
| | | | THQGLSSPVTKSFNRGEC |
| SEQ | 47 | HC_Knob_TfR_ | EVQLQQSGPELVKPGASVKMSCKASGYTFTSYVMHWVKQKPGQGLEWIG |
| ID | | (51A165) | YVNPYNDDTKYNEKFRGKATLTSDKSSSTAYMELSSLTSEDSAVYYCAR |
| NO: | | IgG1/anti-biotin | SYYYYSMDNWGQGTSVTVSSASTKGPSVFPLAPSSKSTSGGTAALGCLV |
| | | | KDYFPEPVTVSWNSGALTSGVHTFPAVLQSSGLYSLSSVVTVPSSSLGT |
| | | | QTYICNVNHKPSNTKVDKKVEPKSCDKTHTCPPCPAPEAAGGPSVFLFP |
| | | | PKPKDTLMISRTPEVTCVVVDVSHEDPEVKFNWYVDGVEVHNAKTKPRE |
| | | | EQYNSTYRVVSVLTVLHQDWLNGKEYKCKVSNKALGAPIEKTISKAKGQ |
| | | | PREPQVYTLPPCRDELTKNQVSLWCLVKGFYPSDIAVEWESNGQPENNY |
| | | | KTTPPVLDSDGSFFLYSKLTVDKSRWQQGNVFSCSVMHEALHNHYTQKS |
| | | | LSLSPGKGGGGSGGGGSDIQMTQSPSSLSASVGDRVTITCRASGNIHNY |
| | | | LSWYQQKPGKVPKLLIYSAKTLADGVPSRFSGSGSGTDFTLTISSLQPE |
| | | | DVATYYCQHFWSSIYTFGCGTKLEIKRGGGGSGGGGSGGGGSGGGGSQV |
| | | | QLVQSGAEVKKPGSSVKVSCKSSGFNNKDTFFQWVRQAPGQCLEWMGRI |
| | | | DPANGFTKYAQKFQGRVTITADTSTSTAYMELSSLRSEDTAVYYCARWD |
| | | | TYGAAWFAYWGQGTLVTVSS |
| SEQ | 48 | LC TfR | QIVLTQSPAIMSASPGEKVTVTCSASSSIRYMHWYQQKPGTSPKRWIYD |
| ID | | (51A165) | TSELASGVPARFSGSGSGTSYSLTINSMEAEDAATYYCHQRSSYPFTFG |
| NO: | | | GGTKLEIKRTVAAPSVFIFPPSDEQLKSGTASVVCLLNNFYPREAKVQW |
| | | | KVDNALQSGNSQESVTEQDSKDSTYSLSSTLTLSKADYEKHKVYACEVT |
| | | | HQGLSSPVTKSFNRGEC |
| SEQ | 49 | HC_cross_ | QVQLVQSGVEVKKPGASVKVSCKASGYTFTNYYMYWVRQAPGQGLEWMG |
| ID | | Hole_Pembro | GINPSNGGTNFNEKFKNRVTLTTDSSTTTAYMELKSLQFDDTAVYYCAR |
| NO: | | IgG1/anti-biotin | RDYRFDMGFDYWGQGTTVTVSSASVAAPSVFIFPPSDEQLKSGTASVVC |
| | | | LLNNFYPREAKVQWKVDNALQSGNSQESVTEQDSKDSTYSLSSTLTLSK |
| | | | ADYEKHKVYACEVTHQGLSSPVTKSFNRGECDKTHTCPPCPAPEAAGGP |
| | | | SVFLFPPKPKDTLMISRTPEVTCVVVDVSHEDPEVKFNWYVDGVEVHNA |
| | | | KTKPREEQYNSTYRVVSVLTVLHQDWLNGKEYKCKVSNKALGAPIEKTI |
| | | | SKAKGQPREPQVCTLPPSRDELTKNQVSLSCAVKGFYPSDIAVEWESNG |
| | | | QPENNYKTTPPVLDSDGSFFLVSKLTVDKSRWQQGNVFSCSVMHEALHN |
| | | | HYTQKSLSLSPGKGGGGSGGGGSDIQMTQSPSSLSASVGDRVTITCRAS |
| | | | GNIHNYLSWYQQKPGKVPKLLIYSAKTLADGVPSRFSGSGSGTDFTLTI |
| | | | SSLQPEDVATYYCQHFWSSIYTFGCGTKLEIKRGGGGSGGGGSGGGGSG |
| | | | GGGSQVQLVQSGAEVKKPGSSVKVSCKSSGFNNKDTFFQWVRQAPGQCL |
| | | | EWMGRIDPANGFTKYAQKFQGRVTITADTSTSTAYMELSSLRSEDTAVY |
| | | | YCARWDTYGAAWFAYWGQGTLVTVSS |
| SEQ | 50 | LC_cross_ | EIVLTQSPATLSLSPGERATLSCRASKGVSTSGYSYLHWYQQKPGQAPR |
| ID | | Pembro | LLIYLASYLESGVPARFSGSGSGTDFTLTISSLEPEDFAVYYCQHSRDL |
| NO: | | | PLTFGGGTKVEIKSSASTKGPSVFPLAPSSKSTSGGTAALGCLVKDYFP |
| | | | EPVTVSWNSGALTSGVHTFPAVLQSSGLYSLSSVVTVPSSSLGTQTYIC |
| | | | NVNHKPSNTKVDKKVEPKSC |
| SEQ | 51 | HC_Knob_Nada_ | EVQLVESGGGLVQPGGSLRLSCAASGFSIAGTAIHWVRQAPGKGLEWVA |
| ID | | IgG1/anti- | SISPGGGSTAYADSVKGRFTISADTSKNTAYLQMNSLRAEDTAVYYCSR |
| NO: | | biotin | SGGSGASAMDYWGQGTLVTVSSASTKGPSVFPLAPSSKSTSGGTAALGC |
| | | | LVKDYFPEPVTVSWNSGALTSGVHTFPAVLQSSGLYSLSSVVTVPSSSL |
| | | | GTQTYICNVNHKPSNTKVDKKVEPKSCDKTHTCPPCPAPEAAGGPSVEL |
| | | | FPPKPKDTLMISRTPEVTCVVVDVSHEDPEVKFNWYVDGVEVHNAKTKP |
| | | | REEQYNSTYRVVSVLTVLHQDWLNGKEYKCKVSNKALGAPIEKTISKAK |
| | | | GQPREPQVYTLPPCRDELTKNQVSLWCLVKGFYPSDIAVEWESNGQPEN |
| | | | NYKTTPPVLDSDGSFFLYSKLTVDKSRWQQGNVFSCSVMHEALHNHYTQ |
| | | | KSLSLSPGKGGGGSGGGGSDIQMTQSPSSLSASVGDRVTITCRASGNIH |
| | | | NYLSWYQQKPGKVPKLLIYSAKTLADGVPSRFSGSGSGTDFTLTISSLQ |
| | | | PEDVATYYCQHFWSSIYTFGCGTKLEIKRGGGGSGGGGSGGGGSGGGGS |
| | | | QVQLVQSGAEVKKPGSSVKVSCKSSGFNNKDTFFQWVRQAPGQCLEWMG |
| | | | RIDPANGFTKYAQKFQGRVTITADTSTSTAYMELSSLRSEDTAVYYCAR |
| | | | WDTYGAAWFAYWGQGTLVTVSS |
| SEQ | 52 | LC_Nada | DIQMTQSPSSLSASVGDRVTITCRASQDVGSGVAWYQQKPGKAPKLLIG |
| ID | | | SASGLYSGVPSRFSGSRSGTDFTLTISSLQPEDFATYYCQQSASGGSTF |
| NO: | | | GQGTKVEIKRTVAAPSVFIFPPSDEQLKSGTASVVCLLNNFYPREAKVQ |
| | | | WKVDNALQSGNSQESVTEQDSKDSTYSLSSTLTLSKADYEKHKVYACEV |
| | | | THQGLSSPVTKSFNRGEC |
| SEQ | 53 | HC_Cross_Hole_ | EVQLVESGGGLVQPGGSLRLSCAASGFSIAGTAIHWVRQAPGKGLEWVA |
| ID | | Nada | SISPGGGSTAYADSVKGRFTISADTSKNTAYLQMNSLRAEDTAVYYCSR |
| NO: | | IgG1/anti-biotin | SGGSGASAMDYWGQGTLVTVSSASVAAPSVFIFPPSDEQLKSGTASVVC |
| | | | LLNNFYPREAKVQWKVDNALQSGNSQESVTEQDSKDSTYSLSSTLTLSK |
| | | | ADYEKHKVYACEVTHQGLSSPVTKSFNRGECDKTHTCPPCPAPEAAGGP |
| | | | SVFLFPPKPKDTLMISRTPEVTCVVVDVSHEDPEVKFNWYVDGVEVHNA |
| | | | KTKPREEQYNSTYRVVSVLTVLHQDWLNGKEYKCKVSNKALGAPIEKTI |
| | | | SKAKGQPREPQVCTLPPSRDELTKNQVSLSCAVKGFYPSDIAVEWESNG |
| | | | QPENNYKTTPPVLDSDGSFFLVSKLTVDKSRWQQGNVFSCSVMHEALHN |
| | | | HYTQKSLSLSPGKGGGGSGGGGSDIQMTQSPSSLSASVGDRVTITCRAS |
| | | | GNIHNYLSWYQQKPGKVPKLLIYSAKTLADGVPSRFSGSGSGTDFTLTI |
| | | | SSLQPEDVATYYCQHFWSSIYTFGCGTKLEIKRGGGGSGGGGSGGGGSG |
| | | | GGGSQVQLVQSGAEVKKPGSSVKVSCKSSGENNKDTFFQWVRQAPGQCL |
| | | | EWMGRIDPANGFTKYAQKFQGRVTITADTSTSTAYMELSSLRSEDTAVY |
| | | | YCARWDTYGAAWFAYWGQGTLVTVSS |
| SEQ | 54 | LC_cross_Nada | DIQMTQSPSSLSASVGDRVTITCRASQDVGSGVAWYQQKPGKAPKLLIG |
| ID | | | SASGLYSGVPSRFSGSRSGTDFTLTISSLQPEDFATYYCQQSASGGSTF |
| NO: | | | GQGTKVEIKSSASTKGPSVFPLAPSSKSTSGGTAALGCLVKDYFPEPVT |
| | | | VSWNSGALTSGVHTFPAVLQSSGLYSLSSVVTVPSSSLGTQTYICNVNH |
| | | | KPSNTKVDKKVEPKSC |
| SEQ | 55 | HC_Anti CD33 | EVQLVQSGAEVKKPGSSVKVSCKASGYTITDSNIHWVRQAPGQSLEWIG |
| ID | | anti biotin | YIYPYNGGTDYNQKFKNRATLTVDNPTNTAYMELSSLRSEDTAFYYCVN |
| NO: | | | GNPWLAYWGQGTLVTVSSASTKGPSVFPLAPSSKSTSGGTAALGCLVKD |
| | | | YFPEPVTVSWNSGALTSGVHTFPAVLQSSGLYSLSSVVTVPSSSLGTQT |
| | | | YICNVNHKPSNTKVDKKVEPKSCDKTHTCPPCPAPELLGGPSVFLFPPK |
| | | | PKDTLMISRTPEVTCVVVDVSHEDPEVKFNWYVDGVEVHNAKTKPREEQ |
| | | | YNSTYRVVSVLTVLHQDWLNGKEYKCKVSNKALPAPIEKTISKAKGQPR |
| | | | EPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLS |
| | | | LSPGKGGGGSGGGGSDIQMTQSPSSLSASVGDRVTITCRASGNIHNYLS |
| | | | WYQQKPGKVPKLLIYSAKTLADGVPSRFSGSGSGTDFTLTISSLQPEDV |
| | | | ATYYCQHFWSSIYTFGCGTKLEIKRGGGGSGGGGSGGGGSGGGGSQVQL |
| | | | VQSGAEVKKPGSSVKVSCKSSGFNNKDTFFQWVRQAPGQCLEWMGRIDP |
| | | | ANGFTKYAQKFQGRVTITADTSTSTAYMELSSLRSEDTAVYYCARWDTY |
| | | | GAAWFAYWGQGTLVTVSS |
| SEQ | 56 | LC_Anti CD33 | DIQLTQSPSTLSASVGDRVTITCRASESLDNYGIRFLTWFQQKPGKAPK |
| ID | | | LLMYAASNQGSGVPSRFSGSGSGTEFTLTISSLQPDDFATYYCQQTKEV |
| NO: | | | PWSFGQGTKVEVKRTVAAPSVFIFPPSDEQLKSGTASVVCLLNNFYPRE |
| | | | AKVQWKVDNALQSGNSQESVTEQDSKDSTYSLSSTLTLSKADYEKHKVY |
| | | | ACEVTHQGLSSPVTKSFNRGEC |

TABLE 9
Full length heavy and light chains used for 2:1 antibody formats
(TCB-format and BBB-format; hypervariable regions underlined and bold)

| | | | |
|---|---|---|---|
| SEQ | 57 | LC_cross_PD1- | EVQLLESGGGLVQPGGSLRLSCAASGFSFSSYTMSWVRQAPGKGL |
| ID | | 0103-0312_VH | EWVATISGGGRDIYYPDSVKGRFTISRDNSKNTLYLQMNSLRAED |
| NO: | | | TAVYYCVLLTGRVYFALDSWGQGTLVTVSSASVAAPSVFIFPPSD |
| | | | EQLKSGTASVVCLLNNFYPREAKVQWKVDNALQSGNSQESVTEQD |
| | | | SKDSTYSLSSTLTLSKADYEKHKVYACEVTHQGLSSPVTKSENRG |
| | | | EC |
| SEQ | 58 | LC_RK_charge_ | QIVLTQSPAIMSASPGEKVTVTCSASSSIRYMHWYQQKPGTSPKR |
| ID | | TfR_(51A165) | WIYDTSELASGVPARFSGSGSGTSYSLTINSMEAEDAATYYCHQR |
| NO: | | | SSYPFTFGGGTKLEIKRTVAAPSVFIFPPSDRKLKSGTASVVCLL |
| | | | NNFYPREAKVQWKVDNALQSGNSQESVTEQDSKDSTYSLSSTLTL |
| | | | SKADYEKHKVYACEVTHQGLSSPVTKSFNRGEC |
| SEQ | 59 | HC_Cross_Hole | DIVMTQSPDSLAVSLGERATINCKASESVDTSDNSFIHWYQQKPG |
| ID | | PD1-0103-0312_ | QSPKLLIYRSSTLESGVPDRFSGSGSGTDFTLTISSLQAEDVAVY |
| NO: | | VL BBB-format | YCQQNYDVPWTFGQGTKVEIKSSASTKGPSVFPLAPSSKSTSGGT |
| | | TfR_(51A165) | AALGCLVKDYFPEPVTVSWNSGALTSGVHTFPAVLQSSGLYSLSS |
| | | | VVTVPSSSLGTQTYICNVNHKPSNTKVDKKVEPKSCDKTHTCPPC |
| | | | PAPEAAGGPSVFLFPPKPKDTLMISRTPEVTCVVVDVSHEDPEVK |
| | | | FNWYVDGVEVHNAKTKPREEQYNSTYRVVSVLTVLHQDWLNGKEY |
| | | | KCKVSNKALGAPIEKTISKAKGQPREPQVCTLPPSRDELTKNQVS |
| | | | LSCAVKGFYPSDIAVEWESNGQPENNYKTTPPVLDSDGSFFLVSK |
| | | | LTVDKSRWQQGNVFSCSVMHEALHNHYTQKSLSLSPGKGGGGSGG |
| | | | GGSEVQLQQSGPELVKPGASVKMSCKASGYTFTSYVMHWVKQKPG |
| | | | QGLEWIGYVNPYNDDTKYNEKFRGKATLTSDKSSSTAYMELSSLT |
| | | | SEDSAVYYCARSYYYYSMDNWGQGTSVTVSSASTKGPSVFPLAPS |
| | | | SKSTSGGTAALGCLVEDYFPEPVTVSWNSGALTSGVHTFPAVLQS |
| | | | SGLYSLSSVVTVPSSSLGTQTYICNVNHKPSNTKVDEKVEPKSCD |
| SEQ | 60 | HC_Cross_Knob | DIVMTQSPDSLAVSLGERATINCKASESVDTSDNSFIHWYQQKPG |
| ID | | PD1-0103- | QSPKLLIYRSSTLESGVPDRFSGSGSGTDFTLTISSLQAEDVAVY |
| NO: | | 0312_VL | YCQQNYDVPWTFGQGTKVEIKSSASTKGPSVFPLAPSSKSTSGGT |
| | | | AALGCLVKDYFPEPVTVSWNSGALTSGVHTFPAVLQSSGLYSLSS |
| | | | VVTVPSSSLGTQTYICNVNHKPSNTKVDKKVEPKSCDKTHTCPPC |
| | | | PAPEAAGGPSVFLFPPKPKDTLMISRTPEVTCVVVDVSHEDPEVK |
| | | | FNWYVDGVEVHNAKTKPREEQYNSTYRVVSVLTVLHQDWLNGKEY |
| | | | KCKVSNKALGAPIEKTISKAKGQPREPQVYTLPPCRDELTKNQVS |
| | | | LWCLVKGFYPSDIAVEWESNGQPENNYKTTPPVLDSDGSFFLYSK |
| | | | LTVDKSRWQQGNVFSCSVMHEALHNHYTQKSLSLSP |
| SEQ | 61 | HC_Cross_Hole | DIVMTQSPDSLAVSLGERATINCKASESVDTSDNSFIHWYQQKPG |
| ID | | PD1-0103-0312_ | QSPKLLIYRSSTLESGVPDRFSGSGSGTDFTLTISSLQAEDVAVY |
| NO: | | VL_TCB- | YCQQNYDVPWTFGQGTKVEIKSSASTKGPSVFPLAPSSKSTSGGT |
| | | format- | AALGCLVKDYFPEPVTVSWNSGALTSGVHTFPAVLQSSGLYSLSS |
| | | TfR(51A165) | VVTVPSSSLGTQTYICNVNHKPSNTKVDKKVEPKSCDGGGGSGGG |
| | | | GSEVQLQQSGPELVKPGASVKMSCKASGYTFTSYVMHWVKQKPGQ |
| | | | GLEWIGYVNPYNDDTKYNEKFRGKATLTSDKSSSTAYMELSSLTS |
| | | | EDSAVYYCARSYYYYSMDNWGQGTSVTVSSASTKGPSVFPLAPSS |
| | | | KSTSGGTAALGCLVEDYFPEPVTVSWNSGALTSGVHTFPAVLQSS |
| | | | GLYSLSSVVTVPSSSLGTQTYICNVNHKPSNTKVDEKVEPKSCDK |
| | | | THTCPPCPAPEAAGGPSVFLFPPKPKDTLMISRTPEVTCVVVDVS |
| | | | HEDPEVKFNWYVDGVEVHNAKTKPREEQYNSTYRVVSVLTVLHQD |
| | | | WLNGKEYKCKVSNKALGAPIEKTISKAKGQPREPQVCTLPPSRDE |
| | | | LTKNQVSLSCAVKGFYPSDIAVEWESNGQPENNYKTTPPVLDSDG |
| | | | SFFLVSKLTVDKSRWQQGNVFSCSVMHEALHNHYTQKSLSLSP |
| SEQ | 62 | LC_ | DIQMTQSPSSLSASVGDRVTITCRASQDVGSGVAWYQQKPGKAPK |
| ID | | RK_charge_Nada | LLIGSASGLYSGVPSRFSGSRSGTDFTLTISSLQPEDFATYYCQQ |
| NO: | | | SASGGSTFGQGTKVEIKRTVAAPSVFIFPPSDRKLKSGTASVVCL |
| | | | LNNFYPREAKVQWKVDNALQSGNSQESVTEQDSKDSTYSLSSTLT |
| | | | LSKADYEKHKVYACEVTHQGLSSPVTKSFNRGEC |
| SEQ | 63 | HC_Cross_Hole | DIVMTQSPDSLAVSLGERATINCKASESVDTSDNSFIHWYQQKPG |
| ID | | PD1-0103- | QSPKLLIYRSSTLESGVPDRFSGSGSGTDFTLTISSLQAEDVAVY |
| NO: | | 0312_VL_BBB- | YCQQNYDVPWTFGQGTKVEIKSSASTKGPSVFPLAPSSKSTSGGT |
| | | format_Nada | AALGCLVKDYFPEPVTVSWNSGALTSGVHTFPAVLQSSGLYSLSS |
| | | | VVTVPSSSLGTQTYICNVNHKPSNTKVDKKVEPKSCDKTHTCPPC |
| | | | PAPEAAGGPSVFLFPPKPKDTLMISRTPEVTCVVVDVSHEDPEVK |
| | | | FNWYVDGVEVHNAKTKPREEQYNSTYRVVSVLTVLHQDWLNGKEY |
| | | | KCKVSNKALGAPIEKTISKAKGQPREPQVCTLPPSRDELTKNQVS |
| | | | LSCAVKGFYPSDIAVEWESNGQPENNYKTTPPVLDSDGSFFLVSK |
| | | | LTVDKSRWQQGNVFSCSVMHEALHNHYTQKSLSLSPGKGGGGSGG |
| | | | GGSEVQLVESGGGLVQPGGSLRLSCAASGFSIAGTAIHWVRQAPG |
| | | | KGLEWVASISPGGGSTAYADSVKGRFTISADTSKNTAYLQMNSLR |
| | | | AEDTAVYYCSRSGGSGASAMDYWGQGTLVTVSSASTKGPSVFPLA |
| | | | PSSKSTSGGTAALGCLVEDYFPEPVTVSWNSGALTSGVHTFPAVL |
| | | | QSSGLYSLSSVVTVPSSSLGTQTYICNVNHKPSNTKVDEKVEPKS |
| | | | CD |
| SEQ | 64 | HC_Cross Hole | DIVMTQSPDSLAVSLGERATINCKASESVDTSDNSFIHWYQQKPG |
| ID | | PD1-0103- | QSPKLLIYRSSTLESGVPDRFSGSGSGTDFTLTISSLQAEDVAVY |
| NO: | | 0312_VL_TCB- | YCQQNYDVPWTFGQGTKVEIKSSASTKGPSVFPLAPSSKSTSGGT |
| | | format Nada | AALGCLVKDYFPEPVTVSWNSGALTSGVHTFPAVLQSSGLYSLSS |
| | | | VVTVPSSSLGTQTYICNVNHKPSNTKVDKKVEPKSCDGGGGSGGG |
| | | | GSEVQLVESGGGLVQPGGSLRLSCAASGESIAGTAIHWVRQAPGK |
| | | | GLEWVASISPGGGSTAYADSVKGRFTISADTSKNTAYLQMNSLRA |
| | | | EDTAVYYCSRSGGSGASAMDYWGQGTLVTVSSASTKGPSVFPLAP |
| | | | SSKSTSGGTAALGCLVEDYFPEPVTVSWNSGALTSGVHTFPAVLQ |
| | | | SSGLYSLSSVVTVPSSSLGTQTYICNVNHKPSNTKVDEKVEPKSC |
| | | | DKTHTCPPCPAPEAAGGPSVFLFPPKPKDTLMISRTPEVTCVVVD |
| | | | VSHEDPEVKFNWYVDGVEVHNAKTKPREEQYNSTYRVVSVLTVLH |
| | | | QDWLNGKEYKCKVSNKALGAPIEKTISKAKGQPREPQVCTLPPSR |
| | | | DELTKNQVSLSCAVKGFYPSDIAVEWESNGQPENNYKTTPPVLDS |
| | | | DGSFFLVSKLTVDKSRWQQGNVFSCSVMHEALHNHYTQKSLSLSP |
| SEQ | 96 | huPD1-478 | EVILVESGGGLVKPGGSLKLSCAASGFSFSSYTMSWVRQTPEKRL |
| ID | | crossmab LC | DWVATISGGGRDIYYPDSVKGRFTISRDNAKNTLYLEMSSLMSED |
| NO: | | | TALYYCVLLTGRVYFALDSWGQGTSVTVSSASDAAPTVSIFPPSS |
| | | | EQLTSGGASVVCFLNNFYPKDINVKWKIDGSERQNGVLNSWTDQD |
| | | | SKDSTYSMSSTLTLTKDEYERHNSYTCEATHKTSTSPIVKSENRN |
| | | | EC |
| SEQ | 97 | huPD1-478 | KIVLTQSPASLPVSLGQRATISCRASESVDTSDNSFIHWYQQRPG |
| ID | | crossmab HC | QSPKLLIYRSSTLESGVPARFSGSGSRTDFTLTIDPVEADDVATY |
| NO: | | | YCQQNYDVPWTFGGGTKLEIKSSAKTTPPSVYPLAPGSAAQTNSM |
| | | | VTLGCLVKGYFPEPVTVTWNSGSLSSGVHTFPAVLQSDLYTLSSS |
| | | | VTVPSSTWPSQTVTCNVAHPASSTKVDKKIVPRDCGCKPCICTVP |
| | | | EVSSVFIFPPKPKDVLTITLTPKVTCVVVAISKDDPEVQFSWFVD |
| | | | DVEVHTAQTKPREEQINSTFRSVSELPIMHQDWLNGKEFKCRVNS |
| | | | AAFGAPIEKTISKTKGRPKAPQVYTIPPPKEQMAKDKVSLTCMIT |
| | | | NFFPEDITVEWQWNGQPAENYDNTQPIMDTDGSYFVYSDLNVQKS |
| | | | NWEAGNTFTCSVLHEGLHNHHTEKSLSHSPGK |
| SEQ | 98 | mTfR-001 LC | DIQMTQSPASLSASLEEIVTITCQASQDIGNWLAWYQQKPGKSPQ |
| ID | | | LLIYGATSLADGVPSRFSGSRSGTQFSLKISRVQVEDIGIYYCLQ |
| NO: | | | AYNTPWTFGGGTKVEIKRADAAPTVSIFPPSSRKLTSGGASVVCF |
| | | | LNNFYPKDINVKWKIDGSERQNGVLNSWTDQDSKDSTYSMSSTLT |
| | | | LTKDEYERHNSYTCEATHKTSTSPIVKSENRNEC |
| SEQ | 99 | mTfR-001 HC | KIVLTQSPASLPVSLGQRATISCRASESVDTSDNSFIHWYQQRPG |
| ID | | with huPD1-478 | QSPKLLIYRSSTLESGVPARFSGSGSRTDFTLTIDPVEADDVATY |
| NO: | | crossmab TCB | YCQQNYDVPWTFGGGTKLEIKSSAKTTPPSVYPLAPGSAAQTNSM |
| | | format | VTLGCLVKGYFPEPVTVTWNSGSLSSGVHTFPAVLQSDLYTLSSS |
| | | | VTVPSSTWPSQTVTCNVAHPASSTKVDKKIVPRDCGGGGSGGGGS |
| | | | EVQLVESGGGLVQPGNSLTLSCVASGFTFSNYGMHWIRQAPKKGL |
| | | | EWIAMIYYDSSKMNYADTVKGRFTISRDNSKNTLYLEMNSLRSED |
| | | | TAMYYCAVPTSHYVVDVWGQGVSVTVSSAKTTPPSVYPLAPGSAA |
| | | | QTNSMVTLGCLVEGYFPEPVTVTWNSGSLSSGVHTFPAVLQSDLY |
| | | | TLSSSVTVPSSTWPSQTVTCNVAHPASSTKVDEKIVPRDCGCKPC |
| | | | ICTVPEVSSVFIFPPKPKDVLTITLTPKVTCVVVAISKDDPEVQF |
| | | | SWFVDDVEVHTAQTKPREEQINSTFRSVSELPIMHQDWLNGKEFK |
| | | | CRVNSAAFGAPIEKTISKTKGRPKAPQVYTIPPPKKQMAKDKVSL |
| | | | TCMITNFFPEDITVEWQWNGQPAENYKNTQPIMKTDGSYFVYSKL |
| | | | NVQKSNWEAGNTFTCSVLHEGLHNHHTEKSLSHSPGK |
| SEQ | 100 | huPD1-478 | KIVLTQSPASLPVSLGQRATISCRASESVDTSDNSFIHWYQQRPG |
| ID | | crossmab HC | QSPKLLIYRSSTLESGVPARFSGSGSRTDFTLTIDPVEADDVATY |
| NO: | | with mTfR-001 | YCQQNYDVPWTFGGGTKLEIKSSAKTTPPSVYPLAPGSAAQTNSM |
| | | Fab BBB format | VTLGCLVKGYFPEPVTVTWNSGSLSSGVHTFPAVLQSDLYTLSSS |
| | | | VTVPSSTWPSQTVTCNVAHPASSTKVDKKIVPRDCGCKPCICTVP |
| | | | EVSSVFIFPPKPKDVLTITLTPKVTCVVVAISKDDPEVQFSWFVD |
| | | | DVEVHTAQTKPREEQINSTFRSVSELPIMHQDWLNGKEFKCRVNS |
| | | | AAFGAPIEKTISKTKGRPKAPQVYTIPPPKKQMAKDKVSLTCMIT |
| | | | NFFPEDITVEWQWNGQPAENYKNTQPIMKTDGSYFVYSKLNVQKS |
| | | | NWEAGNTFTCSVLHEGLHNHHTEKSLSHSPGKGGGGSGGGGSEVQ |
| | | | LVESGGGLVQPGNSLTLSCVASGFTFSNYGMHWIRQAPKKGLEWI |
| | | | AMIYYDSSKMNYADTVKGRFTISRDNSKNTLYLEMNSLRSEDTAM |
| | | | YYCAVPTSHYVVDVWGQGVSVTVSSAKTTPPSVYPLAPGSAAQTN |
| | | | SMVTLGCLVEGYFPEPVTVTWNSGSLSSGVHTFPAVLQSDLYTLS |
| | | | SSVTVPSSTWPSQTVTCNVAHPASSTKVDEKIVPRDC |
| SEQ | 101 | Nada LC | DIQMTQSPSSLSASVGDRVTITCRASQDVGSGVAWYQQKPGKAPK |
| ID | | | LLIGSASGLYSGVPSRESGSRSGTDFTLTISSLQPEDFATYYCQQ |
| NO: | | | SASGGSTFGQGTKVEIKRADAAPTVSIFPPSSRKLTSGGASVVCF |
| | | | LNNFYPKDINVKWKIDGSERQNGVLNSWTDQDSKDSTYSMSSTLT |
| | | | LTKDEYERHNSYTCEATHKTSTSPIVKSENRNEC |
| SEQ | 102 | Nada HC with | KIVLTQSPASLPVSLGQRATISCRASESVDTSDNSFIHWYQQRPG |
| ID | | huPD1-478 | QSPKLLIYRSSTLESGVPARFSGSGSRTDFTLTIDPVEADDVATY |
| NO: | | crossmab TCB | YCQQNYDVPWTFGGGTKLEIKSSAKTTPPSVYPLAPGSAAQTNSM |
| | | format | VTLGCLVKGYFPEPVTVTWNSGSLSSGVHTFPAVLQSDLYTLSSS |
| | | | VTVPSSTWPSQTVTCNVAHPASSTKVDKKIVPRDCGGGGSGGGGS |
| | | | EVQLVESGGGLVQPGGSLRLSCAASGFSIAGTAIHWVRQAPGKGL |
| | | | EWVASISPGGGSTAYADSVKGRFTISADTSKNTAYLQMNSLRAED |
| | | | TAVYYCSRSGGSGASAMDYWGQGTLVTVSSAKTTPPSVYPLAPGS |
| | | | AAQTNSMVTLGCLVEGYFPEPVTVTWNSGSLSSGVHTFPAVLQSD |
| | | | LYTLSSSVTVPSSTWPSQTVTCNVAHPASSTKVDEKIVPRDCGCK |
| | | | PCICTVPEVSSVFIFPPKPKDVLTITLTPKVTCVVVAISKDDPEV |
| | | | QFSWFVDDVEVHTAQTKPREEQINSTFRSVSELPIMHQDWLNGKE |
| | | | FKCRVNSAAFGAPIEKTISKTKGRPKAPQVYTIPPPKKQMAKDKV |
| | | | SLTCMITNFFPEDITVEWQWNGQPAENYKNTQPIMKTDGSYFVYS |
| | | | KLNVQKSNWEAGNTFTCSVLHEGLHNHHTEKSLSHSPGK |
| SEQ | 103 | huPD1-478 HC | KIVLTQSPASLPVSLGQRATISCRASESVDTSDNSFIHWYQQRPG |
| ID | | with Nada | QSPKLLIYRSSTLESGVPARFSGSGSRTDFTLTIDPVEADDVATY |
| NO: | | CrossFab BBB | YCQQNYDVPWTFGGGTKLEIKSSAKTTPPSVYPLAPGSAAQTNSM |
| | | format | VTLGCLVKGYFPEPVTVTWNSGSLSSGVHTFPAVLQSDLYTLSSS |
| | | | VTVPSSTWPSQTVTCNVAHPASSTKVDKKIVPRDCGCKPCICTVP |
| | | | EVSSVFIFPPKPKDVLTITLTPKVTCVVVAISKDDPEVQFSWFVD |
| | | | DVEVHTAQTKPREEQINSTFRSVSELPIMHQDWLNGKEFKCRVNS |
| | | | AAFGAPIEKTISKTKGRPKAPQVYTIPPPKKQMAKDKVSLTCMIT |
| | | | NFFPEDITVEWQWNGQPAENYKNTQPIMKTDGSYFVYSKLNVQKS |
| | | | NWEAGNTFTCSVLHEGLHNHHTEKSLSHSPGKGGGGSGGGGSEVQ |
| | | | LVESGGGLVQPGGSLRLSCAASGFSIAGTAIHWVRQAPGKGLEWV |
| | | | ASISPGGGSTAYADSVKGRFTISADTSKNTAYLQMNSLRAEDTAV |
| | | | YYCSRSGGSGASAMDYWGQGTLVTVSSAKTTPPSVYPLAPGSAAQ |
| | | | TNSMVTLGCLVEGYFPEPVTVTWNSGSLSSGVHTFPAVLQSDLYT |
| | | | LSSSVTVPSSTWPSQTVTCNVAHPASSTKVDEKIVPRDC |
| SEQ | 104 | Nada crossmab | EVQLVESGGGLVQPGGSLRLSCAASGFSIAGTAIHWVRQAPGKGL |
| ID | | LC | EWVASISPGGGSTAYADSVKGRFTISADTSKNTAYLQMNSLRAED |
| NO: | | | TAVYYCSRSGGSGASAMDYWGQGTLVTVSSASDAAPTVSIFPPSS |
| | | | EQLTSGGASVVCFLNNFYPKDINVKWKIDGSERQNGVLNSWTDQD |
| | | | SKDSTYSMSSTLTLTKDEYERHNSYTCEATHKTSTSPIVKSENRN |
| | | | EC |
| SEQ | 105 | Nada crossmab | DIQMTQSPSSLSASVGDRVTITCRASQDVGSGVAWYQQKPGKAPK |
| ID | | HC | LLIGSASGLYSGVPSRFSGSRSGTDFTLTISSLQPEDFATYYCQQ |
| NO: | | | SASGGSTFGQGTKVEIKSSAKTTPPSVYPLAPGSAAQTNSMVTLG |
| | | | CLVKGYFPEPVTVTWNSGSLSSGVHTFPAVLQSDLYTLSSSVTVP |
| | | | SSTWPSQTVTCNVAHPASSTKVDKKIVPRDCGCKPCICTVPEVSS |
| | | | VFIFPPKPKDVLTITLTPKVTCVVVAISKDDPEVQFSWFVDDVEV |
| | | | HTAQTKPREEQINSTFRSVSELPIMHQDWLNGKEFKCRVNSAAFG |
| | | | APIEKTISKTKGRPKAPQVYTIPPPKEQMAKDKVSLTCMITNFFP |
| | | | EDITVEWQWNGQPAENYDNTQPIMDTDGSYFVYSDLNVQKSNWEA |
| | | | GNTFTCSVLHEGLHNHHTEKSLSHSPGK |
| SEQ | 106 | mTfR-001 HC | DIQMTQSPSSLSASVGDRVTITCRASQDVGSGVAWYQQKPGKAPK |
| ID | | with Nada | LLIGSASGLYSGVPSRFSGSRSGTDFTLTISSLQPEDFATYYCQQ |
| NO: | | crossmab TCB | SASGGSTFGQGTKVEIKSSAKTTPPSVYPLAPGSAAQTNSMVTLG |
| | | format | CLVKGYFPEPVTVTWNSGSLSSGVHTFPAVLQSDLYTLSSSVTVP |
| | | | SSTWPSQTVTCNVAHPASSTKVDKKIVPRDCGGGGSGGGGSEVQL |
| | | | VESGGGLVQPGNSLTLSCVASGFTFSNYGMHWIRQAPKKGLEWIA |
| | | | MIYYDSSKMNYADTVKGRFTISRDNSKNTLYLEMNSLRSEDTAMY |
| | | | YCAVPTSHYVVDVWGQGVSVTVSSAKTTPPSVYPLAPGSAAQTNS |
| | | | MVTLGCLVEGYFPEPVTVTWNSGSLSSGVHTFPAVLQSDLYTLSS |
| | | | SVTVPSSTWPSQTVTCNVAHPASSTKVDEKIVPRDCGCKPCICTV |
| | | | PEVSSVFIFPPKPKDVLTITLTPKVTCVVVAISKDDPEVQFSWFV |
| | | | DDVEVHTAQTKPREEQINSTFRSVSELPIMHQDWLNGKEFKCRVN |
| | | | SAAFGAPIEKTISKTKGRPKAPQVYTIPPPKKQMAKDKVSLTCMI |
| | | | TNFFPEDITVEWQWNGQPAENYKNTQPIMKTDGSYFVYSKLNVQK |
| | | | SNWEAGNTFTCSVLHEGLHNHHTEKSLSHSPGK |
| SEQ | 107 | Nada crossmab | DIQMTQSPSSLSASVGDRVTITCRASQDVGSGVAWYQQKPGKAPK |
| ID | | HC with mTfR- | LLIGSASGLYSGVPSRFSGSRSGTDFTLTISSLQPEDFATYYCQQ |
| NO: | | 001 Fab BBB | SASGGSTFGQGTKVEIKSSAKTTPPSVYPLAPGSAAQTNSMVTLG |
| | | format | CLVKGYFPEPVTVTWNSGSLSSGVHTFPAVLQSDLYTLSSSVTVP |
| | | | SSTWPSQTVTCNVAHPASSTKVDKKIVPRDCGCKPCICTVPEVSS |
| | | | VFIFPPKPKDVLTITLTPKVTCVVVAISKDDPEVQFSWFVDDVEV |
| | | | HTAQTKPREEQINSTFRSVSELPIMHQDWLNGKEFKCRVNSAAFG |
| | | | APIEKTISKTKGRPKAPQVYTIPPPKKQMAKDKVSLTCMITNFFP |
| | | | EDITVEWQWNGQPAENYKNTQPIMKTDGSYFVYSKLNVQKSNWEA |
| | | | GNTFTCSVLHEGLHNHHTEKSLSHSPGKGGGGSGGGGSEVQLVES |
| | | | GGGLVQPGNSLTLSCVASGFTFSNYGMHWIRQAPKKGLEWIAMIY |
| | | | YDSSKMNYADTVKGRFTISRDNSKNTLYLEMNSLRSEDTAMYYCA |
| | | | VPTSHYVVDVWGQGVSVTVSSAKTTPPSVYPLAPGSAAQTNSMVT |
| | | | LGCLVEGYFPEPVTVTWNSGSLSSGVHTFPAVLQSDLYTLSSSVT |
| | | | VPSSTWPSQTVTCNVAHPASSTKVDEKIVPRDC |

TABLE 10
Exemplary sequences of wildtype TfR and PD1

| | | | |
|---|---|---|---|
| SEQ | 65 | exemplary human | PGWFLDSPDRPWNPPTESPALLVVTEGDNATFTCSFSNTSESFVL |
| ID | | PD1 sequence | NWYRMSPSNQTDKLAAFPEDRSQPGQDCRFRVTQLPNGRDFHMSV |
| NO: | | | VRARRNDSGTYLCGAISLAPKAQIKESLRAELRVTERRAEVPTAH |
| | | | PSPSPRPAGQFQTLVVGVVGGLLGSLVLLVWVLAVICSRAARGTI |
| | | | GARRTGQPLKEDPSAVPVFSVDYGELDFQWREKTPEPPVPCVPEQ |
| | | | TEYATIVFPSGMGTSSPARRGSADGPRSAQPLRPEDGHCSWPL |
| SEQ | 66 | Exemplary human | MMDQARSAFSNLFGGEPLSYTRESLARQVDGDNSHVEMKLAVDEE |
| ID | | TfR sequence | ENADNNTKANVTKPKRCSGSICYGTIAVIVFFLIGFMIGYLGYCK |
| NO: | | | GVEPKTECERLAGTESPVREEPGEDFPAARRLYWDDLKRKLSEKL |
| | | | DSTDFTGTIKLLNENSYVPREAGSQKDENLALYVENQFREFKLSK |
| | | | VWRDQHFVKIQVKDSAQNSVIIVDKNGRLVYLVENPGGYVAYSKA |
| | | | ATVTGKLVHANFGTKKDFEDLYTPVNGSIVIVRAGKITFAEKVAN |
| | | | AESLNAIGVLIYMDQTKFPIVNAELSFFGHAHLGTGDPYTPGFPS |
| | | | FNHTQFPPSRSSGLPNIPVQTISRAAAEKLFGNMEGDCPSDWKTD |
| | | | STCRMVTSESKNVKLTVSNVLKEIKILNIFGVIKGFVEPDHYVVV |
| | | | GAQRDAWGPGAAKSGVGTALLLKLAQMFSDMVLKDGFQPSRSIIF |
| | | | ASWSAGDFGSVGATEWLEGYLSSLHLKAFTYINLDKAVLGTSNEK |
| | | | VSASPLLYTLIEKTMQNVKHPVTGQFLYQDSNWASKVEKLTLDNA |
| | | | AFPFLAYSGIPAVSFCFCEDTDYPYLGTTMDTYKELIERIPELNK |
| | | | VARAAAEVAGQFVIKLTHDVELNLDYERYNSQLLSFVRDLNQYRA |
| | | | DIKEMGLSLQWLYSARGDFFRATSRLTTDFGNAEKTDRFVMKKLN |
| | | | DRVMRVEYHFLSPYVSPKESPFRHVFWGSGSHTLPALLENLKLRK |
| | | | QNNGAFNETLERNQLALATWTIQGAANALSGDVWDIDNEF |

TABLE 11
Exemplary constant heavy and light chain regions

| | | | |
|---|---|---|---|
| SEQ | 67 | exemplary human | RTVAAPSVFIFPPSDEQLKSGTASVVCLLNNFYPREAKVQWKVDN |
| ID | | kappa light chain | ALQSGNSQESVTEQDSKDSTYSLSSTLTLSKADYEKHKVYACEVT |
| NO: | | constant region | HQGLSSPVTKSFNRGEC |
| SEQ | 68 | exemplary human | QPKAAPSVTLEPPSSEELQANKATLVCLISDFYPGAVTVAWKADS |
| ID | | lambda light | SPVKAGVETTTPSKQSNNKYAASSYLSLTPEQWKSHRSYSCQVTH |
| NO: | | chain constant | EGSTVEKTVAPTECS |
| | | region | |
| SEQ | 69 | exemplary human | ASTKGPSVFPLAPSSKSTSGGTAALGCLVKDYFPEPVTVSWNSGA |
| ID | | heavy chain | LTSGVHTFPAVLQSSGLYSLSSVVTVPSSSLGTQTYICNVNHKPS |
| NO: | | constant region | NTKVDKKVEPKSCDKTHTCPPCPAPELLGGPSVFLFPPKPKDTLM |
| | | derived from | ISRTPEVTCVVVDVSHEDPEVKFNWYVDGVEVHNAKTKPREEQYN |
| | | IgG1 | STYRVVSVLTVLHQDWLNGKEYKCKVSNKALPAPIEKTISKAKGQ |
| | | | PREPQVYTLPPSRDELTKNQVSLTCLVKGFYPSDIAVEWESNGQP |
| | | | ENNYKTTPPVLDSDGSFFLYSKLTVDKSRWQQGNVFSCSVMHEAL |
| | | | HNHYTQKSLSLSP |
| SEQ | 70 | exemplary human | ASTKGPSVFPLAPSSKSTSGGTAALGCLVEDYFPEPVTVSWNSGA |
| ID | | heavy chain | LTSGVHTFPAVLQSSGLYSLSSVVTVPSSSLGTQTYICNVNHKPS |
| NO: | | constant region | NTKVDEKVEPKSCDKTHTCPPCPAPEAAGGPSVFLFPPKPKDTLM |
| | | derived from | ISRTPEVTCVVVDVSHEDPEVKFNWYVDGVEVHNAKTKPREEQYN |
| | | IgG1 with | STYRVVSVLTVLHQDWLNGKEYKCKVSNKAGAPIEKTISKAKGQP |
| | | mutations L234A, | REPQVCTLPPSRDELTKNQVSLSCAVKGFYPSDIAVEWESNGQPE |
| | | L235A and | NNYKTTPPVLDSDGSFFLVSKLTVDKSRWQQGNVFSCSVMHEALH |
| | | P329G (Fc | NHYTQKSLSLSP |
| | | gamma receptor | |
| | | silenced) | |

### III. Examples

The following are examples of methods and compositions of the invention. It is understood that various other embodiments may be practiced, given the general description provided above.

Standard methods were used to manipulate DNA as described in Sambrook, J. et al., Molecular cloning: A laboratory manual; Cold Spring Harbor Laboratory Press, Cold Spring Harbor, New York, 1989. The molecular biological reagents were used according to the manufacturer's instructions. RPMI medium supplemented with 10% FBS and 2.5 mM L-Glutamine was used for cell culture and cellular assays of all types. Medium for transduced Jurkat cells additionally contained 50 μg/mL of Zeocin and 1.5 μg/mL Puromycin. To generate the following bi- and multispecific antibodies, the CrossMab technology described in WO2016/016299 was used, in which VH/VL have been exchanged in one antibody arm and the CH1/CL interface of the other antibody arm has been modified by charge modifications, in combination with the knobs-into-holes technology in the CH3/CH3 interface to foster heterodimerization, as described in Regula et al. (2018) Protein Engineering, Design and Selection, 31 (7-8): 289-299.

The generation and full-length antibody sequences of PD1-0103-0312 are described e.g. in WO2017/55443 A1. The generation and full-length sequences of pembrolizumab are described e.g. in WO2008/156712 A1.

### Example 1: Manufacture of a Bispecific Antigen-Binding Molecule Binding TIR and PD1

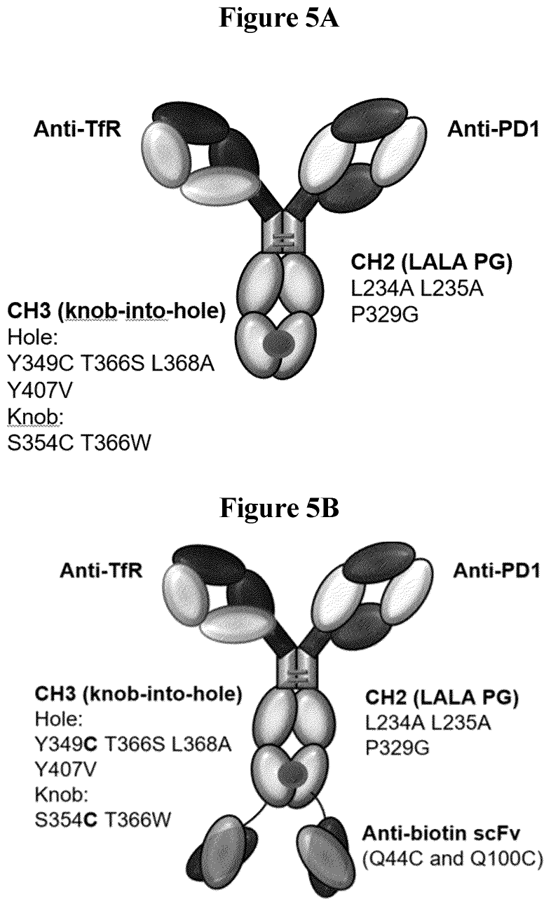

Several bispecific CrossMabs targeting TfR (regular Fab) and PD1 (cross-Fab) were engineered using the knob-into-hole mutations Y349C, T366S, L368A, Y407V (hole) and S354C, T366W (knob) (FIG. 5A; full length sequences indicated in Table 12 in combination with Table 8). The arm comprising the PD1 binding domain contained either the variable region amino acid sequences of the anti-PD1 antibody described in WO2017/55443 A1 (heavy chain variable domain SEQ ID NO: 23 and light chain variable domain SEQ ID NO: 24), hereinafter called PD1-0103-0312, or the variable region amino acid sequence of the anti-PD1 antibody Pembrolizumab (heavy chain variable domain SEQ ID NO: 31 and light chain variable domain SEQ ID NO: 32), a bivalent anti-PD1 antibody approved for the treatment of cancer and described e.g. in WO2008/156712 A1. The arm comprising the TfR binding domain contained either the variable region amino acid sequences of the anti-TfR antibody described in WO2016/207240 A1 (heavy chain variable domain SEQ ID NO: 7 and light chain variable domain SEQ ID NO: 8), hereinafter called 1026, or the variable region amino acid sequence of an unpublished anti-TfR antibody, hereinafter called 51A165 (heavy chain variable domain SEQ ID NO: 15 and light chain variable domain SEQ ID NO: 16). Immune effector functions were abolished using the LALA PG mutations (L234A, L235A and P329G; Schlothauer et al. (2016) Protein Engineering, Design & Selection, vol. 29 no. 10, pp. 457-466).

TABLE 12
Bispecific and Trispecific TfR/PD1 CrossMab sequences (1 + 1 Format)

| | | | | HC | LC |
|---|---|---|---|---|---|
| | | HC | LC | cross | cross |
| | Name of variable regions | (SEQ | (SEQ | (SEQ | (SEQ |
| Alias | (VHs/VLs) | ID NO:) | ID NO:) | ID NO:) | ID NO:) |
| 8012 | PD1-0103-0312/51A165 | 37 | 38 | 39 | 40 |
| 8013 | PD1-0103-0312/1026 | 35 | 36 | 39 | 40 |
| 8014 | PD1-0103-0312/Nada | 45 | 46 | 39 | 40 |
| 8015 | Nada/51A165 | 37 | 38 | 43 | 44 |
| 8016 | Nada/1026 | 35 | 36 | 43 | 44 |
| 8017 | Pembrolizumab/1026 | 35 | 36 | 41 | 42 |
| 8018 | Pembrolizumab/51A165 | 37 | 38 | 41 | 42 |
| 8019 | Pembrolizumab/Nada | 45 | 46 | 41 | 42 |
| 1129 | TfR/Pembrolizumab/anti-Biotin | 47 | 48 | 49 | 50 |
| | (“Trispecific CrossMAb”) | | | | |
| 9903 | TfR/Nada/anti-Biotin | 47 | 48 | 53 | 54 |
| 9904 | Nada/Pembrolizumab/anti-Biotin | 51 | 52 | 49 | 50 |
| 0784 | Anti-CD33/anti-CD33/anti-Biotin | 55 | 56 | 55 | 56 |

DNA sequences for the antibodies were optimized with in-house tools and ordered from GeneArt or Twist Bioscience. The DNA fragments encoding for the amino acid sequences shown in Table 12 were cloned into established expression vectors, which were transfected into HEK293 suspension cells using PEIpro® (Polyplus) and cultured at 37° C. in a humidified incubator with 8% CO₂. Supernatants were harvested through centrifugation at 3500 g after six to seven days and the supernatant filtered through a 0.22 μm filter unit (Thermo Fisher Scientific). Antibodies were purified from cell supernatants by protein A and size exclusion chromatography. The antibody molecular mass of the antibodies was verified by Caliper LabChips and mass spectrometry.

### Example 2: Construction of a Trispecific CrossMab that can Bind to a Biotinylated Cytotoxic Agent as Payload

A trispecific CrossMAb with the Alias 1129 and targeting TfR (regular Fab), PD1 (cross-Fab) and Biotin was engineered by fusing an anti-biotin scFV as third binding entity to the C-terminus of the bispecific CrossMab 8018 described in Example 1 for delivery of a payload. The anti-biotin scFv contained a cysteine bond at Q44C and Q100C and was fused to the C-Terminus of the CrossMab via a (G4S) 4 linker. The trispecific CrossMab 1129 (for sequences see Table 12 in combination with Table 8) was recombinantly produced in HEK293 cells as described in Example 1. A schematic representation of antibody 1129 is shown in FIG. 5B.

As control molecules, antibodies were engineered in which the binding sequences in the variable antibody regions of the light and heavy chain of either the TfR-binding arm (Antibody 9904 in Table 12) or the PD1 binding arm (Antibody 9903 in Table 12), were replaced by a non-binding sequence (denoted in the following by the term “Nada”). As a double-non-binding control where both arms were replaced with an entity that binds neither PD1 nor TfR, an anti-CD33 antibody with a C-terminally attached anti-biotin scFv was used (“anti-CD33 anti-CD33 anti-Biotin”; antibody 0784 in Table 12). The binding affinities of the trispecific CrossMAb molecule of Example 2 were confirmed to be comparable to the corresponding control molecule with one Nada fab by Biacore SPR (see Table 14 in Example 4).

### Example 3: Construction of Two Different CrossMab Bispecific Antibodies that Bind PD1 in a Bivalent and TfR in a Monovalent Manner (2+1 Format)

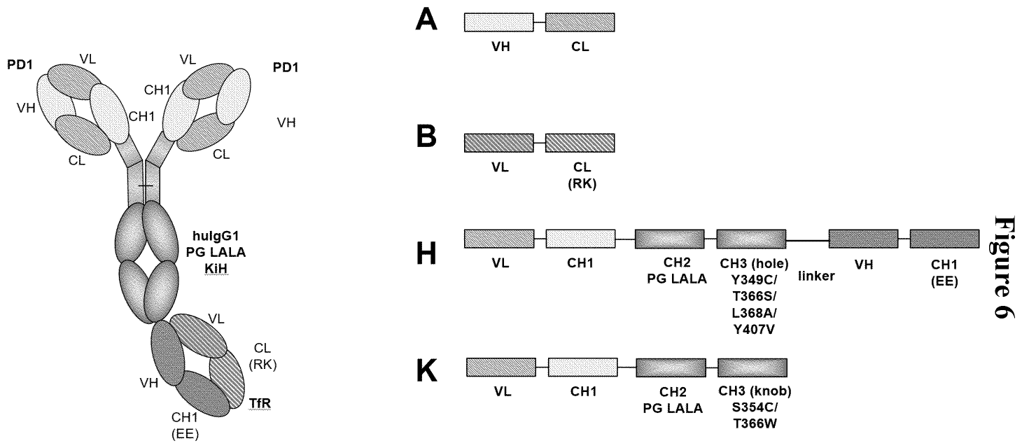

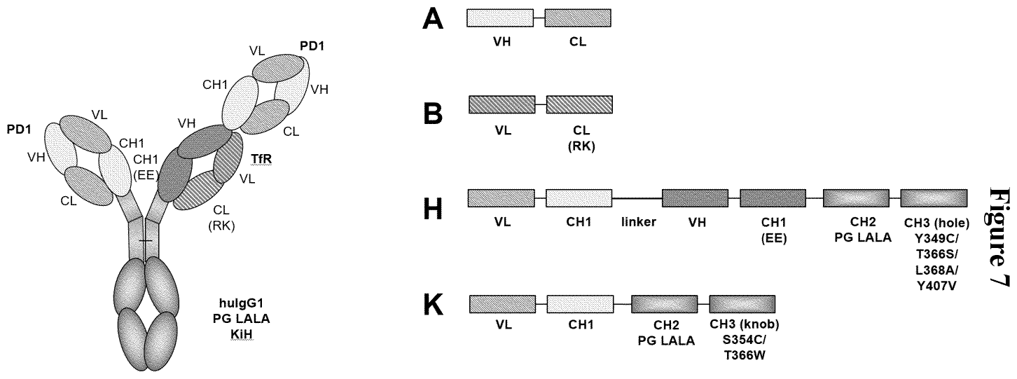

Two CrossMAbs targeting PD1 in a bivalent and TfR in a monovalent manner (Antibodies 8156 and 8157, sequences shown in Table 13) were engineered as shown in FIG. 6 and FIG. 7.

Complex antibody formats that comprise one binding domain binding to a first target and two binding domains binding to a second target are called 2+1 format herein. Complex antibody formats that bind PD1 twice and TfR once were generated in two different 2+1 formats by transient expression and purified applying the same methods as described in Examples 1 and 2. The composition of these molecules is schematically depicted in FIGS. 6 and 7. The full-length sequences are indicated in Table 12 in combination with Table 9.

The first 2+1 format comprises two PD1-binding CrossFabs (a Fab wherein VH and VL were replaced by each other) as arms of an IgG, with a TfR-binding Fab attached via the N-terminus of its VH to the C-terminus of the asymmetrical (knob-into-hole) CH3 domain (Antibody 8156, FIG. 6). This format is herein also called BBB-Format.

The second 2+1 format comprises one PD1-binding entity as a CrossFab arm and one TfR-binding entity as Fab arm in IgG configuration and a second PD1-binding CrossFab arm, on top′ (i.e. attached at the N-Terminus) of the TfR-binding Fab which precedes the hinge of the other side of an knob-into-hole Fc heterodimer (Antibody 8157, FIG. 7). This format is herein also called TCB-format.

The Fc domains of both formats were engineered using the knob-into-hole mutations Y349C, T366S, L368A, Y407V (hole) and S354C, T366W (knob). Immune effector functions were abolished using the LALA PG mutations (L234A, L235A and P329G; Schlothauer et al. supra).

Each of the two complex antibody formats is formed by four different amino acid chains, schematically depicted in FIGS. 6 and 7 and denoted as chains A, B, H (heavy chain with “hole” mutations) and K (heavy chain with “knob” mutations). The individual amino acid sequences of these amino acid chains are listed as SEQ ID NO: 57, SEQ ID NO: 58, SEQ ID NO: 59, SEQ ID NO: 60 and SEQ ID NO: 61.

Control molecules were generated in the same formats and compositions, except that in the control molecules the variable regions of the TfR binding domain were exchanged for non-binding sequences (termed ‘Nada’) (Antibody 8158 and 8159, sequences shown in Table 13). The individual amino acid sequences of those molecules are listed as SEQ ID NO: 57, SEQ ID NO: 60, SEQ ID NO: 62, SEQ ID NO: 63 and SEQ ID NO: 64.

The bispecific CrossMabs 8156, 8157, 8158 and 8159 were recombinantly produced in HEK293 cells as described in Example 1.

TABLE 13
Bispecific anti-TfR anti-PD1 CrossMAb sequences (2 + 1 Format)

| | | Chain A | Chain B | Chain H | Chain K |
|---|---|---|---|---|---|
| | Name of variable region | (SEQ ID | (SEQ ID | (SEQ ID | (SEQ ID |
| Alias | (VHs/VLs) | NO:) | NO:) | NO:) | NO:) |
| 8156 | PD1-0103-0312/ | 57 | 58 | 59 | 60 |
| | TfR(51A165) BBB-format | | | | |
| 8157 | PD1-0103-0312/ | 57 | 58 | 61 | 60 |
| | TfR(51A165) TCB-format | | | | |
| 8158 | PD1-0103-0312/Nada | 57 | 62 | 63 | 60 |
| | BBB-format | | | | |
| 8159 | PD1-0103-0312/Nada | 57 | 62 | 64 | 60 |
| | TCB-format | | | | |

### Example 4: Blocking of PD1/PD-L1-Mediated Signaling and Binding Affinities of the Trispecific Anti-PD1 Anti-TfR Anti-Biotin CrossMab Molecule

Similar to the blocking antibody Pembrolizumab, the trispecific anti-PD1 anti-TfR anti-biotin CrossMab 1129 blocked PD1/PD-L1-mediated inhibition of TCR signaling in a co-culture assay. For the PD1/PD-L1 blockade bioassay (Promega), the effector cells were Jurkat T cells expressing human PD1 and a luciferase reporter system driven by an NFAT response element (NFAT-RE). NFAT-RE activation is induced upon TCR activation. The interaction between human PD1 (effector cell) and PD-L1 (target cell) interrupts the TCR downstream signal and prevents NFAT-RE activation. Upon either PD1 or PD-L1 blockade, the inhibitory signal is removed and NFAT-RE becomes activated, resulting in a Luminescence readout.

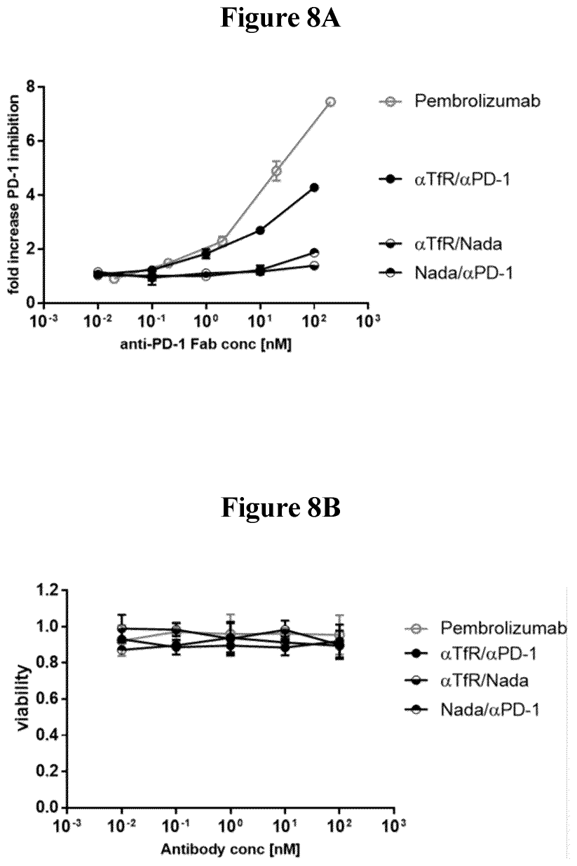

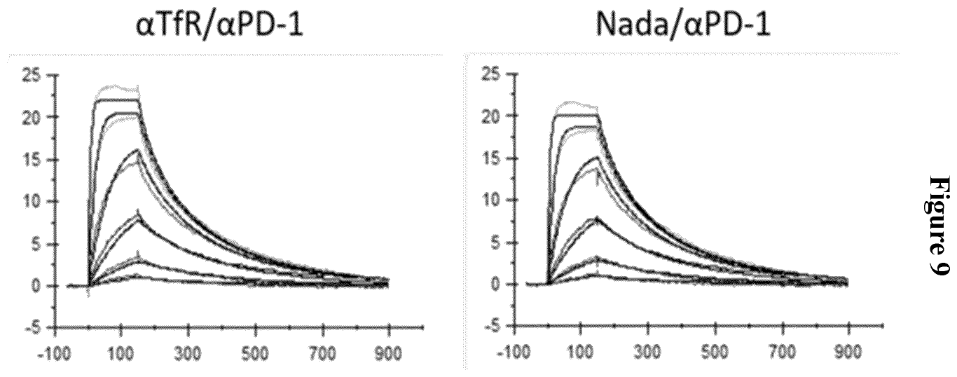

For the setup, 5,000 PD-L1-expressing CHO-K1 cells were seeded in 96-well plates overnight. 50,000 PD1-expressing Jurkat-PD1-NFAT cells were pre-incubated with antibodies for 30 min at 37° C., washed with media once and then added to the activator cells for 5 h. Inhibition of the TCR activation by PD1 signaling was measured by the luminescent signal after addition of Bio-Glo™ Luciferase Assay Substrate. The anti-TfR anti-PD1 anti-biotin antibody 1129 (“αTfR/αPD1”) showed significantly increased inhibition of PD1 (more than 2 times increased at 10 nM) compared to the anti-Nada anti-PD1 anti-Biotin control antibody 9904 (“Nada/αPD1”), but not quite as high as Pembrolizumab which showed even higher blockade at high concentrations in this 5 h set at the same Fab concentration (FIG. 8A).

That the increased inhibition of PD1 using the anti-TfR anti-PD1 anti-biotin antibody compared to the anti-Nada anti-PD1 anti-Biotin control antibody was not due to any differences in target binding is also supported by the comparable K_{D}, kₐ and k_{d} values measured by Biacore SPR (FIG. 9 and Table 14). Both molecules perform very similarly with regard to all three parameters, suggesting that any differences in PD1 inhibition must be due to presence or absence of the TfR binder.

For the Biacore SPR, around 16.000 resonance units (RU) of the capturing system (20 μg/ml anti-PGLALA Ab (mAb<PG LALA>M-17.24-IgG manufactured in-house) were coupled on a CM5 chip (GE Healthcare BR-1005-30) at pH 5.0 by using an amine coupling kit supplied by the GE Healthcare. The sample and system buffer was PBS-T (10 mM phosphate buffered saline including 0.05% Tween20) pH 7,4. The flow cell was set to 25° C.—and the sample block set to 12° C.—and primed with running buffer twice. The appropriate antibody was captured by injecting a 5 nM solution for 30 sec at a flow of 10 μl/min.

Association was measured by injection of PD1 in various concentrations in solution for 150 sec at a flow of 30 μl/min starting with 400 nM in 1:3 dilutions to 0.5 nM. The dissociation phase was monitored for up to 750 sec and triggered by switching from the sample solution to running buffer. The surface was regenerated by 40 sec washing with 10 mM NaOH at a flow rate of 30 μl/min. Bulk refractive index differences were corrected by subtracting the response obtained from an anti PGLALA Ab surface. Blank injections are also subtracted (=double referencing). For calculation of apparent KD and other kinetic parameters the Langmuir 1:1 model was used. Resulting K_{D}, kₐ and k_{d} values are shown in Table 14.

TABLE 14
Off-rates in Biacore SPR assay for PD1

| Alias | kₐ (1/Ms) | k_{d} (1/s) | K_{D} (M) | t_{1/2} (s) |
|---|---|---|---|---|
| 1129 (αTfR/αPD1) | 2.0 · 10⁶ | 1.0 · 10⁻² | 5.2 · 10⁻⁹ | 68 |
| 9904 (Nada/αPD1) | 1.9 · 10⁶ | 9.0 · 10⁻³ | 4.8 · 10⁻⁹ | 77 |

The cell viability in the co-culture assay was not affected by the addition of any antibody at the concentrations applied (FIG. 8B).

### Example 5: Avidity-Enhanced Binding of a Trispecific Anti-PD1 Anti-TfR Anti-Biotin CrossMab Depends on PD1 Expression

Avidity-enhanced binding of the trispecific anti-PD1 anti-TfR anti-biotin CrossMab 1129 was assessed by Jurkat cell assay. The NFAT-bla Jurkat cells (Thermo Fisher Scientific) were transduced lentivirally with PD1 using the Lenti-X HTX Packaging System (Clontech Laboratories). Single clones were generated that expressed human PD1 at low and high levels (a low expression level corresponded to ca. 3,000 bound anti-PD1 antibodies/cell, while high expression level corresponded to ca. 20,000 bound anti-PD1 antibodies/cell). As control, non-transduced wildtype Jurkat cells were used.

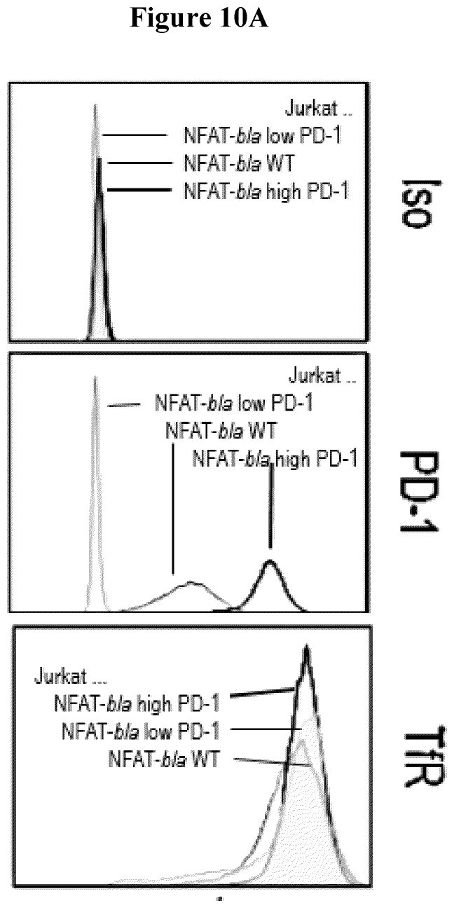

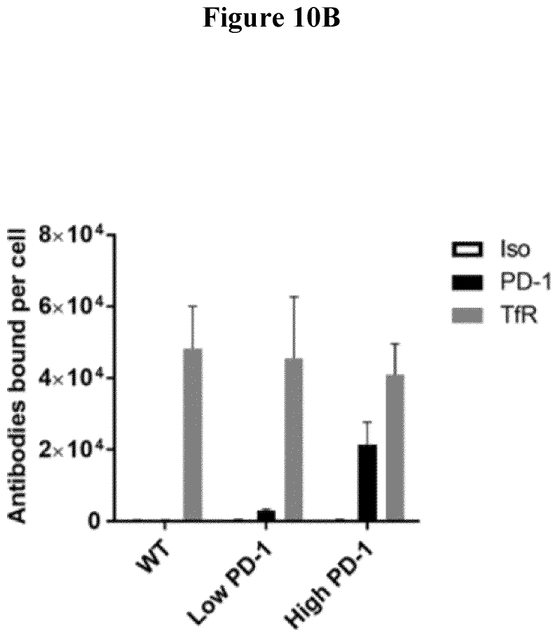

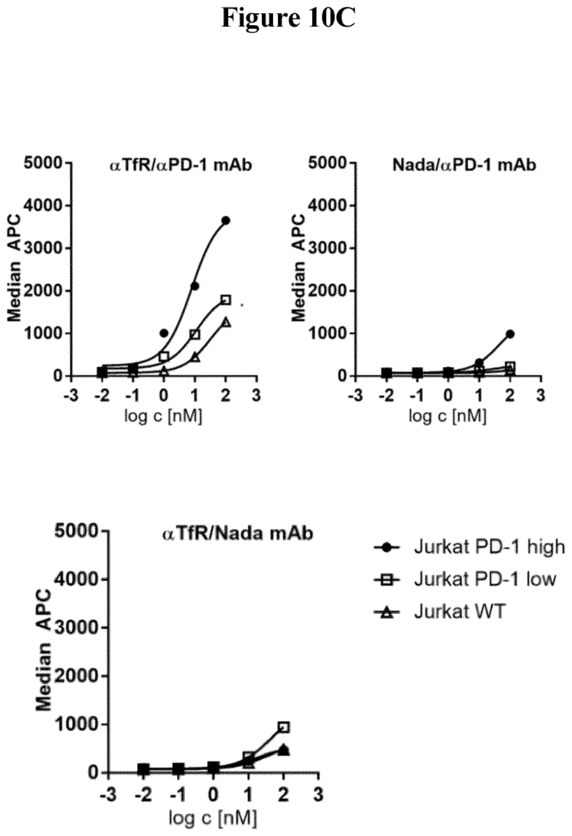

PD1 and TfR expression were measured using the PE-labelled antibodies PE anti-human PD1 (Clone NAT105) and PE anti-human TfR (Clone CY1G4) (BioLegend) and flow cytometry (BD, Canto II). A PE labelled mouse IgG1 binding to an irrelevant antigen was used as isotype control (Iso). The NFAT-bla Jurkat cell lines transduced with PD1 at low and high surface expression showed similar expression of TfR (FIGS. 10A and 10B).

In order to assess whether the binding of trispecific anti-PD1 anti-TfR anti-biotin CrossMab 1129 to the Jurkat cells is dependent on PD1 expression levels, 250,000 cells each of wildtype, PD1 low and PD1 high Jurkat cells were seeded in 96 well plates and incubated with antibody 1129 or with control antibodies 9903 or 9904 for 2 h on ice. Biotinylated Cy5 (bio-Cy5, made in-house) was added at 200 nM for 10 min, followed by 2 washes in ice-cold PBS. Cells were analyzed for fluorophore binding by flow cytometry. Antibody 1129 was detected to higher extent (by biotinylated Cy5) on Jurkat cells that expressed high levels of PD1 on the cell surface (FIG. 10C, upper left). This effect correlated with PD1 expression on the cell surface, as evidenced by cells which expressed low levels of PD1 on the cell surface and which had less bound antibody 1129. In contrast, the control antibodies 9904 and 9903 with one Nada domain showed very little binding to the cell surface even at the highest concentrations tested (FIG. 10C, upper right and lower left).

### Example 6: Anti-PD1 Anti-TfR Bispecific Antibodies are Internalized by Activated T Cells

The mechanism of internalization induced via the Transferrin receptor is crucial for the anti-PD1 anti-TfR bispecific antibody in order to remove PD1 receptors from the T cell surface. Therefore, PD1 receptor internalization was assessed upon binding of the bispecific anti-Tfr anti-PD1 antibodies 8012, 8013 or 8014, or control antibodies 8015, 8016, 8017, 8018 or 8019 (see Table 12 for sequences) to 3 days polyclonally activated CD4+ T cells by flow cytometry.

CD4+ T cells were exposed to different molecules at 4° C. for 30 minutes before either staining with an anti-LALAPG PE-conjugated antibody followed by fixation, or further incubated at 37° C. for additional 3 hours to allow for internalization of the molecules. CD4 T cells incubated at 4° C. are used as reference, since internalization at 4° C. is negligible.

For that, CD4+ T cells from healthy donors were enriched (Miltenyi Biotec, 130-045-101) and polyclonally activated for three days in the presence of CD3 (1 μg/ml plate bound) and CD28 (1 mg/μl soluble). The cells were incubated with molecules 8012-8019 as shown in in Table 12 for 30 min at 4° C., washed and split into two groups. One group is immediately stained with secondary anti-LALAPG PE-conjugated antibody, before fixation (BD Cell fix). The second group was resuspended in medium and incubated for 3 hours at 37° C. before anti-LALAPG-PE staining and fixation. The cells were acquired at the LSR Fortessa (BD Biosciences) and the analysis performed with Flowjo (Treestar).

The Geometric Mean Fluorescence Intensity (GMFI) and the frequency of PE⁺ CD4+ T cells is then compared between the two groups and the percentage of internalization calculated with the following formula:

$${\%}_{Internalization} = 100 - ( ( {GMFI}^{PE + CD 4 + T {cells}_{37 ° C .}} \div {GMFI}^{PE + CD 4 + T {cells}_{4 ° C .}} ) * 100 )$$

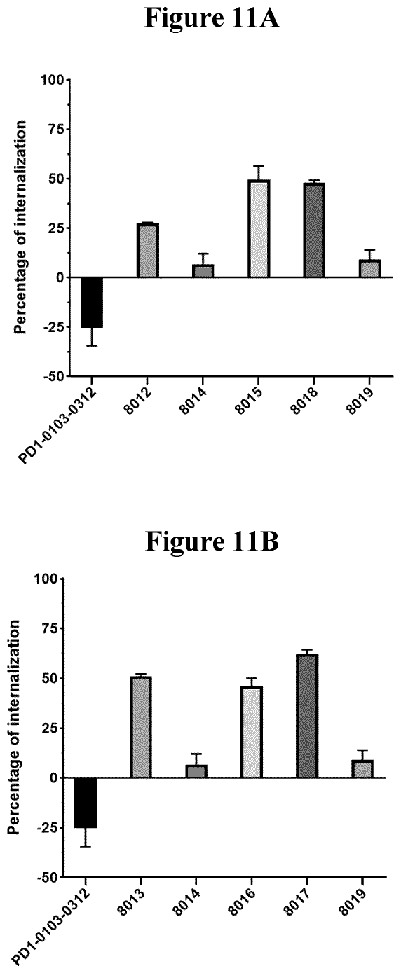

FIGS. 11A and 11B displaying anti-PD1 anti-TfR bispecific antibodies 8012 (PD1-0103-0312/TfR (51A165)) and 8018 (Pembro/TfR (51A165)) (FIG. 11A) and 8013 (PD1-0103-0312/TfR (1026)) and 8017 (Pembro/TfR (1026)) (FIG. 11B) in comparison to the corresponding controls demonstrate that neither the bivalent anti-PD1 antibody (PD1-0103-0312) nor the corresponding monovalent control anti-PD1/anti-Nada antibodies (8014 and 8019) are being internalized, whereas the PD1-0103-0312-derived bispecific antibodies 8012 and 8013 and the Pembrolizumab derived bispecific antibodies 8017 and 8018 show internalization, just as the monovalent TfR constructs 8015 and 8016 that do not have a PD1 binding domain.

### Example 7: Trispecific Anti-TfR Anti-PD1 Anti-Biotin CrossMAb with Bound Payload Internalizes into Jurkat Cells

In order to assess the localization of the trispecific CrossMab in relation to the cell, the NFAT-bla Jurkat cells (Thermo Fisher Scientific) were transduced lentivirally with monomeric enhanced (mE) GFP-(G₄S)₂-PD1 fusion protein using the Lenti-X HTX Packaging System (Clontech Laboratories). For live cell imaging, mEGFP-PD1 expressing Jurkat cells were seeded into 8-well chamber slides (Lab-Tek™, Thermo Fisher) at a density of 50,000 cells/well in phenol-red free RPMI medium containing 10% FCS.

For membrane staining, live cells were pre-incubated with PKH26 according to the manufacturer's instructions (PKH26GL-1KT, Sigma Aldrich). In brief, 1×10⁶ cells were pelleted, resuspended in 200 μl diluent and mixed with 200 μl diluent containing 0.4 μl PKH26 dye. After 2-3 minutes, the labelling reaction was stopped by adding 200 μl FCS and cells were again pelleted and resuspended in phenol-red free RPMI medium. To follow the internalization of antibodies, 10 nM of trispecific anti-TfR anti-PD1 anti-biotin antibody 1129, control antibodies 9903 (TfR/Nada/anti-Biotin), 9904 (Nada/Pembrolizumab/anti-Biotin) and 0784 (antiCD33-antiBio) were complexed with 50 nM biotinylated Cy5 (Bio-Cy5) for 30 min in PBS at 37° C. The complexes were added to the cells and allowed to internalize for 3 hours.

Internalization of PD1-GFP and of the antibodies was followed by fluorescence microscopy carried out on a Leica SP8 laser scanning confocal microscope using a 63×/1.2NA water immersion objective lens (Leica). Temperature, CO₂ level and humidity were maintained at 37° C. and 5% CO₂ using a stage-top incubation chamber (Oko-touch, Okolab). Sequential scans were performed at 600 Hz (bidirectional scan) with 7× zoom and 2× line average (384 px×384 px, pixel size: 69 nm). Z-stacks were acquired with a step size of 350 nm. A white light laser was used for excitation at 488 nm, 561 nm and 633 nm. Fluorescence emission was detected at 495-560 nm (GFP), and 643-715 nm (Cy5) using HyD detectors. Images were processed with the 3D viewer of the Leica LAS AF software.

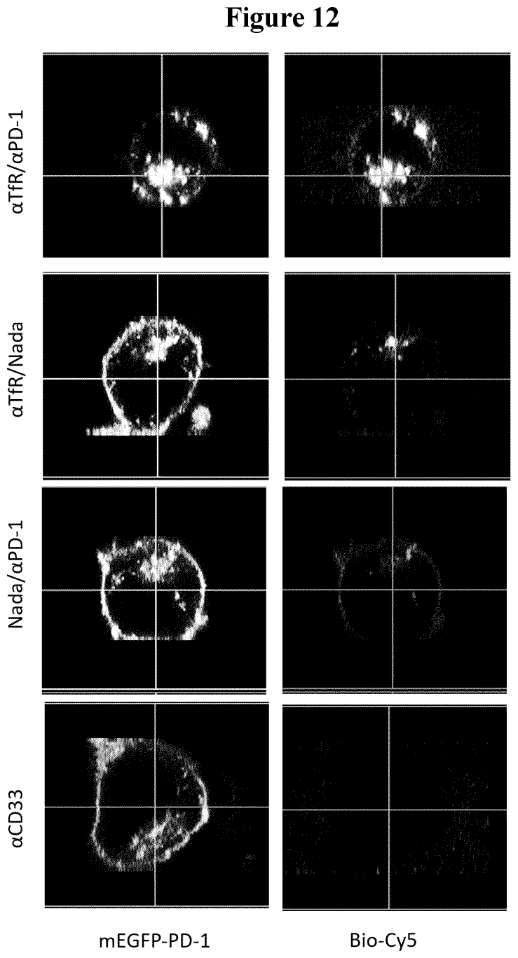

Using mEGFP-PD1 transduced Jurkat cells and confocal microscopy it could be shown that the trispecific anti-TfR anti-PD1 anti-biotin CrossMAb (“αTfR/αPD1”) delivers biotinylated Cy5 inside the cells. Moreover, the payload co-localized in vesicles along with PD1. For the two Nada (“Nada/αPD1” and “αTfR/Nada”) and the anti-CD33 (“αCD33”) control molecules, only very limited amounts of biotinylated Cy5 or PD1 were found inside the cells (FIG. 12).

### Example 8: Vesicular mEGFP-PD1 Accumulation after Contacting of Transduced Jurkat Cells with Trispecific Anti-TfR Anti-PD1 Anti-Biotin CrossMAb

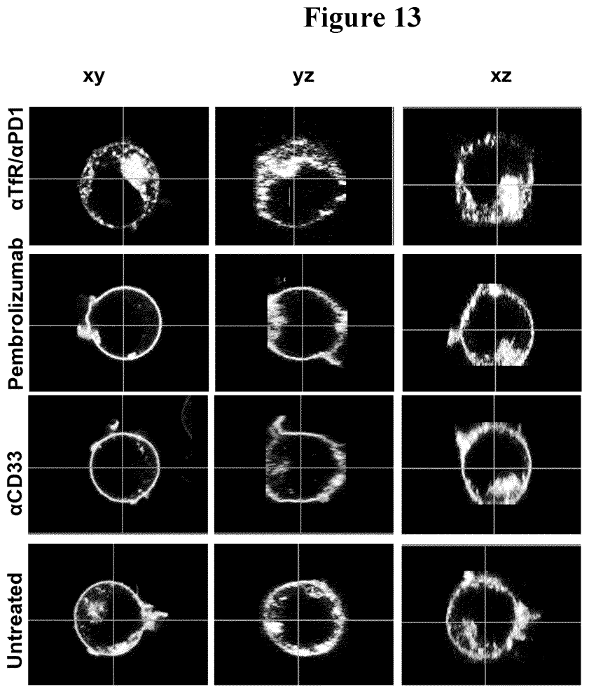

To test whether the vesicular mEGFP-PD1 accumulation depended on the simultaneous binding of PD1 and TfR, the trispecific anti-TfR anti-PD1 anti-biotin CrossMAb (“αTfR/αPD1”) was compared with Pembrolizumab. In contrast to the trispecific anti-TfR anti-PD1 anti-biotin CrossMAb, PD1 remains on the cell surface following the treatment with Pembrolizumab (FIG. 13).

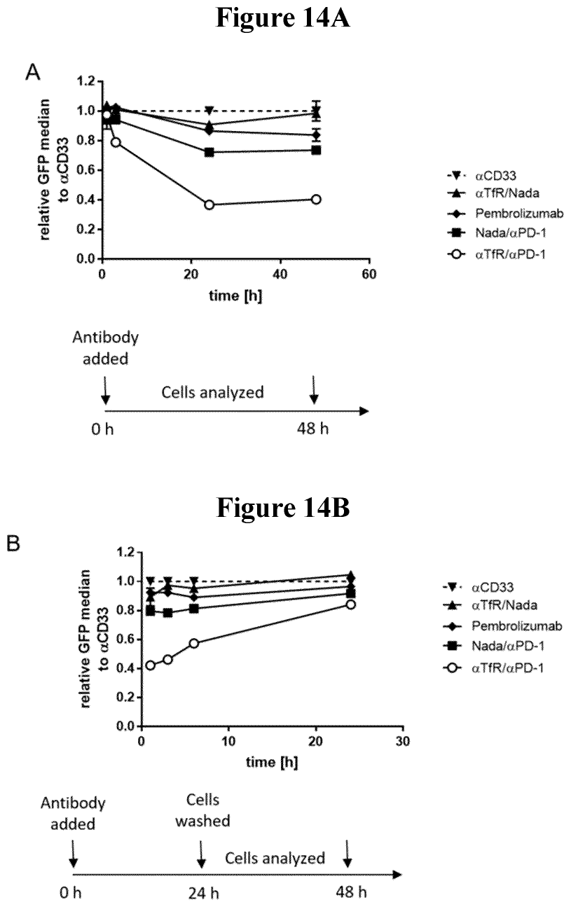

The PD1 internalization with the trispecific anti-TfR anti-PD1 anti-biotin CrossMAb was further confirmed by flow cytometry of Jurkat cells transduced with mEGFP-PD1. The mEGFP signal dropped to 40% over 24 h upon addition of the trispecific anti-TfR anti-PD1 anti-biotin CrossMAb (“αTfR/αPD1”) and remained at this level at least up to 48 h when the antibody was left in solution (FIG. 14A). The monovalent Nada/anti-PD1 CrossMAb (“Nada/αPD1”) reduced PD1 levels to around 80% after 24 h, whereas the bivalent Pembrolizumab showed slow reduction to only 85% after 48 h. Interestingly, the mEGFP-PD1 signal on this transduced Jurkat cell line recovered rather quickly whenever the trispecific anti-TfR anti-PD1 anti-biotin CrossMAb antibody was removed to around 80% within another 24 h (FIG. 14B).

### Example 9: Avidity-Enhanced Delivery of Truncated Pseudomonas Exotoxin (PE25)

To confirm intracellular delivery using this platform, the truncated pseudomonas exotoxin PE25 was used, which requires endo-/lysosomal delivery to unfold its cytotoxic properties. PE25 was produced in E. coli and purified by methods previously described for truncated Pseudomonas exotoxin derivatives in WO2015101589A1. The truncated pseudomonas exotoxin was then biotinylated using 20-fold excess of Sulfo-NHS-LC-Biotin following the instructions of the EZ-Link Sulfo-NHS-Biotinylation Kit (Thermo Scientific). Successful biotinylation of the toxin was verified by Western Blot. The biotinylated PE25 was complexed with the trispecific anti-TfR anti-PD1 anti-biotin CrossMAb 1129 or control antibodies 9903 or 9904 for 10 min in PBS and incubated with wildtype, PD1 low and PD1 high Jurkat cells for 48 h before the cell viability was assessed by the CellTiter Glo viability luminescent assay (Promega).

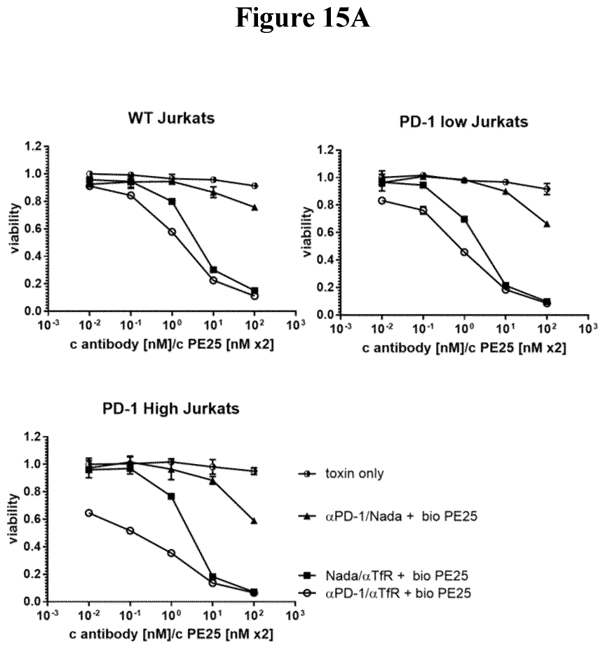

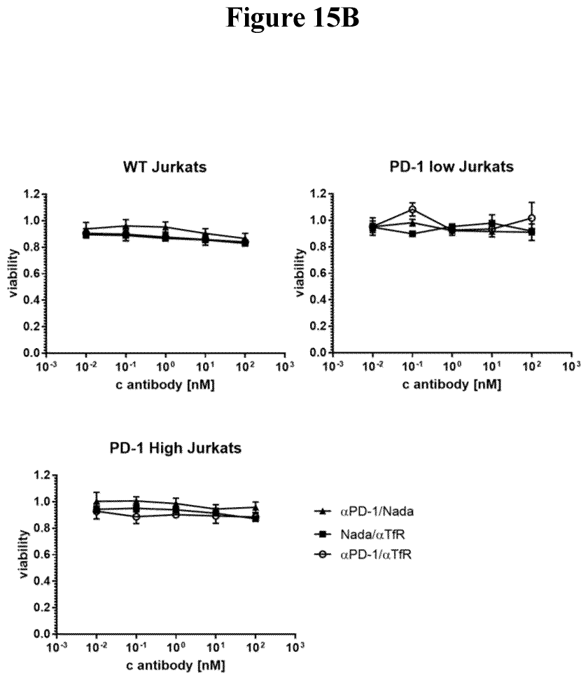

As expected, the biotinylated PE25 by itself is not toxic at the applied concentrations (FIG. 15A). When delivered via the anti-PD1/Nada antibody 9904 (“αPD1/Nada+bio PE25”), the cell viability decreases with increasing concentrations and this effect is more pronounced with high PD1 expression. However, it requires 100 nM of antibody to achieve significant cytotoxicity. Due to the fast internalizing nature of TfR, the Nada/anti-TfR antibodies (“Nada/αTfR+bio PE25”) delivered the toxin and reduced the cell viability dose-dependently. This effect could also be seen for the trispecific anti-TfR anti-PD1 anti-biotin CrossMab (“αPD1/αTfR+bio PE25”), but in contrast to the Nada/anti-TfR antibody, the trispecific anti-TfR anti-PD1 anti-biotin CrossMab reduced the cell viability of high PD1 expressing Jurkat cells at up to 100 fold lower concentrations than the anti-TfR/Nada when the antibody were added at concentrations below 10 nM. Without biotinylated PE25, none of the antibodies showed any toxicity in the applied concentrations over the assay timeframe of 48 h (FIG. 15B).

### Example 10: Avidity-Enhanced Binding and Internalization of Trispecific Anti-PD1 Anti-TfR Anti-Biotin CrossMab in Activated Human T Cells

The antibodies were further assessed for interactions with primary human T cells. For the isolation of T cells, fresh blood from healthy human donors was processed using Ficoll® Paque Plus (GE Healthcare) according to the manufacturer's recommendations and Leucosep™ centrifuge tubes (Greiner Bio-one). Human PBMCs (Peripheral Blood Mononuclear Cells) were activated with plate-bound anti-CD3 and soluble anti-CD28, which led to the expression of ˜200,000 molecules of TfR and ˜8,000 molecules of PD1 on the T cell surface.

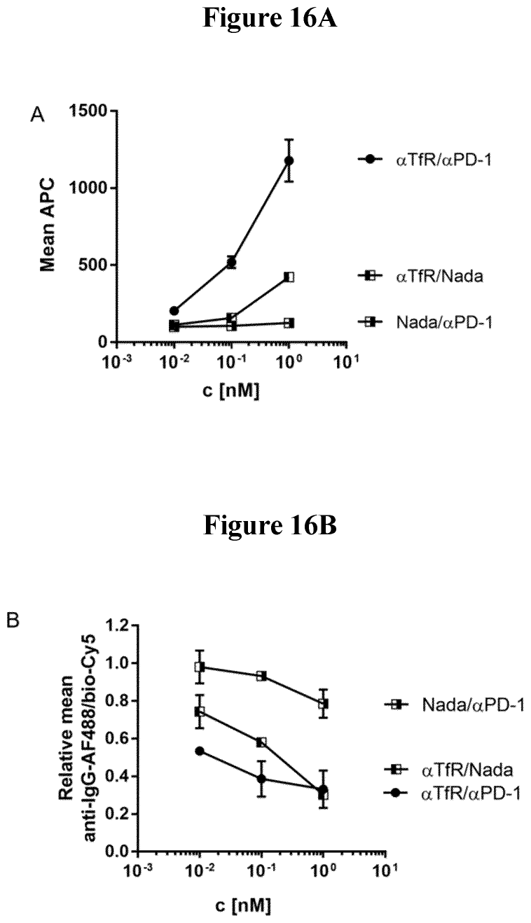

The trispecific anti-PD1 anti-TfR anti-biotin CrossMab 1129 (“αTfR/αPD1”) was detected by biotinylated Cy5 (since the Cy5 dye is spectrally equivalent to APC (allophytocyanin), the flow cytometer's APC channel can be used to detect Cy5) at concentrations at which the control antibodies 9903 (“αTfR/Nada”) and 9904 (“Nada/αPD1”) showed only very little binding to activated T cells (FIG. 16A). Through post-staining of IgG on the cell surface using a AF488 labelled secondary antibody at time point 0 h and 1 h, significant internalization was observed for the two anti-TfR containing antibodies 1129 (“αTfR/αPD1”) and—to a lesser degree—9903 (“αTfR/Nada”), but not for the Nada/anti-PD1 antibody 9904 (“Nada/αPD1”) (FIG. 16B).

### Example 11: Avidity-Mediated Killing of Activated T Cells in Graft-Versus-Host Disease

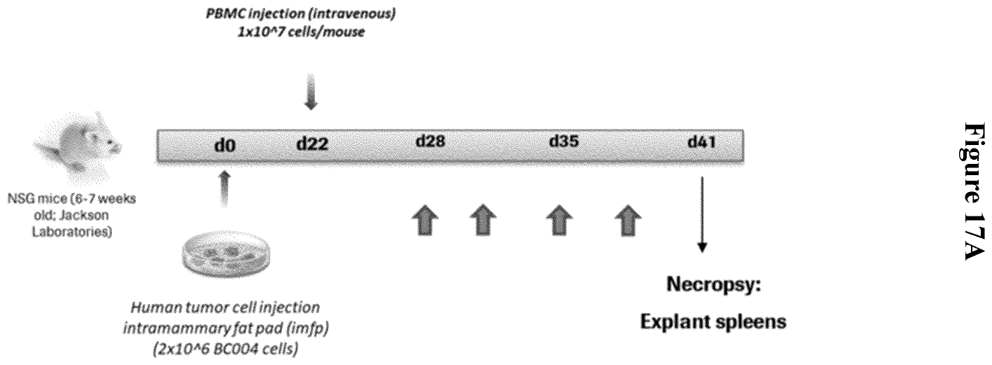

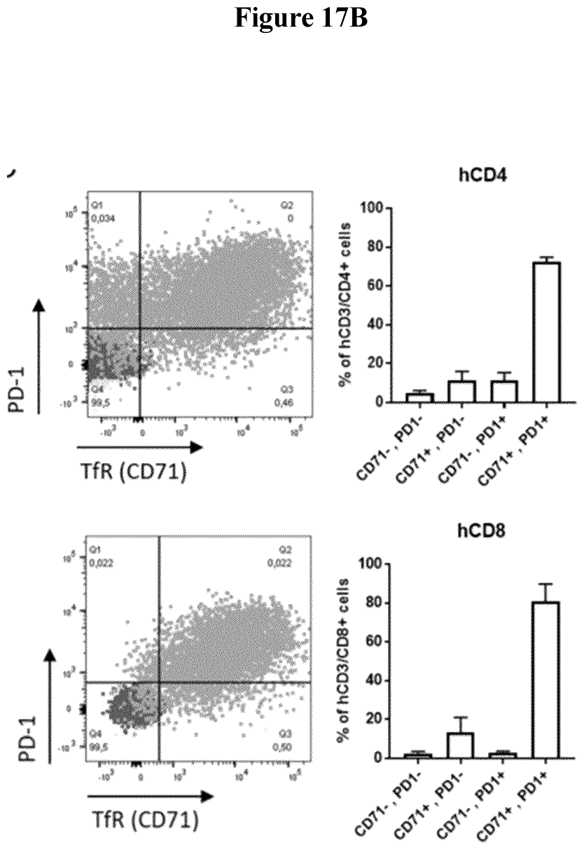

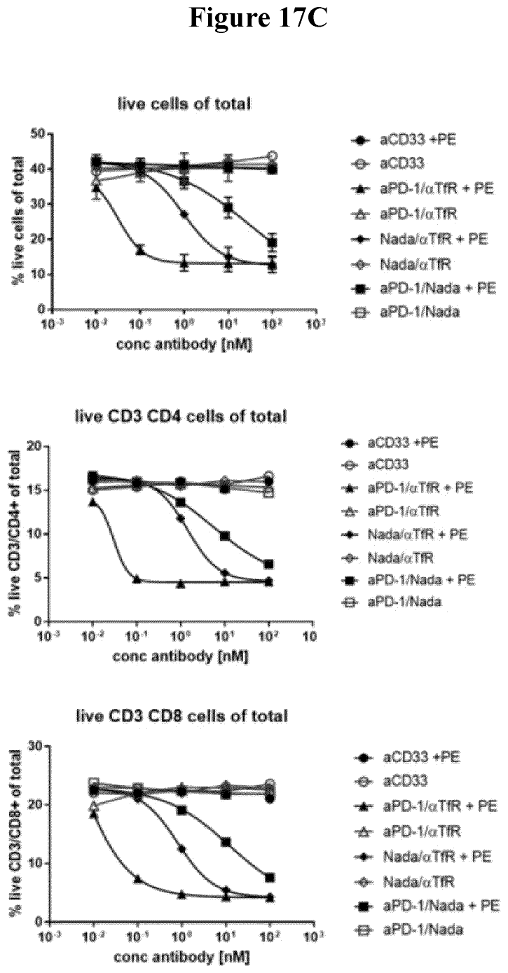

As a therapeutic application, Graft-versus-Host disease (GvHD) was investigated, for which a short-term eradication of highly activated host T cells could be of significant impact. The splenic T cells from tumor-bearing mice that had PBMCs engrafted for 20 days and usually die of GvHD in due course (FIG. 17A) were assessed. For this purpose, human PBMCs from healthy volunteer donors were isolated using human lymphocyte separating tubes (human Pancoll, PAN-Biotech, Aidenbach, Germany) according to the manufacturer's instructions. NSG mice (NOD.Cg-Prkdc^{scid} Il2rg^{tm1Wjl}/SzJ; JAX stock #005557) were ordered from Jackson Laboratories. Tumor-bearing mice were inoculated intravenously via the tail vein with 107 human PBMCs in 100 μL PBS. 20 days later the spleens were dissected and the cells separated through 70 μm restrainers. Cells were stained for CD3, CD4, CD8, PD1 and TfR, and analysed by flow cytometry to assess TfR and PD1 expression on infiltrated human T cells.

More than 70% of the human T cells (including CD4 and CD8 T cells) detected amongst the murine spleenocytes were double-positive for TfR (here denoted as “CD71”) and PD1 (FIG. 17B).

Treatment of these spleenocytes with the trispecific anti-TfR anti-PD1 anti-biotin CrossMab complexed with PE25 (“αPD1/αTfR+PE”) showed a 10-1000 fold reduction in dose required to reduce the number of human T cells in this pool of cells (FIG. 17C).

### Example 12: Enhanced Internalization of Bispecific Antibodies that Bind PD1 in a Bivalent and TfR in a Monovalent Manner (2+1 Format)

The PD1 binding antibodies that are currently applied in cancer therapy are typically bivalent molecules, i.e. they carry two binding domains for PD1. TfR-binding bispecific antibodies were tested in formats that enable bivalent binding of PD1 to see whether they confer enhanced internalization and thereby enhanced potency. The antibodies tested here were generated as described in Example 3.

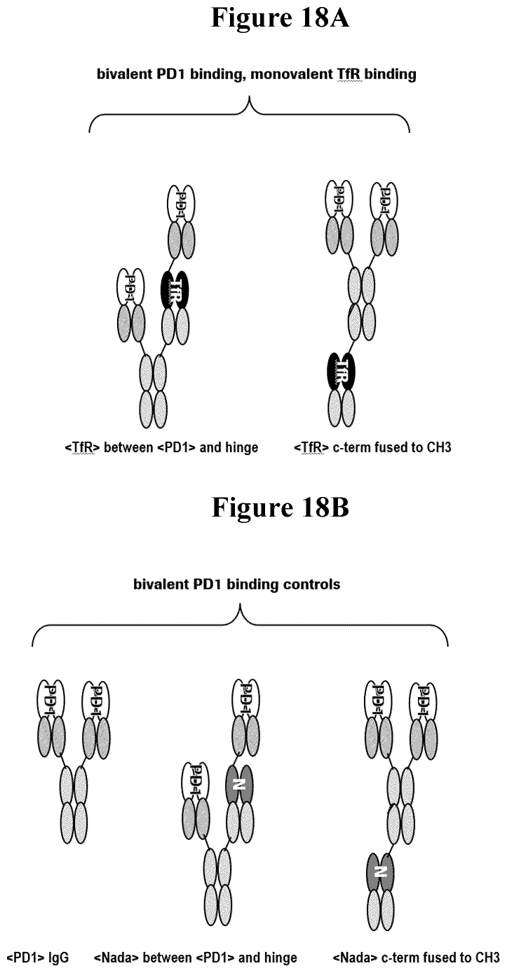

The composition of the tested molecules is schematically depicted in FIG. 18A. The first 2+1 format has one PD1-binding entity as a CrossFab arm in IgG configuration and a second PD1-binding CrossFab arm ‘on top’ (i.e. attached at the N-Terminus) of a TfR-binding Fab which is fused to the hinge of the second subunit of an knob-into-hole Fc heterodimer (antibody 8157). The second 2+1 format has two PD1-binding CrossFab arms as arms of an IgG, with a TfR-binding Fab attached to the C-terminus of the “hole” subunit of the asymmetrical (knob-into-hole) CH3 domain (antibody 8156). Control molecules were generated in the same formats and compositions, except that in those the variable regions of the TfR binder were exchanged to non-binding sequences termed “Nada” (FIG. 18B). A further control was the parent bivalent PD1-binding IgG (PD1-0103-0312) without addition of another binder (FIG. 18B, left side).

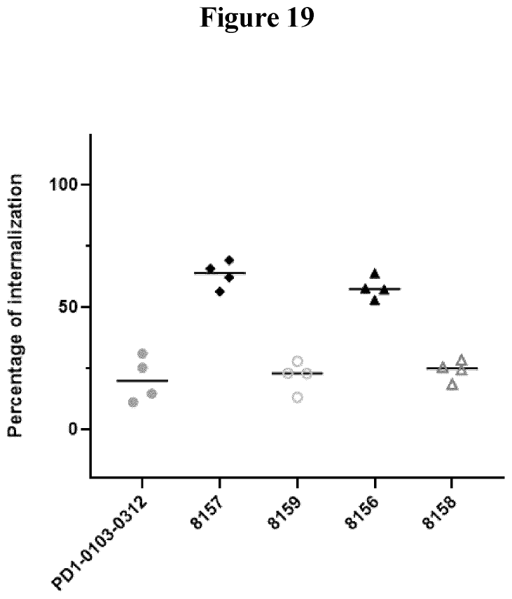

The bispecific antibodies that bind PD1 in a bivalent and TfR in a monovalent manner were used for internalization assays. The experimental setup was the same as in Example 6. The results are shown in FIG. 19. All three control molecules that bind PD1 in a bivalent manner but do not harbor a TfR binder showed rather poor internalization, irrespective of their format. In contrast, the bispecific antibody formats that bind PD1 in a bivalent and TfR in a monovalent manner showed strikingly increased internalization rates.

All tested constructs in FIG. 19 are based on the same parental bivalent anti-PD1 antibody. The parental anti-PD1 antibody PD1-0103-0312 as well as the Nada sequence containing molecules (8159 & 8158) showed no increased tendency to internalization. Antibodies 8156 and 8157 on the other hand demonstrated a strong degree of internalization after 3 hours.

### Example 13: Equal T Cell Effector Functionality of Bispecific CrossMabs that Bind PD1- and TfR in a Monovalent Manner (1+1 Format) Compared to Parental Bivalent Anti-PD1 Antibody

The minimal mixed lymphocyte reaction (mMLR) was used to assess the effects of anti-TfR anti-PD1 bispecific antibodies in an allogeneic setting and specifically on allo-specific T cell cytotoxicity upon exposure to the various molecules. This setup consisted of freshly isolated CD4+ T cells, which were co-cultured for 5 days with allogeneic monocyte derived mature dendritic cells (mDCs).

The generation of mDCs required the isolation of monocytes via magnetic beads (Miltenyi Biotec, 130-050-201) followed by a 5 day cultivation in medium containing GM-CSF (50 ng/ml) and IL4 (100 ng/ml) to induce immature dendritic cells formation from monocytes. TNF-α, IL-1β and IL-6 (50 ng/ml each) were added for 2 additional days to induce DC maturation.

On day one of the mMLR, CD4 T cells from unrelated donors were purified via positive selection with CD4 beads following manufacturer instructions (Miltenyi Biotec, 130-045-101), and labeled with 5 μM of Carboxy-Fluorescein-Succinimidyl Ester (CFSE), prior to co-culture with allogeneic mDCs. CD4 T cells and mDCs were then seeded in a 96 well plate at the ratio of 5:1 with 10⁵ CD4 T cells and 2×10⁴ allogeneic mDCs per well. Seven step dilution series of bivalent anti-PD1 antibody PD1-0103.0312, bispecific anti-Tfr anti-PD1 antibodies 8012, 8013 and 8014, and control antibodies 8015, 8016, 8017, 8018 and 8019 were added with the highest concentration being 10 μg/ml and the lowest 10 μg/ml.

After 5 days of co-culture, half of the supernatant was removed and replaced by fresh medium containing Golgi transport inhibitors, Golgi Plug (Brefeldin A) and Golgi Stop (Monesin), followed by additional 5 hours incubation at 37° C. to allow for cytokine accumulation in the cellular cytoplasm. The cells were then washed and stained with anti-human CD4 (BV605, BioLegend) and Live/Dead fixable dye Aqua (Invitrogen) followed by fixation/permeabilization with Fix/Perm Buffer (BD BioScience). Finally, the intracellular staining for Granzyme B (Alexa Fluor 647, BD BioScience) was performed. The values of EC₅₀ are calculated based on the dose response (stimulation) analysis: log (agonist) vs response (three parameters) and the AUC via the Area-under-curve analysis within the GraphPad Prism 8.4.2 software.

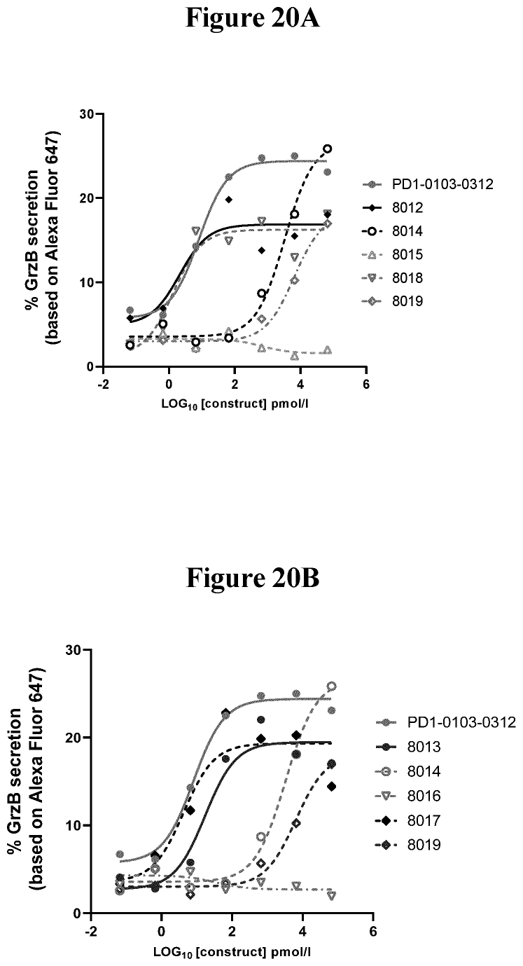

The results of the mixed lymphocyte reactions displayed T cell effector function as Granzyme B production (FIG. 20A and FIG. 20B). While monovalent TfR binding by the binders Nada/51A165 (8015) and Nada/1026 (8016) did not induce any Granzyme B secretion, monovalent anti-PD1 construct PD1-0103-0312/Nada (8014) and Pembrolizumab/Nada (8019) led to modest Granzyme B secretion but not reaching the EC₅₀ values achieved with bivalent PD1-0103-0312.

Although the PD1-0103-0312/TfR bispecific antibodies (8012, 8013) and the Pembrolizumab/TfR bispecific antibodies (8017, 8018) are binding to PD1 only in a monovalent way, they were in the same range with regard to potency as the bivalent anti-PD1 blocking antibody PD1-0103-0312 in causing the secretion of Granzyme B, resulting in comparable effector T cell functionality (FIG. 20A and FIG. 20B). Table 15 and Table 16 show the improvement of activity (as measured by EC₅₀ values) compared to the corresponding control molecules (expressed as fold change versus the control molecule). As can be seen from Table 15 and Table 16, the combination of one PD1 binding entity with one TfR binding entity in one bispecific antibody improved antibody activity by factors of between ca. 100-5000-fold, compared to the corresponding monovalent control (anti-PD1 anti-Nada).

TABLE 15
Fold activity improvement vs. Controls

| | (Bivalent) PD1-IgG | |
|---|---|---|
| Tested molecule | control molecule | Fold activity vs. control |
| 8012 | PD1-0103-312 | 3.82 |
| 8013 | | 0.47 |
| 8017 | | 1.92 |
| 8018 | | 5.97 |

TABLE 16
Fold activity improvement vs. Controls

| | (Monovalent) anti-PD1/ | |
|---|---|---|
| Tested molecule | anti-Nada control molecule | Fold activity vs. control |
| 8012 | 8014 | 1557.76 |
| 8013 | | 191.09 |
| 8017 | 8019 | 1514.78 |
| 8018 | | 4724.19 |

### Example 14: Enhanced T Cell Effector Function Induced by TCB-& BBB-Format Bispecific 2+1 Antibody Formats Compared to Bivalent Anti-PD1 Antibody

The experimental setup was the same as for Example 13, with a 6-step dilution series of PD1-0103-0312 and antibodies 8156-8159 with the highest concentration being 10 μg/ml and the lowest 100 pg/ml.

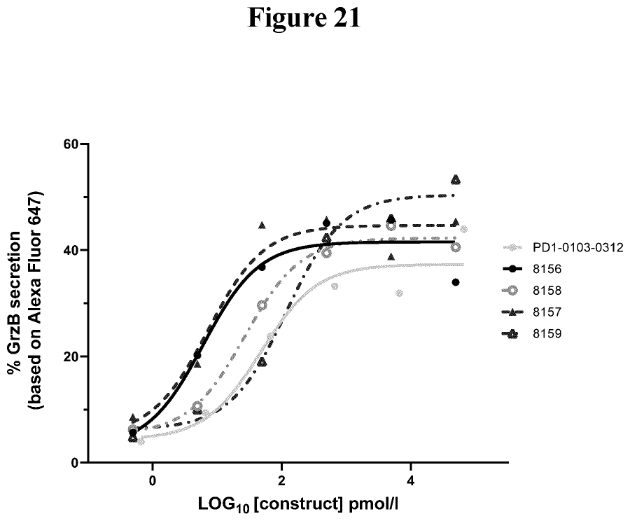

The results of mixed lymphocyte reaction upon treatment with the TCB and BBB formats (8156 & 8157) demonstrated ca. 7 times lower EC₅₀ values than the bivalent parental anti-PD1 antibody and also decreased EC₅₀ values compared to the respective controls (8158 & 18159), resulting in an increased Granzyme B secretion, and therefore increased T cell effector functions, induced by of these formats (FIG. 21). Table 17 and Table 18 shows the improvement of activity (based on the measured EC₅₀ values) in comparison to the corresponding control molecules.

TABLE 17
Fold activity improvement vs. controls

| | (Bivalent) PD1-IgG | |
|---|---|---|
| Tested molecule | control molecule | Fold activity vs. control |
| 8156 | PD1-0103-0312 | 7.83 |
| 8157 | | 6.60 |

TABLE 18
Fold activity improvement vs. controls

| | (Bivalent) anti-PD1/ | |
|---|---|---|
| Tested molecule | anti-Nada control molecule | Fold activity vs. control |
| 8156 | 8158 | 4.44 |
| 8157 | 8159 | 16.33 |

As can be seen from Tables 17 and 18, the combination of two PD1 binding entities with one TfR binding entity in a single 2+1 format bispecific antibody improved antibody activity by factors of between ca. 7-8-fold compared to the bivalent parental anti-PD1 antibody and by factors of ca. 4,5-16-fold compared to the corresponding 2+1 control (anti-PD1: anti-Nada=2:1).

### Example 15: Comparison of T Cell Effector Function Induced by 2+1 and by 1+1 Format Bispecific Antibodies

In order to allow for direct comparison between the observed results, the experimental setup from Example 13 was repeated, with a 6-step dilution series of PD1-0103-0312, pembrolizumab and antibodies 8012-8014, 8017-8019 and 8156-8159 in one single experiment, with the highest concentration being 10 μg/ml and the lowest 100 μg/ml.

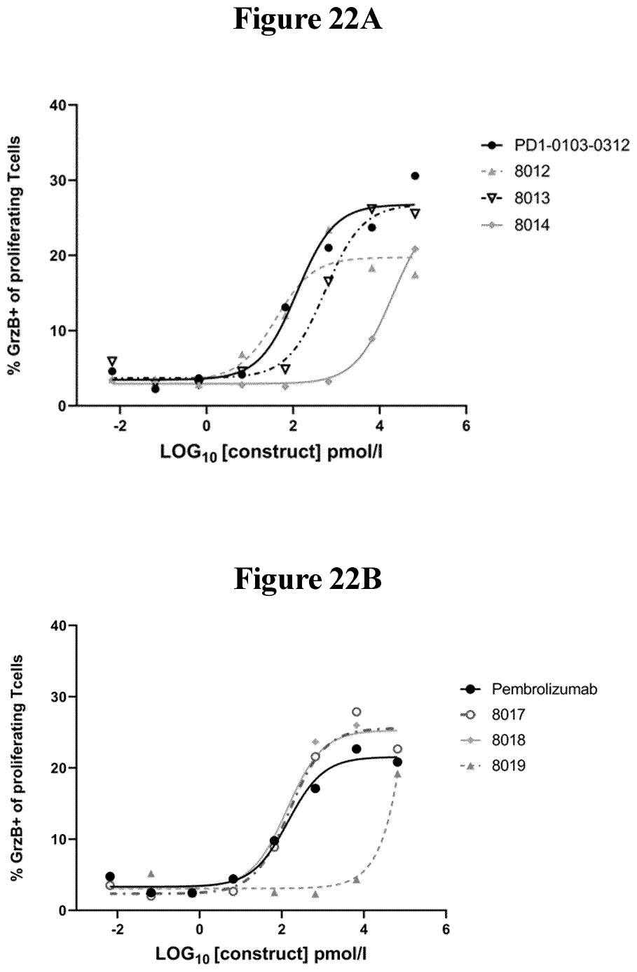

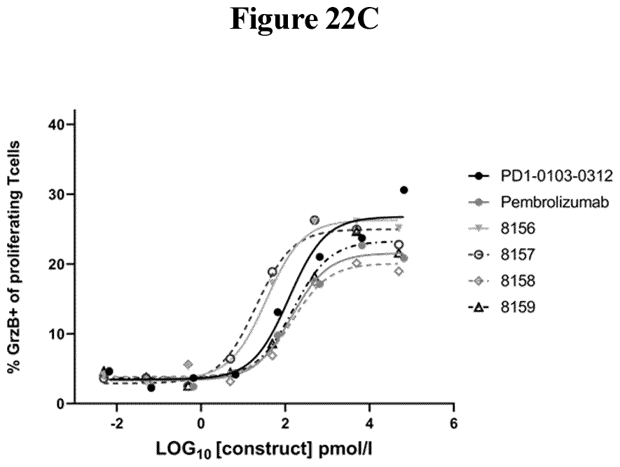

The results of the mixed lymphocyte reactions display Granzyme B production as surrogate for T cell effector function. For better readability, the results have been split into three different figures, although they were obtained from one single experiment (FIGS. 22A-C). Upon treatment with the monovalent 1+1 formats (8012, 8013, 8017, 8018), Granzyme B secretion by T cells is in a range that is comparable to what is achieved with the respective bivalent anti-PD1-antibodies, PD1-0103-0312 (FIG. 22A) and Pembrolizumab (FIG. 22 B), respectively.

When T cells were treated with the 2+1 formats (8156, 8158), Granzyme B secretion was increased, compared to the secretion triggered by Pembrolizumab and PD1-103-0312, indicating more enhanced T cell effector functions induced by these formats (FIG. 22C). The two control molecules 8158 and 8159 which have two PD-1 binding domains and one Nada binding domain showed comparable Granzyme B secretion as the bivalent PD1 binders PD1-0103-0312 and Pembrolizumab.

TABLE 19

| Tested molecule | EC50 (pM) |
|---|---|
| PD1-0103-0312 | 109.7 |
| Pembrolizumab | 175.1 |
| 8012 | 34.01 |
| 8013 | 387.5 |
| 8014 | 13115 |
| 8017 | 120.6 |
| 8018 | 139.4 |
| 8019 | n.d. |
| 8156 | 25.8 |
| 8157 | 12.01 |
| 8158 | 110.8 |
| 8159 | 117.3 |

As can been seen from Table 19 showing the EC₅₀ values of all tested molecules, molecule 8157 was found to be approximately 9- to 10-fold more potent than either PD1-0103-0312 or the respective control molecule 8159, while molecule 8156 managed to achieve a 4-fold higher potency than the two control molecules PD1-0103-0312 and 8158 in terms of enhancing T cell effector function. 8156 and 8157 were also several times more potent than pembrolizumab.

### Example 16: Internalization of Murinized Anti-PD1-TfR Constructs and Controls

For in vivo experiments, murinized surrogate anti-PD1 anti-TfR bispecific antibodies and control molecules are generated (for sequences see Table 20).

TABLE 20
Surrogate molecules for murine colorectal syngeneic model

| | | Chain A | Chain B | Chain M | Chain P |
|---|---|---|---|---|---|
| | Name of variable region | (SEQ ID | (SEQ ID | (SEQ ID | (SEQ ID |
| Alias | (VHs/VLs) | NO) | NO) | NO) | NO) |
| 6768 | mTfR-001/huPD1-478 TCB | 96 | 98 | 97 | 99 |
| | format | | | | |
| 6769 | Nada/huPD1-478 TCB format | 96 | 101 | 97 | 102 |
| 6794 | mTfR-001/Nada TCB format | 104 | 98 | 105 | 106 |
| 6757 | mTfR-001/huPD1-478 BBB | 96 | 98 | 97 | 100 |
| | format | | | | |
| 6761 | Nada/huPD1-478 BBB format | 96 | 101 | 97 | 103 |
| 6793 | mTfR-001/Nada BBB format | 98 | 104 | 105 | 107 |

Prior to the in vivo experiments, the ability of molecules 6768 and 6794 to internalize into cells was tested in a cell-based assay. The experimental setup was essentially as described in Example 6. Molecule 6769 served as a negative control. Since the murinized molecules bind to murine TfR, the activated human CD4 cells were substituted here with the BA/F3 cell line (RNCB Accession ID: CL003201) which expresses murine TfR.

For detection, an AlexaFluor647-conjugated anti-DAPG antibody was used. The Geometric Mean Fluorescence Intensity (GMFI) and the frequency of AF647+ BA/F3 cells was then compared between the group that was stained and fixed immediately after 30 min incubation with the respective molecule at 4° C. and the group that was incubated for a further 3 hours at 37° C., and the percentage of internalization calculated with the following formula:

$${\%}_{Internalization} = 100 - ( ( {GMFI}^{AF 647 + BA / F 3 {cells}_{37 ° C .}} \div {GMFI}^{AF 647 + BA / F 3 {cells}_{4 ° C .}} ) * 100 )$$

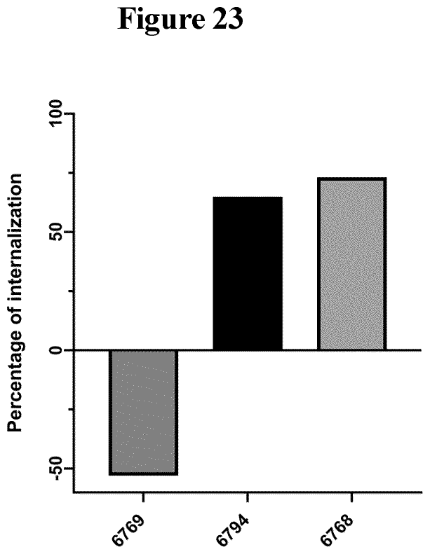

As shown in FIG. 23, it can be stated regarding the biological functionality that both molecules containing the 6768 (mTfR-001/huPD1-478 TCB format) and 6794 (mTfR-001/Nada TCB format) showed good internalization rates of approximately 70% after three hours, while 6769 (huPD1/Nada) showed no internalization, but instead accumulated on the cell surface.

### Example 17: Ability of the Murinized PD1-TfR Molecules to Block PD1/PDL1-Mediated Signaling

The ability of the murinized molecules 6768 and 6769 (see Table 20) to block PD1/PD-L1 mediated signaling was tested. Molecule 6794 was used as a negative control, anti-PD1 antibody 0103-0312 was used as a positive control. Since the murinized constructs 6768 and 6769 contain an anti-human-PD1 antigen-binding domain, their functionality in terms of PD1-PDL1 signaling pathway blockade was determined using the same experimental setup as described in Example 4.

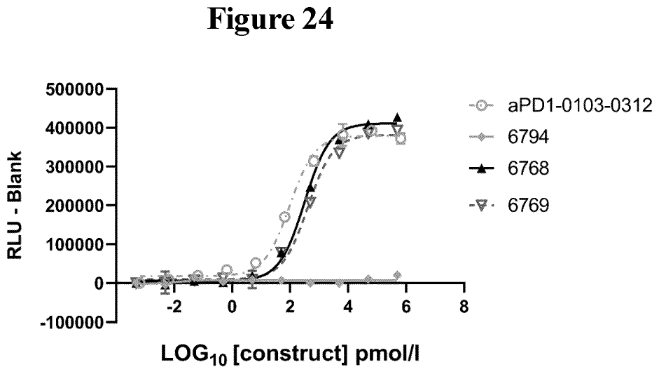

As can be seen in FIG. 24, control molecule 6794 (mTfR-001/Nada, TCB format), which does not contain an anti-PD1 antigen-binding domain, did not show any blocking of PD1/PD-L1 mediated signaling. Molecules 6768 (mTfR-001/huPD1-478, TCB format) and 6769 (Nada/huPD1-478, TCB format) contain bivalent anti-PD1 binding domains like the anti-PD1 antibody PD1-0103-0312 and demonstrated a comparable functionality in terms of blocking the PD1-PDL1 pathway.

### Example 18: Establishment of a Murine Subcutaneous Colorectal Syngeneic Model

A subcutaneous colorectal syngeneic model is used to assess the in vivo efficacy of muPD1-TfR 2+1 format compounds compared to muPD1 and muPD-1-NADA in C57BL/6J mice at the CRO Antineo (Lyon, France). The sequences of murinized surrogate molecules that are used for testing are shown in Table 20. The readout for this subcutaneous model is tumor growth inhibition. Briefly, 6-8 week old female C57BL/6J mice are inoculated with 5×10⁵ MC38 cells injected subcutaneously. Mice are maintained under specific-pathogen-free conditions with continuous health monitoring according to guidelines.

Mice are randomized into different treatment groups and therapy is started when tumors reach an average of 100 mm³ volume as measured by caliper in the subcutaneous model. All treatments are administered intravenously and doses of muPD1-TfR, muPD1 and muPD-1-NADA in the range of 1 to 10 mg/kg are investigated. Tumor volume is measured using a caliper and calculated with the formula:

$$Tumor volume = Length \times Width \times Depth \times 4 / 3 π$$

Tumor growth inhibition is used as read-out and to test for significant differences in group means for multiple comparisons, the standard analysis of variance (One-way ANOVA) is used with Dunnett's method. JMP statistic software program is used for analyses.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. A bispecific antibody comprising a first antigen-binding domain that specifically binds to TfR and a second antigen-binding domain that specifically binds to PD1, and, optionally, a third antigen binding domain that specifically binds to PD1, wherein
the first antigen-binding domain comprises
i. a heavy chain variable domain (VH) comprising
a) CDR-H1 comprising the amino acid sequence of SEQ ID NO: 1,
b) CDR-H2 comprising the amino acid sequence of SEQ ID NO: 2, and
c) CDR-H3 comprising the amino acid sequence of SEQ ID NO: 3, and
a light chain variable domain (VL) comprising
d) CDR-L1 comprising the amino acid sequence of SEQ ID NO: 4,
e) CDR-L2 comprising the amino acid sequence of SEQ ID NO: 5, and
f) CDR-L3 comprising the amino acid sequence of SEQ ID NO: 6,
or
ii. a heavy chain variable domain (VH) comprising
a) CDR-H1 comprising the amino acid sequence of SEQ ID NO: 9,
b) CDR-H2 comprising the amino acid sequence of SEQ ID NO: 10, and
c) CDR-H3 comprising the amino acid sequence of SEQ ID NO: 11, and
a light chain variable domain (VL) comprising
d) CDR-L1 comprising the amino acid sequence of SEQ ID NO: 12,
e) CDR-L2 comprising the amino acid sequence of SEQ ID NO: 13, and
f) CDR-L3 comprising the amino acid sequence of SEQ ID NO: 14.

2. (canceled)

3. The bispecific antibody according to claim 1, wherein at least one of the antigen-binding domains is a Fab fragment.

4. The bispecific antibody according to claim 3, comprising an Fc domain composed of a first and a second subunit, wherein the at least one Fab fragment is fused to the Fc domain and wherein the Fc domain is an IgG Fc domain, optionally an IgG1 Fc domain or an IgG4 Fc domain.

5. The bispecific antibody according to claim 1 wherein the first, the second and, where present, the third antigen-binding domain are each a Fab fragment and the antibody comprises an Fc domain composed of a first and a second subunit; and wherein either (i) the second antigen-binding domain is fused at the C-terminus of its Fab heavy chain to the N-terminus of the Fab heavy chain of the first antigen-binding domain and the first antigen-binding domain is fused at the C-terminus of its Fab heavy chain to the N-terminus of the first subunit of the Fc domain, or (ii) the first antigen-binding domain is fused at the C-terminus of its Fab heavy chain to the N-terminus of the Fab heavy chain of the second antigen-binding domain and the second antigen-binding domain is fused at the C-terminus of the Fab heavy chain to the N-terminus of the first subunit of the Fc domain; and wherein the third antigen-binding domain, where present, is fused at the C-terminus of its Fab heavy chain to the N-terminus of the second subunit of the Fc domain.

6. The bispecific antibody according to claim 1, wherein the first, the second and the third antigen-binding domain are each a Fab fragment and the antibody comprises an Fc domain composed of a first and a second subunit; and wherein
the first antigen-binding domain is fused at the C-terminus of its Fab heavy chain to the N-terminus of the first subunit of the Fc domain,
the second antigen-binding domain is fused at the C-terminus of its Fab heavy chain to the N-terminus of the second subunit of the Fc domain and
the third antigen-binding domain is fused at the N-terminus of its Fab heavy chain to the C-terminus of the first or second subunit of the Fc domain.

7. The bispecific antibody according to claim 1, wherein the bispecific antibody
a) binds to TfR and to PD1 on the surface of a cell which expresses TfR and PD1 and wherein the bispecific antibody is internalized into the cell, and/or
b) PD1 is depleted from the surface of the cell upon binding of the bispecific antibody to TfR and to PD1 displayed on the surface of the cell.

8. The bispecific antibody according to claim 1, comprising at least two heavy chains and at least two light chains and wherein
a) the heavy chains of the bispecific antibody are of the γ type (IgG), in particular of the γ1 type, and/or
b) the light chains of the bispecific antibody are selected from the kappa (κ) and/or lambda (λ) subtype.

9. The bispecific antibody according to claim 4, wherein the Fc domain comprises
i. one or more amino acid substitutions that reduce binding to an Fc receptor, in particular towards Fcγ receptor, and/or
ii. a modification promoting the association of the first and second subunit of the Fc domain.

10. The bispecific antibody according to claim 3, wherein in one of the Fab fragments the variable domains VL and VH are replaced by each other so that the VH domain is part of the light chain and the VL domain is part of the heavy chain.

11. (canceled)

12. The bispecific antibody according to claim 1, wherein the second antigen-binding domain and/or, where present, the third antigen-binding domain specifically binding PD1 comprise(s)
i. a heavy chain variable domain (VH) comprising
a) CDR-H1 comprising the amino acid sequence of SEQ ID NO: 17,
b) CDR-H2 comprising the amino acid sequence of SEQ ID NO: 18, and
c) CDR-H3 comprising the amino acid sequence of SEQ ID NO: 19, and
a light chain variable domain (VL) comprising
d) CDR-L1 comprising the amino acid sequence of SEQ ID NO: 20,
e) CDR-L2 comprising the amino acid sequence of SEQ ID NO: 21, and
f) CDR-L3 comprising the amino acid sequence of SEQ ID NO: 22, or
ii. a heavy chain variable domain (VH) comprising
a) CDR-H1 comprising the amino acid sequence of SEQ ID NO: 25,
b) CDR-H2 comprising the amino acid sequence of SEQ ID NO: 26, and
c) CDR-H3 comprising the amino acid sequence of SEQ ID NO: 27, and
a light chain variable domain (VL) comprising
d) CDR-L1 comprising the amino acid sequence of SEQ ID NO: 28,
e) CDR-L2 comprising the amino acid sequence of SEQ ID NO: 29, and
f) CDR-L3 comprising the amino acid sequence of SEQ ID NO: 30.

13. The bispecific antibody according to claim 1, wherein
i. said first antigen-binding domain specifically binding to TfR comprises
a) a VH domain comprising the amino acid sequence of SEQ ID NO: 7 and a VL domain comprising the amino acid sequence of SEQ ID NO: 8, or
b) a VH domain comprising the amino acid sequence of SEQ ID NO: 15 and a VL domain comprising the amino acid sequence of SEQ ID NO: 16,
and
ii. said second antigen-binding domain and/or, where present, said third antigen-binding domain specifically binding to PD1 comprises
a) a VH domain comprising the amino acid sequence of SEQ ID NO: 23 and a VL domain comprising the amino acid sequence of SEQ ID NO: 24 or
b) a VH domain comprising the amino acid sequence of SEQ ID NO: 31 and a VL domain comprising the amino acid sequence of SEQ ID NO: 32.

14. A pharmaceutical composition comprising the bispecific antibody of claim 1 and a pharmaceutically acceptable carrier.

15. The bispecific antibody according to claim 1 for use in the prevention or treatment of cancer.
